SEQUENCE LISTING (845 sequences)

SEQ ID NO: 1 - protein, 38 aa, synthetic construct
  VARIANT 9..28: note = X can be G, A, or deleted
RRRRRRRRXXXXXXXXXXXXXXXXXXXXYNELRVAPEE

SEQ ID NO: 2 - protein, 38 aa, synthetic construct
  VARIANT 9..28: note = X can be G, A, or deleted
RRRRRRRRXXXXXXXXXXXXXXXXXXXXQIMFETFNTP

SEQ ID NO: 3 - protein, 38 aa, synthetic construct
  VARIANT 9..28: note = X can be G, A, or deleted
RRRRRRRRXXXXXXXXXXXXXXXXXXXXLPHAILRLDL

SEQ ID NO: 4 - protein, 38 aa, synthetic construct
  VARIANT 9..28: note = X can be G, A, or deleted
RRRRRRRRXXXXXXXXXXXXXXXXXXXXAGRDLTDYLM

SEQ ID NO: 5 - protein, 38 aa, synthetic construct
  VARIANT 9..28: note = X can be G, A, or deleted
RRRRRRRRXXXXXXXXXXXXXXXXXXXXLCYVALDFEQ

SEQ ID NO: 6 - protein, 38 aa, synthetic construct
  VARIANT 9..28: note = X can be G, A, or deleted
RRRRRRRRXXXXXXXXXXXXXXXXXXXXKYSVWIGGSI

SEQ ID NO: 7 - protein, 48 aa, synthetic construct
  VARIANT 9..28: note = X can be G, A, or deleted
RRRRRRRRXXXXXXXXXXXXXXXXXXXXYNELRVAPEEHPVLLTEAPL

SEQ ID NO: 8 - protein, 48 aa, synthetic construct
  VARIANT 9..28: note = X can be G, A, or deleted
RRRRRRRRXXXXXXXXXXXXXXXXXXXXNPKANREKMTQIMFETFNTP

SEQ ID NO: 9 - protein, 48 aa, synthetic construct
  VARIANT 9..28: note = X can be G, A, or deleted
RRRRRRRRXXXXXXXXXXXXXXXXXXXXQIMFETFNTPAMYVAIQAVL

SEQ ID NO: 10 - protein, 48 aa, synthetic construct
  VARIANT 9..28: note = X can be G, A, or deleted
RRRRRRRRXXXXXXXXXXXXXXXXXXXXHTVPIYEGYALPHAILRLDL

SEQ ID NO: 11 - protein, 48 aa, synthetic construct
  VARIANT 9..28: note = X can be G, A, or deleted
RRRRRRRRXXXXXXXXXXXXXXXXXXXXLPHAILRLDLAGRDLTDYLM

SEQ ID NO: 12 - protein, 48 aa, synthetic construct
  VARIANT 9..28: note = X can be G, A, or deleted
RRRRRRRRXXXXXXXXXXXXXXXXXXXXAGRDLTDYLMKILTERGYSF

SEQ ID NO: 13 - protein, 48 aa, synthetic construct
  VARIANT 9..28: note = X can be G, A, or deleted
RRRRRRRRXXXXXXXXXXXXXXXXXXXXDIKEKLCYVALDFEQEMATA

SEQ ID NO: 14 - protein, 48 aa, synthetic construct
  VARIANT 9..28: note = X can be G, A, or deleted
RRRRRRRRXXXXXXXXXXXXXXXXXXXXAPPERKYSVWIGGSILASLS

SEQ ID NO: 15 - protein, 50 aa, synthetic construct
  VARIANT 1..20: note = X can be any naturally-occurring amino acid or any unnatural amino acid or deleted
  VARIANT 21: note = X can be any naturally-occurring amino acid or any unnatural amino acid
  VARIANT 31..50: note = X can be any naturally-occurring amino acid or any unnatural amino acid or deleted
XXXXXXXXXXXXXXXXXXXXXNELRVAPEEXXXXXXXXXXXXXXXXXXXX

SEQ ID NO: 16 - protein, 50 aa, synthetic construct
  VARIANT 1..20: note = X can be G, A, or deleted
  VARIANT 22: note = X can be any naturally-occurring amino acid or any unnatural amino acid
  VARIANT 31..50: note = X can be G, A, or deleted
XXXXXXXXXXXXXXXXXXXXYXELRVAPEEXXXXXXXXXXXXXXXXXXXX

SEQ ID NO: 17 - protein, 50 aa, synthetic construct
  VARIANT 1..20: note = X can be G, A, or deleted
  VARIANT 23: note = X can be any naturally-occurring amino acid or any unnatural amino acid
  VARIANT 31..50: note = X can be G, A, or deleted
XXXXXXXXXXXXXXXXXXXXYNXLRVAPEEXXXXXXXXXXXXXXXXXXXX

SEQ ID NO: 18 - protein, 50 aa, synthetic construct
  VARIANT 1..20: note = X can be G, A, or deleted
  VARIANT 24: note = X can be any naturally-occurring amino acid or any unnatural amino acid
  VARIANT 31..50: note = X can be G, A, or deleted
XXXXXXXXXXXXXXXXXXXXYNEXRVAPEEXXXXXXXXXXXXXXXXXXXX

SEQ ID NO: 19 - protein, 50 aa, synthetic construct
  VARIANT 1..20: note = X can be G, A, or deleted
  VARIANT 25: note = X can be any naturally-occurring amino acid or any unnatural amino acid
  VARIANT 31..50: note = X can be G, A, or deleted
XXXXXXXXXXXXXXXXXXXXYNELXVAPEEXXXXXXXXXXXXXXXXXXXX

SEQ ID NO: 20 - protein, 50 aa, synthetic construct
  VARIANT 1..20: note = X can be G, A, or deleted
  VARIANT 26: note = X can be any naturally-occurring amino acid or any unnatural amino acid
  VARIANT 31..50: note = X can be G, A, or deleted
XXXXXXXXXXXXXXXXXXXXYNELRXAPEEXXXXXXXXXXXXXXXXXXXX

SEQ ID NO: 21 - protein, 50 aa, synthetic construct
  VARIANT 1..20: note = X can be G, A, or deleted
  VARIANT 27: note = X can be any naturally-occurring amino acid or any unnatural amino acid
  VARIANT 31..50: note = X can be G, A, or deleted
XXXXXXXXXXXXXXXXXXXXYNELRVXPEEXXXXXXXXXXXXXXXXXXXX

SEQ ID NO: 22 - protein, 50 aa, synthetic construct
  VARIANT 1..20: note = X can be G, A, or deleted
  VARIANT 28: note = X can be any naturally-occurring amino acid or any unnatural amino acid
  VARIANT 31..50: note = X can be G, A, or deleted
XXXXXXXXXXXXXXXXXXXXYNELRVAXEEXXXXXXXXXXXXXXXXXXXX

SEQ ID NO: 23 - protein, 50 aa, synthetic construct
  VARIANT 1..20: note = X can be G, A, or deleted
  VARIANT 29: note = X can be any naturally-occurring amino acid or any unnatural amino acid
  VARIANT 31..50: note = X can be G, A, or deleted
XXXXXXXXXXXXXXXXXXXXYNELRVAPXEXXXXXXXXXXXXXXXXXXXX

SEQ ID NO: 24 - protein, 50 aa, synthetic construct
  VARIANT 1..20: note = X can be G, A, or deleted
  VARIANT 30: note = X can be any naturally-occurring amino acid or any unnatural amino acid
  VARIANT 31..50: note = X can be G, A, or deleted
XXXXXXXXXXXXXXXXXXXXYNELRVAPEXXXXXXXXXXXXXXXXXXXXX

SEQ ID NO: 25 - protein, 50 aa, synthetic construct
  VARIANT 1..20: note = X can be any naturally-occurring amino acid or any unnatural amino acid or deleted
  VARIANT 31..50: note = X can be any naturally-occurring amino acid or any unnatural amino acid or deleted
XXXXXXXXXXXXXXXXXXXXWHHTFYNELRXXXXXXXXXXXXXXXXXXXX

SEQ ID NO: 26 - protein, 50 aa, synthetic construct
  VARIANT 1..20: note = X can be G, A, or deleted
  VARIANT 22: note = X can be any naturally-occurring amino acid or any unnatural amino acid
  VARIANT 31..50: note = X can be G, A, or deleted
XXXXXXXXXXXXXXXXXXXXWXHTFYNELRXXXXXXXXXXXXXXXXXXXX

SEQ ID NO: 27 - protein, 50 aa, synthetic construct
  VARIANT 1..20: note = X can be G, A, or deleted
  VARIANT 23: note = X can be any naturally-occurring amino acid or any unnatural amino acid
  VARIANT 31..50: note = X can be G, A, or deleted
XXXXXXXXXXXXXXXXXXXXWHXTFYNELRXXXXXXXXXXXXXXXXXXXX

SEQ ID NO: 28 - protein, 50 aa, synthetic construct
  VARIANT 1..20: note = X can be G, A, or deleted
  VARIANT 24: note = X can be any naturally-occurring amino acid or any unnatural amino acid
  VARIANT 31..50: note = X can be G, A, or deleted
XXXXXXXXXXXXXXXXXXXXWHHXFYNELRXXXXXXXXXXXXXXXXXXXX

SEQ ID NO: 29 - protein, 50 aa, synthetic construct
  VARIANT 1..20: note = X can be G, A, or deleted
  VARIANT 25: note = X can be any naturally-occurring amino acid or any unnatural amino acid
  VARIANT 31..50: note = X can be G, A, or deleted
XXXXXXXXXXXXXXXXXXXXWHHTXYNELRXXXXXXXXXXXXXXXXXXXX

SEQ ID NO: 30 - protein, 50 aa, synthetic construct
  VARIANT 1..20: note = X can be G, A, or deleted
  VARIANT 26: note = X can be any naturally-occurring amino acid or any unnatural amino acid
  VARIANT 31..50: note = X can be G, A, or deleted
XXXXXXXXXXXXXXXXXXXXWHHTFXNELRXXXXXXXXXXXXXXXXXXXX

SEQ ID NO: 31 - protein, 50 aa, synthetic construct
  VARIANT 1..20: note = X can be G, A, or deleted
  VARIANT 27: note = X can be any naturally-occurring amino acid or any unnatural amino acid
  VARIANT 31..50: note = X can be G, A, or deleted
XXXXXXXXXXXXXXXXXXXXWHHTFYXELRXXXXXXXXXXXXXXXXXXXX

SEQ ID NO: 32 - protein, 50 aa, synthetic construct
  VARIANT 1..20: note = X can be G, A, or deleted
  VARIANT 28: note = X can be any naturally-occurring amino acid or any unnatural amino acid
  VARIANT 31..50: note = X can be G, A, or deleted
XXXXXXXXXXXXXXXXXXXXWHHTFYNXLRXXXXXXXXXXXXXXXXXXXX

SEQ ID NO: 33 - protein, 50 aa, synthetic construct
  VARIANT 1..20: note = X can be G, A, or deleted
  VARIANT 29: note = X can be any naturally-occurring amino acid or any unnatural amino acid
  VARIANT 31..50: note = X can be G, A, or deleted
XXXXXXXXXXXXXXXXXXXXWHHTFYNEXRXXXXXXXXXXXXXXXXXXXX

SEQ ID NO: 34 - protein, 50 aa, synthetic construct
  VARIANT 1..20: note = X can be G, A, or deleted
  VARIANT 30: note = X can be any naturally-occurring amino acid or any unnatural amino acid
  VARIANT 31..50: note = X can be G, A, or deleted
XXXXXXXXXXXXXXXXXXXXWHHTFYNELXXXXXXXXXXXXXXXXXXXXX

SEQ ID NO: 35 - protein, 50 aa, synthetic construct
  VARIANT 1..20: note = X can be G, A, or deleted
  VARIANT 31..50: note = X can be G, A, or deleted
XXXXXXXXXXXXXXXXXXXXVTNWDDMEKIXXXXXXXXXXXXXXXXXXXX

SEQ ID NO: 36 - protein, 50 aa, synthetic construct
  VARIANT 1..20: note = X can be G, A, or deleted
  VARIANT 31..50: note = X can be G, A, or deleted
XXXXXXXXXXXXXXXXXXXXTNWDDMEKIWXXXXXXXXXXXXXXXXXXXX

SEQ ID NO: 37 - protein, 50 aa, synthetic construct
  VARIANT 1..20: note = X can be G, A, or deleted
  VARIANT 31..50: note = X can be G, A, or deleted
XXXXXXXXXXXXXXXXXXXXNWDDMEKIWHXXXXXXXXXXXXXXXXXXXX

SEQ ID NO: 38 - protein, 50 aa, synthetic construct
  VARIANT 1..20: note = X can be G, A, or deleted
  VARIANT 31..50: note = X can be G, A, or deleted
XXXXXXXXXXXXXXXXXXXXWDDMEKIWHHXXXXXXXXXXXXXXXXXXXX

SEQ ID NO: 39 - protein, 50 aa, synthetic construct
  VARIANT 1..20: note = X can be G, A, or deleted
  VARIANT 31..50: note = X can be G, A, or deleted
XXXXXXXXXXXXXXXXXXXXDDMEKIWHHTXXXXXXXXXXXXXXXXXXXX

SEQ ID NO: 40 - protein, 50 aa, synthetic construct
  VARIANT 1..20: note = X can be G, A, or deleted
  VARIANT 31..50: note = X can be G, A, or deleted
XXXXXXXXXXXXXXXXXXXXDMEKIWHHTFXXXXXXXXXXXXXXXXXXXX

SEQ ID NO: 41 - protein, 50 aa, synthetic construct
  VARIANT 1..20: note = X can be G, A, or deleted
  VARIANT 31..50: note = X can be G, A, or deleted
XXXXXXXXXXXXXXXXXXXXMEKIWHHTFYXXXXXXXXXXXXXXXXXXXX

SEQ ID NO: 42 - protein, 50 aa, synthetic construct
  VARIANT 1..20: note = X can be G, A, or deleted
  VARIANT 31..50: note = X can be G, A, or deleted
XXXXXXXXXXXXXXXXXXXXEKIWHHTFYNXXXXXXXXXXXXXXXXXXXX

SEQ ID NO: 43 - protein, 50 aa, synthetic construct
  VARIANT 1..20: note = X can be G, A, or deleted
  VARIANT 31..50: note = X can be G, A, or deleted
XXXXXXXXXXXXXXXXXXXXKIWHHTFYNEXXXXXXXXXXXXXXXXXXXX

SEQ ID NO: 44 - protein, 50 aa, synthetic construct
  VARIANT 1..20: note = X can be G, A, or deleted
  VARIANT 31..50: note = X can be G, A, or deleted
XXXXXXXXXXXXXXXXXXXXIWHHTFYNELXXXXXXXXXXXXXXXXXXXX

SEQ ID NO: 45 - protein, 50 aa, synthetic construct; identical to SEQ ID NO: 25
  VARIANT 1..20: note = X can be G, A, or deleted
  VARIANT 31..50: note = X can be G, A, or deleted

SEQ ID NO: 46 - protein, 50 aa, synthetic construct
  VARIANT 1..20: note = X can be G, A, or deleted
  VARIANT 31..50: note = X can be G, A, or deleted
XXXXXXXXXXXXXXXXXXXXHHTFYNELRVXXXXXXXXXXXXXXXXXXXX

SEQ ID NO: 47 - protein, 50 aa, synthetic construct
  VARIANT 1..20: note = X can be G, A, or deleted
  VARIANT 31..50: note = X can be G, A, or deleted
XXXXXXXXXXXXXXXXXXXXHTFYNELRVAXXXXXXXXXXXXXXXXXXXX

SEQ ID NO: 48 - protein, 50 aa, synthetic construct
  VARIANT 1..20: note = X can be G, A, or deleted
  VARIANT 31..50: note = X can be G, A, or deleted
XXXXXXXXXXXXXXXXXXXXTFYNELRVAPXXXXXXXXXXXXXXXXXXXX

SEQ ID NO: 49 - protein, 50 aa, synthetic construct
  VARIANT 1..20: note = X can be G, A, or deleted
  VARIANT 31..50: note = X can be G, A, or deleted
XXXXXXXXXXXXXXXXXXXXFYNELRVAPEXXXXXXXXXXXXXXXXXXXX

SEQ ID NO: 50 - protein, 50 aa, synthetic construct
  VARIANT 1..20: note = X can be G, A, or deleted
  VARIANT 31..50: note = X can be G, A, or deleted
XXXXXXXXXXXXXXXXXXXXYNELRVAPEEXXXXXXXXXXXXXXXXXXXX

SEQ ID NO: 51 - protein, 50 aa, synthetic construct
  VARIANT 1..20: note = X can be G, A, or deleted
  VARIANT 31..50: note = X can be G, A, or deleted
XXXXXXXXXXXXXXXXXXXXNELRVAPEEHXXXXXXXXXXXXXXXXXXXX

SEQ ID NO: 52 - protein, 50 aa, synthetic construct
  VARIANT 1..20: note = X can be G, A, or deleted
  VARIANT 31..50: note = X can be G, A, or deleted
XXXXXXXXXXXXXXXXXXXXELRVAPEEHPXXXXXXXXXXXXXXXXXXXX

SEQ ID NO: 53 - protein, 50 aa, synthetic construct
  VARIANT 1..20: note = X can be G, A, or deleted
  VARIANT 31..50: note = X can be G, A, or deleted
XXXXXXXXXXXXXXXXXXXXLRVAPEEHPVXXXXXXXXXXXXXXXXXXXX

SEQ ID NO: 54 - protein, 50 aa, synthetic construct
  VARIANT 1..20: note = X can be G, A, or deleted
  VARIANT 31..50: note = X can be G, A, or deleted
XXXXXXXXXXXXXXXXXXXXRVAPEEHPVLXXXXXXXXXXXXXXXXXXXX

SEQ ID NO: 55 - protein, 50 aa, synthetic construct
  VARIANT 1..20: note = X can be G, A, or deleted
  VARIANT 31..50: note = X can be G, A, or deleted
XXXXXXXXXXXXXXXXXXXXVAPEEHPVLLXXXXXXXXXXXXXXXXXXXX

SEQ ID NO: 56 - protein, 50 aa, synthetic construct
  VARIANT 1..20: note = X can be G, A, or deleted
  VARIANT 31..50: note = X can be G, A, or deleted
XXXXXXXXXXXXXXXXXXXXAPEEHPVLLTXXXXXXXXXXXXXXXXXXXX

SEQ ID NO: 57 - protein, 50 aa, synthetic construct
  VARIANT 1..20: note = X can be G, A, or deleted
  VARIANT 31..50: note = X can be G, A, or deleted
XXXXXXXXXXXXXXXXXXXXPEEHPVLLTEXXXXXXXXXXXXXXXXXXXX

SEQ ID NO: 58 - protein, 50 aa, synthetic construct
  VARIANT 1..20: note = X can be G, A, or deleted
  VARIANT 31..50: note = X can be G, A, or deleted
XXXXXXXXXXXXXXXXXXXXEEHPVLLTEAXXXXXXXXXXXXXXXXXXXX

SEQ ID NO: 59 - protein, 50 aa, synthetic construct
  VARIANT 1..20: note = X can be G, A, or deleted
  VARIANT 31..50: note = X can be G, A, or deleted
XXXXXXXXXXXXXXXXXXXXEHPVLLTEAPXXXXXXXXXXXXXXXXXXXX

SEQ ID NO: 60 - protein, 50 aa, synthetic construct
  VARIANT 1..20: note = X can be G, A, or deleted
  VARIANT 31..50: note = X can be G, A, or deleted
XXXXXXXXXXXXXXXXXXXXHPVLLTEAPLXXXXXXXXXXXXXXXXXXXX

SEQ ID NO: 61 - protein, 50 aa, synthetic construct
  VARIANT 1..20: note = X can be G, A, or deleted
  VARIANT 31..50: note = X can be G, A, or deleted
XXXXXXXXXXXXXXXXXXXXPVLLTEAPLNXXXXXXXXXXXXXXXXXXXX

SEQ ID NO: 62 - protein, 50 aa, synthetic construct
  VARIANT 1..20: note = X can be G, A, or deleted
  VARIANT 31..50: note = X can be G, A, or deleted
XXXXXXXXXXXXXXXXXXXXVLLTEAPLNPXXXXXXXXXXXXXXXXXXXX

SEQ ID NO: 63 - protein, 50 aa, synthetic construct
  VARIANT 1..20: note = X can be G, A, or deleted
  VARIANT 31..50: note = X can be G, A, or deleted
XXXXXXXXXXXXXXXXXXXXLLTEAPLNPKXXXXXXXXXXXXXXXXXXXX

SEQ ID NO: 64 - protein, 50 aa, synthetic construct
  VARIANT 1..20: note = X can be G, A, or deleted
  VARIANT 31..50: note = X can be G, A, or deleted
XXXXXXXXXXXXXXXXXXXXLTEAPLNPKAXXXXXXXXXXXXXXXXXXXX

SEQ ID NO: 65 - protein, 50 aa, synthetic construct
  VARIANT 1..20: note = X can be G, A, or deleted
  VARIANT 31..50: note = X can be G, A, or deleted
XXXXXXXXXXXXXXXXXXXXTEAPLNPKANXXXXXXXXXXXXXXXXXXXX

SEQ ID NO: 66 - protein, 60 aa, synthetic construct
  VARIANT 1..20: note = X can be G, A, or deleted
  VARIANT 41..60: note = X can be G, A, or deleted
XXXXXXXXXXXXXXXXXXXXEHGIVTNWDDMEKIWHHTFYXXXXXXXXXXXXXXXXXXXX

SEQ ID NO: 67 - protein, 60 aa, synthetic construct
  VARIANT 1..20: note = X can be G, A, or deleted
  VARIANT 41..60: note = X can be G, A, or deleted
XXXXXXXXXXXXXXXXXXXXHGIVTNWDDMEKIWHHTFYNXXXXXXXXXXXXXXXXXXXX

SEQ ID NO: 68 - protein, 60 aa, synthetic construct
  VARIANT 1..20: note = X can be G, A, or deleted
  VARIANT 41..60: note = X can be G, A, or deleted
XXXXXXXXXXXXXXXXXXXXGIVTNWDDMEKIWHHTFYNEXXXXXXXXXXXXXXXXXXXX

SEQ ID NO: 69 - protein, 60 aa, synthetic construct
  VARIANT 1..20: note = X can be G, A, or deleted
  VARIANT 41..60: note = X can be G, A, or deleted
XXXXXXXXXXXXXXXXXXXXIVTNWDDMEKIWHHTFYNELXXXXXXXXXXXXXXXXXXXX

SEQ ID NO: 70 - protein, 60 aa, synthetic construct
  VARIANT 1..20: note = X can be G, A, or deleted
  VARIANT 41..60: note = X can be G, A, or deleted
XXXXXXXXXXXXXXXXXXXXVTNWDDMEKIWHHTFYNELRXXXXXXXXXXXXXXXXXXXX

SEQ ID NO: 71 - protein, 60 aa, synthetic construct
  VARIANT 1..20: note = X can be G, A, or deleted
  VARIANT 41..60: note = X can be G, A, or deleted
XXXXXXXXXXXXXXXXXXXXTNWDDMEKIWHHTFYNELRVXXXXXXXXXXXXXXXXXXXX

SEQ ID NO: 72 - protein, 60 aa, synthetic construct
  VARIANT 1..20: note = X can be G, A, or deleted
  VARIANT 41..60: note = X can be G, A, or deleted
XXXXXXXXXXXXXXXXXXXXNWDDMEKIWHHTFYNELRVAXXXXXXXXXXXXXXXXXXXX

SEQ ID NO: 73 - protein, 60 aa, synthetic construct
  VARIANT 1..20: note = X can be G, A, or deleted
  VARIANT 41..60: note = X can be G, A, or deleted
XXXXXXXXXXXXXXXXXXXXWDDMEKIWHHTFYNELRVAPXXXXXXXXXXXXXXXXXXXX

SEQ ID NO: 74 - protein, 60 aa, synthetic construct
  VARIANT 1..20: note = X can be G, A, or deleted
  VARIANT 41..60: note = X can be G, A, or deleted
XXXXXXXXXXXXXXXXXXXXDDMEKIWHHTFYNELRVAPEXXXXXXXXXXXXXXXXXXXX

SEQ ID NO: 75 - protein, 60 aa, synthetic construct
  VARIANT 1..20: note = X can be G, A, or deleted
  VARIANT 41..60: note = X can be G, A, or deleted
XXXXXXXXXXXXXXXXXXXXDMEKIWHHTFYNELRVAPEEXXXXXXXXXXXXXXXXXXXX

SEQ ID NO: 76 - protein, 60 aa, synthetic construct
  VARIANT 1..20: note = X can be G, A, or deleted
  VARIANT 41..60: note = X can be G, A, or deleted
XXXXXXXXXXXXXXXXXXXXMEKIWHHTFYNELRVAPEEHXXXXXXXXXXXXXXXXXXXX

SEQ ID NO: 77 - protein, 60 aa, synthetic construct
  VARIANT 1..20: note = X can be G, A, or deleted
  VARIANT 41..60: note = X can be G, A, or deleted
XXXXXXXXXXXXXXXXXXXXEKIWHHTFYNELRVAPEEHPXXXXXXXXXXXXXXXXXXXX

SEQ ID NO: 78 - protein, 60 aa, synthetic construct
  VARIANT 1..20: note = X can be G, A, or deleted
  VARIANT 41..60: note = X can be G, A, or deleted
XXXXXXXXXXXXXXXXXXXXKIWHHTFYNELRVAPEEHPVXXXXXXXXXXXXXXXXXXXX

SEQ ID NO: 79 - protein, 60 aa, synthetic construct
  VARIANT 1..20: note = X can be G, A, or deleted
  VARIANT 41..60: note = X can be G, A, or deleted
XXXXXXXXXXXXXXXXXXXXIWHHTFYNELRVAPEEHPVLXXXXXXXXXXXXXXXXXXXX

SEQ ID NO: 80 - protein, 60 aa, synthetic construct
  VARIANT 1..20: note = X can be G, A, or deleted
  VARIANT 41..60: note = X can be G, A, or deleted
XXXXXXXXXXXXXXXXXXXXWHHTFYNELRVAPEEHPVLLXXXXXXXXXXXXXXXXXXXX

SEQ ID NO: 81 - protein, 60 aa, synthetic construct
  VARIANT 1..20: note = X can be G, A, or deleted
  VARIANT 41..60: note = X can be G, A, or deleted
XXXXXXXXXXXXXXXXXXXXHHTFYNELRVAPEEHPVLLTXXXXXXXXXXXXXXXXXXXX

SEQ ID NO: 82 - protein, 60 aa, synthetic construct
  VARIANT 1..20: note = X can be G, A, or deleted
  VARIANT 41..60: note = X can be G, A, or deleted
XXXXXXXXXXXXXXXXXXXXHTFYNELRVAPEEHPVLLTEXXXXXXXXXXXXXXXXXXXX

SEQ ID NO: 83 - protein, 60 aa, synthetic construct
  VARIANT 1..20: note = X can be G, A, or deleted
  VARIANT 41..60: note = X can be G, A, or deleted
XXXXXXXXXXXXXXXXXXXXTFYNELRVAPEEHPVLLTEAXXXXXXXXXXXXXXXXXXXX

SEQ ID NO: 84 - protein, 60 aa, synthetic construct
  VARIANT 1..20: note = X can be G, A, or deleted
  VARIANT 41..60: note = X can be G, A, or deleted
XXXXXXXXXXXXXXXXXXXXFYNELRVAPEEHPVLLTEAPXXXXXXXXXXXXXXXXXXXX

SEQ ID NO: 85 - protein, 60 aa, synthetic construct
  VARIANT 1..20: note = X can be G, A, or deleted
  VARIANT 41..60: note = X can be G, A, or deleted
XXXXXXXXXXXXXXXXXXXXYNELRVAPEEHPVLLTEAPLXXXXXXXXXXXXXXXXXXXX

SEQ ID NO: 86 - protein, 60 aa, synthetic construct
  VARIANT 1..20: note = X can be G, A, or deleted
  VARIANT 21: note = X can be any naturally-occurring amino acid or any unnatural amino acid
  VARIANT 41..60: note = X can be G, A, or deleted
XXXXXXXXXXXXXXXXXXXXXMEKIWHHTFYNELRVAPEEXXXXXXXXXXXXXXXXXXXX

SEQ ID NO: 87 - protein, 60 aa, synthetic construct
  VARIANT 1..20: note = X can be G, A, or deleted
  VARIANT 22: note = X can be any naturally-occurring amino acid or any unnatural amino acid
  VARIANT 41..60: note = X can be G, A, or deleted
XXXXXXXXXXXXXXXXXXXXDXEKIWHHTFYNELRVAPEEXXXXXXXXXXXXXXXXXXXX

SEQ ID NO: 88 - protein, 60 aa, synthetic construct
  VARIANT 1..20: note = X can be G, A, or deleted
  VARIANT 23: note = X can be any naturally-occurring amino acid or any unnatural amino acid
  VARIANT 41..60: note = X can be G, A, or deleted
XXXXXXXXXXXXXXXXXXXXDMXKIWHHTFYNELRVAPEEXXXXXXXXXXXXXXXXXXXX

SEQ ID NO: 89 - protein, 60 aa, synthetic construct
  VARIANT 1..20: note = X can be G, A, or deleted
  VARIANT 24: note = X can be any naturally-occurring amino acid or any unnatural amino acid
  VARIANT 41..60: note = X can be G, A, or deleted
XXXXXXXXXXXXXXXXXXXXDMEXIWHHTFYNELRVAPEEXXXXXXXXXXXXXXXXXXXX

SEQ ID NO: 90 - protein, 60 aa, synthetic construct
  VARIANT 1..20: note = X can be G, A, or deleted
  VARIANT 25: note = X can be any naturally-occurring amino acid or any unnatural amino acid
  VARIANT 41..60: note = X can be G, A, or deleted
XXXXXXXXXXXXXXXXXXXXDMEKXWHHTFYNELRVAPEEXXXXXXXXXXXXXXXXXXXX

SEQ ID NO: 91 - protein, 60 aa, synthetic construct
  VARIANT 1..20: note = X can be G, A, or deleted
  VARIANT 26: note = X can be any naturally-occurring amino acid or any unnatural amino acid
  VARIANT 41..60: note = X can be G, A, or deleted
XXXXXXXXXXXXXXXXXXXXDMEKIXHHTFYNELRVAPEEXXXXXXXXXXXXXXXXXXXX

SEQ ID NO: 92 - protein, 60 aa, synthetic construct
  VARIANT 1..20: note = X can be G, A, or deleted
  VARIANT 27: note = X can be any naturally-occurring amino acid or any unnatural amino acid
  VARIANT 41..60: note = X can be G, A, or deleted
XXXXXXXXXXXXXXXXXXXXDMEKIWXHTFYNELRVAPEEXXXXXXXXXXXXXXXXXXXX

SEQ ID NO: 93 - protein, 60 aa, synthetic construct
  VARIANT 1..20: note = X can be G, A, or deleted
  VARIANT 28: note = X can be any naturally-occurring amino acid or any unnatural amino acid
  VARIANT 41..60: note = X can be G, A, or deleted
XXXXXXXXXXXXXXXXXXXXDMEKIWHXTFYNELRVAPEEXXXXXXXXXXXXXXXXXXXX

SEQ ID NO: 94 - protein, 60 aa, synthetic construct
  VARIANT 1..20: note = X can be G, A, or deleted
  VARIANT 29: note = X can be any naturally-occurring amino acid or any unnatural amino acid
  VARIANT 41..60: note = X can be G, A, or deleted
XXXXXXXXXXXXXXXXXXXXDMEKIWHHXFYNELRVAPEEXXXXXXXXXXXXXXXXXXXX

SEQ ID NO: 95 - protein, 60 aa, synthetic construct
  VARIANT 1..20: note = X can be G, A, or deleted
  VARIANT 30: note = X can be any naturally-occurring amino acid or any unnatural amino acid
  VARIANT 41..60: note = X can be G, A, or deleted
XXXXXXXXXXXXXXXXXXXXDMEKIWHHTXYNELRVAPEEXXXXXXXXXXXXXXXXXXXX

SEQ ID NO: 96 - protein, 60 aa, synthetic construct
  VARIANT 1..20: note = X can be G, A, or deleted
  VARIANT 31: note = X can be any naturally-occurring amino acid or any unnatural amino acid
  VARIANT 41..60: note = X can be G, A, or deleted
XXXXXXXXXXXXXXXXXXXXDMEKIWHHTFXNELRVAPEEXXXXXXXXXXXXXXXXXXXX

SEQ ID NO: 97 - protein, 60 aa, synthetic construct
  VARIANT 1..20: note = X can be G, A, or deleted
  VARIANT 32: note = X can be any naturally-occurring amino acid or any unnatural amino acid
  VARIANT 41..60: note = X can be G, A, or deleted
XXXXXXXXXXXXXXXXXXXXDMEKIWHHTFYXELRVAPEEXXXXXXXXXXXXXXXXXXXX

SEQ ID NO: 98 - protein, 60 aa, synthetic construct
  VARIANT 1..20: note = X can be G, A, or deleted
  VARIANT 33: note = X can be any naturally-occurring amino acid or any unnatural amino acid
  VARIANT 41..60: note = X can be G, A, or deleted
XXXXXXXXXXXXXXXXXXXXDMEKIWHHTFYNXLRVAPEEXXXXXXXXXXXXXXXXXXXX

SEQ ID NO: 99 - protein, 80 aa, synthetic construct
  VARIANT 1..20: note = X can be G, A, or deleted
  VARIANT 21: note = X can be any naturally-occurring amino acid or any unnatural amino acid
  VARIANT 61..80: note = X can be G, A, or deleted
XXXXXXXXXXXXXXXXXXXXXEHGIVTNWDDMEKIWHHTFYNELRVAPEEHPVLLTEAPLXXXXXXXXXXXXXXXXXXXX

SEQ ID NO: 100 - protein, 80 aa, synthetic construct
  VARIANT 1..20: note = X can be G, A, or deleted
  VARIANT 22: note = X can be any naturally-occurring amino acid or any unnatural amino acid
  VARIANT 61..80: note = X can be G, A, or deleted
XXXXXXXXXXXXXXXXXXXXIXHGIVTNWDDMEKIWHHTFYNELRVAPEEHPVLLTEAPLXXXXXXXXXXXXXXXXXXXX

SEQ ID NO: 101 - protein, 80 aa, synthetic construct
  VARIANT 1..20: note = X can be G, A, or deleted
  VARIANT 23: note = X can be any naturally-occurring amino acid or any unnatural amino acid
  VARIANT 61..80: note = X can be G, A, or deleted
XXXXXXXXXXXXXXXXXXXXIEXGIVTNWDDMEKIWHHTFYNELRVAPEEHPVLLTEAPLXXXXXXXXXXXXXXXXXXXX

SEQ ID NO: 102 - protein, 80 aa, synthetic construct
  VARIANT 1..20: note = X can be G, A, or deleted
  VARIANT 24: note = X can be any naturally-occurring amino acid or any unnatural amino acid
  VARIANT 61..80: note = X can be G, A, or deleted
XXXXXXXXXXXXXXXXXXXXIEHXIVTNWDDMEKIWHHTFYNELRVAPEEHPVLLTEAPLXXXXXXXXXXXXXXXXXXXX

SEQ ID NO: 103 - protein, 80 aa, synthetic construct
  VARIANT 1..20: note = X can be G, A, or deleted
  VARIANT 25: note = X can be any naturally-occurring amino acid or any unnatural amino acid
  VARIANT 61..80: note = X can be G, A, or deleted
XXXXXXXXXXXXXXXXXXXXIEHGXVTNWDDMEKIWHHTFYNELRVAPEEHPVLLTEAPLXXXXXXXXXXXXXXXXXXXX

SEQ ID NO: 104 - protein, 80 aa, synthetic construct
  VARIANT 1..20: note = X can be G, A, or deleted
  VARIANT 26: note = X can be any naturally-occurring amino acid or any unnatural amino acid
  VARIANT 61..80: note = X can be G, A, or deleted
XXXXXXXXXXXXXXXXXXXXIEHGIXTNWDDMEKIWHHTFYNELRVAPEEHPVLLTEAPLXXXXXXXXXXXXXXXXXXXX

SEQ ID NO: 105 - protein, 80 aa, synthetic construct
  VARIANT 1..20: note = X can be G, A, or deleted
  VARIANT 27: note = X can be any naturally-occurring amino acid or any unnatural amino acid
  VARIANT 61..80: note = X can be G, A, or deleted
XXXXXXXXXXXXXXXXXXXXIEHGIVXNWDDMEKIWHHTFYNELRVAPEEHPVLLTEAPLXXXXXXXXXXXXXXXXXXXX

SEQ ID NO: 106 - protein, 80 aa, synthetic construct
  VARIANT 1..20: note = X can be G, A, or deleted
  VARIANT 28: note = X can be any naturally-occurring amino acid or any unnatural amino acid
  VARIANT 61..80: note = X can be G, A, or deleted
XXXXXXXXXXXXXXXXXXXXIEHGIVTXWDDMEKIWHHTFYNELRVAPEEHPVLLTEAPLXXXXXXXXXXXXXXXXXXXX

SEQ ID NO: 107 - protein, 80 aa, synthetic construct
  VARIANT 1..20: note = X can be G, A, or deleted
  VARIANT 29: note = X can be any naturally-occurring amino acid or any unnatural amino acid
  VARIANT 61..80: note = X can be G, A, or deleted
XXXXXXXXXXXXXXXXXXXXIEHGIVTNXDDMEKIWHHTFYNELRVAPEEHPVLLTEAPLXXXXXXXXXXXXXXXXXXXX

SEQ ID NO: 108 - protein, 80 aa, synthetic construct
  VARIANT 1..20: note = X can be G, A, or deleted
  VARIANT 30: note = X can be any naturally-occurring amino acid or any unnatural amino acid
  VARIANT 61..80: note = X can be G, A, or deleted
XXXXXXXXXXXXXXXXXXXXIEHGIVTNWXDMEKIWHHTFYNELRVAPEEHPVLLTEAPLXXXXXXXXXXXXXXXXXXXX

SEQ ID NO: 109 - protein, 80 aa, synthetic construct
  VARIANT 1..20: note = X can be G, A, or deleted
  VARIANT 31: note = X can be any naturally-occurring amino acid or any unnatural amino acid
  VARIANT 61..80: note = X can be G, A, or deleted
XXXXXXXXXXXXXXXXXXXXIEHGIVTNWDXMEKIWHHTFYNELRVAPEEHPVLLTEAPLXXXXXXXXXXXXXXXXXXXX

SEQ ID NO: 110 - protein, 80 aa, synthetic construct
  VARIANT 1..20: note = X can be G, A, or deleted
  VARIANT 32: note = X can be any naturally-occurring amino acid or any unnatural amino acid
  VARIANT 61..80: note = X can be G, A, or deleted
XXXXXXXXXXXXXXXXXXXXIEHGIVTNWDDXEKIWHHTFYNELRVAPEEHPVLLTEAPLXXXXXXXXXXXXXXXXXXXX

SEQ ID NO: 111 - protein, 80 aa, synthetic construct
  VARIANT 1..20: note = X can be G, A, or deleted
  VARIANT 33: note = X can be any naturally-occurring amino acid or any unnatural amino acid
  VARIANT 61..80: note = X can be G, A, or deleted
XXXXXXXXXXXXXXXXXXXXIEHGIVTNWDDMXKIWHHTFYNELRVAPEEHPVLLTEAPLXXXXXXXXXXXXXXXXXXXX

SEQ ID NO: 112 - protein, 80 aa, synthetic construct
  VARIANT 1..20: note = X can be G, A, or deleted
  VARIANT 34: note = X can be any naturally-occurring amino acid or any unnatural amino acid
  VARIANT 61..80: note = X can be G, A, or deleted
XXXXXXXXXXXXXXXXXXXXIEHGIVTNWDDMEXIWHHTFYNELRVAPEEHPVLLTEAPLXXXXXXXXXXXXXXXXXXXX

SEQ ID NO: 113 - protein, 80 aa, synthetic construct
  VARIANT 1..20: note = X can be G, A, or deleted
  VARIANT 35: note = X can be any naturally-occurring amino acid or any unnatural amino acid
  VARIANT 61..80: note = X can be G, A, or deleted
XXXXXXXXXXXXXXXXXXXXIEHGIVTNWDDMEKXWHHTFYNELRVAPEEHPVLLTEAPLXXXXXXXXXXXXXXXXXXXX

SEQ ID NO: 114 - protein, 80 aa, synthetic construct
  VARIANT 1..20: note = X can be G, A, or deleted
  VARIANT 36: note = X can be any naturally-occurring amino acid or any unnatural amino acid
  VARIANT 61..80: note = X can be G, A, or deleted
XXXXXXXXXXXXXXXXXXXXIEHGIVTNWDDMEKIXHHTFYNELRVAPEEHPVLLTEAPLXXXXXXXXXXXXXXXXXXXX

SEQ ID NO: 115 - protein, 80 aa, synthetic construct
  VARIANT 1..20: note = X can be G, A, or deleted
  VARIANT 37: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWXHTFYNELRVAPEEHPVLLTEAPLXXXXXXXXXXXXXXXXXXXX

SEQ ID NO: 116 - protein, 80 aa, synthetic construct
  VARIANT 1..20: note = X can be G, A, or deleted
  VARIANT 38: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHXTFYNELRVAPEEHPVLLTEAPLXXXXXXXXXXXXXXXXXXXX

SEQ ID NO: 117 - protein, 80 aa, synthetic construct
  VARIANT 1..20: note = X can be G, A, or deleted
  VARIANT 39: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHXFYNELRVAPEEHPVLLTEAPLXXXXXXXXXXXXXXXXXXXX

SEQ ID NO: 118 - protein, 80 aa, synthetic construct
  VARIANT 1..20: note = X can be G, A, or deleted
  VARIANT 40: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTXYNELRVAPEEHPVLLTEAPLXXXXXXXXXXXXXXXXXXXX

SEQ ID NO: 119 - protein, 80 aa, synthetic construct
  VARIANT 1..20: note = X can be G, A, or deleted
  VARIANT 41: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFXNELRVAPEEHPVLLTEAPLXXXXXXXXXXXXXXXXXXXX

SEQ ID NO: 120 - protein, 80 aa, synthetic construct
  VARIANT 1..20: note = X can be G, A, or deleted
  VARIANT 42: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYXELRVAPEEHPVLLTEAPLXXXXXXXXXXXXXXXXXXXX

SEQ ID NO: 121 - protein, 80 aa, synthetic construct
  VARIANT 1..20: note = X can be G, A, or deleted
  VARIANT 43: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNXLRVAPEEHPVLLTEAPLXXXXXXXXXXXXXXXXXXXX

SEQ ID NO: 122 - protein, 80 aa, synthetic construct
  VARIANT 1..20: note = X can be G, A, or deleted
  VARIANT 44: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNEXRVAPEEHPVLLTEAPLXXXXXXXXXXXXXXXXXXXX

SEQ ID NO: 123 - protein, 80 aa, synthetic construct
  VARIANT 1..20: note = X can be G, A, or deleted
  VARIANT 45: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNELXVAPEEHPVLLTEAPLXXXXXXXXXXXXXXXXXXXX

SEQ ID NO: 124 - protein, 80 aa, synthetic construct
  VARIANT 1..20: note = X can be G, A, or deleted
  VARIANT 46: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNELRXAPEEHPVLLTEAPLXXXXXXXXXXXXXXXXXXXX

SEQ ID NO: 125 - protein, 80 aa, synthetic construct
  VARIANT 1..20: note = X can be G, A, or deleted
  VARIANT 47: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNELRVXPEEHPVLLTEAPLXXXXXXXXXXXXXXXXXXXX

SEQ ID NO: 126 - protein, 80 aa, synthetic construct
  VARIANT 1..20: note = X can be G, A, or deleted
  VARIANT 48: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNELRVAXEEHPVLLTEAPLXXXXXXXXXXXXXXXXXXXX

SEQ ID NO: 127 - protein, 80 aa, synthetic construct
  VARIANT 1..20: note = X can be G, A, or deleted
  VARIANT 49: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNELRVAPXEHPVLLTEAPLXXXXXXXXXXXXXXXXXXXX

SEQ ID NO: 128 - protein, 80 aa, synthetic construct
  VARIANT 1..20: note = X can be G, A, or deleted
  VARIANT 50: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNELRVAPEXHPVLLTEAPLXXXXXXXXXXXXXXXXXXXX

SEQ ID NO: 129 - protein, 80 aa, synthetic construct
  VARIANT 1..20: note = X can be G, A, or deleted
  VARIANT 51: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNELRVAPEEXPVLLTEAPLXXXXXXXXXXXXXXXXXXXX

SEQ ID NO: 130 - protein, 80 aa, synthetic construct
  VARIANT 1..20: note = X can be G, A, or deleted
  VARIANT 52: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNELRVAPEEHXVLLTEAPLXXXXXXXXXXXXXXXXXXXX

SEQ ID NO: 131 - protein, 80 aa, synthetic construct
  VARIANT 1..20: note = X can be G, A, or deleted
  VARIANT 53: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNELRVAPEEHPXLLTEAPLXXXXXXXXXXXXXXXXXXXX

SEQ ID NO: 132 - protein, 80 aa, synthetic construct
  VARIANT 1..20: note = X can be G, A, or deleted
  VARIANT 54: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNELRVAPEEHPVXLTEAPLXXXXXXXXXXXXXXXXXXXX

SEQ ID NO: 133 - protein, 80 aa, synthetic construct
  VARIANT 1..20: note = X can be G, A, or deleted
  VARIANT 55: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNELRVAPEEHPVLXTEAPLXXXXXXXXXXXXXXXXXXXX

SEQ ID NO: 134 - protein, 80 aa, synthetic construct
  VARIANT 1..20: note = X can be G, A, or deleted
  VARIANT 56: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNELRVAPEEHPVLLXEAPLXXXXXXXXXXXXXXXXXXXX

SEQ ID NO: 135 - protein, 80 aa, synthetic construct
  VARIANT 1..20: note = X can be G, A, or deleted
  VARIANT 57: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNELRVAPEEHPVLLTXAPLXXXXXXXXXXXXXXXXXXXX

SEQ ID NO: 136 - protein, 80 aa, synthetic construct
  VARIANT 1..20: note = X can be G, A, or deleted
  VARIANT 58: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNELRVAPEEHPVLLTEXPLXXXXXXXXXXXXXXXXXXXX

SEQ ID NO: 137 - protein, 80 aa, synthetic construct
  VARIANT 1..20: note = X can be G, A, or deleted
  VARIANT 59: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNELRVAPEEHPVLLTEAXLXXXXXXXXXXXXXXXXXXXX

SEQ ID NO: 138 - protein, 80 aa, synthetic construct
  VARIANT 1..20: note = X can be G, A, or deleted
  VARIANT 60: note = X can be any naturally-occurring amino acid or any unnatural amino acid
  VARIANT 61..80: note = X can be G, A, or deleted
XXXXXXXXXXXXXXXXXXXXIEHGIVTNWDDMEKIWHHTFYNELRVAPEEHPVLLTEAPXXXXXXXXXXXXXXXXXXXXX

SEQ ID NO: 139 - protein, 50 aa, synthetic construct
  VARIANT 1..20: note = X can be any naturally-occurring amino acid or any unnatural amino acid or deleted
  VARIANT 21: note = X can be any naturally-occurring amino acid or any unnatural amino acid
  VARIANT 31..50: note = X can be any naturally-occurring amino acid or any unnatural amino acid or deleted
XXXXXXXXXXXXXXXXXXXXXIMFETFNTPXXXXXXXXXXXXXXXXXXXX

SEQ ID NO: 140 - protein, 50 aa, synthetic construct
  VARIANT 1..20: note = X can be G, A, or deleted
  VARIANT 22: note = X can be any naturally-occurring amino acid or any unnatural amino acid
  VARIANT 31..50: note = X can be G, A, or deleted
XXXXXXXXXXXXXXXXXXXXQXMFETFNTPXXXXXXXXXXXXXXXXXXXX

SEQ ID NO: 141 - protein, 50 aa, synthetic construct
  VARIANT 1..20: note = X can be G, A, or deleted
  VARIANT 23: note = X can be any naturally-occurring amino acid or any unnatural amino acid
  VARIANT 31..50: note = X can be G, A, or deleted
XXXXXXXXXXXXXXXXXXXXQIXFETFNTPXXXXXXXXXXXXXXXXXXXX

SEQ ID NO: 142 - protein, 50 aa, synthetic construct
  VARIANT 1..20: note = X can be G, A, or deleted
  VARIANT 24: note = X can be any naturally-occurring amino acid or any unnatural amino acid
  VARIANT 31..50: note = X can be G, A, or deleted
XXXXXXXXXXXXXXXXXXXXQIMXETFNTPXXXXXXXXXXXXXXXXXXXX

SEQ ID NO: 143 - protein, 50 aa, synthetic construct
  VARIANT 1..20: note = X can be G, A, or deleted
  VARIANT 25: note = X can be any naturally-occurring amino acid or any unnatural amino acid
  VARIANT 31..50: note = X can be G, A, or deleted
XXXXXXXXXXXXXXXXXXXXQIMFXTFNTPXXXXXXXXXXXXXXXXXXXX

SEQ ID NO: 144 - protein, 50 aa, synthetic construct
  VARIANT 1..20: note = X can be G, A, or deleted
  VARIANT 26: note = X can be any naturally-occurring amino acid or any unnatural amino acid
  VARIANT 31..50: note = X can be G, A, or deleted
XXXXXXXXXXXXXXXXXXXXQIMFEXFNTPXXXXXXXXXXXXXXXXXXXX

SEQ ID NO: 145 - protein, 50 aa, synthetic construct
  VARIANT 1..20: note = X can be G, A, or deleted
  VARIANT 27: note = X can be any naturally-occurring amino acid or any unnatural amino acid
  VARIANT 31..50: note = X can be G, A, or deleted
XXXXXXXXXXXXXXXXXXXXQIMFETXNTPXXXXXXXXXXXXXXXXXXXX

SEQ ID NO: 146 - protein, 50 aa, synthetic construct
  VARIANT 1..20: note = X can be G, A, or deleted
  VARIANT 28: note = X can be any naturally-occurring amino acid or any unnatural amino acid
  VARIANT 31..50: note = X can be G, A, or deleted
XXXXXXXXXXXXXXXXXXXXQIMFETFXTPXXXXXXXXXXXXXXXXXXXX

SEQ ID NO: 147 - protein, 50 aa, synthetic construct
  VARIANT 1..20: note = X can be G, A, or deleted
  VARIANT 29: note = X can be any naturally-occurring amino acid or any unnatural amino acid
  VARIANT 31..50: note = X can be G, A, or deleted
XXXXXXXXXXXXXXXXXXXXQIMFETFNXPXXXXXXXXXXXXXXXXXXXX

SEQ ID NO: 148 - protein, 50 aa, synthetic construct
  VARIANT 1..20: note = X can be G, A, or deleted
  VARIANT 30: note = X can be any naturally-occurring amino acid or any unnatural amino acid
  VARIANT 31..50: note = X can be G, A, or deleted
XXXXXXXXXXXXXXXXXXXXQIMFETFNTXXXXXXXXXXXXXXXXXXXXX

SEQ ID NO: 149 - protein, 50 aa, synthetic construct
  VARIANT 1..20: note = X can be any naturally-occurring amino acid or any unnatural amino acid or deleted
  VARIANT 24: note = X can be any naturally-occurring amino acid or any unnatural amino acid
  VARIANT 31..50: note = X can be any naturally-occurring amino acid or any unnatural amino acid or deleted
XXXXXXXXXXXXXXXXXXXXTFNXPAMYVAXXXXXXXXXXXXXXXXXXXX

SEQ ID NO: 150 - protein, 50 aa, synthetic construct
  VARIANT 1..20: note = X can be G, A, or deleted
  VARIANT 21: note = X can be any naturally-occurring amino acid or any unnatural amino acid
  VARIANT 24: note = X can be any naturally-occurring amino acid or any unnatural amino acid
  VARIANT 31..50: note = X can be G, A, or deleted
XXXXXXXXXXXXXXXXXXXXXFNXPAMYVAXXXXXXXXXXXXXXXXXXXX

SEQ ID NO: 151 - protein, 50 aa, synthetic construct
  VARIANT 1..20: note = X can be G, A, or deleted
  VARIANT 22: note = X can be any naturally-occurring amino acid or any unnatural amino acid
  VARIANT 24: note = X can be any naturally-occurring amino acid or any unnatural amino acid
  VARIANT 31..50: note = X can be G, A, or deleted
XXXXXXXXXXXXXXXXXXXXTXNXPAMYVAXXXXXXXXXXXXXXXXXXXX

SEQ ID NO: 152 - protein, 50 aa, synthetic construct
  VARIANT 1..20: note = X can be G, A, or deleted
  VARIANT 23..24: note = X can be any naturally-occurring amino acid or any unnatural amino acid
  VARIANT 31..50: note = X can be G, A, or deleted
XXXXXXXXXXXXXXXXXXXXTFXXPAMYVAXXXXXXXXXXXXXXXXXXXX

SEQ ID NO: 153 - protein, 50 aa, synthetic construct
  VARIANT 1..20: note = X can be G, A, or deleted
  VARIANT 24..25: note = X can be any naturally-occurring amino acid or any unnatural amino acid
  VARIANT 31..50: note = X can be G, A, or deleted
XXXXXXXXXXXXXXXXXXXXTFNXXAMYVAXXXXXXXXXXXXXXXXXXXX

SEQ ID NO: 154 - protein, 50 aa, synthetic construct
  VARIANT 1..20: note = X can be G, A, or deleted
  VARIANT 24: note = X can be any naturally-occurring amino acid or any unnatural amino acid
  VARIANT 26: note = X can be any naturally-occurring amino acid or any unnatural amino acid
  VARIANT 31..50: note = X can be G, A, or deleted
XXXXXXXXXXXXXXXXXXXXTFNXPXMYVAXXXXXXXXXXXXXXXXXXXX

SEQ ID NO: 155 - protein, 50 aa, synthetic construct
  VARIANT 1..20: note = X can be G, A, or deleted
  VARIANT 24: note = X can be any naturally-occurring amino acid or any unnatural amino acid
  VARIANT 27: note = X can be any naturally-occurring amino acid or any unnatural amino acid
  VARIANT 31..50: note = X can be G, A, or deleted
XXXXXXXXXXXXXXXXXXXXTFNXPAXYVAXXXXXXXXXXXXXXXXXXXX

SEQ ID NO: 156 - protein, 50 aa, synthetic construct
  VARIANT 1..20: note = X can be G, A, or deleted
  VARIANT 24: note = X can be any naturally-occurring amino acid or any unnatural amino acid
  VARIANT 28: note = X can be any naturally-occurring amino acid or any unnatural amino acid
  VARIANT 31..50: note = X can be G, A, or deleted
XXXXXXXXXXXXXXXXXXXXTFNXPAMXVAXXXXXXXXXXXXXXXXXXXX

SEQ ID NO: 157 - protein, 50 aa, synthetic construct
  VARIANT 1..20: note = X can be G, A, or deleted
  VARIANT 24: note = X can be any naturally-occurring amino acid or any unnatural amino acid
  VARIANT 29: note = X can be any naturally-occurring amino acid or any unnatural amino acid
  VARIANT 31..50: note = X can be G, A, or deleted
XXXXXXXXXXXXXXXXXXXXTFNXPAMYXAXXXXXXXXXXXXXXXXXXXX

SEQ ID NO: 158 - protein, 50 aa, synthetic construct
  VARIANT 24: note = X can be any naturally-occurring amino acid or any unnatural amino acid
  VARIANT 30: note = X can be any naturally-occurring amino acid or any unnatural amino acid
  VARIANT 31..50: note = X can be G, A, or deleted
  VARIANT 1..20: note = X can be G, A, or deleted
XXXXXXXXXXXXXXXXXXXXTFNXPAMYVXXXXXXXXXXXXXXXXXXXXX

SEQ ID NO: 159 - protein, 50 aa, synthetic construct; identical to SEQ ID NO: 65
  VARIANT 1..20: note = X can be G, A, or deleted
  VARIANT 31..50: note = X can be G, A, or deleted

SEQ ID NO: 160 - protein, 50 aa, synthetic construct
  VARIANT 1..20: note = X can be G, A, or deleted
  VARIANT 31..50: note = X can be G, A, or deleted
XXXXXXXXXXXXXXXXXXXXEAPLNPKANRXXXXXXXXXXXXXXXXXXXX

SEQ ID NO: 161 - protein, 50 aa, synthetic construct
  VARIANT 1..20: note = X can be G, A, or deleted
  VARIANT 31..50: note = X can be G, A, or deleted
XXXXXXXXXXXXXXXXXXXXAPLNPKANREXXXXXXXXXXXXXXXXXXXX

SEQ ID NO: 162 - protein, 50 aa, synthetic construct
  VARIANT 1..20: note = X can be G, A, or deleted
  VARIANT 31..50: note = X can be G, A, or deleted
XXXXXXXXXXXXXXXXXXXXPLNPKANREKXXXXXXXXXXXXXXXXXXXX

SEQ ID NO: 163 - protein, 50 aa, synthetic construct
  VARIANT 1..20: note = X can be G, A, or deleted
  VARIANT 31..50: note = X can be G, A, or deleted
XXXXXXXXXXXXXXXXXXXXLNPKANREKMXXXXXXXXXXXXXXXXXXXX

SEQ ID NO: 164 - protein, 50 aa, synthetic construct
  VARIANT 1..20: note = X can be G, A, or deleted
  VARIANT 31..50: note = X can be G, A, or deleted
XXXXXXXXXXXXXXXXXXXXNPKANREKMTXXXXXXXXXXXXXXXXXXXX

SEQ ID NO: 165 - protein, 50 aa, synthetic construct
  VARIANT 1..20: note = X can be G, A, or deleted
  VARIANT 31..50: note = X can be G, A, or deleted
XXXXXXXXXXXXXXXXXXXXPKANREKMTQXXXXXXXXXXXXXXXXXXXX

SEQ ID NO: 166 - protein, 50 aa, synthetic construct
  VARIANT 1..20: note = X can be G, A, or deleted
  VARIANT 31..50: note = X can be G, A, or deleted
XXXXXXXXXXXXXXXXXXXXKANREKMTQIXXXXXXXXXXXXXXXXXXXX

SEQ ID NO: 167 - protein, 50 aa, synthetic construct
  VARIANT 1..20: note = X can be G, A, or deleted
  VARIANT 31..50: note = X can be G, A, or deleted
XXXXXXXXXXXXXXXXXXXXANREKMTQIMXXXXXXXXXXXXXXXXXXXX

SEQ ID NO: 168 - protein, 50 aa, synthetic construct
  VARIANT 1..20: note = X can be G, A, or deleted
  VARIANT 31..50: note = X can be G, A, or deleted
XXXXXXXXXXXXXXXXXXXXNREKMTQIMFXXXXXXXXXXXXXXXXXXXX

SEQ ID NO: 169 - protein, 50 aa, synthetic construct
  VARIANT 1..20: note = X can be G, A, or deleted
  VARIANT 31..50: note = X can be G, A, or deleted
XXXXXXXXXXXXXXXXXXXXREKMTQIMFEXXXXXXXXXXXXXXXXXXXX

SEQ ID NO: 170 - protein, 50 aa, synthetic construct
  VARIANT 1..20: note = X can be G, A, or deleted
  VARIANT 31..50: note = X can be G, A, or deleted
XXXXXXXXXXXXXXXXXXXXEKMTQIMFETXXXXXXXXXXXXXXXXXXXX

SEQ ID NO: 171 - protein, 50 aa, synthetic construct
  VARIANT 1..20: note = X can be G, A, or deleted
  VARIANT 31..50: note = X can be G, A, or deleted
XXXXXXXXXXXXXXXXXXXXKMTQIMFETFXXXXXXXXXXXXXXXXXXXX

SEQ ID NO: 172 - protein, 50 aa, synthetic construct
  VARIANT 1..20: note = X can be G, A, or deleted
  VARIANT 31..50: note = X can be G, A, or deleted
XXXXXXXXXXXXXXXXXXXXMTQIMFETFNXXXXXXXXXXXXXXXXXXXX

SEQ ID NO: 173 - protein, 50 aa, synthetic construct
  VARIANT 1..20: note = X can be G, A, or deleted
  VARIANT 30: note = X can be any naturally-occurring amino acid or any unnatural amino acid
  VARIANT 31..50: note = X can be G, A, or deleted
XXXXXXXXXXXXXXXXXXXXTQIMFETFNXXXXXXXXXXXXXXXXXXXXX

SEQ ID NO: 174 - protein, 50 aa, synthetic construct; identical to SEQ ID NO: 147
  VARIANT 1..20: note = X can be G, A, or deleted
  VARIANT 29: note = X can be any naturally-occurring amino acid or any unnatural amino acid
  VARIANT 31..50: note = X can be G, A, or deleted

SEQ ID NO: 175 - protein, 50 aa, synthetic construct
  VARIANT 1..20: note = X can be G, A, or deleted
  VARIANT 28: note = X can be any naturally-occurring amino acid or any unnatural amino acid
  VARIANT 31..50: note = X can be G, A, or deleted
XXXXXXXXXXXXXXXXXXXXIMFETFNXPAXXXXXXXXXXXXXXXXXXXX

SEQ ID NO: 176 - protein, 50 aa, synthetic construct
  VARIANT 1..20: note = X can be G, A, or deleted
  VARIANT 27: note = X can be any naturally-occurring amino acid or any unnatural amino acid
  VARIANT 31..50: note = X can be G, A, or deleted
XXXXXXXXXXXXXXXXXXXXMFETFNXPAMXXXXXXXXXXXXXXXXXXXX

SEQ ID NO: 177 - protein, 50 aa, synthetic construct
  VARIANT 1..20: note = X can be G, A, or deleted
  VARIANT 26: note = X can be any naturally-occurring amino acid or any unnatural amino acid
  VARIANT 31..50: note = X can be G, A, or deleted
XXXXXXXXXXXXXXXXXXXXFETFNXPAMYXXXXXXXXXXXXXXXXXXXX

SEQ ID NO: 178 - protein, 50 aa, synthetic construct
  VARIANT 1..20: note = X can be G, A, or deleted
  VARIANT 25: note = X can be any naturally-occurring amino acid or any unnatural amino acid
  VARIANT 31..50: note = X can be G, A, or deleted
XXXXXXXXXXXXXXXXXXXXETFNXPAMYVXXXXXXXXXXXXXXXXXXXX

SEQ ID NO: 179 - protein, 50 aa, synthetic construct; identical to SEQ ID NO: 149
  VARIANT 1..20: note = X can be G, A, or deleted
  VARIANT 24: note = X can be any naturally-occurring amino acid or any unnatural amino acid
  VARIANT 31..50: note = X can be G, A, or deleted

SEQ ID NO: 180 - protein, 50 aa, synthetic construct
  VARIANT 1..20: note = X can be G, A, or deleted
  VARIANT 23: note = X can be any naturally-occurring amino acid or any unnatural amino acid
  VARIANT 31..50: note = X can be G, A, or deleted
XXXXXXXXXXXXXXXXXXXXFNXPAMYVAIXXXXXXXXXXXXXXXXXXXX

SEQ ID NO: 181 - protein, 50 aa, synthetic construct
  VARIANT 1..20: note = X can be G, A, or deleted
  VARIANT 22: note = X can be any naturally-occurring amino acid or any unnatural amino acid
  VARIANT 31..50: note = X can be G, A, or deleted
XXXXXXXXXXXXXXXXXXXXNXPAMYVAIQXXXXXXXXXXXXXXXXXXXX

SEQ ID NO: 182 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXPAMYVAIQAXXXXXXXXXXXXXXXXXXXX

SEQ ID NO: 183 - protein, 50 aa, synthetic construct
  VARIANT 1..20: note = X can be G, A, or deleted
  VARIANT 31..50: note = X can be G, A, or deleted
XXXXXXXXXXXXXXXXXXXXPAMYVAIQAVXXXXXXXXXXXXXXXXXXXX

SEQ ID NO: 184 - protein, 50 aa, synthetic construct
  VARIANT 1..20: note = X can be G, A, or deleted
  VARIANT 31..50: note = X can be G, A, or deleted
XXXXXXXXXXXXXXXXXXXXAMYVAIQAVLXXXXXXXXXXXXXXXXXXXX

SEQ ID NO: 185 - protein, 50 aa, synthetic construct
  VARIANT 1..20: note = X can be G, A, or deleted
  VARIANT 31..50: note = X can be G, A, or deleted
XXXXXXXXXXXXXXXXXXXXMYVAIQAVLSXXXXXXXXXXXXXXXXXXXX

SEQ ID NO: 186 - protein, 50 aa, synthetic construct
  VARIANT 1..20: note = X can be G, A, or deleted
  VARIANT 31..50: note = X can be G, A, or deleted
XXXXXXXXXXXXXXXXXXXXYVAIQAVLSLXXXXXXXXXXXXXXXXXXXX

SEQ ID NO: 187 - protein, 50 aa, synthetic construct
  VARIANT 1..20: note = X can be G, A, or deleted
  VARIANT 31..50: note = X can be G, A, or deleted
XXXXXXXXXXXXXXXXXXXXVAIQAVLSLYXXXXXXXXXXXXXXXXXXXX

SEQ ID NO: 188 - protein, 50 aa, synthetic construct
  VARIANT 1..20: note = X can be G, A, or deleted
  VARIANT 31..50: note = X can be G, A, or deleted
XXXXXXXXXXXXXXXXXXXXAIQAVLSLYAXXXXXXXXXXXXXXXXXXXX

SEQ ID NO: 189 - protein, 50 aa, synthetic construct
  VARIANT 1..20: note = X can be G, A, or deleted
  VARIANT 31..50: note = X can be G, A, or deleted
XXXXXXXXXXXXXXXXXXXXIQAVLSLYASXXXXXXXXXXXXXXXXXXXX

SEQ ID NO: 190 - protein, 50 aa, synthetic construct
  VARIANT 1..20: note = X can be G, A, or deleted
  VARIANT 31..50: note = X can be G, A, or deleted
XXXXXXXXXXXXXXXXXXXXQAVLSLYASGXXXXXXXXXXXXXXXXXXXX

SEQ ID NO: 191 - protein, 60 aa, synthetic construct
  VARIANT 1..20: note = X can be G, A, or deleted
  VARIANT 41..60: note = X can be G, A, or deleted
XXXXXXXXXXXXXXXXXXXXTEAPLNPKANREKMTQIMFEXXXXXXXXXXXXXXXXXXXX

SEQ ID NO: 192 - protein, 60 aa, synthetic construct
  VARIANT 1..20: note = X can be G, A, or deleted
  VARIANT 41..60: note = X can be G, A, or deleted
XXXXXXXXXXXXXXXXXXXXEAPLNPKANREKMTQIMFETXXXXXXXXXXXXXXXXXXXX

SEQ ID NO: 193 - protein, 60 aa, synthetic construct
  VARIANT 1..20: note = X can be G, A, or deleted
  VARIANT 41..60: note = X can be G, A, or deleted
XXXXXXXXXXXXXXXXXXXXAPLNPKANREKMTQIMFETFXXXXXXXXXXXXXXXXXXXX

SEQ ID NO: 194 - protein, 60 aa, synthetic construct
  VARIANT 1..20: note = X can be G, A, or deleted
  VARIANT 41..60: note = X can be G, A, or deleted
XXXXXXXXXXXXXXXXXXXXPLNPKANREKMTQIMFETFNXXXXXXXXXXXXXXXXXXXX

SEQ ID NO: 195 - protein, 60 aa, synthetic construct
  VARIANT 1..20: note = X can be G, A, or deleted
  VARIANT 40: note = X can be any naturally-occurring amino acid or any unnatural amino acid
  VARIANT 41..60: note = X can be G, A, or deleted
XXXXXXXXXXXXXXXXXXXXLNPKANREKMTQIMFETFNXXXXXXXXXXXXXXXXXXXXX

SEQ ID NO: 196 - protein, 60 aa, synthetic construct
  VARIANT 1..20: note = X can be G, A, or deleted
  VARIANT 39: note = X can be any naturally-occurring amino acid or any unnatural amino acid
  VARIANT 41..60: note = X can be G, A, or deleted
XXXXXXXXXXXXXXXXXXXXNPKANREKMTQIMFETFNXPXXXXXXXXXXXXXXXXXXXX

SEQ ID NO: 197 - protein, 60 aa, synthetic construct
  VARIANT 1..20: note = X can be G, A, or deleted
  VARIANT 38: note = X can be any naturally-occurring amino acid or any unnatural amino acid
  VARIANT 41..60: note = X can be G, A, or deleted
XXXXXXXXXXXXXXXXXXXXPKANREKMTQIMFETFNXPAXXXXXXXXXXXXXXXXXXXX

SEQ ID NO: 198 - protein, 60 aa, synthetic construct
  VARIANT 1..20: note = X can be G, A, or deleted
  VARIANT 37: note = X can be any naturally-occurring amino acid or any unnatural amino acid
  VARIANT 41..60: note = X can be G, A, or deleted
XXXXXXXXXXXXXXXXXXXXKANREKMTQIMFETFNXPAMXXXXXXXXXXXXXXXXXXXX

SEQ ID NO: 199 - protein, 60 aa, synthetic construct
  VARIANT 1..20: note = X can be G, A, or deleted
  VARIANT 36: note = X can be any naturally-occurring amino acid or any unnatural amino acid
  VARIANT 41..60: note = X can be G, A, or deleted
XXXXXXXXXXXXXXXXXXXXANREKMTQIMFETFNXPAMYXXXXXXXXXXXXXXXXXXXX

SEQ ID NO: 200 - protein, 60 aa, synthetic construct
  VARIANT 1..20: note = X can be G, A, or deleted
  VARIANT 35: note = X can be any naturally-occurring amino acid or any unnatural amino acid
  VARIANT 41..60: note = X can be G, A, or deleted
XXXXXXXXXXXXXXXXXXXXNREKMTQIMFETFNXPAMYVXXXXXXXXXXXXXXXXXXXX

SEQ ID NO: 201 - protein, 60 aa, synthetic construct
  VARIANT 1..20: note = X can be G, A, or deleted
  VARIANT 34: note = X can be any naturally-occurring amino acid or any unnatural amino acid
  VARIANT 41..60: note = X can be G, A, or deleted
XXXXXXXXXXXXXXXXXXXXREKMTQIMFETFNXPAMYVAXXXXXXXXXXXXXXXXXXXX

SEQ ID NO: 202 - protein, 60 aa, synthetic construct
  VARIANT 1..20: note = X can be G, A, or deleted
  VARIANT 33: note = X can be any naturally-occurring amino acid or any unnatural amino acid
  VARIANT 41..60: note = X can be G, A, or deleted
XXXXXXXXXXXXXXXXXXXXEKMTQIMFETFNXPAMYVAIXXXXXXXXXXXXXXXXXXXX

SEQ ID NO: 203 - protein, 60 aa, synthetic construct
  VARIANT 1..20: note = X can be G, A, or deleted
  VARIANT 32: note = X can be any naturally-occurring amino acid or any unnatural amino acid
  VARIANT 41..60: note = X can be G, A, or deleted
XXXXXXXXXXXXXXXXXXXXKMTQIMFETFNXPAMYVAIQXXXXXXXXXXXXXXXXXXXX

SEQ ID NO: 204 - protein, 60 aa, synthetic construct
  VARIANT 1..20: note = X can be G, A, or deleted
  VARIANT 31: note = X can be any naturally-occurring amino acid or any unnatural amino acid
  VARIANT 41..60: note = X can be G, A, or deleted
XXXXXXXXXXXXXXXXXXXXMTQIMFETFNXPAMYVAIQAXXXXXXXXXXXXXXXXXXXX

SEQ ID NO: 205 - protein, 60 aa, synthetic construct
  VARIANT 1..20: note = X can be G, A, or deleted
  VARIANT 30: note = X can be any naturally-occurring amino acid or any unnatural amino acid
  VARIANT 41..60: note = X can be G, A, or deleted
XXXXXXXXXXXXXXXXXXXXTQIMFETFNXPAMYVAIQAVXXXXXXXXXXXXXXXXXXXX

SEQ ID NO: 206 - protein, 60 aa, synthetic construct
  VARIANT 1..20: note = X can be G, A, or deleted
  VARIANT 29: note = X can be any naturally-occurring amino acid or any unnatural amino acid
  VARIANT 41..60: note = X can be G, A, or deleted
XXXXXXXXXXXXXXXXXXXXQIMFETFNXPAMYVAIQAVLXXXXXXXXXXXXXXXXXXXX

SEQ ID NO: 207 - protein, 60 aa, synthetic construct
  VARIANT 1..20: note = X can be G, A, or deleted
  VARIANT 28: note = X can be any naturally-occurring amino acid or any unnatural amino acid
  VARIANT 41..60: note = X can be G, A, or deleted
XXXXXXXXXXXXXXXXXXXXIMFETFNXPAMYVAIQAVLSXXXXXXXXXXXXXXXXXXXX

SEQ ID NO: 208 - protein, 60 aa, synthetic construct
  VARIANT 1..20: note = X can be G, A, or deleted
  VARIANT 27: note = X can be any naturally-occurring amino acid or any unnatural amino acid
  VARIANT 41..60: note = X can be G, A, or deleted
XXXXXXXXXXXXXXXXXXXXMFETFNXPAMYVAIQAVLSLXXXXXXXXXXXXXXXXXXXX

SEQ ID NO: 209 - protein, 60 aa, synthetic construct
  VARIANT 1..20: note = X can be G, A, or deleted
  VARIANT 26: note = X can be any naturally-occurring amino acid or any unnatural amino acid
  VARIANT 41..60: note = X can be G, A, or deleted
XXXXXXXXXXXXXXXXXXXXFETFNXPAMYVAIQAVLSLYXXXXXXXXXXXXXXXXXXXX

SEQ ID NO: 210 - protein, 60 aa, synthetic construct
  VARIANT 1..20: note = X can be G, A, or deleted
  VARIANT 25: note = X can be any naturally-occurring amino acid or any unnatural amino acid
  VARIANT 41..60: note = X can be G, A, or deleted
XXXXXXXXXXXXXXXXXXXXETFNXPAMYVAIQAVLSLYAXXXXXXXXXXXXXXXXXXXX

SEQ ID NO: 211 - protein, 60 aa, synthetic construct
  VARIANT 1..20: note = X can be G, A, or deleted
  VARIANT 24: note = X can be any naturally-occurring amino acid or any unnatural amino acid
  VARIANT 41..60: note = X can be G, A, or deleted
XXXXXXXXXXXXXXXXXXXXTFNXPAMYVAIQAVLSLYASXXXXXXXXXXXXXXXXXXXX

SEQ ID NO: 212 - protein, 61 aa, synthetic construct
  VARIANT 1..20: note = X can be G, A, or deleted
  VARIANT 21: note = X can be any naturally-occurring amino acid or any unnatural amino acid
  VARIANT 30: note = X can be any naturally-occurring amino acid or any unnatural amino acid
  VARIANT 42..61: note = X can be G, A, or deleted
XXXXXXXXXXXXXXXXXXXXXQIMFETFNXPAMYVAIQAVLXXXXXXXXXXXXXXXXXXXX

SEQ ID NO: 213 - protein, 61 aa, synthetic construct
  VARIANT 1..20: note = X can be G, A, or deleted
  VARIANT 22: note = X can be any naturally-occurring amino acid or any unnatural amino acid
  VARIANT 30: note = X can be any naturally-occurring amino acid or any unnatural amino acid
  VARIANT 42..61: note = X can be G, A, or deleted
XXXXXXXXXXXXXXXXXXXXQXIMFETFNXPAMYVAIQAVLXXXXXXXXXXXXXXXXXXXX

SEQ ID NO: 214 - protein, 61 aa, synthetic construct
  VARIANT 1..20: note = X can be G, A, or deleted
  VARIANT 23: note = X can be any naturally-occurring amino acid or any unnatural amino acid
  VARIANT 30: note = X can be any naturally-occurring amino acid or any unnatural amino acid
  VARIANT 42..61: note = X can be G, A, or deleted
XXXXXXXXXXXXXXXXXXXXQIXMFETFNXPAMYVAIQAVLXXXXXXXXXXXXXXXXXXXX

SEQ ID NO: 215 - protein, 61 aa, synthetic construct
  VARIANT 1..20: note = X can be G, A, or deleted
  VARIANT 24: note = X can be any naturally-occurring amino acid or any unnatural amino acid
  VARIANT 30: note = X can be any naturally-occurring amino acid or any unnatural amino acid
  VARIANT 42..61: note = X can be G, A, or deleted
XXXXXXXXXXXXXXXXXXXXQIMXFETFNXPAMYVAIQAVLXXXXXXXXXXXXXXXXXXXX

SEQ ID NO: 216 - protein, 61 aa, synthetic construct
  VARIANT 1..20: note = X can be G, A, or deleted
  VARIANT 25: note = X can be any naturally-occurring amino acid or any unnatural amino acid
  VARIANT 30: note = X can be any naturally-occurring amino acid or any unnatural amino acid
  VARIANT 42..61: note = X can be G, A, or deleted
XXXXXXXXXXXXXXXXXXXXQIMFXETFNXPAMYVAIQAVLXXXXXXXXXXXXXXXXXXXX

SEQ ID NO: 217 - protein, 61 aa, synthetic construct
  VARIANT 1..20: note = X can be G, A, or deleted
  VARIANT 26: note = X can be any naturally-occurring amino acid or any unnatural amino acid
  VARIANT 30: note = X can be any naturally-occurring amino acid or any unnatural amino acid
  VARIANT 42..61: note = X can be G, A, or deleted
XXXXXXXXXXXXXXXXXXXXQIMFEXTFNXPAMYVAIQAVLXXXXXXXXXXXXXXXXXXXX

SEQ ID NO: 218 - protein, 61 aa, synthetic construct
  VARIANT 1..20: note = X can be G, A, or deleted
  VARIANT 27: note = X can be any naturally-occurring amino acid or any unnatural amino acid
  VARIANT 30: note = X can be any naturally-occurring amino acid or any unnatural amino acid
  VARIANT 42..61: note = X can be G, A, or deleted
XXXXXXXXXXXXXXXXXXXXQIMFETXFNXPAMYVAIQAVLXXXXXXXXXXXXXXXXXXXX

SEQ ID NO: 219 - protein, 61 aa, synthetic construct
  VARIANT 1..20: note = X can be G, A, or deleted
  VARIANT 28: note = X can be any naturally-occurring amino acid or any unnatural amino acid
  VARIANT 30: note = X can be any naturally-occurring amino acid or any unnatural amino acid
  VARIANT 42..61: note = X can be G, A, or deleted
XXXXXXXXXXXXXXXXXXXXQIMFETFXNXPAMYVAIQAVLXXXXXXXXXXXXXXXXXXXX

SEQ ID NO: 220 - protein, 61 aa, synthetic construct
  VARIANT 1..20: note = X can be G, A, or deleted
  VARIANT 29..30: note = X can be any naturally-occurring amino acid or any unnatural amino acid
  VARIANT 42..61: note = X can be G, A, or deleted
XXXXXXXXXXXXXXXXXXXXQIMFETFNXXPAMYVAIQAVLXXXXXXXXXXXXXXXXXXXX

SEQ ID NO: 221 - protein, 61 aa, synthetic construct
  VARIANT 1..20: note = X can be G, A, or deleted
  VARIANT 29: note = X can be any naturally-occurring amino acid or any unnatural amino acid
  VARIANT 31: note = X can be any naturally-occurring amino acid or any unnatural amino acid
  VARIANT 42..61: note = X can be G, A, or deleted
XXXXXXXXXXXXXXXXXXXXQIMFETFNXPXAMYVAIQAVLXXXXXXXXXXXXXXXXXXXX

SEQ ID NO: 222 - protein, 61 aa, synthetic construct
  VARIANT 1..20: note = X can be G, A, or deleted
  VARIANT 29: note = X can be any naturally-occurring amino acid or any unnatural amino acid
  VARIANT 32: note = X can be any naturally-occurring amino acid or any unnatural amino acid
  VARIANT 42..61: note = X can be G, A, or deleted
XXXXXXXXXXXXXXXXXXXXQIMFETFNXPAXMYVAIQAVLXXXXXXXXXXXXXXXXXXXX

SEQ ID NO: 223 - protein, 61 aa, synthetic construct
  VARIANT 1..20: note = X can be G, A, or deleted
  VARIANT 29: note = X can be any naturally-occurring amino acid or any unnatural amino acid
  VARIANT 33: note = X can be any naturally-occurring amino acid or any unnatural amino acid
  VARIANT 42..61: note = X can be G, A, or deleted
XXXXXXXXXXXXXXXXXXXXQIMFETFNXPAMXYVAIQAVLXXXXXXXXXXXXXXXXXXXX

SEQ ID NO: 224 - protein, 61 aa, synthetic construct
  VARIANT 1..20: note = X can be G, A, or deleted
  VARIANT 29: note = X can be any naturally-occurring amino acid or any unnatural amino acid
  VARIANT 34: note = X can be any naturally-occurring amino acid or any unnatural amino acid
  VARIANT 42..61: note = X can be G, A, or deleted
XXXXXXXXXXXXXXXXXXXXQIMFETFNXPAMYXVAIQAVLXXXXXXXXXXXXXXXXXXXX

SEQ ID NO: 225 - protein, 61 aa, synthetic construct
  VARIANT 1..20: note = X can be G, A, or deleted
  VARIANT 29: note = X can be any naturally-occurring amino acid or any unnatural amino acid
  VARIANT 35: note = X can be any naturally-occurring amino acid or any unnatural amino acid
  VARIANT 42..61: note = X can be G, A, or deleted
XXXXXXXXXXXXXXXXXXXXQIMFETFNXPAMYVXAIQAVLXXXXXXXXXXXXXXXXXXXX

SEQ ID NO: 226 - protein, 61 aa, synthetic construct
  VARIANT 1..20: note = X can be G, A, or deleted
  VARIANT 29: note = X can be any naturally-occurring amino acid or any unnatural amino acid
  VARIANT 36: note = X can be any naturally-occurring amino acid or any unnatural amino acid
  VARIANT 42..61: note = X can be G, A, or deleted
XXXXXXXXXXXXXXXXXXXXQIMFETFNXPAMYVAXIQAVLXXXXXXXXXXXXXXXXXXXX

SEQ ID NO: 227 - protein, 61 aa, synthetic construct
  VARIANT 1..20: note = X can be G, A, or deleted
  VARIANT 29: note = X can be any naturally-occurring amino acid or any unnatural amino acid
  VARIANT 37: note = X can be any naturally-occurring amino acid or any unnatural amino acid
  VARIANT 42..61: note = X can be G, A, or deleted
XXXXXXXXXXXXXXXXXXXXQIMFETFNXPAMYVAIXQAVLXXXXXXXXXXXXXXXXXXXX

SEQ ID NO: 228 - protein, 61 aa, synthetic construct
  VARIANT 1..20: note = X can be G, A, or deleted
  VARIANT 29: note = X can be any naturally-occurring amino acid or any unnatural amino acid
  VARIANT 38: note = X can be any naturally-occurring amino acid or any unnatural amino acid
  VARIANT 42..61: note = X can be G, A, or deleted
XXXXXXXXXXXXXXXXXXXXQIMFETFNXPAMYVAIQXAVLXXXXXXXXXXXXXXXXXXXX

SEQ ID NO: 229 - protein, 61 aa, synthetic construct
  VARIANT 1..20: note = X can be G, A, or deleted
  VARIANT 29: note = X can be any naturally-occurring amino acid or any unnatural amino acid
  VARIANT 39: note = X can be any naturally-occurring amino acid or any unnatural amino acid
  VARIANT 42..61: note = X can be G, A, or deleted
XXXXXXXXXXXXXXXXXXXXQIMFETFNXPAMYVAIQAXVLXXXXXXXXXXXXXXXXXXXX

SEQ ID NO: 230 - protein, 61 aa, synthetic construct
  VARIANT 1..20: note = X can be G, A, or deleted
  VARIANT 29: note = X can be any naturally-occurring amino acid or any unnatural amino acid
  VARIANT 40: note = X can be any naturally-occurring amino acid or any unnatural amino acid
  VARIANT 42..61: note = X can be G, A, or deleted
XXXXXXXXXXXXXXXXXXXXQIMFETFNXPAMYVAIQAVXLXXXXXXXXXXXXXXXXXXXX

SEQ ID NO: 231 - protein, 61 aa, synthetic construct
  VARIANT 1..20: note = X can be G, A, or deleted
  VARIANT 29: note = X can be any naturally-occurring amino acid or any unnatural amino acid
  VARIANT 41: note = X can be any naturally-occurring amino acid or any unnatural amino acid
  VARIANT 42..61: note = X can be G, A, or deleted
XXXXXXXXXXXXXXXXXXXXQIMFETFNXPAMYVAIQAVLXXXXXXXXXXXXXXXXXXXXX

SEQ ID NO: 232 - protein, 81 aa, synthetic construct
  VARIANT 1..20: note = X can be G, A, or deleted
  VARIANT 21: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXXPKANREKMTQIMFETFNXPAMYVAIQAVLSLYASGRTTGIXXXXXXXXXXXXXXXXXXXX

SEQ ID NO: 233 - protein, 81 aa, synthetic construct
  VARIANT 1..20: note = X can be G, A, or deleted
  VARIANT 22: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXNXKANREKMTQIMFETFNXPAMYVAIQAVLSLYASGRTTGIXXXXXXXXXXXXXXXXXXXX

SEQ ID NO: 234 - protein, 81 aa, synthetic construct
  VARIANT 1..20: note = X can be G, A, or deleted
  VARIANT 23: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXNPXANREKMTQIMFETFNXPAMYVAIQAVLSLYASGRTTGIXXXXXXXXXXXXXXXXXXXX

SEQ ID NO: 235 - protein, 81 aa, synthetic construct
  VARIANT 1..20: note = X can be G, A, or deleted
  VARIANT 24: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXNPKXNREKMTQIMFETFNXPAMYVAIQAVLSLYASGRTTGIXXXXXXXXXXXXXXXXXXXX

SEQ ID NO: 236 - protein, 81 aa, synthetic construct
  VARIANT 1..20: note = X can be G, A, or deleted
  VARIANT 25: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXNPKAXREKMTQIMFETFNXPAMYVAIQAVLSLYASGRTTGIXXXXXXXXXXXXXXXXXXXX

SEQ ID NO: 237 - protein, 81 aa, synthetic construct
  VARIANT 1..20: note = X can be G, A, or deleted
  VARIANT 26: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXNPKANXEKMTQIMFETFNXPAMYVAIQAVLSLYASGRTTGIXXXXXXXXXXXXXXXXXXXX

SEQ ID NO: 238 - protein, 81 aa, synthetic construct
  VARIANT 1..20: note = X can be G, A, or deleted
  VARIANT 27: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXNPKANRXKMTQIMFETFNXPAMYVAIQAVLSLYASGRTTGIXXXXXXXXXXXXXXXXXXXX

SEQ ID NO: 239 - protein, 81 aa, synthetic construct
  VARIANT 1..20: note = X can be G, A, or deleted
  VARIANT 28: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXNPKANREXMTQIMFETFNXPAMYVAIQAVLSLYASGRTTGIXXXXXXXXXXXXXXXXXXXX

SEQ ID NO: 240 - protein, 81 aa, synthetic construct
  VARIANT 1..20: note = X can be G, A, or deleted
  VARIANT 29: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXNPKANREKXTQIMFETFNXPAMYVAIQAVLSLYASGRTTGIXXXXXXXXXXXXXXXXXXXX

SEQ ID NO: 241 - protein, 81 aa, synthetic construct
  VARIANT 1..20: note = X can be G, A, or deleted
  VARIANT 30: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXNPKANREKMXQIMFETFNXPAMYVAIQAVLSLYASGRTTGIXXXXXXXXXXXXXXXXXXXX

SEQ ID NO: 242 - protein, 81 aa, synthetic construct
  VARIANT 1..20: note = X can be G, A, or deleted
  VARIANT 31: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXNPKANREKMTXIMFETFNXPAMYVAIQAVLSLYASGRTTGIXXXXXXXXXXXXXXXXXXXX

SEQ ID NO: 243 - protein, 81 aa, synthetic construct
  VARIANT 1..20: note = X can be G, A, or deleted
  VARIANT 32: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXNPKANREKMTQXMFETFNXPAMYVAIQAVLSLYASGRTTGIXXXXXXXXXXXXXXXXXXXX

SEQ ID NO: 244 - protein, 81 aa, synthetic construct
  VARIANT 1..20: note = X can be G, A, or deleted
  VARIANT 33: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXNPKANREKMTQIXFETFNXPAMYVAIQAVLSLYASGRTTGIXXXXXXXXXXXXXXXXXXXX

SEQ ID NO: 245 - protein, 81 aa, synthetic construct
  VARIANT 1..20: note = X can be G, A, or deleted
  VARIANT 34: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXNPKANREKMTQIMXETFNXPAMYVAIQAVLSLYASGRTTGIXXXXXXXXXXXXXXXXXXXX

SEQ ID NO: 246 - protein, 81 aa, synthetic construct
  VARIANT 1..20: note = X can be G, A, or deleted
  VARIANT 35: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXNPKANREKMTQIMFXTFNXPAMYVAIQAVLSLYASGRTTGIXXXXXXXXXXXXXXXXXXXX

SEQ ID NO: 247 - protein, 81 aa, synthetic construct
  VARIANT 1..20: note = X can be G, A, or deleted
  VARIANT 36: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXNPKANREKMTQIMFEXFNXPAMYVAIQAVLSLYASGRTTGIXXXXXXXXXXXXXXXXXXXX

SEQ ID NO: 248 - protein, 81 aa, synthetic construct
  VARIANT 1..20: note = X can be G, A, or deleted
  VARIANT 37: note = X can be any naturally-occurring amino acid or any unnatural amino acid
  VARIANT 39: note = X can be any naturally-occurring amino acid or any unnatural amino acid
  VARIANT 62..81: note = X can be G, A, or deleted
XXXXXXXXXXXXXXXXXXXXNPKANREKMTQIMFETXNXPAMYVAIQAVLSLYASGRTTGIXXXXXXXXXXXXXXXXXXXX

SEQ ID NO: 249 - protein, 81 aa, synthetic construct
  VARIANT 1..20: note = X can be G, A, or deleted
  VARIANT 38..39: note = X can be any naturally-occurring amino acid or any unnatural amino acid
  VARIANT 62..81: note = X can be G, A, or deleted
XXXXXXXXXXXXXXXXXXXXNPKANREKMTQIMFETFXXPAMYVAIQAVLSLYASGRTTGIXXXXXXXXXXXXXXXXXXXX

SEQ ID NO: 250 - protein, 81 aa, synthetic construct
  VARIANT 1..20: note = X can be G, A, or deleted
  VARIANT 39..40: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXXAMYVAIQAVLSLYASGRTTGIXXXXXXXXXXXXXXXXXXXX

SEQ ID NO: 251 - protein, 81 aa, synthetic construct
  VARIANT 1..20: note = X can be G, A, or deleted
  VARIANT 39: note = X can be any naturally-occurring amino acid or any unnatural amino acid
  VARIANT 41: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPXMYVAIQAVLSLYASGRTTGIXXXXXXXXXXXXXXXXXXXX

SEQ ID NO: 252 - protein, 81 aa, synthetic construct
  VARIANT 1..20: note = X can be G, A, or deleted
  VARIANT 39: note = X can be any naturally-occurring amino acid or any unnatural amino acid
  VARIANT 42: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAXYVAIQAVLSLYASGRTTGIXXXXXXXXXXXXXXXXXXXX

SEQ ID NO: 253 - protein, 81 aa, synthetic construct
  VARIANT 1..20: note = X can be G, A, or deleted
  VARIANT 39: note = X can be any naturally-occurring amino acid or any unnatural amino acid
  VARIANT 43: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMXVAIQAVLSLYASGRTTGIXXXXXXXXXXXXXXXXXXXX

SEQ ID NO: 254 - protein, 81 aa, synthetic construct
  VARIANT 1..20: note = X can be G, A, or deleted
  VARIANT 39: note = X can be any naturally-occurring amino acid or any unnatural amino acid
  VARIANT 44: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYXAIQAVLSLYASGRTTGIXXXXXXXXXXXXXXXXXXXX

SEQ ID NO: 255 - protein, 81 aa, synthetic construct
  VARIANT 1..20: note = X can be G, A, or deleted
  VARIANT 39: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVXIQAVLSLYASGRTTGIXXXXXXXXXXXXXXXXXXXX

SEQ ID NO: 256 - protein, 81 aa, synthetic construct
  VARIANT 1..20: note = X can be G, A, or deleted
  VARIANT 39: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVAXQAVLSLYASGRTTGIXXXXXXXXXXXXXXXXXXXX

SEQ ID NO: 257 - protein, 81 aa, synthetic construct
  VARIANT 1..20: note = X can be G, A, or deleted
  VARIANT 39: note = X can be any naturally-occurring amino acid or any unnatural amino acid
  VARIANT 47: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVAIXAVLSLYASGRTTGIXXXXXXXXXXXXXXXXXXXX

SEQ ID NO: 258 - protein, 81 aa, synthetic construct
  VARIANT 1..20: note = X can be G, A, or deleted
  VARIANT 39: note = X can be any naturally-occurring amino acid or any unnatural amino acid
  VARIANT 48: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVAIQXVLSLYASGRTTGIXXXXXXXXXXXXXXXXXXXX

SEQ ID NO: 259 - protein, 81 aa, synthetic construct
  VARIANT 1..20: note = X can be G, A, or deleted
  VARIANT 39: note = X can be any naturally-occurring amino acid or any unnatural amino acid
  VARIANT 49: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVAIQAXLSLYASGRTTGIXXXXXXXXXXXXXXXXXXXX

SEQ ID NO: 260 - protein, 81 aa, synthetic construct
  VARIANT 1..20: note = X can be G, A, or deleted
  VARIANT 39: note = X can be any naturally-occurring amino acid or any unnatural amino acid
  VARIANT 50: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVAIQAVXSLYASGRTTGIXXXXXXXXXXXXXXXXXXXX

SEQ ID NO: 261 - protein, 81 aa, synthetic construct
  VARIANT 1..20: note = X can be G, A, or deleted
  VARIANT 39: note = X can be any naturally-occurring amino acid or any unnatural amino acid
  VARIANT 51: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVAIQAVLXLYASGRTTGIXXXXXXXXXXXXXXXXXXXX

SEQ ID NO: 262 - protein, 81 aa, synthetic construct
  VARIANT 1..20: note = X can be G, A, or deleted
  VARIANT 39: note = X can be any naturally-occurring amino acid or any unnatural amino acid
  VARIANT 52: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVAIQAVLSXYASGRTTGIXXXXXXXXXXXXXXXXXXXX

SEQ ID NO: 263 - protein, 81 aa, synthetic construct
  VARIANT 1..20: note = X can be G, A, or deleted
  VARIANT 39: note = X can be any naturally-occurring amino acid or any unnatural amino acid
  VARIANT 53: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVAIQAVLSLXASGRTTGIXXXXXXXXXXXXXXXXXXXX

SEQ ID NO: 264 - protein, 81 aa, synthetic construct
  VARIANT 1..20: note = X can be G, A, or deleted
  VARIANT 39: note = X can be any naturally-occurring amino acid or any unnatural amino acid
  VARIANT 54: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVAIQAVLSLYXSGRTTGIXXXXXXXXXXXXXXXXXXXX

SEQ ID NO: 265 - protein, 81 aa, synthetic construct
  VARIANT 1..20: note = X can be G, A, or deleted
  VARIANT 39: note = X can be any naturally-occurring amino acid or any unnatural amino acid
  VARIANT 55: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVAIQAVLSLYAXGRTTGIXXXXXXXXXXXXXXXXXXXX

SEQ ID NO: 266 - protein, 81 aa, synthetic construct
  VARIANT 1..20: note = X can be G, A, or deleted
  VARIANT 39: note = X can be any naturally-occurring amino acid or any unnatural amino acid
  VARIANT 56: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVAIQAVLSLYASXRTTGIXXXXXXXXXXXXXXXXXXXX

SEQ ID NO: 267 - protein, 81 aa, synthetic construct
  VARIANT 1..20: note = X can be G, A, or deleted
  VARIANT 39: note = X can be any naturally-occurring amino acid or any unnatural amino acid
  VARIANT 57: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVAIQAVLSLYASGXTTGIXXXXXXXXXXXXXXXXXXXX

SEQ ID NO: 268 - protein, 81 aa, synthetic construct
  VARIANT 1..20: note = X can be G, A, or deleted
  VARIANT 39: note = X can be any naturally-occurring amino acid or any unnatural amino acid
  VARIANT 58: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVAIQAVLSLYASGRXTGIXXXXXXXXXXXXXXXXXXXX

SEQ ID NO: 269 - protein, 81 aa, synthetic construct
  VARIANT 1..20: note = X can be G, A, or deleted
  VARIANT 39: note = X can be any naturally-occurring amino acid or any unnatural amino acid
  VARIANT 59: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVAIQAVLSLYASGRTXGIXXXXXXXXXXXXXXXXXXXX

SEQ ID NO: 270 - protein, 81 aa, synthetic construct
  VARIANT 1..20: note = X can be G, A, or deleted
  VARIANT 39: note = X can be any naturally-occurring amino acid or any unnatural amino acid
  VARIANT 60: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVAIQAVLSLYASGRTTXIXXXXXXXXXXXXXXXXXXXX

SEQ ID NO: 271 - protein, 81 aa, synthetic construct
  VARIANT 1..20: note = X can be G, A, or deleted
  VARIANT 39: note = X can be any naturally-occurring amino acid or any unnatural amino acid
  VARIANT 61: note = X can be any naturally-occurring amino acid or any unnatural amino acid
  VARIANT 62..81: note = X can be G, A, or deleted
XXXXXXXXXXXXXXXXXXXXNPKANREKMTQIMFETFNXPAMYVAIQAVLSLYASGRTTGXXXXXXXXXXXXXXXXXXXXX

SEQ ID NO: 272 - protein, 60 aa, synthetic construct
  VARIANT 1..20: note = X can be G, A, or deleted
  VARIANT 21: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXXPKANREKMTQIMFETFNXPXXXXXXXXXXXXXXXXXXXX

SEQ ID NO: 273 - protein, 60 aa, synthetic construct
  VARIANT 1..20: note = X can be G, A, or deleted
  VARIANT 22: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXNXKANREKMTQIMFETFNXPXXXXXXXXXXXXXXXXXXXX

SEQ ID NO: 274 - protein, 60 aa, synthetic construct
  VARIANT 1..20: note = X can be G, A, or deleted
  VARIANT 23: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXNPXANREKMTQIMFETFNXPXXXXXXXXXXXXXXXXXXXX

SEQ ID NO: 275 - protein, 60 aa, synthetic construct
  VARIANT 1..20: note = X can be G, A, or deleted
  VARIANT 24: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXNPKXNREKMTQIMFETFNXPXXXXXXXXXXXXXXXXXXXX

SEQ ID NO: 276 - protein, 60 aa, synthetic construct
  VARIANT 1..20: note = X can be G, A, or deleted
  VARIANT 25: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXNPKAXREKMTQIMFETFNXPXXXXXXXXXXXXXXXXXXXX

SEQ ID NO: 277 - protein, 60 aa, synthetic construct
  VARIANT 1..20: note = X can be G, A, or deleted
  VARIANT 26: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXNPKANXEKMTQIMFETFNXPXXXXXXXXXXXXXXXXXXXX

SEQ ID NO: 278 - protein, 60 aa, synthetic construct
  VARIANT 1..20: note = X can be G, A, or deleted
  VARIANT 27: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXNPKANRXKMTQIMFETFNXPXXXXXXXXXXXXXXXXXXXX

SEQ ID NO: 279 - protein, 60 aa, synthetic construct
  VARIANT 1..20: note = X can be G, A, or deleted
  VARIANT 28: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXNPKANREXMTQIMFETFNXPXXXXXXXXXXXXXXXXXXXX

SEQ ID NO: 280 - protein, 60 aa, synthetic construct
  VARIANT 1..20: note = X can be G, A, or deleted
  VARIANT 29: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXNPKANREKXTQIMFETFNXPXXXXXXXXXXXXXXXXXXXX

SEQ ID NO: 281 - protein, 60 aa, synthetic construct
  VARIANT 1..20: note = X can be G, A, or deleted
  VARIANT 30: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXNPKANREKMXQIMFETFNXPXXXXXXXXXXXXXXXXXXXX

SEQ ID NO: 282 - protein, 60 aa, synthetic construct
  VARIANT 1..20: note = X can be G, A, or deleted
  VARIANT 31: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXNPKANREKMTXIMFETFNXPXXXXXXXXXXXXXXXXXXXX

SEQ ID NO: 283 - protein, 60 aa, synthetic construct
  VARIANT 1..20: note = X can be G, A, or deleted
  VARIANT 32: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXNPKANREKMTQXMFETFNXPXXXXXXXXXXXXXXXXXXXX

SEQ ID NO: 284 - protein, 60 aa, synthetic construct
  VARIANT 1..20: note = X can be G, A, or deleted
  VARIANT 33: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXNPKANREKMTQIXFETFNXPXXXXXXXXXXXXXXXXXXXX

SEQ ID NO: 285 - protein, 60 aa, synthetic construct
  VARIANT 1..20: note = X can be G, A, or deleted
  VARIANT 34: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXNPKANREKMTQIMXETFNXPXXXXXXXXXXXXXXXXXXXX

SEQ ID NO: 286 - protein, 60 aa, synthetic construct
  VARIANT 1..20: note = X can be G, A, or deleted
  VARIANT 35: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXNPKANREKMTQIMFXTFNXPXXXXXXXXXXXXXXXXXXXX

SEQ ID NO: 287 - protein, 60 aa, synthetic construct
  VARIANT 1..20: note = X can be G, A, or deleted
  VARIANT 36: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXNPKANREKMTQIMFEXFNXPXXXXXXXXXXXXXXXXXXXX

SEQ ID NO: 288 - protein, 60 aa, synthetic construct
  VARIANT 1..20: note = X can be G, A, or deleted
  VARIANT 37: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXNPKANREKMTQIMFETXNXPXXXXXXXXXXXXXXXXXXXX

SEQ ID NO: 289 - protein, 60 aa, synthetic construct
  VARIANT 1..20: note = X can be G, A, or deleted
  VARIANT 38..39: note = X can be any naturally-occurring amino acid or any unnatural amino acid
  VARIANT 41..60: note = X can be G, A, or deleted
XXXXXXXXXXXXXXXXXXXXNPKANREKMTQIMFETFXXPXXXXXXXXXXXXXXXXXXXX

SEQ ID NO: 290 - protein, 60 aa, synthetic construct
  VARIANT 1..20: note = X can be G, A, or deleted
  VARIANT 39..40: note = X can be any naturally-occurring amino acid or any unnatural amino acid
  VARIANT 41..60: note = X can be G, A, or deleted
XXXXXXXXXXXXXXXXXXXXNPKANREKMTQIMFETFNXXXXXXXXXXXXXXXXXXXXXX

SEQ ID NO: 291 - protein, 50 aa, synthetic construct
  VARIANT 1..20: note = X can be any naturally-occurring amino acid or any unnatural amino acid or deleted
  VARIANT 21: note = X can be any naturally-occurring amino acid or any unnatural amino acid
  VARIANT 31..50: note = X can be any naturally-occurring amino acid or any unnatural amino acid or deleted
XXXXXXXXXXXXXXXXXXXXXPHAILRLDLXXXXXXXXXXXXXXXXXXXX

SEQ ID NO: 292 - protein, 50 aa, synthetic construct
  VARIANT 1..20: note = X can be G, A, or deleted
  VARIANT 22: note = X can be any naturally-occurring amino acid or any unnatural amino acid
  VARIANT 31..50: note = X can be G, A, or deleted
XXXXXXXXXXXXXXXXXXXXLXHAILRLDLXXXXXXXXXXXXXXXXXXXX

SEQ ID NO: 293 - protein, 50 aa, synthetic construct
  VARIANT 1..20: note = X can be G, A, or deleted
  VARIANT 23: note = X can be any naturally-occurring amino acid or any unnatural amino acid
  VARIANT 31..50: note = X can be G, A, or deleted
XXXXXXXXXXXXXXXXXXXXLPXAILRLDLXXXXXXXXXXXXXXXXXXXX

SEQ ID NO: 294 - protein, 50 aa, synthetic construct
  VARIANT 1..20: note = X can be G, A, or deleted
  VARIANT 24: note = X can be any naturally-occurring amino acid or any unnatural amino acid
  VARIANT 31..50: note = X can be G, A, or deleted
XXXXXXXXXXXXXXXXXXXXLPHXILRLDLXXXXXXXXXXXXXXXXXXXX

SEQ ID NO: 295 - protein, 50 aa, synthetic construct
  VARIANT 1..20: note = X can be G, A, or deleted
  VARIANT 25: note = X can be any naturally-occurring amino acid or any unnatural amino acid
  VARIANT 31..50: note = X can be G, A, or deleted
XXXXXXXXXXXXXXXXXXXXLPHAXLRLDLXXXXXXXXXXXXXXXXXXXX

SEQ ID NO: 296 - protein, 50 aa, synthetic construct
  VARIANT 1..20: note = X can be G, A, or deleted
  VARIANT 26: note = X can be any naturally-occurring amino acid or any unnatural amino acid
  VARIANT 31..50: note = X can be G, A, or deleted
XXXXXXXXXXXXXXXXXXXXLPHAIXRLDLXXXXXXXXXXXXXXXXXXXX

SEQ ID NO: 297 - protein, 50 aa, synthetic construct
  VARIANT 1..20: note = X can be G, A, or deleted
  VARIANT 27: note = X can be any naturally-occurring amino acid or any unnatural amino acid
  VARIANT 31..50: note = X can be G, A, or deleted
XXXXXXXXXXXXXXXXXXXXLPHAILXLDLXXXXXXXXXXXXXXXXXXXX

SEQ ID NO: 298 - protein, 50 aa, synthetic construct
  VARIANT 1..20: note = X can be G, A, or deleted
  VARIANT 29: note = X can be any naturally-occurring amino acid or any unnatural amino acid
  VARIANT 31..50: note = X can be G, A, or deleted
XXXXXXXXXXXXXXXXXXXXLPHAILRLXLXXXXXXXXXXXXXXXXXXXX

SEQ ID NO: 299 - protein, 50 aa, synthetic construct
  VARIANT 1..20: note = X can be G, A, or deleted
  VARIANT 30: note = X can be any naturally-occurring amino acid or any unnatural amino acid
  VARIANT 31..50: note = X can be G, A, or deleted
XXXXXXXXXXXXXXXXXXXXLPHAILRLDXXXXXXXXXXXXXXXXXXXXX

SEQ ID NO: 300 - protein, 50 aa, synthetic construct
  VARIANT 1..20: note = X can be any naturally-occurring amino acid or any unnatural amino acid or deleted
  VARIANT 21: note = X can be any naturally-occurring amino acid or any unnatural amino acid
  VARIANT 29: note = X can be any naturally-occurring amino acid or any unnatural amino acid
  VARIANT 31..50: note = X can be any naturally-occurring amino acid or any unnatural amino acid or deleted
XXXXXXXXXXXXXXXXXXXXXYALPHAIXRXXXXXXXXXXXXXXXXXXXX

SEQ ID NO: 301 - protein, 50 aa, synthetic construct
  VARIANT 1..20: note = X can be G, A, or deleted
  VARIANT 22: note = X can be any naturally-occurring amino acid or any unnatural amino acid
  VARIANT 29: note = X can be any naturally-occurring amino acid or any unnatural amino acid
  VARIANT 31..50: note = X can be G, A, or deleted
XXXXXXXXXXXXXXXXXXXXGXALPHAIXRXXXXXXXXXXXXXXXXXXXX

SEQ ID NO: 302 - protein, 50 aa, synthetic construct
  VARIANT 1..20: note = X can be G, A, or deleted
  VARIANT 23: note = X can be any naturally-occurring amino acid or any unnatural amino acid
  VARIANT 29: note = X can be any naturally-occurring amino acid or any unnatural amino acid
  VARIANT 31..50: note = X can be G, A, or deleted
XXXXXXXXXXXXXXXXXXXXGYXLPHAIXRXXXXXXXXXXXXXXXXXXXX

SEQ ID NO: 303 - protein, 50 aa, synthetic construct
  VARIANT 1..20: note = X can be G, A, or deleted
  VARIANT 24: note = X can be any naturally-occurring amino acid or any unnatural amino acid
  VARIANT 29: note = X can be any naturally-occurring amino acid or any unnatural amino acid
  VARIANT 31..50: note = X can be G, A, or deleted
XXXXXXXXXXXXXXXXXXXXGYAXPHAIXRXXXXXXXXXXXXXXXXXXXX

SEQ ID NO: 304 - protein, 50 aa, synthetic construct
  VARIANT 1..20: note = X can be G, A, or deleted
  VARIANT 25: note = X can be any naturally-occurring amino acid or any unnatural amino acid
  VARIANT 29: note = X can be any naturally-occurring amino acid or any unnatural amino acid
  VARIANT 31..50: note = X can be G, A, or deleted
XXXXXXXXXXXXXXXXXXXXGYALXHAIXRXXXXXXXXXXXXXXXXXXXX

SEQ ID NO: 305 - protein, 50 aa, synthetic construct
  VARIANT 1..20: note = X can be G, A, or deleted
  VARIANT 26: note = X can be any naturally-occurring amino acid or any unnatural amino acid
  VARIANT 29: note = X can be any naturally-occurring amino acid or any unnatural amino acid
  VARIANT 31..50: note = X can be G, A, or deleted
XXXXXXXXXXXXXXXXXXXXGYALPXAIXRXXXXXXXXXXXXXXXXXXXX

SEQ ID NO: 306 - protein, 50 aa, synthetic construct
  VARIANT 1..20: note = X can be G, A, or deleted
  VARIANT 27: note = X can be any naturally-occurring amino acid or any unnatural amino acid
  VARIANT 29: note = X can be any naturally-occurring amino acid or any unnatural amino acid
  VARIANT 31..50: note = X can be G, A, or deleted
XXXXXXXXXXXXXXXXXXXXGYALPHXIXRXXXXXXXXXXXXXXXXXXXX

SEQ ID NO: 307 - protein, 50 aa, synthetic construct
  VARIANT 1..20: note = X can be G, A, or deleted
  VARIANT 28..29: note = X can be any naturally-occurring amino acid or any unnatural amino acid
  VARIANT 31..50: note = X can be G, A, or deleted
XXXXXXXXXXXXXXXXXXXXGYALPHAXXRXXXXXXXXXXXXXXXXXXXX

SEQ ID NO: 308 - protein, 50 aa, synthetic construct
  VARIANT 1..20: note = X can be G, A, or deleted
  VARIANT 29..30: note = X can be any naturally-occurring amino acid or any unnatural amino acid
  VARIANT 31..50: note = X can be G, A, or deleted
XXXXXXXXXXXXXXXXXXXXGYALPHAIXXXXXXXXXXXXXXXXXXXXXX

SEQ ID NO: 309 - protein, 50 aa, synthetic construct
  VARIANT 1..20: note = X can be G, A, or deleted
  VARIANT 27: note = X can be any naturally-occurring amino acid or any unnatural amino acid
  VARIANT 31..50: note = X can be G, A, or deleted
XXXXXXXXXXXXXXXXXXXXGDGVTHXVPIXXXXXXXXXXXXXXXXXXXX

SEQ ID NO: 310 - protein, 50 aa, synthetic construct
  VARIANT 1..20: note = X can be G, A, or deleted
  VARIANT 26: note = X can be any naturally-occurring amino acid or any unnatural amino acid
  VARIANT 31..50: note = X can be G, A, or deleted
XXXXXXXXXXXXXXXXXXXXDGVTHXVPIYXXXXXXXXXXXXXXXXXXXX

SEQ ID NO: 311 - protein, 50 aa, synthetic construct
  VARIANT 1..20: note = X can be G, A, or deleted
  VARIANT 25: note = X can be any naturally-occurring amino acid or any unnatural amino acid
  VARIANT 31..50: note = X can be G, A, or deleted
XXXXXXXXXXXXXXXXXXXXGVTHXVPIYEXXXXXXXXXXXXXXXXXXXX

SEQ ID NO: 312 - protein, 50 aa, synthetic construct
  VARIANT 1..20: note = X can be G, A, or deleted
  VARIANT 24: note = X can be any naturally-occurring amino acid or any unnatural amino acid
  VARIANT 31..50: note = X can be G, A, or deleted
XXXXXXXXXXXXXXXXXXXXVTHXVPIYEGXXXXXXXXXXXXXXXXXXXX

SEQ ID NO: 313 - protein, 50 aa, synthetic construct
  VARIANT 1..20: note = X can be G, A, or deleted
  VARIANT 23: note = X can be any naturally-occurring amino acid or any unnatural amino acid
  VARIANT 31..50: note = X can be G, A, or deleted
XXXXXXXXXXXXXXXXXXXXTHXVPIYEGYXXXXXXXXXXXXXXXXXXXX

SEQ ID NO: 314 - protein, 50 aa, synthetic construct
  VARIANT 1..20: note = X can be G, A, or deleted
  VARIANT 22: note = X can be any naturally-occurring amino acid or any unnatural amino acid
  VARIANT 31..50: note = X can be G, A, or deleted
XXXXXXXXXXXXXXXXXXXXHXVPIYEGYAXXXXXXXXXXXXXXXXXXXX

SEQ ID NO: 315 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXVPIYEGYALXXXXXXXXXXXXXXXXXXXX

SEQ ID NO: 316 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXPIYEGYALPXXXXXXXXXXXXXXXXXXXX

SEQ ID NO: 317 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXIYEGYALPHXXXXXXXXXXXXXXXXXXXX

SEQ ID NO: 318 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXYEGYALPHAXXXXXXXXXXXXXXXXXXXX

SEQ ID NO: 319 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXEGYALPHAIXXXXXXXXXXXXXXXXXXXX

SEQ ID NO: 320 - protein, 50 aa, synthetic construct
  VARIANT 1..20: note = X can be G, A, or deleted
  VARIANT 30: note = X can be any naturally-occurring amino acid or any unnatural amino acid
  VARIANT 31..50: note = X can be G, A, or deleted
XXXXXXXXXXXXXXXXXXXXEGYALPHAIXXXXXXXXXXXXXXXXXXXXX

SEQ ID NO: 321 - protein, 50 aa, synthetic construct
  VARIANT 1..20: note = X can be G, A, or deleted
  VARIANT 29: note = X can be any naturally-occurring amino acid or any unnatural amino acid
  VARIANT 31..50: note = X can be G, A, or deleted
XXXXXXXXXXXXXXXXXXXXGYALPHAIXRXXXXXXXXXXXXXXXXXXXX

SEQ ID NO: 322 - protein, 50 aa, synthetic construct
  VARIANT 1..20: note = X can be G, A, or deleted
  VARIANT 28: note = X can be any naturally-occurring amino acid or any unnatural amino acid
  VARIANT 31..50: note = X can be G, A, or deleted
XXXXXXXXXXXXXXXXXXXXYALPHAIXRLXXXXXXXXXXXXXXXXXXXX

SEQ ID NO: 323 - protein, 50 aa, synthetic construct
  VARIANT 1..20: note = X can be G, A, or deleted
  VARIANT 27: note = X can be any naturally-occurring amino acid or any unnatural amino acid
  VARIANT 31..50: note = X can be G, A, or deleted
XXXXXXXXXXXXXXXXXXXXALPHAIXRLDXXXXXXXXXXXXXXXXXXXX

SEQ ID NO: 324 - protein, 50 aa, synthetic construct; identical to SEQ ID NO: 296
  VARIANT 1..20: note = X can be G, A, or deleted
  VARIANT 26: note = X can be any naturally-occurring amino acid or any unnatural amino acid
  VARIANT 31..50: note = X can be G, A, or deleted

SEQ ID NO: 325 - protein, 50 aa, synthetic construct
  VARIANT 1..20: note = X can be G, A, or deleted
  VARIANT 25: note = X can be any naturally-occurring amino acid or any unnatural amino acid
  VARIANT 31..50: note = X can be G, A, or deleted
XXXXXXXXXXXXXXXXXXXXPHAIXRLDLAXXXXXXXXXXXXXXXXXXXX

SEQ ID NO: 326 - protein, 50 aa, synthetic construct
  VARIANT 1..20: note = X can be G, A, or deleted
  VARIANT 24: note = X can be any naturally-occurring amino acid or any unnatural amino acid
  VARIANT 31..50: note = X can be G, A, or deleted
XXXXXXXXXXXXXXXXXXXXHAIXRLDLAGXXXXXXXXXXXXXXXXXXXX

SEQ ID NO: 327 - protein, 50 aa, synthetic construct
  VARIANT 1..20: note = X can be G, A, or deleted
  VARIANT 23: note = X can be any naturally-occurring amino acid or any unnatural amino acid
  VARIANT 31..50: note = X can be G, A, or deleted
XXXXXXXXXXXXXXXXXXXXAIXRLDLAGRXXXXXXXXXXXXXXXXXXXX

SEQ ID NO: 328 - protein, 50 aa, synthetic construct
  VARIANT 1..20: note = X can be G, A, or deleted
  VARIANT 22: note = X can be any naturally-occurring amino acid or any unnatural amino acid
  VARIANT 31..50: note = X can be G, A, or deleted
XXXXXXXXXXXXXXXXXXXXIXRLDLAGRDXXXXXXXXXXXXXXXXXXXX

SEQ ID NO: 329 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXRLDLAGRDLXXXXXXXXXXXXXXXXXXXX

SEQ ID NO: 330 - protein, 50 aa, synthetic construct
  VARIANT 1..20: note = X can be G, A, or deleted
  VARIANT 31..50: note = X can be G, A, or deleted
XXXXXXXXXXXXXXXXXXXXLDLAGRDLTDXXXXXXXXXXXXXXXXXXXX

SEQ ID NO: 331 - protein, 50 aa, synthetic construct
  VARIANT 1..20: note = X can be G, A, or deleted
  VARIANT 31..50: note = X can be G, A, or deleted
XXXXXXXXXXXXXXXXXXXXDLAGRDLTDYXXXXXXXXXXXXXXXXXXXX

SEQ ID NO: 332 - protein, 50 aa, synthetic construct
  VARIANT 1..20: note = X can be G, A, or deleted
  VARIANT 31..50: note = X can be G, A, or deleted
XXXXXXXXXXXXXXXXXXXXLAGRDLTDYLXXXXXXXXXXXXXXXXXXXX

SEQ ID NO: 333 - protein, 50 aa, synthetic construct
  VARIANT 1..20: note = X can be G, A, or deleted
  VARIANT 31..50: note = X can be G, A, or deleted
XXXXXXXXXXXXXXXXXXXXAGRDLTDYLMXXXXXXXXXXXXXXXXXXXX

SEQ ID NO: 334 - protein, 50 aa, synthetic construct
  VARIANT 1..20: note = X can be G, A, or deleted
  VARIANT 31..50: note = X can be G, A, or deleted
XXXXXXXXXXXXXXXXXXXXGRDLTDYLMKXXXXXXXXXXXXXXXXXXXX

SEQ ID NO: 335 - protein, 50 aa, synthetic construct
  VARIANT 1..20: note = X can be G, A, or deleted
  VARIANT 31..50: note = X can be G, A, or deleted
XXXXXXXXXXXXXXXXXXXXRDLTDYLMKIXXXXXXXXXXXXXXXXXXXX

SEQ ID NO: 336 - protein, 50 aa, synthetic construct
  VARIANT 1..20: note = X can be G, A, or deleted
  VARIANT 31..50: note = X can be G, A, or deleted
XXXXXXXXXXXXXXXXXXXXDLTDYLMKILXXXXXXXXXXXXXXXXXXXX

SEQ ID NO: 337 - protein, 50 aa, synthetic construct
  VARIANT 1..20: note = X can be G, A, or deleted
  VARIANT 31..50: note = X can be G, A, or deleted
XXXXXXXXXXXXXXXXXXXXLTDYLMKILTXXXXXXXXXXXXXXXXXXXX

SEQ ID NO: 338 - protein, 50 aa, synthetic construct
  VARIANT 1..20: note = X can be G, A, or deleted
  VARIANT 31..50: note = X can be G, A, or deleted
XXXXXXXXXXXXXXXXXXXXTDYLMKILTEXXXXXXXXXXXXXXXXXXXX

SEQ ID NO: 339 - protein, 50 aa, synthetic construct
  VARIANT 1..20: note = X can be G, A, or deleted
  VARIANT 31..50: note = X can be G, A, or deleted
XXXXXXXXXXXXXXXXXXXXDYLMKILTERXXXXXXXXXXXXXXXXXXXX

SEQ ID NO: 340 - protein, 50 aa, synthetic construct
  VARIANT 1..20: note = X can be G, A, or deleted
  VARIANT 31..50: note = X can be G, A, or deleted
XXXXXXXXXXXXXXXXXXXXYLMKILTERGXXXXXXXXXXXXXXXXXXXX

SEQ ID NO: 341 - protein, 50 aa, synthetic construct
  VARIANT 1..20: note = X can be G, A, or deleted
  VARIANT 31..50: note = X can be G, A, or deleted
XXXXXXXXXXXXXXXXXXXXLMKILTERGYXXXXXXXXXXXXXXXXXXXX

SEQ ID NO: 342 - protein, 50 aa, synthetic construct
  VARIANT 1..20: note = X can be G, A, or deleted
  VARIANT 31..50: note = X can be G, A, or deleted
XXXXXXXXXXXXXXXXXXXXMKILTERGYSXXXXXXXXXXXXXXXXXXXX

SEQ ID NO: 343 - protein, 50 aa, synthetic construct
  VARIANT 1..20: note = X can be G, A, or deleted
  VARIANT 31..50: note = X can be G, A, or deleted
XXXXXXXXXXXXXXXXXXXXKILTERGYSFXXXXXXXXXXXXXXXXXXXX

SEQ ID NO: 344 - protein, 61 aa, synthetic construct
  VARIANT 1..20: note = X can be G, A, or deleted
  VARIANT 21: note = X can be any naturally-occurring amino acid or any unnatural amino acid
  VARIANT 35: note = X can be any naturally-occurring amino acid or any unnatural amino acid
  VARIANT 42..61: note = X can be G, A, or deleted
XXXXXXXXXXXXXXXXXXXXXVPIYEGYALPHAIXRLDLAGXXXXXXXXXXXXXXXXXXXX

SEQ ID NO: 345 - protein, 60 aa, synthetic construct
  VARIANT 1..20: note = X can be G, A, or deleted
  VARIANT 21: note = X can be any naturally-occurring amino acid or any unnatural amino acid
  VARIANT 34: note = X can be any naturally-occurring amino acid or any unnatural amino acid
  VARIANT 41..60: note = X can be G, A, or deleted
XXXXXXXXXXXXXXXXXXXXXPIYEGYALPHAIXRLDLAGXXXXXXXXXXXXXXXXXXXX

SEQ ID NO: 346 - protein, 60 aa, synthetic construct
  VARIANT 1..20: note = X can be G, A, or deleted
  VARIANT 22: note = X can be any naturally-occurring amino acid or any unnatural amino acid
  VARIANT 34: note = X can be any naturally-occurring amino acid or any unnatural amino acid
  VARIANT 41..60: note = X can be G, A, or deleted
XXXXXXXXXXXXXXXXXXXXVXIYEGYALPHAIXRLDLAGXXXXXXXXXXXXXXXXXXXX

SEQ ID NO: 347 - protein, 60 aa, synthetic construct
  VARIANT 1..20: note = X can be G, A, or deleted
  VARIANT 23: note = X can be any naturally-occurring amino acid or any unnatural amino acid
  VARIANT 34: note = X can be any naturally-occurring amino acid or any unnatural amino acid
  VARIANT 41..60: note = X can be G, A, or deleted
XXXXXXXXXXXXXXXXXXXXVPXYEGYALPHAIXRLDLAGXXXXXXXXXXXXXXXXXXXX

SEQ ID NO: 348 - protein, 60 aa, synthetic construct
  VARIANT 1..20: note = X can be G, A, or deleted
  VARIANT 24: note = X can be any naturally-occurring amino acid or any unnatural amino acid
  VARIANT 34: note = X can be any naturally-occurring amino acid or any unnatural amino acid
  VARIANT 41..60: note = X can be G, A, or deleted
XXXXXXXXXXXXXXXXXXXXVPIXEGYALPHAIXRLDLAGXXXXXXXXXXXXXXXXXXXX

SEQ ID NO: 349 - protein, 60 aa, synthetic construct
  VARIANT 1..20: note = X can be G, A, or deleted
  VARIANT 25: note = X can be any naturally-occurring amino acid or any unnatural amino acid
  VARIANT 34: note = X can be any naturally-occurring amino acid or any unnatural amino acid
  VARIANT 41..60: note = X can be G, A, or deleted
XXXXXXXXXXXXXXXXXXXXVPIYXGYALPHAIXRLDLAGXXXXXXXXXXXXXXXXXXXX

SEQ ID NO: 350 - protein, 60 aa, synthetic construct
  VARIANT 1..20: note = X can be G, A, or deleted
  VARIANT 26: note = X can be any naturally-occurring amino acid or any unnatural amino acid
  VARIANT 34: note = X can be any naturally-occurring amino acid or any unnatural amino acid
  VARIANT 41..60: note = X can be G, A, or deleted
XXXXXXXXXXXXXXXXXXXXVPIYEXYALPHAIXRLDLAGXXXXXXXXXXXXXXXXXXXX

SEQ ID NO: 351 - protein, 60 aa, synthetic construct
  VARIANT 1..20: note = X can be G, A, or deleted
  VARIANT 27: note = X can be any naturally-occurring amino acid or any unnatural amino acid
  VARIANT 34: note = X can be any naturally-occurring amino acid or any unnatural amino acid
  VARIANT 41..60: note = X can be G, A, or deleted
XXXXXXXXXXXXXXXXXXXXVPIYEGXALPHAIXRLDLAGXXXXXXXXXXXXXXXXXXXX

SEQ ID NO: 352 - protein, 60 aa, synthetic construct
  VARIANT 1..20: note = X can be G, A, or deleted
  VARIANT 28: note = X can be any naturally-occurring amino acid or any unnatural amino acid
  VARIANT 34: note = X can be any naturally-occurring amino acid or any unnatural amino acid
  VARIANT 41..60: note = X can be G, A, or deleted
XXXXXXXXXXXXXXXXXXXXVPIYEGYXLPHAIXRLDLAGXXXXXXXXXXXXXXXXXXXX

SEQ ID NO: 353 - protein, 60 aa, synthetic construct
  VARIANT 1..20: note = X can be G, A, or deleted
  VARIANT 29: note = X can be any naturally-occurring amino acid or any unnatural amino acid
  VARIANT 34: note = X can be any naturally-occurring amino acid or any unnatural amino acid
  VARIANT 41..60: note = X can be G, A, or deleted
XXXXXXXXXXXXXXXXXXXXVPIYEGYAXPHAIXRLDLAGXXXXXXXXXXXXXXXXXXXX

SEQ ID NO: 354 - protein, 60 aa, synthetic construct
  VARIANT 1..20: note = X can be G, A, or deleted
  VARIANT 30: note = X can be any naturally-occurring amino acid or any unnatural amino acid
  VARIANT 34: note = X can be any naturally-occurring amino acid or any unnatural amino acid
  VARIANT 41..60: note = X can be G, A, or deleted
XXXXXXXXXXXXXXXXXXXXVPIYEGYALXHAIXRLDLAGXXXXXXXXXXXXXXXXXXXX

SEQ ID NO: 355 - protein, 60 aa, synthetic construct
  VARIANT 1..20: note = X can be G, A, or deleted
  VARIANT 31: note = X can be any naturally-occurring amino acid or any unnatural amino acid
  VARIANT 34: note = X can be any naturally-occurring amino acid or any unnatural amino acid
  VARIANT 41..60: note = X can be G, A, or deleted
XXXXXXXXXXXXXXXXXXXXVPIYEGYALPXAIXRLDLAGXXXXXXXXXXXXXXXXXXXX

SEQ ID NO: 356 - protein, 60 aa, synthetic construct
  VARIANT 1..20: note = X can be G, A, or deleted
  VARIANT 32: note = X can be any naturally-occurring amino acid or any unnatural amino acid
  VARIANT 34: note = X can be any naturally-occurring amino acid or any unnatural amino acid
  VARIANT 41..60: note = X can be G, A, or deleted
XXXXXXXXXXXXXXXXXXXXVPIYEGYALPHXIXRLDLAGXXXXXXXXXXXXXXXXXXXX

SEQ ID NO: 357 - protein, 60 aa, synthetic construct
  VARIANT 1..20: note = X can be G, A, or deleted
  VARIANT 33..34: note = X can be any naturally-occurring amino acid or any unnatural amino acid
  VARIANT 41..60: note = X can be G, A, or deleted
XXXXXXXXXXXXXXXXXXXXVPIYEGYALPHAXXRLDLAGXXXXXXXXXXXXXXXXXXXX

SEQ ID NO: 358 - protein, 60 aa, synthetic construct
  VARIANT 1..20: note = X can be G, A, or deleted
  VARIANT 34..35: note = X can be any naturally-occurring amino acid or any unnatural amino acid
  VARIANT 41..60: note = X can be G, A, or deleted
XXXXXXXXXXXXXXXXXXXXVPIYEGYALPHAIXXLDLAGXXXXXXXXXXXXXXXXXXXX

SEQ ID NO: 359 - protein, 60 aa, synthetic construct
  VARIANT 1..20: note = X can be G, A, or deleted
  VARIANT 34: note = X can be any naturally-occurring amino acid or any unnatural amino acid
  VARIANT 36: note = X can be any naturally-occurring amino acid or any unnatural amino acid
  VARIANT 41..60: note = X can be G, A, or deleted
XXXXXXXXXXXXXXXXXXXXVPIYEGYALPHAIXRXDLAGXXXXXXXXXXXXXXXXXXXX

SEQ ID NO: 360 - protein, 60 aa, synthetic construct
  VARIANT 1..20: note = X can be G, A, or deleted
  VARIANT 34: note = X can be any naturally-occurring amino acid or any unnatural amino acid
  VARIANT 37: note = X can be any naturally-occurring amino acid or any unnatural amino acid
  VARIANT 41..60: note = X can be G, A, or deleted
XXXXXXXXXXXXXXXXXXXXVPIYEGYALPHAIXRLXLAGXXXXXXXXXXXXXXXXXXXX

SEQ ID NO: 361 - protein, 60 aa, synthetic construct
  VARIANT 1..20: note = X can be G, A, or deleted
  VARIANT 34: note = X can be any naturally-occurring amino acid or any unnatural amino acid
  VARIANT 38: note = X can be any naturally-occurring amino acid or any unnatural amino acid
  VARIANT 41..60: note = X can be G, A, or deleted
XXXXXXXXXXXXXXXXXXXXVPIYEGYALPHAIXRLDXAGXXXXXXXXXXXXXXXXXXXX

SEQ ID NO: 362 - protein, 60 aa, synthetic construct
  VARIANT 1..20: note = X can be G, A, or deleted
  VARIANT 34: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXVPIYEGYALPHAIXRLDLXGXXXXXXXXXXXXXXXXXXXX

SEQ ID NO: 363 - protein, 60 aa, synthetic construct
  VARIANT 1..20: note = X can be G, A, or deleted
  VARIANT 34: note = X can be any naturally-occurring amino acid or any unnatural amino acid
  VARIANT 40: note = X can be any naturally-occurring amino acid or any unnatural amino acid
  VARIANT 41..60: note = X can be G, A, or deleted
XXXXXXXXXXXXXXXXXXXXVPIYEGYALPHAIXRLDLAXXXXXXXXXXXXXXXXXXXXX

SEQ ID NO: 364 - protein, 60 aa, synthetic construct
  VARIANT 1..20: note = X can be G, A, or deleted
  VARIANT 21: note = X can be any naturally-occurring amino acid or any unnatural amino acid
  VARIANT 26: note = X can be any naturally-occurring amino acid or any unnatural amino acid
  VARIANT 41..60: note = X can be G, A, or deleted
XXXXXXXXXXXXXXXXXXXXXPHAIXRLDLAGRDLTDYLMXXXXXXXXXXXXXXXXXXXX

SEQ ID NO: 365 - protein, 60 aa, synthetic construct
  VARIANT 1..20: note = X can be G, A, or deleted
  VARIANT 22: note = X can be any naturally-occurring amino acid or any unnatural amino acid
  VARIANT 26: note = X can be any naturally-occurring amino acid or any unnatural amino acid
  VARIANT 41..60: note = X can be G, A, or deleted
XXXXXXXXXXXXXXXXXXXXLXHAIXRLDLAGRDLTDYLMXXXXXXXXXXXXXXXXXXXX

SEQ ID NO: 366 - protein, 60 aa, synthetic construct
  VARIANT 1..20: note = X can be G, A, or deleted
  VARIANT 23: note = X can be any naturally-occurring amino acid or any unnatural amino acid
  VARIANT 26: note = X can be any naturally-occurring amino acid or any unnatural amino acid
  VARIANT 41..60: note = X can be G, A, or deleted
XXXXXXXXXXXXXXXXXXXXLPXAIXRLDLAGRDLTDYLMXXXXXXXXXXXXXXXXXXXX

SEQ ID NO: 367 - protein, 60 aa, synthetic construct
  VARIANT 1..20: note = X can be G, A, or deleted
  VARIANT 24: note = X can be any naturally-occurring amino acid or any unnatural amino acid
  VARIANT 26: note = X can be any naturally-occurring amino acid or any unnatural amino acid
  VARIANT 41..60: note = X can be G, A, or deleted
XXXXXXXXXXXXXXXXXXXXLPHXIXRLDLAGRDLTDYLMXXXXXXXXXXXXXXXXXXXX

SEQ ID NO: 368 - protein, 60 aa, synthetic construct
  VARIANT 1..20: note = X can be G, A, or deleted
  VARIANT 25..26: note = X can be any naturally-occurring amino acid or any unnatural amino acid
  VARIANT 41..60: note = X can be G, A, or deleted
XXXXXXXXXXXXXXXXXXXXLPHAXXRLDLAGRDLTDYLMXXXXXXXXXXXXXXXXXXXX

SEQ ID NO: 369 - protein, 60 aa, synthetic construct
  VARIANT 1..20: note = X can be G, A, or deleted
  VARIANT 26..27: note = X can be any naturally-occurring amino acid or any unnatural amino acid
  VARIANT 41..60: note = X can be G, A, or deleted
XXXXXXXXXXXXXXXXXXXXLPHAIXXLDLAGRDLTDYLMXXXXXXXXXXXXXXXXXXXX

SEQ ID NO: 370 - protein, 60 aa, synthetic construct
  VARIANT 1..20: note = X can be G, A, or deleted
  VARIANT 26: note = X can be any naturally-occurring amino acid or any unnatural amino acid
  VARIANT 28: note = X can be any naturally-occurring amino acid or any unnatural amino acid
  VARIANT 41..60: note = X can be G, A, or deleted
XXXXXXXXXXXXXXXXXXXXLPHAIXRXDLAGRDLTDYLMXXXXXXXXXXXXXXXXXXXX

SEQ ID NO: 371 - protein, 60 aa, synthetic construct
  VARIANT 1..20: note = X can be G, A, or deleted
  VARIANT 26: note = X can be any naturally-occurring amino acid or any unnatural amino acid
  VARIANT 29: note = X can be any naturally-occurring amino acid or any unnatural amino acid
  VARIANT 41..60: note = X can be G, A, or deleted
XXXXXXXXXXXXXXXXXXXXLPHAIXRLXLAGRDLTDYLMXXXXXXXXXXXXXXXXXXXX

SEQ ID NO: 372 - protein, 60 aa, synthetic construct
  VARIANT 1..20: note = X can be G, A, or deleted
  VARIANT 26: note = X can be any naturally-occurring amino acid or any unnatural amino acid
  VARIANT 30: note = X can be any naturally-occurring amino acid or any unnatural amino acid
  VARIANT 41..60: note = X can be G, A, or deleted
XXXXXXXXXXXXXXXXXXXXLPHAIXRLDXAGRDLTDYLMXXXXXXXXXXXXXXXXXXXX

SEQ ID NO: 373 - protein, 60 aa, synthetic construct
  VARIANT 1..20: note = X can be G, A, or deleted
  VARIANT 26: note = X can be any naturally-occurring amino acid or any unnatural amino acid
  VARIANT 31: note = X can be any naturally-occurring amino acid or any unnatural amino acid
  VARIANT 41..60: note = X can be G, A, or deleted
XXXXXXXXXXXXXXXXXXXXLPHAIXRLDLXGRDLTDYLMXXXXXXXXXXXXXXXXXXXX

SEQ ID NO: 374 - protein, 60 aa, synthetic construct
  VARIANT 1..20: note = X can be G, A, or deleted
  VARIANT 26: note = X can be any naturally-occurring amino acid or any unnatural amino acid
  VARIANT 32: note = X can be any naturally-occurring amino acid or any unnatural amino acid
  VARIANT 41..60: note = X can be G, A, or deleted
XXXXXXXXXXXXXXXXXXXXLPHAIXRLDLAXRDLTDYLMXXXXXXXXXXXXXXXXXXXX

SEQ ID NO: 375 - protein, 60 aa, synthetic construct
  VARIANT 1..20: note = X can be G, A, or deleted
  VARIANT 26: note = X can be any naturally-occurring amino acid or any unnatural amino acid
  VARIANT 33: note = X can be any naturally-occurring amino acid or any unnatural amino acid
  VARIANT 41..60: note = X can be G, A, or deleted
XXXXXXXXXXXXXXXXXXXXLPHAIXRLDLAGXDLTDYLMXXXXXXXXXXXXXXXXXXXX

SEQ ID NO: 376 - protein, 60 aa, synthetic construct
  VARIANT 1..20: note = X can be G, A, or deleted
  VARIANT 26: note = X can be any naturally-occurring amino acid or any unnatural amino acid
  VARIANT 34: note = X can be any naturally-occurring amino acid or any unnatural amino acid
  VARIANT 41..60: note = X can be G, A, or deleted
XXXXXXXXXXXXXXXXXXXXLPHAIXRLDLAGRXLTDYLMXXXXXXXXXXXXXXXXXXXX

SEQ ID NO: 377 - protein, 60 aa, synthetic construct
  VARIANT 1..20: note = X can be G, A, or deleted
  VARIANT 26: note = X can be any naturally-occurring amino acid or any unnatural amino acid
  VARIANT 35: note = X can be any naturally-occurring amino acid or any unnatural amino acid
  VARIANT 41..60: note = X can be G, A, or deleted
XXXXXXXXXXXXXXXXXXXXLPHAIXRLDLAGRDXTDYLMXXXXXXXXXXXXXXXXXXXX

SEQ ID NO: 378 - protein, 60 aa, synthetic construct
  VARIANT 1..20: note = X can be G, A, or deleted
  VARIANT 26: note = X can be any naturally-occurring amino acid or any unnatural amino acid
  VARIANT 36: note = X can be any naturally-occurring amino acid or any unnatural amino acid
  VARIANT 41..60: note = X can be G, A, or deleted
XXXXXXXXXXXXXXXXXXXXLPHAIXRLDLAGRDLXDYLMXXXXXXXXXXXXXXXXXXXX

SEQ ID NO: 379 - protein, 60 aa, synthetic construct
  VARIANT 1..20: note = X can be G, A, or deleted
  VARIANT 26: note = X can be any naturally-occurring amino acid or any unnatural amino acid
  VARIANT 37: note = X can be any naturally-occurring amino acid or any unnatural amino acid
  VARIANT 41..60: note = X can be G, A, or deleted
XXXXXXXXXXXXXXXXXXXXLPHAIXRLDLAGRDLTXYLMXXXXXXXXXXXXXXXXXXXX

SEQ ID NO: 380 - protein, 60 aa, synthetic construct
  VARIANT 1..20: note = X can be G, A, or deleted
  VARIANT 26: note = X can be any naturally-occurring amino acid or any unnatural amino acid
  VARIANT 38: note = X can be any naturally-occurring amino acid or any unnatural amino acid
  VARIANT 41..60: note = X can be G, A, or deleted
XXXXXXXXXXXXXXXXXXXXLPHAIXRLDLAGRDLTDXLMXXXXXXXXXXXXXXXXXXXX

SEQ ID NO: 381 - protein, 60 aa, synthetic construct
  VARIANT 1..20: note = X can be G, A, or deleted
  VARIANT 26: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXLPHAIXRLDLAGRDLTDYXMXXXXXXXXXXXXXXXXXXXX

SEQ ID NO: 382 - protein, 60 aa, synthetic construct
  VARIANT 1..20: note = X can be G, A, or deleted
  VARIANT 26: note = X can be any naturally-occurring amino acid or any unnatural amino acid
  VARIANT 40: note = X can be any naturally-occurring amino acid or any unnatural amino acid
  VARIANT 41..60: note = X can be G, A, or deleted
XXXXXXXXXXXXXXXXXXXXLPHAIXRLDLAGRDLTDYLXXXXXXXXXXXXXXXXXXXXX

SEQ ID NO: 383 - protein, 60 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 41..60: note = X can be G, A, or deleted
XXXXXXXXXXXXXXXXXXXXVXDSGDGVTHXVPIYEGYALXXXXXXXXXXXXXXXXXXXX

SEQ ID NO: 384 - protein, 60 aa, synthetic construct
  VARIANT 1..20: note = X can be G, A, or deleted
  VARIANT 21: note = X can be any naturally-occurring amino acid or any unnatural amino acid
  VARIANT 30: note = X can be any naturally-occurring amino acid or any unnatural amino acid
  VARIANT 41..60: note = X can be G, A, or deleted
XXXXXXXXXXXXXXXXXXXXXDSGDGVTHXVPIYEGYALPXXXXXXXXXXXXXXXXXXXX

SEQ ID NO: 385 - protein, 60 aa, synthetic construct
  VARIANT 1..20: note = X can be G, A, or deleted
  VARIANT 29: note = X can be any naturally-occurring amino acid or any unnatural amino acid
  VARIANT 41..60: note = X can be G, A, or deleted
XXXXXXXXXXXXXXXXXXXXDSGDGVTHXVPIYEGYALPHXXXXXXXXXXXXXXXXXXXX

SEQ ID NO: 386 - protein, 60 aa, synthetic construct
  VARIANT 1..20: note = X can be G, A, or deleted
  VARIANT 28: note = X can be any naturally-occurring amino acid or any unnatural amino acid
  VARIANT 41..60: note = X can be G, A, or deleted
XXXXXXXXXXXXXXXXXXXXSGDGVTHXVPIYEGYALPHAXXXXXXXXXXXXXXXXXXXX

SEQ ID NO: 387 - protein, 60 aa, synthetic construct
  VARIANT 1..20: note = X can be G, A, or deleted
  VARIANT 27: note = X can be any naturally-occurring amino acid or any unnatural amino acid
  VARIANT 41..60: note = X can be G, A, or deleted
XXXXXXXXXXXXXXXXXXXXGDGVTHXVPIYEGYALPHAIXXXXXXXXXXXXXXXXXXXX

SEQ ID NO: 388 - protein, 60 aa, synthetic construct
  VARIANT 1..20: note = X can be G, A, or deleted
  VARIANT 26: note = X can be any naturally-occurring amino acid or any unnatural amino acid
  VARIANT 40: note = X can be any naturally-occurring amino acid or any unnatural amino acid
  VARIANT 41..60: note = X can be G, A, or deleted
XXXXXXXXXXXXXXXXXXXXDGVTHXVPIYEGYALPHAIXXXXXXXXXXXXXXXXXXXXX

SEQ ID NO: 389 - protein, 60 aa, synthetic construct
  VARIANT 1..20: note = X can be G, A, or deleted
  VARIANT 25: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXGVTHXVPIYEGYALPHAIXRXXXXXXXXXXXXXXXXXXXX

SEQ ID NO: 390 - protein, 60 aa, synthetic construct
  VARIANT 1..20: note = X can be G, A, or deleted
  VARIANT 24: note = X can be any naturally-occurring amino acid or any unnatural amino acid
  VARIANT 38: note = X can be any naturally-occurring amino acid or any unnatural amino acid
  VARIANT 41..60: note = X can be G, A, or deleted
XXXXXXXXXXXXXXXXXXXXVTHXVPIYEGYALPHAIXRLXXXXXXXXXXXXXXXXXXXX

SEQ ID NO: 391 - protein, 60 aa, synthetic construct
  VARIANT 1..20: note = X can be G, A, or deleted
  VARIANT 23: note = X can be any naturally-occurring amino acid or any unnatural amino acid
  VARIANT 37: note = X can be any naturally-occurring amino acid or any unnatural amino acid
  VARIANT 41..60: note = X can be G, A, or deleted
XXXXXXXXXXXXXXXXXXXXTHXVPIYEGYALPHAIXRLDXXXXXXXXXXXXXXXXXXXX

SEQ ID NO: 392 - protein, 60 aa, synthetic construct
  VARIANT 1..20: note = X can be G, A, or deleted
  VARIANT 22: note = X can be any naturally-occurring amino acid or any unnatural amino acid
  VARIANT 36: note = X can be any naturally-occurring amino acid or any unnatural amino acid
  VARIANT 41..60: note = X can be G, A, or deleted
XXXXXXXXXXXXXXXXXXXXHXVPIYEGYALPHAIXRLDLXXXXXXXXXXXXXXXXXXXX

SEQ ID NO: 393 - protein, 60 aa, synthetic construct
  VARIANT 1..20: note = X can be G, A, or deleted
  VARIANT 21: note = X can be any naturally-occurring amino acid or any unnatural amino acid
  VARIANT 35: note = X can be any naturally-occurring amino acid or any unnatural amino acid
  VARIANT 41..60: note = X can be G, A, or deleted
XXXXXXXXXXXXXXXXXXXXXVPIYEGYALPHAIXRLDLAXXXXXXXXXXXXXXXXXXXX

SEQ ID NO: 394 - protein, 60 aa, synthetic construct
  VARIANT 1..20: note = X can be G, A, or deleted
  VARIANT 34: note = X can be any naturally-occurring amino acid or any unnatural amino acid
  VARIANT 41..60: note = X can be G, A, or deleted
XXXXXXXXXXXXXXXXXXXXVPIYEGYALPHAIXRLDLAGXXXXXXXXXXXXXXXXXXXX

SEQ ID NO: 395 - protein, 60 aa, synthetic construct
  VARIANT 1..20: note = X can be G, A, or deleted
  VARIANT 33: note = X can be any naturally-occurring amino acid or any unnatural amino acid
  VARIANT 41..60: note = X can be G, A, or deleted
XXXXXXXXXXXXXXXXXXXXPIYEGYALPHAIXRLDLAGRXXXXXXXXXXXXXXXXXXXX

SEQ ID NO: 396 - protein, 60 aa, synthetic construct
  VARIANT 1..20: note = X can be G, A, or deleted
  VARIANT 32: note = X can be any naturally-occurring amino acid or any unnatural amino acid
  VARIANT 41..60: note = X can be G, A, or deleted
XXXXXXXXXXXXXXXXXXXXIYEGYALPHAIXRLDLAGRDXXXXXXXXXXXXXXXXXXXX

SEQ ID NO: 397 - protein, 60 aa, synthetic construct
  VARIANT 1..20: note = X can be G, A, or deleted
  VARIANT 31: note = X can be any naturally-occurring amino acid or any unnatural amino acid
  VARIANT 41..60: note = X can be G, A, or deleted
XXXXXXXXXXXXXXXXXXXXYEGYALPHAIXRLDLAGRDLXXXXXXXXXXXXXXXXXXXX

SEQ ID NO: 398 - protein, 60 aa, synthetic construct
  VARIANT 1..20: note = X can be G, A, or deleted
  VARIANT 30: note = X can be any naturally-occurring amino acid or any unnatural amino acid
  VARIANT 41..60: note = X can be G, A, or deleted
XXXXXXXXXXXXXXXXXXXXEGYALPHAIXRLDLAGRDLTXXXXXXXXXXXXXXXXXXXX

SEQ ID NO: 399 - protein, 60 aa, synthetic construct
  VARIANT 1..20: note = X can be G, A, or deleted
  VARIANT 29: note = X can be any naturally-occurring amino acid or any unnatural amino acid
  VARIANT 41..60: note = X can be G, A, or deleted
XXXXXXXXXXXXXXXXXXXXGYALPHAIXRLDLAGRDLTDXXXXXXXXXXXXXXXXXXXX

SEQ ID NO: 400 - protein, 60 aa, synthetic construct
  VARIANT 1..20: note = X can be G, A, or deleted
  VARIANT 28: note = X can be any naturally-occurring amino acid or any unnatural amino acid
  VARIANT 41..60: note = X can be G, A, or deleted
XXXXXXXXXXXXXXXXXXXXYALPHAIXRLDLAGRDLTDYXXXXXXXXXXXXXXXXXXXX

SEQ ID NO: 401 - protein, 60 aa, synthetic construct
  VARIANT 1..20: note = X can be G, A, or deleted
  VARIANT 27: note = X can be any naturally-occurring amino acid or any unnatural amino acid
  VARIANT 41..60: note = X can be G, A, or deleted
XXXXXXXXXXXXXXXXXXXXALPHAIXRLDLAGRDLTDYLXXXXXXXXXXXXXXXXXXXX

SEQ ID NO: 402 - protein, 60 aa, synthetic construct
  VARIANT 1..20: note = X can be G, A, or deleted
  VARIANT 26: note = X can be any naturally-occurring amino acid or any unnatural amino acid
  VARIANT 41..60: note = X can be G, A, or deleted
XXXXXXXXXXXXXXXXXXXXLPHAIXRLDLAGRDLTDYLMXXXXXXXXXXXXXXXXXXXX

SEQ ID NO: 403 - protein, 60 aa, synthetic construct
  VARIANT 1..20: note = X can be G, A, or deleted
  VARIANT 25: note = X can be any naturally-occurring amino acid or any unnatural amino acid
  VARIANT 41..60: note = X can be G, A, or deleted
XXXXXXXXXXXXXXXXXXXXPHAIXRLDLAGRDLTDYLMKXXXXXXXXXXXXXXXXXXXX

SEQ ID NO: 404 - protein, 60 aa, synthetic construct
  VARIANT 1..20: note = X can be G, A, or deleted
  VARIANT 24: note = X can be any naturally-occurring amino acid or any unnatural amino acid
  VARIANT 41..60: note = X can be G, A, or deleted
XXXXXXXXXXXXXXXXXXXXHAIXRLDLAGRDLTDYLMKIXXXXXXXXXXXXXXXXXXXX

SEQ ID NO: 405 - protein, 60 aa, synthetic construct
  VARIANT 1..20: note = X can be G, A, or deleted
  VARIANT 23: note = X can be any naturally-occurring amino acid or any unnatural amino acid
  VARIANT 41..60: note = X can be G, A, or deleted
XXXXXXXXXXXXXXXXXXXXAIXRLDLAGRDLTDYLMKILXXXXXXXXXXXXXXXXXXXX

SEQ ID NO: 406 - protein, 60 aa, synthetic construct
  VARIANT 1..20: note = X can be G, A, or deleted
  VARIANT 22: note = X can be any naturally-occurring amino acid or any unnatural amino acid
  VARIANT 41..60: note = X can be G, A, or deleted
XXXXXXXXXXXXXXXXXXXXIXRLDLAGRDLTDYLMKILTXXXXXXXXXXXXXXXXXXXX

SEQ ID NO: 407 - protein, 60 aa, synthetic construct
  VARIANT 1..20: note = X can be G, A, or deleted
  VARIANT 21: note = X can be any naturally-occurring amino acid or any unnatural amino acid
  VARIANT 41..60: note = X can be G, A, or deleted
XXXXXXXXXXXXXXXXXXXXXRLDLAGRDLTDYLMKILTEXXXXXXXXXXXXXXXXXXXX

SEQ ID NO: 408 - protein, 60 aa, synthetic construct
  VARIANT 1..20: note = X can be G, A, or deleted
  VARIANT 21: note = X can be any naturally-occurring amino acid or any unnatural amino acid
  VARIANT 41..60: note = X can be G, A, or deleted
XXXXXXXXXXXXXXXXXXXXXLDLAGRDLTDYLMKILTERXXXXXXXXXXXXXXXXXXXX

SEQ ID NO: 409 - protein, 60 aa, synthetic construct
  VARIANT 1..20: note = X can be G, A, or deleted
  VARIANT 21: note = X can be any naturally-occurring amino acid or any unnatural amino acid
  VARIANT 41..60: note = X can be G, A, or deleted
XXXXXXXXXXXXXXXXXXXXXDLAGRDLTDYLMKILTERGXXXXXXXXXXXXXXXXXXXX

SEQ ID NO: 410 - protein, 60 aa, synthetic construct
  VARIANT 1..20: note = X can be G, A, or deleted
  VARIANT 21: note = X can be any naturally-occurring amino acid or any unnatural amino acid
  VARIANT 41..60: note = X can be G, A, or deleted
XXXXXXXXXXXXXXXXXXXXXLAGRDLTDYLMKILTERGYXXXXXXXXXXXXXXXXXXXX

SEQ ID NO: 411 - protein, 60 aa, synthetic construct
  VARIANT 1..20: note = X can be G, A, or deleted
  VARIANT 21: note = X can be any naturally-occurring amino acid or any unnatural amino acid
  VARIANT 41..60: note = X can be G, A, or deleted
XXXXXXXXXXXXXXXXXXXXXAGRDLTDYLMKILTERGYSXXXXXXXXXXXXXXXXXXXX

SEQ ID NO: 412 - protein, 60 aa, synthetic construct
  VARIANT 1..20: note = X can be G, A, or deleted
  VARIANT 21: note = X can be any naturally-occurring amino acid or any unnatural amino acid
  VARIANT 41..60: note = X can be G, A, or deleted
XXXXXXXXXXXXXXXXXXXXXGRDLTDYLMKILTERGYSFXXXXXXXXXXXXXXXXXXXX

SEQ ID NO: 413 - protein, 60 aa, synthetic construct; identical to SEQ ID NO: 364
  VARIANT 1..20: note = X can be G, A, or deleted
  VARIANT 21: note = X can be any naturally-occurring amino acid or any unnatural amino acid
  VARIANT 26: note = X can be any naturally-occurring amino acid or any unnatural amino acid
  VARIANT 41..60: note = X can be G, A, or deleted

SEQ ID NO: 414 - protein, 60 aa, synthetic construct; identical to SEQ ID NO: 365
  VARIANT 1..20: note = X can be G, A, or deleted
  VARIANT 22: note = X can be any naturally-occurring amino acid or any unnatural amino acid
  VARIANT 26: note = X can be any naturally-occurring amino acid or any unnatural amino acid
  VARIANT 41..60: note = X can be G, A, or deleted

SEQ ID NO: 415 - protein, 60 aa, synthetic construct; identical to SEQ ID NO: 366
  VARIANT 1..20: note = X can be G, A, or deleted
  VARIANT 23: note = X can be any naturally-occurring amino acid or any unnatural amino acid
  VARIANT 26: note = X can be any naturally-occurring amino acid or any unnatural amino acid
  VARIANT 41..60: note = X can be G, A, or deleted

SEQ ID NO: 416 - protein, 60 aa, synthetic construct; identical to SEQ ID NO: 367
  VARIANT 1..20: note = X can be G, A, or deleted
  VARIANT 24: note = X can be any naturally-occurring amino acid or any unnatural amino acid
  VARIANT 26: note = X can be any naturally-occurring amino acid or any unnatural amino acid
  VARIANT 41..60: note = X can be G, A, or deleted

SEQ ID NO: 417 - protein, 60 aa, synthetic construct; identical to SEQ ID NO: 368
  VARIANT 1..20: note = X can be G, A, or deleted
  VARIANT 25..26: note = X can be any naturally-occurring amino acid or any unnatural amino acid
  VARIANT 41..60: note = X can be G, A, or deleted

SEQ ID NO: 418 - protein, 60 aa, synthetic construct; identical to SEQ ID NO: 368
  VARIANT 1..20: note = X can be G, A, or deleted
  VARIANT 25..26: note = X can be any naturally-occurring amino acid or any unnatural amino acid
  VARIANT 41..60: note = X can be G, A, or deleted

SEQ ID NO: 419 - protein, 60 aa, synthetic construct; identical to SEQ ID NO: 369
  VARIANT 1..20: note = X can be G, A, or deleted
  VARIANT 26..27: note = X can be any naturally-occurring amino acid or any unnatural amino acid
  VARIANT 41..60: note = X can be G, A, or deleted

SEQ ID NO: 420 - protein, 60 aa, synthetic construct; identical to SEQ ID NO: 370
  VARIANT 1..20: note = X can be G, A, or deleted
  VARIANT 26: note = X can be any naturally-occurring amino acid or any unnatural amino acid
  VARIANT 28: note = X can be any naturally-occurring amino acid or any unnatural amino acid
  VARIANT 41..60: note = X can be G, A, or deleted

SEQ ID NO: 421 - protein, 60 aa, synthetic construct; identical to SEQ ID NO: 371
  VARIANT 1..20: note = X can be G, A, or deleted
  VARIANT 26: note = X can be any naturally-occurring amino acid or any unnatural amino acid
  VARIANT 29: note = X can be any naturally-occurring amino acid or any unnatural amino acid
  VARIANT 41..60: note = X can be G, A, or deleted

SEQ ID NO: 422 - protein, 60 aa, synthetic construct; identical to SEQ ID NO: 372
  VARIANT 1..20: note = X can be G, A, or deleted
  VARIANT 26: note = X can be any naturally-occurring amino acid or any unnatural amino acid
  VARIANT 30: note = X can be any naturally-occurring amino acid or any unnatural amino acid
  VARIANT 41..60: note = X can be G, A, or deleted

SEQ ID NO: 423 - protein, 60 aa, synthetic construct; identical to SEQ ID NO: 373
  VARIANT 1..20: note = X can be G, A, or deleted
  VARIANT 26: note = X can be any naturally-occurring amino acid or any unnatural amino acid
  VARIANT 31: note = X can be any naturally-occurring amino acid or any unnatural amino acid
  VARIANT 41..60: note = X can be G, A, or deleted

SEQ ID NO: 424 - protein, 60 aa, synthetic construct; identical to SEQ ID NO: 374
  VARIANT 1..20: note = X can be G, A, or deleted
  VARIANT 26: note = X can be any naturally-occurring amino acid or any unnatural amino acid
  VARIANT 32: note = X can be any naturally-occurring amino acid or any unnatural amino acid
  VARIANT 41..60: note = X can be G, A, or deleted

SEQ ID NO: 425 - protein, 60 aa, synthetic construct; identical to SEQ ID NO: 375
  VARIANT 1..20: note = X can be G, A, or deleted
  VARIANT 26: note = X can be any naturally-occurring amino acid or any unnatural amino acid
  VARIANT 33: note = X can be any naturally-occurring amino acid or any unnatural amino acid
  VARIANT 41..60: note = X can be G, A, or deleted

SEQ ID NO: 426 - protein, 60 aa, synthetic construct; identical to SEQ ID NO: 376
  VARIANT 1..20: note = X can be G, A, or deleted
  VARIANT 26: note = X can be any naturally-occurring amino acid or any unnatural amino acid
  VARIANT 34: note = X can be any naturally-occurring amino acid or any unnatural amino acid
  VARIANT 41..60: note = X can be G, A, or deleted

SEQ ID NO: 427 - protein, 60 aa, synthetic construct; identical to SEQ ID NO: 377
  VARIANT 1..20: note = X can be G, A, or deleted
  VARIANT 26: note = X can be any naturally-occurring amino acid or any unnatural amino acid
  VARIANT 35: note = X can be any naturally-occurring amino acid or any unnatural amino acid
  VARIANT 41..60: note = X can be G, A, or deleted

SEQ ID NO: 428 - protein, 60 aa, synthetic construct; identical to SEQ ID NO: 378
  VARIANT 1..20: note = X can be G, A, or deleted
  VARIANT 26: note = X can be any naturally-occurring amino acid or any unnatural amino acid
  VARIANT 36: note = X can be any naturally-occurring amino acid or any unnatural amino acid
  VARIANT 41..60: note = X can be G, A, or deleted

SEQ ID NO: 429 - protein, 60 aa, synthetic construct; identical to SEQ ID NO: 379
  VARIANT 1..20: note = X can be G, A, or deleted
  VARIANT 26: note = X can be any naturally-occurring amino acid or any unnatural amino acid
  VARIANT 37: note = X can be any naturally-occurring amino acid or any unnatural amino acid
  VARIANT 41..60: note = X can be G, A, or deleted

SEQ ID NO: 430 - protein, 60 aa, synthetic construct; identical to SEQ ID NO: 380
  VARIANT 1..20: note = X can be G, A, or deleted
  VARIANT 26: note = X can be any naturally-occurring amino acid or any unnatural amino acid
  VARIANT 38: note = X can be any naturally-occurring amino acid or any unnatural amino acid
  VARIANT 41..60: note = X can be G, A, or deleted

SEQ ID NO: 431 - protein, 60 aa, synthetic construct; identical to SEQ ID NO: 381
  VARIANT 1..20: note = X can be G, A, or deleted
  VARIANT 26: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted

SEQ ID NO: 432 - protein, 60 aa, synthetic construct; identical to SEQ ID NO: 382
  VARIANT 1..20: note = X can be G, A, or deleted
  VARIANT 26: note = X can be any naturally-occurring amino acid or any unnatural amino acid
  VARIANT 40: note = X can be any naturally-occurring amino acid or any unnatural amino acid
  VARIANT 41..60: note = X can be G, A, or deleted

SEQ ID NO: 433 - protein, 81 aa, synthetic construct
  VARIANT 1..20: note = X can be G, A, or deleted
  VARIANT 21..22: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXXXDSGDGVTHXVPIYEGYALPHAIXRLDLAGRDLTDYLMKIXXXXXXXXXXXXXXXXXXXX

SEQ ID NO: 434 - protein, 81 aa, synthetic construct
  VARIANT 1..20: note = X can be G, A, or deleted
  VARIANT 22..23: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXXSGDGVTHXVPIYEGYALPHAIXRLDLAGRDLTDYLMKIXXXXXXXXXXXXXXXXXXXX

SEQ ID NO: 435 - protein, 81 aa, synthetic construct
  VARIANT 1..20: note = X can be G, A, or deleted
  VARIANT 22: note = X can be any naturally-occurring amino acid or any unnatural amino acid
  VARIANT 24: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDXGDGVTHXVPIYEGYALPHAIXRLDLAGRDLTDYLMKIXXXXXXXXXXXXXXXXXXXX

SEQ ID NO: 436 - protein, 81 aa, synthetic construct
  VARIANT 1..20: note = X can be G, A, or deleted
  VARIANT 22: note = X can be any naturally-occurring amino acid or any unnatural amino acid
  VARIANT 25: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSXDGVTHXVPIYEGYALPHAIXRLDLAGRDLTDYLMKIXXXXXXXXXXXXXXXXXXXX

SEQ ID NO: 437 - protein, 81 aa, synthetic construct
  VARIANT 1..20: note = X can be G, A, or deleted
  VARIANT 22: note = X can be any naturally-occurring amino acid or any unnatural amino acid
  VARIANT 26: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGXGVTHXVPIYEGYALPHAIXRLDLAGRDLTDYLMKIXXXXXXXXXXXXXXXXXXXX

SEQ ID NO: 438 - protein, 81 aa, synthetic construct
  VARIANT 1..20: note = X can be G, A, or deleted
  VARIANT 22: note = X can be any naturally-occurring amino acid or any unnatural amino acid
  VARIANT 27: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGDXVTHXVPIYEGYALPHAIXRLDLAGRDLTDYLMKIXXXXXXXXXXXXXXXXXXXX

SEQ ID NO: 439 - protein, 81 aa, synthetic construct
  VARIANT 1..20: note = X can be G, A, or deleted
  VARIANT 22: note = X can be any naturally-occurring amino acid or any unnatural amino acid
  VARIANT 28: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGDGXTHXVPIYEGYALPHAIXRLDLAGRDLTDYLMKIXXXXXXXXXXXXXXXXXXXX

SEQ ID NO: 440 - protein, 81 aa, synthetic construct
  VARIANT 1..20: note = X can be G, A, or deleted
  VARIANT 22: note = X can be any naturally-occurring amino acid or any unnatural amino acid
  VARIANT 29: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGDGVXHXVPIYEGYALPHAIXRLDLAGRDLTDYLMKIXXXXXXXXXXXXXXXXXXXX

SEQ ID NO: 441 - protein, 81 aa, synthetic construct
  VARIANT 1..20: note = X can be G, A, or deleted
  VARIANT 22: note = X can be any naturally-occurring amino acid or any unnatural amino acid
  VARIANT 30..31: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGDGVTXXVPIYEGYALPHAIXRLDLAGRDLTDYLMKIXXXXXXXXXXXXXXXXXXXX

SEQ ID NO: 442 - protein, 81 aa, synthetic construct
  VARIANT 1..20: note = X can be G, A, or deleted
  VARIANT 22: note = X can be any naturally-occurring amino acid or any unnatural amino acid
  VARIANT 31..32: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGDGVTHXXPIYEGYALPHAIXRLDLAGRDLTDYLMKIXXXXXXXXXXXXXXXXXXXX

SEQ ID NO: 443 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 33: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGDGVTHXVXIYEGYALPHAIXRLDLAGRDLTDYLMKIXXXXXXXXXXXXXXXXXXXX

SEQ ID NO: 444 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 34: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGDGVTHXVPXYEGYALPHAIXRLDLAGRDLTDYLMKIXXXXXXXXXXXXXXXXXXXX

SEQ ID NO: 445 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 35: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGDGVTHXVPIXEGYALPHAIXRLDLAGRDLTDYLMKIXXXXXXXXXXXXXXXXXXXX

SEQ ID NO: 446 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 36: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGDGVTHXVPIYXGYALPHAIXRLDLAGRDLTDYLMKIXXXXXXXXXXXXXXXXXXXX

SEQ ID NO: 447 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 37: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEXYALPHAIXRLDLAGRDLTDYLMKIXXXXXXXXXXXXXXXXXXXX

SEQ ID NO: 448 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 38: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGXALPHAIXRLDLAGRDLTDYLMKIXXXXXXXXXXXXXXXXXXXX

SEQ ID NO: 449 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 39: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYXLPHAIXRLDLAGRDLTDYLMKIXXXXXXXXXXXXXXXXXXXX

SEQ ID NO: 450 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 40: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYAXPHAIXRLDLAGRDLTDYLMKIXXXXXXXXXXXXXXXXXXXX

SEQ ID NO: 451 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 41: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALXHAIXRLDLAGRDLTDYLMKIXXXXXXXXXXXXXXXXXXXX

SEQ ID NO: 452 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 42: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPXAIXRLDLAGRDLTDYLMKIXXXXXXXXXXXXXXXXXXXX

SEQ ID NO: 453 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 43: note = X can be any naturally-occurring amino acid or any unnatural amino acid
  VARIANT 45: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHXIXRLDLAGRDLTDYLMKIXXXXXXXXXXXXXXXXXXXX

SEQ ID NO: 454 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 44..45: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAXXRLDLAGRDLTDYLMKIXXXXXXXXXXXXXXXXXXXX

SEQ ID NO: 455 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 45..46: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAIXXLDLAGRDLTDYLMKIXXXXXXXXXXXXXXXXXXXX

SEQ ID NO: 456 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 47: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAIXRXDLAGRDLTDYLMKIXXXXXXXXXXXXXXXXXXXX

SEQ ID NO: 457 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 48: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAIXRLXLAGRDLTDYLMKIXXXXXXXXXXXXXXXXXXXX

SEQ ID NO: 458 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 49: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAIXRLDXAGRDLTDYLMKIXXXXXXXXXXXXXXXXXXXX

SEQ ID NO: 459 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 50: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAIXRLDLXGRDLTDYLMKIXXXXXXXXXXXXXXXXXXXX

SEQ ID NO: 460 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 51: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAIXRLDLAXRDLTDYLMKIXXXXXXXXXXXXXXXXXXXX

SEQ ID NO: 461 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 52: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAIXRLDLAGXDLTDYLMKIXXXXXXXXXXXXXXXXXXXX

SEQ ID NO: 462 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 53: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAIXRLDLAGRXLTDYLMKIXXXXXXXXXXXXXXXXXXXX

SEQ ID NO: 463 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 54: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAIXRLDLAGRDXTDYLMKIXXXXXXXXXXXXXXXXXXXX

SEQ ID NO: 464 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 55: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAIXRLDLAGRDLXDYLMKIXXXXXXXXXXXXXXXXXXXX

SEQ ID NO: 465 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 56: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAIXRLDLAGRDLTXYLMKIXXXXXXXXXXXXXXXXXXXX

SEQ ID NO: 466 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 57: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAIXRLDLAGRDLTDXLMKIXXXXXXXXXXXXXXXXXXXX

SEQ ID NO: 467 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 58: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAIXRLDLAGRDLTDYXMKIXXXXXXXXXXXXXXXXXXXX

SEQ ID NO: 468 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 59: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAIXRLDLAGRDLTDYLXKIXXXXXXXXXXXXXXXXXXXX

SEQ ID NO: 469 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 60: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAIXRLDLAGRDLTDYLMXIXXXXXXXXXXXXXXXXXXXX

SEQ ID NO: 470 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 45: note = X can be any naturally-occurring amino acid or any unnatural amino acid
  VARIANT 61: note = X can be any naturally-occurring amino acid or any unnatural amino acid
  VARIANT 62..81: note = X can be G, A, or deleted
XXXXXXXXXXXXXXXXXXXXVXDSGDGVTHXVPIYEGYALPHAIXRLDLAGRDLTDYLMKXXXXXXXXXXXXXXXXXXXXX

SEQ ID NO: 471 - protein, 50 aa, synthetic construct; identical to SEQ ID NO: 333
  VARIANT 1..20: note = X can be any naturally-occurring amino acid or any unnatural amino acid or deleted
  VARIANT 31..50: note = X can be any naturally-occurring amino acid or any unnatural amino acid or deleted

SEQ ID NO: 472 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXGRDLTDYLMXXXXXXXXXXXXXXXXXXXX

SEQ ID NO: 473 - protein, 50 aa, synthetic construct
  VARIANT 1..20: note = X can be G, A, or deleted
  VARIANT 22: note = X can be any naturally-occurring amino acid or any unnatural amino acid
  VARIANT 31..50: note = X can be G, A, or deleted
XXXXXXXXXXXXXXXXXXXXAXRDLTDYLMXXXXXXXXXXXXXXXXXXXX

SEQ ID NO: 474 - protein, 50 aa, synthetic construct
  VARIANT 1..20: note = X can be G, A, or deleted
  VARIANT 23: note = X can be any naturally-occurring amino acid or any unnatural amino acid
  VARIANT 31..50: note = X can be G, A, or deleted
XXXXXXXXXXXXXXXXXXXXAGXDLTDYLMXXXXXXXXXXXXXXXXXXXX

SEQ ID NO: 475 - protein, 50 aa, synthetic construct
  VARIANT 1..20: note = X can be G, A, or deleted
  VARIANT 24: note = X can be any naturally-occurring amino acid or any unnatural amino acid
  VARIANT 31..50: note = X can be G, A, or deleted
XXXXXXXXXXXXXXXXXXXXAGRXLTDYLMXXXXXXXXXXXXXXXXXXXX

SEQ ID NO: 476 - protein, 50 aa, synthetic construct
  VARIANT 1..20: note = X can be G, A, or deleted
  VARIANT 25: note = X can be any naturally-occurring amino acid or any unnatural amino acid
  VARIANT 31..50: note = X can be G, A, or deleted
XXXXXXXXXXXXXXXXXXXXAGRDXTDYLMXXXXXXXXXXXXXXXXXXXX

SEQ ID NO: 477 - protein, 50 aa, synthetic construct
  VARIANT 1..20: note = X can be G, A, or deleted
  VARIANT 26: note = X can be any naturally-occurring amino acid or any unnatural amino acid
  VARIANT 31..50: note = X can be G, A, or deleted
XXXXXXXXXXXXXXXXXXXXAGRDLXDYLMXXXXXXXXXXXXXXXXXXXX

SEQ ID NO: 478 - protein, 50 aa, synthetic construct
  VARIANT 1..20: note = X can be G, A, or deleted
  VARIANT 27: note = X can be any naturally-occurring amino acid or any unnatural amino acid
  VARIANT 31..50: note = X can be G, A, or deleted
XXXXXXXXXXXXXXXXXXXXAGRDLTXYLMXXXXXXXXXXXXXXXXXXXX

SEQ ID NO: 479 - protein, 50 aa, synthetic construct
  VARIANT 1..20: note = X can be G, A, or deleted
  VARIANT 28: note = X can be any naturally-occurring amino acid or any unnatural amino acid
  VARIANT 31..50: note = X can be G, A, or deleted
XXXXXXXXXXXXXXXXXXXXAGRDLTDXLMXXXXXXXXXXXXXXXXXXXX

SEQ ID NO: 480 - protein, 50 aa, synthetic construct
  VARIANT 1..20: note = X can be G, A, or deleted
  VARIANT 29: note = X can be any naturally-occurring amino acid or any unnatural amino acid
  VARIANT 31..50: note = X can be G, A, or deleted
XXXXXXXXXXXXXXXXXXXXAGRDLTDYXMXXXXXXXXXXXXXXXXXXXX

SEQ ID NO: 481 - protein, 50 aa, synthetic construct
  VARIANT 1..20: note = X can be G, A, or deleted
  VARIANT 30: note = X can be any naturally-occurring amino acid or any unnatural amino acid
  VARIANT 31..50: note = X can be G, A, or deleted
XXXXXXXXXXXXXXXXXXXXAGRDLTDYLXXXXXXXXXXXXXXXXXXXXX

SEQ ID NO: 482 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXDYLMKILTEXXXXXXXXXXXXXXXXXXXX

SEQ ID NO: 483 - protein, 50 aa, synthetic construct
  VARIANT 1..20: note = X can be G, A, or deleted
  VARIANT 22: note = X can be any naturally-occurring amino acid or any unnatural amino acid
  VARIANT 31..50: note = X can be G, A, or deleted
XXXXXXXXXXXXXXXXXXXXTXYLMKILTEXXXXXXXXXXXXXXXXXXXX

SEQ ID NO: 484 - protein, 50 aa, synthetic construct
  VARIANT 1..20: note = X can be G, A, or deleted
  VARIANT 23: note = X can be any naturally-occurring amino acid or any unnatural amino acid
  VARIANT 31..50: note = X can be G, A, or deleted
XXXXXXXXXXXXXXXXXXXXTDXLMKILTEXXXXXXXXXXXXXXXXXXXX

SEQ ID NO: 485 - protein, 50 aa, synthetic construct
  VARIANT 1..20: note = X can be G, A, or deleted
  VARIANT 24: note = X can be any naturally-occurring amino acid or any unnatural amino acid
  VARIANT 31..50: note = X can be G, A, or deleted
XXXXXXXXXXXXXXXXXXXXTDYXMKILTEXXXXXXXXXXXXXXXXXXXX

SEQ ID NO: 486 - protein, 50 aa, synthetic construct
  VARIANT 1..20: note = X can be G, A, or deleted
  VARIANT 25: note = X can be any naturally-occurring amino acid or any unnatural amino acid
  VARIANT 31..50: note = X can be G, A, or deleted
XXXXXXXXXXXXXXXXXXXXTDYLXKILTEXXXXXXXXXXXXXXXXXXXX

SEQ ID NO: 487 - protein, 50 aa, synthetic construct
  VARIANT 1..20: note = X can be G, A, or deleted
  VARIANT 26: note = X can be any naturally-occurring amino acid or any unnatural amino acid
  VARIANT 31..50: note = X can be G, A, or deleted
XXXXXXXXXXXXXXXXXXXXTDYLMXILTEXXXXXXXXXXXXXXXXXXXX

SEQ ID NO: 488 - protein, 50 aa, synthetic construct
  VARIANT 1..20: note = X can be G, A, or deleted
  VARIANT 27: note = X can be any naturally-occurring amino acid or any unnatural amino acid
  VARIANT 31..50: note = X can be G, A, or deleted
XXXXXXXXXXXXXXXXXXXXTDYLMKXLTEXXXXXXXXXXXXXXXXXXXX

SEQ ID NO: 489 - protein, 50 aa, synthetic construct
  VARIANT 1..20: note = X can be G, A, or deleted
  VARIANT 28: note = X can be any naturally-occurring amino acid or any unnatural amino acid
  VARIANT 31..50: note = X can be G, A, or deleted
XXXXXXXXXXXXXXXXXXXXTDYLMKIXTEXXXXXXXXXXXXXXXXXXXX

SEQ ID NO: 490 - protein, 50 aa, synthetic construct
  VARIANT 1..20: note = X can be G, A, or deleted
  VARIANT 29: note = X can be any naturally-occurring amino acid or any unnatural amino acid
  VARIANT 31..50: note = X can be G, A, or deleted
XXXXXXXXXXXXXXXXXXXXTDYLMKILXEXXXXXXXXXXXXXXXXXXXX

SEQ ID NO: 491 - protein, 50 aa, synthetic construct
  VARIANT 1..20: note = X can be G, A, or deleted
  VARIANT 30: note = X can be any naturally-occurring amino acid or any unnatural amino acid
  VARIANT 31..50: note = X can be G, A, or deleted
XXXXXXXXXXXXXXXXXXXXTDYLMKILTXXXXXXXXXXXXXXXXXXXXX

SEQ ID NO: 492 - protein, 50 aa, synthetic construct; identical to SEQ ID NO: 296
  VARIANT 1..20: note = X can be G, A, or deleted
  VARIANT 26: note = X can be any naturally-occurring amino acid or any unnatural amino acid
  VARIANT 31..50: note = X can be G, A, or deleted

SEQ ID NO: 493 - protein, 50 aa, synthetic construct; identical to SEQ ID NO: 325
  VARIANT 1..20: note = X can be G, A, or deleted
  VARIANT 25: note = X can be any naturally-occurring amino acid or any unnatural amino acid
  VARIANT 31..50: note = X can be G, A, or deleted

SEQ ID NO: 494 - protein, 50 aa, synthetic construct; identical to SEQ ID NO: 326
  VARIANT 1..20: note = X can be G, A, or deleted
  VARIANT 24: note = X can be any naturally-occurring amino acid or any unnatural amino acid
  VARIANT 31..50: note = X can be G, A, or deleted

SEQ ID NO: 495 - protein, 50 aa, synthetic construct; identical to SEQ ID NO: 327
  VARIANT 1..20: note = X can be G, A, or deleted
  VARIANT 23: note = X can be any naturally-occurring amino acid or any unnatural amino acid
  VARIANT 31..50: note = X can be G, A, or deleted

SEQ ID NO: 496 - protein, 50 aa, synthetic construct; identical to SEQ ID NO: 328
  VARIANT 1..20: note = X can be G, A, or deleted
  VARIANT 22: note = X can be any naturally-occurring amino acid or any unnatural amino acid
  VARIANT 31..50: note = X can be G, A, or deleted

SEQ ID NO: 497 - protein, 50 aa, synthetic construct; identical to SEQ ID NO: 329
  VARIANT 1..20: note = X can be G, A, or deleted
  VARIANT 21: note = X can be any naturally-occurring amino acid or any unnatural amino acid
  VARIANT 31..50: note = X can be G, A, or deleted

SEQ ID NO: 498 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXLDLAGRDLTXXXXXXXXXXXXXXXXXXXX

SEQ ID NO: 499 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXDLAGRDLTDXXXXXXXXXXXXXXXXXXXX

SEQ ID NO: 500 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXLAGRDLTDYXXXXXXXXXXXXXXXXXXXX

SEQ ID NO: 501 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXAGRDLTDYLXXXXXXXXXXXXXXXXXXXX

SEQ ID NO: 502 - protein, 50 aa, synthetic construct; identical to SEQ ID NO: 472
  VARIANT 1..20: note = X can be G, A, or deleted
  VARIANT 21: note = X can be any naturally-occurring amino acid or any unnatural amino acid
  VARIANT 31..50: note = X can be G, A, or deleted

SEQ ID NO: 503 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXRDLTDYLMKXXXXXXXXXXXXXXXXXXXX

SEQ ID NO: 504 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXDLTDYLMKIXXXXXXXXXXXXXXXXXXXX

SEQ ID NO: 505 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXLTDYLMKILXXXXXXXXXXXXXXXXXXXX

SEQ ID NO: 506 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXTDYLMKILTXXXXXXXXXXXXXXXXXXXX

SEQ ID NO: 507 - protein, 50 aa, synthetic construct; identical to SEQ ID NO: 482
  VARIANT 1..20: note = X can be G, A, or deleted
  VARIANT 21: note = X can be any naturally-occurring amino acid or any unnatural amino acid
  VARIANT 31..50: note = X can be G, A, or deleted

SEQ ID NO: 508 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXYLMKILTERXXXXXXXXXXXXXXXXXXXX

SEQ ID NO: 509 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXLMKILTERGXXXXXXXXXXXXXXXXXXXX

SEQ ID NO: 510 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXMKILTERGYXXXXXXXXXXXXXXXXXXXX

SEQ ID NO: 511 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXKILTERGYSXXXXXXXXXXXXXXXXXXXX

SEQ ID NO: 512 - protein, 60 aa, synthetic construct
  VARIANT 1..20: note = X can be G, A, or deleted
  VARIANT 21: note = X can be any naturally-occurring amino acid or any unnatural amino acid
  VARIANT 31: note = X can be any naturally-occurring amino acid or any unnatural amino acid
  VARIANT 41..60: note = X can be G, A, or deleted
XXXXXXXXXXXXXXXXXXXXXILTERGYSFXTTAEREIVRXXXXXXXXXXXXXXXXXXXX

SEQ ID NO: 513 - protein, 50 aa, synthetic construct
  VARIANT 1..20: note = X can be G, A, or deleted
  VARIANT 30: note = X can be any naturally-occurring amino acid or any unnatural amino acid
  VARIANT 31..50: note = X can be G, A, or deleted
XXXXXXXXXXXXXXXXXXXXILTERGYSFXXXXXXXXXXXXXXXXXXXXX

SEQ ID NO: 514 - protein, 50 aa, synthetic construct
  VARIANT 1..20: note = X can be G, A, or deleted
  VARIANT 29: note = X can be any naturally-occurring amino acid or any unnatural amino acid
  VARIANT 31..50: note = X can be G, A, or deleted
XXXXXXXXXXXXXXXXXXXXLTERGYSFXTXXXXXXXXXXXXXXXXXXXX

SEQ ID NO: 515 - protein, 50 aa, synthetic construct
  VARIANT 1..20: note = X can be G, A, or deleted
  VARIANT 28: note = X can be any naturally-occurring amino acid or any unnatural amino acid
  VARIANT 31..50: note = X can be G, A, or deleted
XXXXXXXXXXXXXXXXXXXXTERGYSFXTTXXXXXXXXXXXXXXXXXXXX

SEQ ID NO: 516 - protein, 50 aa, synthetic construct
  VARIANT 1..20: note = X can be G, A, or deleted
  VARIANT 27: note = X can be any naturally-occurring amino acid or any unnatural amino acid
  VARIANT 31..50: note = X can be G, A, or deleted
XXXXXXXXXXXXXXXXXXXXERGYSFXTTAXXXXXXXXXXXXXXXXXXXX

SEQ ID NO: 517 - protein, 50 aa, synthetic construct
  VARIANT 1..20: note = X can be G, A, or deleted
  VARIANT 26: note = X can be any naturally-occurring amino acid or any unnatural amino acid
  VARIANT 31..50: note = X can be G, A, or deleted
XXXXXXXXXXXXXXXXXXXXRGYSFXTTAEXXXXXXXXXXXXXXXXXXXX

SEQ ID NO: 518 - protein, 50 aa, synthetic construct
  VARIANT 1..20: note = X can be G, A, or deleted
  VARIANT 25: note = X can be any naturally-occurring amino acid or any unnatural amino acid
  VARIANT 31..50: note = X can be G, A, or deleted
XXXXXXXXXXXXXXXXXXXXGYSFXTTAERXXXXXXXXXXXXXXXXXXXX

SEQ ID NO: 519 - protein, 50 aa, synthetic construct
  VARIANT 1..20: note = X can be G, A, or deleted
  VARIANT 24: note = X can be any naturally-occurring amino acid or any unnatural amino acid
  VARIANT 31..50: note = X can be G, A, or deleted
XXXXXXXXXXXXXXXXXXXXYSFXTTAEREXXXXXXXXXXXXXXXXXXXX

SEQ ID NO: 520 - protein, 50 aa, synthetic construct
  VARIANT 1..20: note = X can be G, A, or deleted
  VARIANT 23: note = X can be any naturally-occurring amino acid or any unnatural amino acid
  VARIANT 31..50: note = X can be G, A, or deleted
XXXXXXXXXXXXXXXXXXXXSFXTTAEREIXXXXXXXXXXXXXXXXXXXX

SEQ ID NO: 521 - protein, 50 aa, synthetic construct
  VARIANT 1..20: note = X can be G, A, or deleted
  VARIANT 22: note = X can be any naturally-occurring amino acid or any unnatural amino acid
  VARIANT 31..50: note = X can be G, A, or deleted
XXXXXXXXXXXXXXXXXXXXFXTTAEREIVXXXXXXXXXXXXXXXXXXXX

SEQ ID NO: 522 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXTTAEREIVRXXXXXXXXXXXXXXXXXXXX

SEQ ID NO: 523 - protein, 60 aa, synthetic construct; identical to SEQ ID NO: 412
  VARIANT 1..20: note = X can be G, A, or deleted
  VARIANT 21: note = X can be any naturally-occurring amino acid or any unnatural amino acid
  VARIANT 41..60: note = X can be G, A, or deleted

SEQ ID NO: 524 - protein, 60 aa, synthetic construct
  VARIANT 1..20: note = X can be G, A, or deleted
  VARIANT 22: note = X can be any naturally-occurring amino acid or any unnatural amino acid
  VARIANT 41..60: note = X can be G, A, or deleted
XXXXXXXXXXXXXXXXXXXXAXRDLTDYLMKILTERGYSFXXXXXXXXXXXXXXXXXXXX

SEQ ID NO: 525 - protein, 60 aa, synthetic construct
  VARIANT 1..20: note = X can be G, A, or deleted
  VARIANT 23: note = X can be any naturally-occurring amino acid or any unnatural amino acid
  VARIANT 41..60: note = X can be G, A, or deleted
XXXXXXXXXXXXXXXXXXXXAGXDLTDYLMKILTERGYSFXXXXXXXXXXXXXXXXXXXX

SEQ ID NO: 526 - protein, 60 aa, synthetic construct
  VARIANT 1..20: note = X can be G, A, or deleted
  VARIANT 24: note = X can be any naturally-occurring amino acid or any unnatural amino acid
  VARIANT 41..60: note = X can be G, A, or deleted
XXXXXXXXXXXXXXXXXXXXAGRXLTDYLMKILTERGYSFXXXXXXXXXXXXXXXXXXXX

SEQ ID NO: 527 - protein, 60 aa, synthetic construct
  VARIANT 1..20: note = X can be G, A, or deleted
  VARIANT 25: note = X can be any naturally-occurring amino acid or any unnatural amino acid
  VARIANT 41..60: note = X can be G, A, or deleted
XXXXXXXXXXXXXXXXXXXXAGRDXTDYLMKILTERGYSFXXXXXXXXXXXXXXXXXXXX

SEQ ID NO: 528 - protein, 60 aa, synthetic construct
  VARIANT 1..20: note = X can be G, A, or deleted
  VARIANT 26: note = X can be any naturally-occurring amino acid or any unnatural amino acid
  VARIANT 41..60: note = X can be G, A, or deleted
XXXXXXXXXXXXXXXXXXXXAGRDLXDYLMKILTERGYSFXXXXXXXXXXXXXXXXXXXX

SEQ ID NO: 529 - protein, 60 aa, synthetic construct
  VARIANT 1..20: note = X can be G, A, or deleted
  VARIANT 27: note = X can be any naturally-occurring amino acid or any unnatural amino acid
  VARIANT 41..60: note = X can be G, A, or deleted
XXXXXXXXXXXXXXXXXXXXAGRDLTXYLMKILTERGYSFXXXXXXXXXXXXXXXXXXXX

SEQ ID NO: 530 - protein, 60 aa, synthetic construct
  VARIANT 1..20: note = X can be G, A, or deleted
  VARIANT 28: note = X can be any naturally-occurring amino acid or any unnatural amino acid
  VARIANT 41..60: note = X can be G, A, or deleted
XXXXXXXXXXXXXXXXXXXXAGRDLTDXLMKILTERGYSFXXXXXXXXXXXXXXXXXXXX

SEQ ID NO: 531 - protein, 60 aa, synthetic construct
  VARIANT 1..20: note = X can be G, A, or deleted
  VARIANT 29: note = X can be any naturally-occurring amino acid or any unnatural amino acid
  VARIANT 41..60: note = X can be G, A, or deleted
XXXXXXXXXXXXXXXXXXXXAGRDLTDYXMKILTERGYSFXXXXXXXXXXXXXXXXXXXX

SEQ ID NO: 532 - protein, 60 aa, synthetic construct
  VARIANT 1..20: note = X can be G, A, or deleted
  VARIANT 30: note = X can be any naturally-occurring amino acid or any unnatural amino acid
  VARIANT 41..60: note = X can be G, A, or deleted
XXXXXXXXXXXXXXXXXXXXAGRDLTDYLXKILTERGYSFXXXXXXXXXXXXXXXXXXXX

SEQ ID NO: 533 - protein, 60 aa, synthetic construct
  VARIANT 1..20: note = X can be G, A, or deleted
  VARIANT 31: note = X can be any naturally-occurring amino acid or any unnatural amino acid
  VARIANT 41..60: note = X can be G, A, or deleted
XXXXXXXXXXXXXXXXXXXXAGRDLTDYLMXILTERGYSFXXXXXXXXXXXXXXXXXXXX

SEQ ID NO: 534 - protein, 60 aa, synthetic construct
  VARIANT 1..20: note = X can be G, A, or deleted
  VARIANT 32: note = X can be any naturally-occurring amino acid or any unnatural amino acid
  VARIANT 41..60: note = X can be G, A, or deleted
XXXXXXXXXXXXXXXXXXXXAGRDLTDYLMKXLTERGYSFXXXXXXXXXXXXXXXXXXXX

SEQ ID NO: 535 - protein, 60 aa, synthetic construct
  VARIANT 1..20: note = X can be G, A, or deleted
  VARIANT 33: note = X can be any naturally-occurring amino acid or any unnatural amino acid
  VARIANT 41..60: note = X can be G, A, or deleted
XXXXXXXXXXXXXXXXXXXXAGRDLTDYLMKIXTERGYSFXXXXXXXXXXXXXXXXXXXX

SEQ ID NO: 536 - protein, 60 aa, synthetic construct
  VARIANT 1..20: note = X can be G, A, or deleted
  VARIANT 34: note = X can be any naturally-occurring amino acid or any unnatural amino acid
  VARIANT 41..60: note = X can be G, A, or deleted
XXXXXXXXXXXXXXXXXXXXAGRDLTDYLMKILXERGYSFXXXXXXXXXXXXXXXXXXXX

SEQ ID NO: 537 - protein, 60 aa, synthetic construct
  VARIANT 1..20: note = X can be G, A, or deleted
  VARIANT 35: note = X can be any naturally-occurring amino acid or any unnatural amino acid
  VARIANT 41..60: note = X can be G, A, or deleted
XXXXXXXXXXXXXXXXXXXXAGRDLTDYLMKILTXRGYSFXXXXXXXXXXXXXXXXXXXX

SEQ ID NO: 538 - protein, 60 aa, synthetic construct
  VARIANT 1..20: note = X can be G, A, or deleted
  VARIANT 36: note = X can be any naturally-occurring amino acid or any unnatural amino acid
  VARIANT 41..60: note = X can be G, A, or deleted
XXXXXXXXXXXXXXXXXXXXAGRDLTDYLMKILTEXGYSFXXXXXXXXXXXXXXXXXXXX

SEQ ID NO: 539 - protein, 60 aa, synthetic construct
  VARIANT 1..20: note = X can be G, A, or deleted
  VARIANT 37: note = X can be any naturally-occurring amino acid or any unnatural amino acid
  VARIANT 41..60: note = X can be G, A, or deleted
XXXXXXXXXXXXXXXXXXXXAGRDLTDYLMKILTERXYSFXXXXXXXXXXXXXXXXXXXX

SEQ ID NO: 540 - protein, 60 aa, synthetic construct
  VARIANT 1..20: note = X can be G, A, or deleted
  VARIANT 38: note = X can be any naturally-occurring amino acid or any unnatural amino acid
  VARIANT 41..60: note = X can be G, A, or deleted
XXXXXXXXXXXXXXXXXXXXAGRDLTDYLMKILTERGXSFXXXXXXXXXXXXXXXXXXXX

SEQ ID NO: 541 - protein, 60 aa, synthetic construct
  VARIANT 1..20: note = X can be G, A, or deleted
  VARIANT 39: note = X can be any naturally-occurring amino acid or any unnatural amino acid
  VARIANT 41..60: note = X can be G, A, or deleted
XXXXXXXXXXXXXXXXXXXXAGRDLTDYLMKILTERGYXFXXXXXXXXXXXXXXXXXXXX

SEQ ID NO: 542 - protein, 60 aa, synthetic construct
  VARIANT 1..20: note = X can be G, A, or deleted
  VARIANT 40: note = X can be any naturally-occurring amino acid or any unnatural amino acid
  VARIANT 41..60: note = X can be G, A, or deleted
XXXXXXXXXXXXXXXXXXXXAGRDLTDYLMKILTERGYSXXXXXXXXXXXXXXXXXXXXX

SEQ ID NO: 543 - protein, 60 aa, synthetic construct; identical to SEQ ID NO: 402
  VARIANT 1..20: note = X can be G, A, or deleted
  VARIANT 26: note = X can be any naturally-occurring amino acid or any unnatural amino acid
  VARIANT 41..60: note = X can be G, A, or deleted

SEQ ID NO: 544 - protein, 60 aa, synthetic construct; identical to SEQ ID NO: 403
  VARIANT 1..20: note = X can be G, A, or deleted
  VARIANT 25: note = X can be any naturally-occurring amino acid or any unnatural amino acid
  VARIANT 41..60: note = X can be G, A, or deleted

SEQ ID NO: 545 - protein, 60 aa, synthetic construct; identical to SEQ ID NO: 404
  VARIANT 1..20: note = X can be G, A, or deleted
  VARIANT 24: note = X can be any naturally-occurring amino acid or any unnatural amino acid
  VARIANT 41..60: note = X can be G, A, or deleted

SEQ ID NO: 546 - protein, 60 aa, synthetic construct; identical to SEQ ID NO: 405
  VARIANT 1..20: note = X can be G, A, or deleted
  VARIANT 23: note = X can be any naturally-occurring amino acid or any unnatural amino acid
  VARIANT 41..60: note = X can be G, A, or deleted

SEQ ID NO: 547 - protein, 60 aa, synthetic construct; identical to SEQ ID NO: 406
  VARIANT 1..20: note = X can be G, A, or deleted
  VARIANT 22: note = X can be any naturally-occurring amino acid or any unnatural amino acid
  VARIANT 41..60: note = X can be G, A, or deleted

SEQ ID NO: 548 - protein, 60 aa, synthetic construct; identical to SEQ ID NO: 407
  VARIANT 1..20: note = X can be G, A, or deleted
  VARIANT 21: note = X can be any naturally-occurring amino acid or any unnatural amino acid
  VARIANT 41..60: note = X can be G, A, or deleted

SEQ ID NO: 549 - protein, 60 aa, synthetic construct
  VARIANT 1..20: note = X can be G, A, or deleted
  VARIANT 41..60: note = X can be G, A, or deleted
XXXXXXXXXXXXXXXXXXXXRLDLAGRDLTDYLMKILTERXXXXXXXXXXXXXXXXXXXX

SEQ ID NO: 550 - protein, 60 aa, synthetic construct
  VARIANT 1..20: note = X can be G, A, or deleted
  VARIANT 41..60: note = X can be G, A, or deleted
XXXXXXXXXXXXXXXXXXXXLDLAGRDLTDYLMKILTERGXXXXXXXXXXXXXXXXXXXX

SEQ ID NO: 551 - protein, 60 aa, synthetic construct
  VARIANT 1..20: note = X can be G, A, or deleted
  VARIANT 41..60: note = X can be G, A, or deleted
XXXXXXXXXXXXXXXXXXXXDLAGRDLTDYLMKILTERGYXXXXXXXXXXXXXXXXXXXX

SEQ ID NO: 552 - protein, 60 aa, synthetic construct
  VARIANT 1..20: note = X can be G, A, or deleted
  VARIANT 41..60: note = X can be G, A, or deleted
XXXXXXXXXXXXXXXXXXXXLAGRDLTDYLMKILTERGYSXXXXXXXXXXXXXXXXXXXX

SEQ ID NO: 553 - protein, 60 aa, synthetic construct
  VARIANT 1..20: note = X can be G, A, or deleted
  VARIANT 41..60: note = X can be G, A, or deleted
XXXXXXXXXXXXXXXXXXXXAGRDLTDYLMKILTERGYSFXXXXXXXXXXXXXXXXXXXX

SEQ ID NO: 554 - protein, 60 aa, synthetic construct
  VARIANT 1..20: note = X can be G, A, or deleted
  VARIANT 40: note = X can be any naturally-occurring amino acid or any unnatural amino acid
  VARIANT 41..60: note = X can be G, A, or deleted
XXXXXXXXXXXXXXXXXXXXGRDLTDYLMKILTERGYSFXXXXXXXXXXXXXXXXXXXXX

SEQ ID NO: 555 - protein, 60 aa, synthetic construct
  VARIANT 1..20: note = X can be G, A, or deleted
  VARIANT 39: note = X can be any naturally-occurring amino acid or any unnatural amino acid
  VARIANT 41..60: note = X can be G, A, or deleted
XXXXXXXXXXXXXXXXXXXXRDLTDYLMKILTERGYSFXTXXXXXXXXXXXXXXXXXXXX

SEQ ID NO: 556 - protein, 60 aa, synthetic construct
  VARIANT 1..20: note = X can be G, A, or deleted
  VARIANT 38: note = X can be any naturally-occurring amino acid or any unnatural amino acid
  VARIANT 41..60: note = X can be G, A, or deleted
XXXXXXXXXXXXXXXXXXXXDLTDYLMKILTERGYSFXTTXXXXXXXXXXXXXXXXXXXX

SEQ ID NO: 557 - protein, 60 aa, synthetic construct
  VARIANT 1..20: note = X can be G, A, or deleted
  VARIANT 37: note = X can be any naturally-occurring amino acid or any unnatural amino acid
  VARIANT 41..60: note = X can be G, A, or deleted
XXXXXXXXXXXXXXXXXXXXLTDYLMKILTERGYSFXTTAXXXXXXXXXXXXXXXXXXXX

SEQ ID NO: 558 - protein, 60 aa, synthetic construct
  VARIANT 1..20: note = X can be G, A, or deleted
  VARIANT 36: note = X can be any naturally-occurring amino acid or any unnatural amino acid
  VARIANT 41..60: note = X can be G, A, or deleted
XXXXXXXXXXXXXXXXXXXXTDYLMKILTERGYSFXTTAEXXXXXXXXXXXXXXXXXXXX

SEQ ID NO: 559 - protein, 60 aa, synthetic construct
  VARIANT 1..20: note = X can be G, A, or deleted
  VARIANT 35: note = X can be any naturally-occurring amino acid or any unnatural amino acid
  VARIANT 41..60: note = X can be G, A, or deleted
XXXXXXXXXXXXXXXXXXXXDYLMKILTERGYSFXTTAERXXXXXXXXXXXXXXXXXXXX

SEQ ID NO: 560 - protein, 60 aa, synthetic construct
  VARIANT 1..20: note = X can be G, A, or deleted
  VARIANT 34: note = X can be any naturally-occurring amino acid or any unnatural amino acid
  VARIANT 41..60: note = X can be G, A, or deleted
XXXXXXXXXXXXXXXXXXXXYLMKILTERGYSFXTTAEREXXXXXXXXXXXXXXXXXXXX

SEQ ID NO: 561 - protein, 60 aa, synthetic construct
  VARIANT 1..20: note = X can be G, A, or deleted
  VARIANT 33: note = X can be any naturally-occurring amino acid or any unnatural amino acid
  VARIANT 41..60: note = X can be G, A, or deleted
XXXXXXXXXXXXXXXXXXXXLMKILTERGYSFXTTAEREIXXXXXXXXXXXXXXXXXXXX

SEQ ID NO: 562 - protein, 60 aa, synthetic construct
  VARIANT 1..20: note = X can be G, A, or deleted
  VARIANT 32: note = X can be any naturally-occurring amino acid or any unnatural amino acid
  VARIANT 41..60: note = X can be G, A, or deleted
XXXXXXXXXXXXXXXXXXXXMKILTERGYSFXTTAEREIVXXXXXXXXXXXXXXXXXXXX

SEQ ID NO: 563 - protein, 60 aa, synthetic construct
  VARIANT 1..20: note = X can be G, A, or deleted
  VARIANT 31: note = X can be any naturally-occurring amino acid or any unnatural amino acid
  VARIANT 41..60: note = X can be G, A, or deleted
XXXXXXXXXXXXXXXXXXXXKILTERGYSFXTTAEREIVRXXXXXXXXXXXXXXXXXXXX

SEQ ID NO: 564 - protein, 80 aa, synthetic construct
  VARIANT 1..20: note = X can be G, A, or deleted
  VARIANT 21: note = X can be any naturally-occurring amino acid or any unnatural amino acid
  VARIANT 26: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXXPHAIXRLDLAGRDLTDYLMKILTERGYSFXTTAEREIVRXXXXXXXXXXXXXXXXXXXX

SEQ ID NO: 565 - protein, 80 aa, synthetic construct
  VARIANT 1..20: note = X can be G, A, or deleted
  VARIANT 22: note = X can be any naturally-occurring amino acid or any unnatural amino acid
  VARIANT 26: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLXHAIXRLDLAGRDLTDYLMKILTERGYSFXTTAEREIVRXXXXXXXXXXXXXXXXXXXX

SEQ ID NO: 566 - protein, 80 aa, synthetic construct
  VARIANT 1..20: note = X can be G, A, or deleted
  VARIANT 23: note = X can be any naturally-occurring amino acid or any unnatural amino acid
  VARIANT 26: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPXAIXRLDLAGRDLTDYLMKILTERGYSFXTTAEREIVRXXXXXXXXXXXXXXXXXXXX

SEQ ID NO: 567 - protein, 80 aa, synthetic construct
  VARIANT 1..20: note = X can be G, A, or deleted
  VARIANT 24: note = X can be any naturally-occurring amino acid or any unnatural amino acid
  VARIANT 26: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHXIXRLDLAGRDLTDYLMKILTERGYSFXTTAEREIVRXXXXXXXXXXXXXXXXXXXX

SEQ ID NO: 568 - protein, 80 aa, synthetic construct
  VARIANT 1..20: note = X can be G, A, or deleted
  VARIANT 25..26: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAXXRLDLAGRDLTDYLMKILTERGYSFXTTAEREIVRXXXXXXXXXXXXXXXXXXXX

SEQ ID NO: 569 - protein, 80 aa, synthetic construct
  VARIANT 1..20: note = X can be G, A, or deleted
  VARIANT 26..27: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXXLDLAGRDLTDYLMKILTERGYSFXTTAEREIVRXXXXXXXXXXXXXXXXXXXX

SEQ ID NO: 570 - protein, 80 aa, synthetic construct
  VARIANT 1..20: note = X can be G, A, or deleted
  VARIANT 26: note = X can be any naturally-occurring amino acid or any unnatural amino acid
  VARIANT 28: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRXDLAGRDLTDYLMKILTERGYSFXTTAEREIVRXXXXXXXXXXXXXXXXXXXX

SEQ ID NO: 571 - protein, 80 aa, synthetic construct
  VARIANT 1..20: note = X can be G, A, or deleted
  VARIANT 26: note = X can be any naturally-occurring amino acid or any unnatural amino acid
  VARIANT 29: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLXLAGRDLTDYLMKILTERGYSFXTTAEREIVRXXXXXXXXXXXXXXXXXXXX

SEQ ID NO: 572 - protein, 80 aa, synthetic construct
  VARIANT 1..20: note = X can be G, A, or deleted
  VARIANT 26: note = X can be any naturally-occurring amino acid or any unnatural amino acid
  VARIANT 30: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDXAGRDLTDYLMKILTERGYSFXTTAEREIVRXXXXXXXXXXXXXXXXXXXX

SEQ ID NO: 573 - protein, 80 aa, synthetic construct
  VARIANT 1..20: note = X can be G, A, or deleted
  VARIANT 26: note = X can be any naturally-occurring amino acid or any unnatural amino acid
  VARIANT 31: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLXGRDLTDYLMKILTERGYSFXTTAEREIVRXXXXXXXXXXXXXXXXXXXX

SEQ ID NO: 574 - protein, 80 aa, synthetic construct
  VARIANT 1..20: note = X can be G, A, or deleted
  VARIANT 26: note = X can be any naturally-occurring amino acid or any unnatural amino acid
  VARIANT 32: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAXRDLTDYLMKILTERGYSFXTTAEREIVRXXXXXXXXXXXXXXXXXXXX

SEQ ID NO: 575 - protein, 80 aa, synthetic construct
  VARIANT 1..20: note = X can be G, A, or deleted
  VARIANT 26: note = X can be any naturally-occurring amino acid or any unnatural amino acid
  VARIANT 33: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGXDLTDYLMKILTERGYSFXTTAEREIVRXXXXXXXXXXXXXXXXXXXX

SEQ ID NO: 576 - protein, 80 aa, synthetic construct
  VARIANT 1..20: note = X can be G, A, or deleted
  VARIANT 26: note = X can be any naturally-occurring amino acid or any unnatural amino acid
  VARIANT 34: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGRXLTDYLMKILTERGYSFXTTAEREIVRXXXXXXXXXXXXXXXXXXXX

SEQ ID NO: 577 - protein, 80 aa, synthetic construct
  VARIANT 1..20: note = X can be G, A, or deleted
  VARIANT 26: note = X can be any naturally-occurring amino acid or any unnatural amino acid
  VARIANT 35: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGRDXTDYLMKILTERGYSFXTTAEREIVRXXXXXXXXXXXXXXXXXXXX

SEQ ID NO: 578 - protein, 80 aa, synthetic construct
  VARIANT 1..20: note = X can be G, A, or deleted
  VARIANT 26: note = X can be any naturally-occurring amino acid or any unnatural amino acid
  VARIANT 36: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGRDLXDYLMKILTERGYSFXTTAEREIVRXXXXXXXXXXXXXXXXXXXX

SEQ ID NO: 579 - protein, 80 aa, synthetic construct
  VARIANT 1..20: note = X can be G, A, or deleted
  VARIANT 26: note = X can be any naturally-occurring amino acid or any unnatural amino acid
  VARIANT 37: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTXYLMKILTERGYSFXTTAEREIVRXXXXXXXXXXXXXXXXXXXX

SEQ ID NO: 580 - protein, 80 aa, synthetic construct
  VARIANT 1..20: note = X can be G, A, or deleted
  VARIANT 26: note = X can be any naturally-occurring amino acid or any unnatural amino acid
  VARIANT 38: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDXLMKILTERGYSFXTTAEREIVRXXXXXXXXXXXXXXXXXXXX

SEQ ID NO: 581 - protein, 80 aa, synthetic construct
  VARIANT 1..20: note = X can be G, A, or deleted
  VARIANT 26: note = X can be any naturally-occurring amino acid or any unnatural amino acid
  VARIANT 39: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYXMKILTERGYSFXTTAEREIVRXXXXXXXXXXXXXXXXXXXX

SEQ ID NO: 582 - protein, 80 aa, synthetic construct
  VARIANT 1..20: note = X can be G, A, or deleted
  VARIANT 26: note = X can be any naturally-occurring amino acid or any unnatural amino acid
  VARIANT 40: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLXKILTERGYSFXTTAEREIVRXXXXXXXXXXXXXXXXXXXX

SEQ ID NO: 583 - protein, 80 aa, synthetic construct
  VARIANT 1..20: note = X can be G, A, or deleted
  VARIANT 26: note = X can be any naturally-occurring amino acid or any unnatural amino acid
  VARIANT 41: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMXILTERGYSFXTTAEREIVRXXXXXXXXXXXXXXXXXXXX

SEQ ID NO: 584 - protein, 80 aa, synthetic construct
  VARIANT 1..20: note = X can be G, A, or deleted
  VARIANT 26: note = X can be any naturally-occurring amino acid or any unnatural amino acid
  VARIANT 42: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKXLTERGYSFXTTAEREIVRXXXXXXXXXXXXXXXXXXXX

SEQ ID NO: 585 - protein, 80 aa, synthetic construct
  VARIANT 1..20: note = X can be G, A, or deleted
  VARIANT 26: note = X can be any naturally-occurring amino acid or any unnatural amino acid
  VARIANT 43: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKIXTERGYSFXTTAEREIVRXXXXXXXXXXXXXXXXXXXX

SEQ ID NO: 586 - protein, 80 aa, synthetic construct
  VARIANT 1..20: note = X can be G, A, or deleted
  VARIANT 26: note = X can be any naturally-occurring amino acid or any unnatural amino acid
  VARIANT 44: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKILXERGYSFXTTAEREIVRXXXXXXXXXXXXXXXXXXXX

SEQ ID NO: 587 - protein, 80 aa, synthetic construct
  VARIANT 1..20: note = X can be G, A, or deleted
  VARIANT 26: note = X can be any naturally-occurring amino acid or any unnatural amino acid
  VARIANT 45: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKILTXRGYSFXTTAEREIVRXXXXXXXXXXXXXXXXXXXX

SEQ ID NO: 588 - protein, 80 aa, synthetic construct
  VARIANT 1..20: note = X can be G, A, or deleted
  VARIANT 26: note = X can be any naturally-occurring amino acid or any unnatural amino acid
  VARIANT 46: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKILTEXGYSFXTTAEREIVRXXXXXXXXXXXXXXXXXXXX

SEQ ID NO: 589 - protein, 80 aa, synthetic construct
  VARIANT 1..20: note = X can be G, A, or deleted
  VARIANT 26: note = X can be any naturally-occurring amino acid or any unnatural amino acid
  VARIANT 47: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKILTERXYSFXTTAEREIVRXXXXXXXXXXXXXXXXXXXX

SEQ ID NO: 590 - protein, 80 aa, synthetic construct
  VARIANT 1..20: note = X can be G, A, or deleted
  VARIANT 26: note = X can be any naturally-occurring amino acid or any unnatural amino acid
  VARIANT 48: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKILTERGXSFXTTAEREIVRXXXXXXXXXXXXXXXXXXXX

SEQ ID NO: 591 - protein, 80 aa, synthetic construct
  VARIANT 1..20: note = X can be G, A, or deleted
  VARIANT 26: note = X can be any naturally-occurring amino acid or any unnatural amino acid
  VARIANT 49: note = X can be any naturally-occurring amino acid or any unnatural amino acid
  VARIANT 51: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKILTERGYXFXTTAEREIVRXXXXXXXXXXXXXXXXXXXX

SEQ ID NO: 592 - protein, 80 aa, synthetic construct
  VARIANT 1..20: note = X can be G, A, or deleted
  VARIANT 26: note = X can be any naturally-occurring amino acid or any unnatural amino acid
  VARIANT 50..51: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKILTERGYSXXTTAEREIVRXXXXXXXXXXXXXXXXXXXX

SEQ ID NO: 593 - protein, 80 aa, synthetic construct
  VARIANT 1..20: note = X can be G, A, or deleted
  VARIANT 26: note = X can be any naturally-occurring amino acid or any unnatural amino acid
  VARIANT 51..52: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKILTERGYSFXXTAEREIVRXXXXXXXXXXXXXXXXXXXX

SEQ ID NO: 594 - protein, 80 aa, synthetic construct
  VARIANT 1..20: note = X can be G, A, or deleted
  VARIANT 26: note = X can be any naturally-occurring amino acid or any unnatural amino acid
  VARIANT 51: note = X can be any naturally-occurring amino acid or any unnatural amino acid
  VARIANT 53: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKILTERGYSFXTXAEREIVRXXXXXXXXXXXXXXXXXXXX

SEQ ID NO: 595 - protein, 80 aa, synthetic construct
  VARIANT 1..20: note = X can be G, A, or deleted
  VARIANT 26: note = X can be any naturally-occurring amino acid or any unnatural amino acid
  VARIANT 51: note = X can be any naturally-occurring amino acid or any unnatural amino acid
  VARIANT 54: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKILTERGYSFXTTXEREIVRXXXXXXXXXXXXXXXXXXXX

SEQ ID NO: 596 - protein, 80 aa, synthetic construct
  VARIANT 1..20: note = X can be G, A, or deleted
  VARIANT 26: note = X can be any naturally-occurring amino acid or any unnatural amino acid
  VARIANT 51: note = X can be any naturally-occurring amino acid or any unnatural amino acid
  VARIANT 55: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKILTERGYSFXTTAXREIVRXXXXXXXXXXXXXXXXXXXX

SEQ ID NO: 597 - protein, 80 aa, synthetic construct
  VARIANT 1..20: note = X can be G, A, or deleted
  VARIANT 26: note = X can be any naturally-occurring amino acid or any unnatural amino acid
  VARIANT 51: note = X can be any naturally-occurring amino acid or any unnatural amino acid
  VARIANT 56: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKILTERGYSFXTTAEXEIVRXXXXXXXXXXXXXXXXXXXX

SEQ ID NO: 598 - protein, 80 aa, synthetic construct
  VARIANT 1..20: note = X can be G, A, or deleted
  VARIANT 26: note = X can be any naturally-occurring amino acid or any unnatural amino acid
  VARIANT 51: note = X can be any naturally-occurring amino acid or any unnatural amino acid
  VARIANT 57: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKILTERGYSFXTTAERXIVRXXXXXXXXXXXXXXXXXXXX

SEQ ID NO: 599 - protein, 80 aa, synthetic construct
  VARIANT 1..20: note = X can be G, A, or deleted
  VARIANT 26: note = X can be any naturally-occurring amino acid or any unnatural amino acid
  VARIANT 51: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKILTERGYSFXTTAEREXVRXXXXXXXXXXXXXXXXXXXX

SEQ ID NO: 600 - protein, 80 aa, synthetic construct
  VARIANT 1..20: note = X can be G, A, or deleted
  VARIANT 26: note = X can be any naturally-occurring amino acid or any unnatural amino acid
  VARIANT 51: note = X can be any naturally-occurring amino acid or any unnatural amino acid
  VARIANT 59: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKILTERGYSFXTTAEREIXRXXXXXXXXXXXXXXXXXXXX

SEQ ID NO: 601 - protein, 80 aa, synthetic construct
  VARIANT 1..20: note = X can be G, A, or deleted
  VARIANT 26: note = X can be any naturally-occurring amino acid or any unnatural amino acid
  VARIANT 51: note = X can be any naturally-occurring amino acid or any unnatural amino acid
  VARIANT 60: note = X can be any naturally-occurring amino acid or any unnatural amino acid
  VARIANT 61..80: note = X can be G, A, or deleted
XXXXXXXXXXXXXXXXXXXXLPHAIXRLDLAGRDLTDYLMKILTERGYSFXTTAEREIVXXXXXXXXXXXXXXXXXXXXX

SEQ ID NO: 602 - protein, 50 aa, synthetic construct
  VARIANT 1..20: note = X can be any naturally-occurring amino acid or any unnatural amino acid or deleted
  VARIANT 31..50: note = X can be any naturally-occurring amino acid or any unnatural amino acid or deleted
XXXXXXXXXXXXXXXXXXXXLCYVALDFEQXXXXXXXXXXXXXXXXXXXX

SEQ ID NO: 603 - protein, 50 aa, synthetic construct
  VARIANT 1..20: note = X can be G, A, or deleted
  VARIANT 21: note = X can be any naturally-occurring amino acid or any unnatural amino acid
  VARIANT 30: note = X can be any naturally-occurring amino acid or any unnatural amino acid
  VARIANT 31..50: note = X can be G, A, or deleted
XXXXXXXXXXXXXXXXXXXXXCYVALDFEXXXXXXXXXXXXXXXXXXXXX

SEQ ID NO: 604 - protein, 50 aa, synthetic construct
  VARIANT 1..20: note = X can be G, A, or deleted
  VARIANT 22: note = X can be any naturally-occurring amino acid or any unnatural amino acid
  VARIANT 30: note = X can be any naturally-occurring amino acid or any unnatural amino acid
  VARIANT 31..50: note = X can be G, A, or deleted
XXXXXXXXXXXXXXXXXXXXLXYVALDFEXXXXXXXXXXXXXXXXXXXXX

SEQ ID NO: 605 - protein, 50 aa, synthetic construct
  VARIANT 1..20: note = X can be G, A, or deleted
  VARIANT 23: note = X can be any naturally-occurring amino acid or any unnatural amino acid
  VARIANT 30: note = X can be any naturally-occurring amino acid or any unnatural amino acid
  VARIANT 31..50: note = X can be G, A, or deleted
XXXXXXXXXXXXXXXXXXXXLCXVALDFEXXXXXXXXXXXXXXXXXXXXX

SEQ ID NO: 606 - protein, 50 aa, synthetic construct
  VARIANT 1..20: note = X can be G, A, or deleted
  VARIANT 24: note = X can be any naturally-occurring amino acid or any unnatural amino acid
  VARIANT 30: note = X can be any naturally-occurring amino acid or any unnatural amino acid
  VARIANT 31..50: note = X can be G, A, or deleted
XXXXXXXXXXXXXXXXXXXXLCYXALDFEXXXXXXXXXXXXXXXXXXXXX

SEQ ID NO: 607 - protein, 50 aa, synthetic construct
  VARIANT 1..20: note = X can be G, A, or deleted
  VARIANT 25: note = X can be any naturally-occurring amino acid or any unnatural amino acid
  VARIANT 30: note = X can be any naturally-occurring amino acid or any unnatural amino acid
  VARIANT 31..50: note = X can be G, A, or deleted
XXXXXXXXXXXXXXXXXXXXLCYVXLDFEXXXXXXXXXXXXXXXXXXXXX

SEQ ID NO: 608 - protein, 50 aa, synthetic construct
  VARIANT 1..20: note = X can be G, A, or deleted
  VARIANT 26: note = X can be any naturally-occurring amino acid or any unnatural amino acid
  VARIANT 30: note = X can be any naturally-occurring amino acid or any unnatural amino acid
  VARIANT 31..50: note = X can be G, A, or deleted
XXXXXXXXXXXXXXXXXXXXLCYVAXDFEXXXXXXXXXXXXXXXXXXXXX

SEQ ID NO: 609 - protein, 50 aa, synthetic construct
  VARIANT 1..20: note = X can be G, A, or deleted
  VARIANT 27: note = X can be any naturally-occurring amino acid or any unnatural amino acid
  VARIANT 30: note = X can be any naturally-occurring amino acid or any unnatural amino acid
  VARIANT 31..50: note = X can be G, A, or deleted
XXXXXXXXXXXXXXXXXXXXLCYVALXFEXXXXXXXXXXXXXXXXXXXXX

SEQ ID NO: 610 - protein, 50 aa, synthetic construct
  VARIANT 1..20: note = X can be G, A, or deleted
  VARIANT 28: note = X can be any naturally-occurring amino acid or any unnatural amino acid
  VARIANT 30: note = X can be any naturally-occurring amino acid or any unnatural amino acid
  VARIANT 31..50: note = X can be G, A, or deleted
XXXXXXXXXXXXXXXXXXXXLCYVALDXEXXXXXXXXXXXXXXXXXXXXX

SEQ ID NO: 611 - protein, 50 aa, synthetic construct
  VARIANT 1..20: note = X can be G, A, or deleted
  VARIANT 29..30: note = X can be any naturally-occurring amino acid or any unnatural amino acid
  VARIANT 31..50: note = X can be G, A, or deleted
XXXXXXXXXXXXXXXXXXXXLCYVALDFXXXXXXXXXXXXXXXXXXXXXX

SEQ ID NO: 612 - protein, 50 aa, synthetic construct; identical to SEQ ID NO: 521
  VARIANT 1..20: note = X can be G, A, or deleted
  VARIANT 22: note = X can be any naturally-occurring amino acid or any unnatural amino acid
  VARIANT 31..50: note = X can be G, A, or deleted

SEQ ID NO: 613 - protein, 50 aa, synthetic construct; identical to SEQ ID NO: 522
  VARIANT 1..20: note = X can be G, A, or deleted
  VARIANT 21: note = X can be any naturally-occurring amino acid or any unnatural amino acid
  VARIANT 31..50: note = X can be G, A, or deleted

SEQ ID NO: 614 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXTAEREIVRDXXXXXXXXXXXXXXXXXXXX

SEQ ID NO: 615 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXAEREIVRDIXXXXXXXXXXXXXXXXXXXX

SEQ ID NO: 616 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXEREIVRDIKXXXXXXXXXXXXXXXXXXXX

SEQ ID NO: 617 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXREIVRDIKEXXXXXXXXXXXXXXXXXXXX

SEQ ID NO: 618 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXEIVRDIKEKXXXXXXXXXXXXXXXXXXXX

SEQ ID NO: 619 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXIVRDIKEKLXXXXXXXXXXXXXXXXXXXX

SEQ ID NO: 620 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXVRDIKEKLCXXXXXXXXXXXXXXXXXXXX

SEQ ID NO: 621 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXRDIKEKLCYXXXXXXXXXXXXXXXXXXXX

SEQ ID NO: 622 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXDIKEKLCYVXXXXXXXXXXXXXXXXXXXX

SEQ ID NO: 623 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXIKEKLCYVAXXXXXXXXXXXXXXXXXXXX

SEQ ID NO: 624 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXKEKLCYVALXXXXXXXXXXXXXXXXXXXX

SEQ ID NO: 625 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXEKLCYVALDXXXXXXXXXXXXXXXXXXXX

SEQ ID NO: 626 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXKLCYVALDFXXXXXXXXXXXXXXXXXXXX

SEQ ID NO: 627 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXLCYVALDFEXXXXXXXXXXXXXXXXXXXX

SEQ ID NO: 628 - protein, 50 aa, synthetic construct
  VARIANT 1..20: note = X can be G, A, or deleted
  VARIANT 30: note = X can be any naturally-occurring amino acid or any unnatural amino acid
  VARIANT 31..50: note = X can be G, A, or deleted
XXXXXXXXXXXXXXXXXXXXLCYVALDFEXXXXXXXXXXXXXXXXXXXXX

SEQ ID NO: 629 - protein, 50 aa, synthetic construct
  VARIANT 1..20: note = X can be G, A, or deleted
  VARIANT 29: note = X can be any naturally-occurring amino acid or any unnatural amino acid
  VARIANT 31..50: note = X can be G, A, or deleted
XXXXXXXXXXXXXXXXXXXXCYVALDFEXEXXXXXXXXXXXXXXXXXXXX

SEQ ID NO: 630 - protein, 50 aa, synthetic construct
  VARIANT 1..20: note = X can be G, A, or deleted
  VARIANT 28: note = X can be any naturally-occurring amino acid or any unnatural amino acid
  VARIANT 31..50: note = X can be G, A, or deleted
XXXXXXXXXXXXXXXXXXXXYVALDFEXEMXXXXXXXXXXXXXXXXXXXX

SEQ ID NO: 631 - protein, 50 aa, synthetic construct
  VARIANT 1..20: note = X can be G, A, or deleted
  VARIANT 27: note = X can be any naturally-occurring amino acid or any unnatural amino acid
  VARIANT 31..50: note = X can be G, A, or deleted
XXXXXXXXXXXXXXXXXXXXVALDFEXEMAXXXXXXXXXXXXXXXXXXXX

SEQ ID NO: 632 - protein, 50 aa, synthetic construct
  VARIANT 1..20: note = X can be G, A, or deleted
  VARIANT 26: note = X can be any naturally-occurring amino acid or any unnatural amino acid
  VARIANT 31..50: note = X can be G, A, or deleted
XXXXXXXXXXXXXXXXXXXXALDFEXEMATXXXXXXXXXXXXXXXXXXXX

SEQ ID NO: 633 - protein, 50 aa, synthetic construct
  VARIANT 1..20: note = X can be G, A, or deleted
  VARIANT 25: note = X can be any naturally-occurring amino acid or any unnatural amino acid
  VARIANT 31..50: note = X can be G, A, or deleted
XXXXXXXXXXXXXXXXXXXXLDFEXEMATAXXXXXXXXXXXXXXXXXXXX

SEQ ID NO: 634 - protein, 50 aa, synthetic construct
  VARIANT 1..20: note = X can be G, A, or deleted
  VARIANT 24: note = X can be any naturally-occurring amino acid or any unnatural amino acid
  VARIANT 31..50: note = X can be G, A, or deleted
XXXXXXXXXXXXXXXXXXXXDFEXEMATAAXXXXXXXXXXXXXXXXXXXX

SEQ ID NO: 635 - protein, 50 aa, synthetic construct
  VARIANT 1..20: note = X can be G, A, or deleted
  VARIANT 23: note = X can be any naturally-occurring amino acid or any unnatural amino acid
  VARIANT 31..50: note = X can be G, A, or deleted
XXXXXXXXXXXXXXXXXXXXFEXEMATAASXXXXXXXXXXXXXXXXXXXX

SEQ ID NO: 636 - protein, 50 aa, synthetic construct
  VARIANT 1..20: note = X can be G, A, or deleted
  VARIANT 22: note = X can be any naturally-occurring amino acid or any unnatural amino acid
  VARIANT 31..50: note = X can be G, A, or deleted
XXXXXXXXXXXXXXXXXXXXEXEMATAASSXXXXXXXXXXXXXXXXXXXX

SEQ ID NO: 637 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXEMATAASSSXXXXXXXXXXXXXXXXXXXX

SEQ ID NO: 638 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXMATAASSSSXXXXXXXXXXXXXXXXXXXX

SEQ ID NO: 639 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXATAASSSSLXXXXXXXXXXXXXXXXXXXX

SEQ ID NO: 640 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXTAASSSSLEXXXXXXXXXXXXXXXXXXXX

SEQ ID NO: 641 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXAASSSSLEKXXXXXXXXXXXXXXXXXXXX

SEQ ID NO: 642 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXASSSSLEKSXXXXXXXXXXXXXXXXXXXX

SEQ ID NO: 643 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXSSSSLEKSYXXXXXXXXXXXXXXXXXXXX

SEQ ID NO: 644 - protein, 60 aa, synthetic construct
  VARIANT 1..20: note = X can be G, A, or deleted
  VARIANT 22: note = X can be any naturally-occurring amino acid or any unnatural amino acid
  VARIANT 41..60: note = X can be G, A, or deleted
XXXXXXXXXXXXXXXXXXXXFXTTAEREIVRDIKEKLCYVXXXXXXXXXXXXXXXXXXXX

SEQ ID NO: 645 - protein, 60 aa, synthetic construct
  VARIANT 1..20: note = X can be G, A, or deleted
  VARIANT 21: note = X can be any naturally-occurring amino acid or any unnatural amino acid
  VARIANT 41..60: note = X can be G, A, or deleted
XXXXXXXXXXXXXXXXXXXXXTTAEREIVRDIKEKLCYVAXXXXXXXXXXXXXXXXXXXX

SEQ ID NO: 646 - protein, 60 aa, synthetic construct
  VARIANT 1..20: note = X can be G, A, or deleted
  VARIANT 21: note = X can be any naturally-occurring amino acid or any unnatural amino acid
  VARIANT 41..60: note = X can be G, A, or deleted
XXXXXXXXXXXXXXXXXXXXXTAEREIVRDIKEKLCYVALXXXXXXXXXXXXXXXXXXXX

SEQ ID NO: 647 - protein, 60 aa, synthetic construct
  VARIANT 1..20: note = X can be G, A, or deleted
  VARIANT 21: note = X can be any naturally-occurring amino acid or any unnatural amino acid
  VARIANT 41..60: note = X can be G, A, or deleted
XXXXXXXXXXXXXXXXXXXXXAEREIVRDIKEKLCYVALDXXXXXXXXXXXXXXXXXXXX

SEQ ID NO: 648 - protein, 60 aa, synthetic construct
  VARIANT 1..20: note = X can be G, A, or deleted
  VARIANT 21: note = X can be any naturally-occurring amino acid or any unnatural amino acid
  VARIANT 41..60: note = X can be G, A, or deleted
XXXXXXXXXXXXXXXXXXXXXEREIVRDIKEKLCYVALDFXXXXXXXXXXXXXXXXXXXX

SEQ ID NO: 649 - protein, 60 aa, synthetic construct
  VARIANT 1..20: note = X can be G, A, or deleted
  VARIANT 21: note = X can be any naturally-occurring amino acid or any unnatural amino acid
  VARIANT 41..60: note = X can be G, A, or deleted
XXXXXXXXXXXXXXXXXXXXXREIVRDIKEKLCYVALDFEXXXXXXXXXXXXXXXXXXXX

SEQ ID NO: 650 - protein, 59 aa, synthetic construct
  VARIANT 1..20: note = X can be G, A, or deleted
  VARIANT 39: note = X can be any naturally-occurring amino acid or any unnatural amino acid
  VARIANT 40..59: note = X can be G, A, or deleted
XXXXXXXXXXXXXXXXXXXXEIVRDIKEKLCYVALDFEXXXXXXXXXXXXXXXXXXXXX

SEQ ID NO: 651 - protein, 60 aa, synthetic construct
  VARIANT 1..20: note = X can be G, A, or deleted
  VARIANT 38: note = X can be any naturally-occurring amino acid or any unnatural amino acid
  VARIANT 41..60: note = X can be G, A, or deleted
XXXXXXXXXXXXXXXXXXXXIVRDIKEKLCYVALDFEXEMXXXXXXXXXXXXXXXXXXXX

SEQ ID NO: 652 - protein, 60 aa, synthetic construct
  VARIANT 1..20: note = X can be G, A, or deleted
  VARIANT 37: note = X can be any naturally-occurring amino acid or any unnatural amino acid
  VARIANT 41..60: note = X can be G, A, or deleted
XXXXXXXXXXXXXXXXXXXXVRDIKEKLCYVALDFEXEMAXXXXXXXXXXXXXXXXXXXX

SEQ ID NO: 653 - protein, 60 aa, synthetic construct
  VARIANT 1..20: note = X can be G, A, or deleted
  VARIANT 36: note = X can be any naturally-occurring amino acid or any unnatural amino acid
  VARIANT 41..60: note = X can be G, A, or deleted
XXXXXXXXXXXXXXXXXXXXRDIKEKLCYVALDFEXEMATXXXXXXXXXXXXXXXXXXXX

SEQ ID NO: 654 - protein, 60 aa, synthetic construct
  VARIANT 1..20: note = X can be G, A, or deleted
  VARIANT 35: note = X can be any naturally-occurring amino acid or any unnatural amino acid
  VARIANT 41..60: note = X can be G, A, or deleted
XXXXXXXXXXXXXXXXXXXXDIKEKLCYVALDFEXEMATAXXXXXXXXXXXXXXXXXXXX

SEQ ID NO: 655 - protein, 60 aa, synthetic construct
  VARIANT 1..20: note = X can be G, A, or deleted
  VARIANT 34: note = X can be any naturally-occurring amino acid or any unnatural amino acid
  VARIANT 41..60: note = X can be G, A, or deleted
XXXXXXXXXXXXXXXXXXXXIKEKLCYVALDFEXEMATAAXXXXXXXXXXXXXXXXXXXX

SEQ ID NO: 656 - protein, 60 aa, synthetic construct
  VARIANT 1..20: note = X can be G, A, or deleted
  VARIANT 33: note = X can be any naturally-occurring amino acid or any unnatural amino acid
  VARIANT 41..60: note = X can be G, A, or deleted
XXXXXXXXXXXXXXXXXXXXKEKLCYVALDFEXEMATAASXXXXXXXXXXXXXXXXXXXX

SEQ ID NO: 657 - protein, 60 aa, synthetic construct
  VARIANT 1..20: note = X can be G, A, or deleted
  VARIANT 32: note = X can be any naturally-occurring amino acid or any unnatural amino acid
  VARIANT 41..60: note = X can be G, A, or deleted
XXXXXXXXXXXXXXXXXXXXEKLCYVALDFEXEMATAASSXXXXXXXXXXXXXXXXXXXX

SEQ ID NO: 658 - protein, 60 aa, synthetic construct
  VARIANT 1..20: note = X can be G, A, or deleted
  VARIANT 31: note = X can be any naturally-occurring amino acid or any unnatural amino acid
  VARIANT 41..60: note = X can be G, A, or deleted
XXXXXXXXXXXXXXXXXXXXKLCYVALDFEXEMATAASSSXXXXXXXXXXXXXXXXXXXX

SEQ ID NO: 659 - protein, 60 aa, synthetic construct
  VARIANT 1..20: note = X can be G, A, or deleted
  VARIANT 30: note = X can be any naturally-occurring amino acid or any unnatural amino acid
  VARIANT 41..60: note = X can be G, A, or deleted
XXXXXXXXXXXXXXXXXXXXLCYVALDFEXEMATAASSSSXXXXXXXXXXXXXXXXXXXX

SEQ ID NO: 660 - protein, 60 aa, synthetic construct
  VARIANT 1..20: note = X can be G, A, or deleted
  VARIANT 29: note = X can be any naturally-occurring amino acid or any unnatural amino acid
  VARIANT 41..60: note = X can be G, A, or deleted
XXXXXXXXXXXXXXXXXXXXCYVALDFEXEMATAASSSSLXXXXXXXXXXXXXXXXXXXX

SEQ ID NO: 661 - protein, 60 aa, synthetic construct
  VARIANT 1..20: note = X can be G, A, or deleted
  VARIANT 28: note = X can be any naturally-occurring amino acid or any unnatural amino acid
  VARIANT 41..60: note = X can be G, A, or deleted
XXXXXXXXXXXXXXXXXXXXYVALDFEXEMATAASSSSLEXXXXXXXXXXXXXXXXXXXX

SEQ ID NO: 662 - protein, 60 aa, synthetic construct
  VARIANT 1..20: note = X can be G, A, or deleted
  VARIANT 27: note = X can be any naturally-occurring amino acid or any unnatural amino acid
  VARIANT 41..60: note = X can be G, A, or deleted
XXXXXXXXXXXXXXXXXXXXVALDFEXEMATAASSSSLEKXXXXXXXXXXXXXXXXXXXX

SEQ ID NO: 663 - protein, 60 aa, synthetic construct
  VARIANT 1..20: note = X can be G, A, or deleted
  VARIANT 26: note = X can be any naturally-occurring amino acid or any unnatural amino acid
  VARIANT 41..60: note = X can be G, A, or deleted
XXXXXXXXXXXXXXXXXXXXALDFEXEMATAASSSSLEKSXXXXXXXXXXXXXXXXXXXX

SEQ ID NO: 664 - protein, 60 aa, synthetic construct
  VARIANT 1..20: note = X can be G, A, or deleted
  VARIANT 25: note = X can be any naturally-occurring amino acid or any unnatural amino acid
  VARIANT 41..60: note = X can be G, A, or deleted
XXXXXXXXXXXXXXXXXXXXLDFEXEMATAASSSSLEKSYXXXXXXXXXXXXXXXXXXXX

SEQ ID NO: 665 - protein, 60 aa, synthetic construct
  VARIANT 1..20: note = X can be G, A, or deleted
  VARIANT 24: note = X can be any naturally-occurring amino acid or any unnatural amino acid
  VARIANT 41..60: note = X can be G, A, or deleted
XXXXXXXXXXXXXXXXXXXXDFEXEMATAASSSSLEKSYEXXXXXXXXXXXXXXXXXXXX

SEQ ID NO: 666 - protein, 60 aa, synthetic construct
  VARIANT 1..20: note = X can be G, A, or deleted
  VARIANT 21: note = X can be any naturally-occurring amino acid or any unnatural amino acid
  VARIANT 35: note = X can be any naturally-occurring amino acid or any unnatural amino acid
  VARIANT 41..60: note = X can be G, A, or deleted
XXXXXXXXXXXXXXXXXXXXXIKEKLCYVALDFEXEMATAXXXXXXXXXXXXXXXXXXXX

SEQ ID NO: 667 - protein, 60 aa, synthetic construct
  VARIANT 1..20: note = X can be G, A, or deleted
  VARIANT 22: note = X can be any naturally-occurring amino acid or any unnatural amino acid
  VARIANT 35: note = X can be any naturally-occurring amino acid or any unnatural amino acid
  VARIANT 41..60: note = X can be G, A, or deleted
XXXXXXXXXXXXXXXXXXXXDXKEKLCYVALDFEXEMATAXXXXXXXXXXXXXXXXXXXX

SEQ ID NO: 668 - protein, 60 aa, synthetic construct
  VARIANT 1..20: note = X can be G, A, or deleted
  VARIANT 23: note = X can be any naturally-occurring amino acid or any unnatural amino acid
  VARIANT 35: note = X can be any naturally-occurring amino acid or any unnatural amino acid
  VARIANT 41..60: note = X can be G, A, or deleted
XXXXXXXXXXXXXXXXXXXXDIXEKLCYVALDFEXEMATAXXXXXXXXXXXXXXXXXXXX

SEQ ID NO: 669 - protein, 60 aa, synthetic construct
  VARIANT 1..20: note = X can be G, A, or deleted
  VARIANT 24: note = X can be any naturally-occurring amino acid or any unnatural amino acid
  VARIANT 35: note = X can be any naturally-occurring amino acid or any unnatural amino acid
  VARIANT 41..60: note = X can be G, A, or deleted
XXXXXXXXXXXXXXXXXXXXDIKXKLCYVALDFEXEMATAXXXXXXXXXXXXXXXXXXXX

SEQ ID NO: 670 - protein, 60 aa, synthetic construct
  VARIANT 1..20: note = X can be G, A, or deleted
  VARIANT 25: note = X can be any naturally-occurring amino acid or any unnatural amino acid
  VARIANT 35: note = X can be any naturally-occurring amino acid or any unnatural amino acid
  VARIANT 41..60: note = X can be G, A, or deleted
XXXXXXXXXXXXXXXXXXXXDIKEXLCYVALDFEXEMATAXXXXXXXXXXXXXXXXXXXX

SEQ ID NO: 671 - protein, 60 aa, synthetic construct
  VARIANT 1..20: note = X can be G, A, or deleted
  VARIANT 26: note = X can be any naturally-occurring amino acid or any unnatural amino acid
  VARIANT 35: note = X can be any naturally-occurring amino acid or any unnatural amino acid
  VARIANT 41..60: note = X can be G, A, or deleted
XXXXXXXXXXXXXXXXXXXXDIKEKXCYVALDFEXEMATAXXXXXXXXXXXXXXXXXXXX

SEQ ID NO: 672 - protein, 60 aa, synthetic construct
  VARIANT 1..20: note = X can be G, A, or deleted
  VARIANT 27: note = X can be any naturally-occurring amino acid or any unnatural amino acid
  VARIANT 35: note = X can be any naturally-occurring amino acid or any unnatural amino acid
  VARIANT 41..60: note = X can be G, A, or deleted
XXXXXXXXXXXXXXXXXXXXDIKEKLXYVALDFEXEMATAXXXXXXXXXXXXXXXXXXXX

SEQ ID NO: 673 - protein, 60 aa, synthetic construct
  VARIANT 1..20: note = X can be G, A, or deleted
  VARIANT 28: note = X can be any naturally-occurring amino acid or any unnatural amino acid
  VARIANT 35: note = X can be any naturally-occurring amino acid or any unnatural amino acid
  VARIANT 41..60: note = X can be G, A, or deleted
XXXXXXXXXXXXXXXXXXXXDIKEKLCXVALDFEXEMATAXXXXXXXXXXXXXXXXXXXX

SEQ ID NO: 674 - protein, 60 aa, synthetic construct
  VARIANT 1..20: note = X can be G, A, or deleted
  VARIANT 29: note = X can be any naturally-occurring amino acid or any unnatural amino acid
  VARIANT 35: note = X can be any naturally-occurring amino acid or any unnatural amino acid
  VARIANT 41..60: note = X can be G, A, or deleted
XXXXXXXXXXXXXXXXXXXXDIKEKLCYXALDFEXEMATAXXXXXXXXXXXXXXXXXXXX

SEQ ID NO: 675 - protein, 60 aa, synthetic construct
  VARIANT 1..20: note = X can be G, A, or deleted
  VARIANT 30: note = X can be any naturally-occurring amino acid or any unnatural amino acid
  VARIANT 35: note = X can be any naturally-occurring amino acid or any unnatural amino acid
  VARIANT 41..60: note = X can be G, A, or deleted
XXXXXXXXXXXXXXXXXXXXDIKEKLCYVXLDFEXEMATAXXXXXXXXXXXXXXXXXXXX

SEQ ID NO: 676 - protein, 60 aa, synthetic construct
  VARIANT 1..20: note = X can be G, A, or deleted
  VARIANT 31: note = X can be any naturally-occurring amino acid or any unnatural amino acid
  VARIANT 35: note = X can be any naturally-occurring amino acid or any unnatural amino acid
  VARIANT 41..60: note = X can be G, A, or deleted
XXXXXXXXXXXXXXXXXXXXDIKEKLCYVAXDFEXEMATAXXXXXXXXXXXXXXXXXXXX

SEQ ID NO: 677 - protein, 60 aa, synthetic construct
  VARIANT 1..20: note = X can be G, A, or deleted
  VARIANT 32: note = X can be any naturally-occurring amino acid or any unnatural amino acid
  VARIANT 35: note = X can be any naturally-occurring amino acid or any unnatural amino acid
  VARIANT 41..60: note = X can be G, A, or deleted
XXXXXXXXXXXXXXXXXXXXDIKEKLCYVALXFEXEMATAXXXXXXXXXXXXXXXXXXXX

SEQ ID NO: 678 - protein, 60 aa, synthetic construct
  VARIANT 1..20: note = X can be G, A, or deleted
  VARIANT 33: note = X can be any naturally-occurring amino acid or any unnatural amino acid
  VARIANT 35: note = X can be any naturally-occurring amino acid or any unnatural amino acid
  VARIANT 41..60: note = X can be G, A, or deleted
XXXXXXXXXXXXXXXXXXXXDIKEKLCYVALDXEXEMATAXXXXXXXXXXXXXXXXXXXX

SEQ ID NO: 679 - protein, 60 aa, synthetic construct
  VARIANT 1..20: note = X can be G, A, or deleted
  VARIANT 34..35: note = X can be any naturally-occurring amino acid or any unnatural amino acid
  VARIANT 41..60: note = X can be G, A, or deleted
XXXXXXXXXXXXXXXXXXXXDIKEKLCYVALDFXXEMATAXXXXXXXXXXXXXXXXXXXX

SEQ ID NO: 680 - protein, 60 aa, synthetic construct
  VARIANT 1..20: note = X can be G, A, or deleted
  VARIANT 35..36: note = X can be any naturally-occurring amino acid or any unnatural amino acid
  VARIANT 41..60: note = X can be G, A, or deleted
XXXXXXXXXXXXXXXXXXXXDIKEKLCYVALDFEXXMATAXXXXXXXXXXXXXXXXXXXX

SEQ ID NO: 681 - protein, 60 aa, synthetic construct
  VARIANT 1..20: note = X can be G, A, or deleted
  VARIANT 35: note = X can be any naturally-occurring amino acid or any unnatural amino acid
  VARIANT 37: note = X can be any naturally-occurring amino acid or any unnatural amino acid
  VARIANT 41..60: note = X can be G, A, or deleted
XXXXXXXXXXXXXXXXXXXXDIKEKLCYVALDFEXEXATAXXXXXXXXXXXXXXXXXXXX

SEQ ID NO: 682 - protein, 60 aa, synthetic construct
  VARIANT 1..20: note = X can be G, A, or deleted
  VARIANT 35: note = X can be any naturally-occurring amino acid or any unnatural amino acid
  VARIANT 38: note = X can be any naturally-occurring amino acid or any unnatural amino acid
  VARIANT 41..60: note = X can be G, A, or deleted
XXXXXXXXXXXXXXXXXXXXDIKEKLCYVALDFEXEMXTAXXXXXXXXXXXXXXXXXXXX

SEQ ID NO: 683 - protein, 60 aa, synthetic construct
  VARIANT 1..20: note = X can be G, A, or deleted
  VARIANT 35: note = X can be any naturally-occurring amino acid or any unnatural amino acid
  VARIANT 39: note = X can be any naturally-occurring amino acid or any unnatural amino acid
  VARIANT 41..60: note = X can be G, A, or deleted
XXXXXXXXXXXXXXXXXXXXDIKEKLCYVALDFEXEMAXAXXXXXXXXXXXXXXXXXXXX

SEQ ID NO: 684 - protein, 60 aa, synthetic construct
  VARIANT 1..20: note = X can be G, A, or deleted
  VARIANT 35: note = X can be any naturally-occurring amino acid or any unnatural amino acid
  VARIANT 40: note = X can be any naturally-occurring amino acid or any unnatural amino acid
  VARIANT 41..60: note = X can be G, A, or deleted
XXXXXXXXXXXXXXXXXXXXDIKEKLCYVALDFEXEMATXXXXXXXXXXXXXXXXXXXXX

SEQ ID NO: 685 - protein, 81 aa, synthetic construct
  VARIANT 1..20: note = X can be G, A, or deleted
  VARIANT 21..22: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXXXTTAEREIVRDIKEKLCYVALDFEXEMATAASSSSLEKSYXXXXXXXXXXXXXXXXXXXX

SEQ ID NO: 686 - protein, 63 aa, synthetic construct
  VARIANT 1..2: note = X can be G, A, or deleted
  VARIANT 4..5: note = X can be any naturally-occurring amino acid or any unnatural amino acid
  VARIANT 28: note = X can be any naturally-occurring amino acid or any unnatural amino acid
  VARIANT 44..63: note = X can be G, A, or deleted
XXFXXTAEREIVRDIKEKLCYVALDFEXEMATAASSSSLEKSYXXXXXXXXXXXXXXXXXXXX

SEQ ID NO: 687 - protein, 81 aa, synthetic construct
  VARIANT 1..20: note = X can be G, A, or deleted
  VARIANT 22: note = X can be any naturally-occurring amino acid or any unnatural amino acid
  VARIANT 24: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTXAEREIVRDIKEKLCYVALDFEXEMATAASSSSLEKSYXXXXXXXXXXXXXXXXXXXX

SEQ ID NO: 688 - protein, 81 aa, synthetic construct
  VARIANT 1..20: note = X can be G, A, or deleted
  VARIANT 22: note = X can be any naturally-occurring amino acid or any unnatural amino acid
  VARIANT 25: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTXEREIVRDIKEKLCYVALDFEXEMATAASSSSLEKSYXXXXXXXXXXXXXXXXXXXX

SEQ ID NO: 689 - protein, 81 aa, synthetic construct
  VARIANT 1..20: note = X can be G, A, or deleted
  VARIANT 22: note = X can be any naturally-occurring amino acid or any unnatural amino acid
  VARIANT 26: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAXREIVRDIKEKLCYVALDFEXEMATAASSSSLEKSYXXXXXXXXXXXXXXXXXXXX

SEQ ID NO: 690 - protein, 81 aa, synthetic construct
  VARIANT 1..20: note = X can be G, A, or deleted
  VARIANT 22: note = X can be any naturally-occurring amino acid or any unnatural amino acid
  VARIANT 27: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEXEIVRDIKEKLCYVALDFEXEMATAASSSSLEKSYXXXXXXXXXXXXXXXXXXXX

SEQ ID NO: 691 - protein, 81 aa, synthetic construct
  VARIANT 1..20: note = X can be G, A, or deleted
  VARIANT 22: note = X can be any naturally-occurring amino acid or any unnatural amino acid
  VARIANT 28: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAERXIVRDIKEKLCYVALDFEXEMATAASSSSLEKSYXXXXXXXXXXXXXXXXXXXX

SEQ ID NO: 692 - protein, 81 aa, synthetic construct
  VARIANT 1..20: note = X can be G, A, or deleted
  VARIANT 22: note = X can be any naturally-occurring amino acid or any unnatural amino acid
  VARIANT 29: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEREXVRDIKEKLCYVALDFEXEMATAASSSSLEKSYXXXXXXXXXXXXXXXXXXXX

SEQ ID NO: 693 - protein, 81 aa, synthetic construct
  VARIANT 1..20: note = X can be G, A, or deleted
  VARIANT 22: note = X can be any naturally-occurring amino acid or any unnatural amino acid
  VARIANT 30: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEREIXRDIKEKLCYVALDFEXEMATAASSSSLEKSYXXXXXXXXXXXXXXXXXXXX

SEQ ID NO: 694 - protein, 81 aa, synthetic construct
  VARIANT 1..20: note = X can be G, A, or deleted
  VARIANT 22: note = X can be any naturally-occurring amino acid or any unnatural amino acid
  VARIANT 31: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEREIVXDIKEKLCYVALDFEXEMATAASSSSLEKSYXXXXXXXXXXXXXXXXXXXX

SEQ ID NO: 695 - protein, 81 aa, synthetic construct
  VARIANT 1..20: note = X can be G, A, or deleted
  VARIANT 22: note = X can be any naturally-occurring amino acid or any unnatural amino acid
  VARIANT 32: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEREIVRXIKEKLCYVALDFEXEMATAASSSSLEKSYXXXXXXXXXXXXXXXXXXXX

SEQ ID NO: 696 - protein, 81 aa, synthetic construct
  VARIANT 1..20: note = X can be G, A, or deleted
  VARIANT 22: note = X can be any naturally-occurring amino acid or any unnatural amino acid
  VARIANT 33: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEREIVRDXKEKLCYVALDFEXEMATAASSSSLEKSYXXXXXXXXXXXXXXXXXXXX

SEQ ID NO: 697 - protein, 81 aa, synthetic construct
  VARIANT 1..20: note = X can be G, A, or deleted
  VARIANT 22: note = X can be any naturally-occurring amino acid or any unnatural amino acid
  VARIANT 34: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEREIVRDIXEKLCYVALDFEXEMATAASSSSLEKSYXXXXXXXXXXXXXXXXXXXX

SEQ ID NO: 698 - protein, 81 aa, synthetic construct
  VARIANT 1..20: note = X can be G, A, or deleted
  VARIANT 22: note = X can be any naturally-occurring amino acid or any unnatural amino acid
  VARIANT 35: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEREIVRDIKXKLCYVALDFEXEMATAASSSSLEKSYXXXXXXXXXXXXXXXXXXXX

SEQ ID NO: 699 - protein, 81 aa, synthetic construct
  VARIANT 1..20: note = X can be G, A, or deleted
  VARIANT 22: note = X can be any naturally-occurring amino acid or any unnatural amino acid
  VARIANT 36: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEREIVRDIKEXLCYVALDFEXEMATAASSSSLEKSYXXXXXXXXXXXXXXXXXXXX

SEQ ID NO: 700 - protein, 81 aa, synthetic construct
  VARIANT 1..20: note = X can be G, A, or deleted
  VARIANT 22: note = X can be any naturally-occurring amino acid or any unnatural amino acid
  VARIANT 37: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEREIVRDIKEKXCYVALDFEXEMATAASSSSLEKSYXXXXXXXXXXXXXXXXXXXX

SEQ ID NO: 701 - protein, 81 aa, synthetic construct
  VARIANT 1..20: note = X can be G, A, or deleted
  VARIANT 22: note = X can be any naturally-occurring amino acid or any unnatural amino acid
  VARIANT 38: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLXYVALDFEXEMATAASSSSLEKSYXXXXXXXXXXXXXXXXXXXX

SEQ ID NO: 702 - protein, 81 aa, synthetic construct
  VARIANT 1..20: note = X can be G, A, or deleted
  VARIANT 22: note = X can be any naturally-occurring amino acid or any unnatural amino acid
  VARIANT 39: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCXVALDFEXEMATAASSSSLEKSYXXXXXXXXXXXXXXXXXXXX

SEQ ID NO: 703 - protein, 81 aa, synthetic construct
  VARIANT 1..20: note = X can be G, A, or deleted
  VARIANT 22: note = X can be any naturally-occurring amino acid or any unnatural amino acid
  VARIANT 40: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYXALDFEXEMATAASSSSLEKSYXXXXXXXXXXXXXXXXXXXX

SEQ ID NO: 704 - protein, 81 aa, synthetic construct
  VARIANT 1..20: note = X can be G, A, or deleted
  VARIANT 22: note = X can be any naturally-occurring amino acid or any unnatural amino acid
  VARIANT 41: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVXLDFEXEMATAASSSSLEKSYXXXXXXXXXXXXXXXXXXXX

SEQ ID NO: 705 - protein, 81 aa, synthetic construct
  VARIANT 1..20: note = X can be G, A, or deleted
  VARIANT 22: note = X can be any naturally-occurring amino acid or any unnatural amino acid
  VARIANT 42: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVAXDFEXEMATAASSSSLEKSYXXXXXXXXXXXXXXXXXXXX

SEQ ID NO: 706 - protein, 81 aa, synthetic construct
  VARIANT 1..20: note = X can be G, A, or deleted
  VARIANT 22: note = X can be any naturally-occurring amino acid or any unnatural amino acid
  VARIANT 43: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALXFEXEMATAASSSSLEKSYXXXXXXXXXXXXXXXXXXXX

SEQ ID NO: 707 - protein, 81 aa, synthetic construct
  VARIANT 1..20: note = X can be G, A, or deleted
  VARIANT 22: note = X can be any naturally-occurring amino acid or any unnatural amino acid
  VARIANT 44: note = X can be any naturally-occurring amino acid or any unnatural amino acid
  VARIANT 46: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDXEXEMATAASSSSLEKSYXXXXXXXXXXXXXXXXXXXX

SEQ ID NO: 708 - protein, 81 aa, synthetic construct
  VARIANT 1..20: note = X can be G, A, or deleted
  VARIANT 22: note = X can be any naturally-occurring amino acid or any unnatural amino acid
  VARIANT 45..46: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDFXXEMATAASSSSLEKSYXXXXXXXXXXXXXXXXXXXX

SEQ ID NO: 709 - protein, 81 aa, synthetic construct
  VARIANT 1..20: note = X can be G, A, or deleted
  VARIANT 22: note = X can be any naturally-occurring amino acid or any unnatural amino acid
  VARIANT 46..47: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDFEXXMATAASSSSLEKSYXXXXXXXXXXXXXXXXXXXX

SEQ ID NO: 710 - protein, 81 aa, synthetic construct
  VARIANT 1..20: note = X can be G, A, or deleted
  VARIANT 22: note = X can be any naturally-occurring amino acid or any unnatural amino acid
  VARIANT 46: note = X can be any naturally-occurring amino acid or any unnatural amino acid
  VARIANT 48: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDFEXEXATAASSSSLEKSYXXXXXXXXXXXXXXXXXXXX

SEQ ID NO: 711 - protein, 81 aa, synthetic construct
  VARIANT 1..20: note = X can be G, A, or deleted
  VARIANT 22: note = X can be any naturally-occurring amino acid or any unnatural amino acid
  VARIANT 46: note = X can be any naturally-occurring amino acid or any unnatural amino acid
  VARIANT 49: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDFEXEMXTAASSSSLEKSYXXXXXXXXXXXXXXXXXXXX

SEQ ID NO: 712 - protein, 81 aa, synthetic construct
  VARIANT 1..20: note = X can be G, A, or deleted
  VARIANT 22: note = X can be any naturally-occurring amino acid or any unnatural amino acid
  VARIANT 46: note = X can be any naturally-occurring amino acid or any unnatural amino acid
  VARIANT 50: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDFEXEMAXAASSSSLEKSYXXXXXXXXXXXXXXXXXXXX

SEQ ID NO: 713 - protein, 81 aa, synthetic construct
  VARIANT 1..20: note = X can be G, A, or deleted
  VARIANT 22: note = X can be any naturally-occurring amino acid or any unnatural amino acid
  VARIANT 46: note = X can be any naturally-occurring amino acid or any unnatural amino acid
  VARIANT 51: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDFEXEMATXASSSSLEKSYXXXXXXXXXXXXXXXXXXXX

SEQ ID NO: 714 - protein, 81 aa, synthetic construct
  VARIANT 1..20: note = X can be G, A, or deleted
  VARIANT 22: note = X can be any naturally-occurring amino acid or any unnatural amino acid
  VARIANT 46: note = X can be any naturally-occurring amino acid or any unnatural amino acid
  VARIANT 52: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDFEXEMATAXSSSSLEKSYXXXXXXXXXXXXXXXXXXXX

SEQ ID NO: 715 - protein, 81 aa, synthetic construct
  VARIANT 1..20: note = X can be G, A, or deleted
  VARIANT 22: note = X can be any naturally-occurring amino acid or any unnatural amino acid
  VARIANT 46: note = X can be any naturally-occurring amino acid or any unnatural amino acid
  VARIANT 53: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDFEXEMATASXSSSLEKSYXXXXXXXXXXXXXXXXXXXX

SEQ ID NO: 716 - protein, 81 aa, synthetic construct
  VARIANT 1..20: note = X can be G, A, or deleted
  VARIANT 22: note = X can be any naturally-occurring amino acid or any unnatural amino acid
  VARIANT 46: note = X can be any naturally-occurring amino acid or any unnatural amino acid
  VARIANT 54: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDFEXEMATASSXSSLEKSYXXXXXXXXXXXXXXXXXXXX

SEQ ID NO: 717 - protein, 81 aa, synthetic construct
  VARIANT 1..20: note = X can be G, A, or deleted
  VARIANT 22: note = X can be any naturally-occurring amino acid or any unnatural amino acid
  VARIANT 46: note = X can be any naturally-occurring amino acid or any unnatural amino acid
  VARIANT 55: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDFEXEMATASSSXSLEKSYXXXXXXXXXXXXXXXXXXXX

SEQ ID NO: 718 - protein, 81 aa, synthetic construct
  VARIANT 1..20: note = X can be G, A, or deleted
  VARIANT 22: note = X can be any naturally-occurring amino acid or any unnatural amino acid
  VARIANT 46: note = X can be any naturally-occurring amino acid or any unnatural amino acid
  VARIANT 56: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDFEXEMATASSSSXLEKSYXXXXXXXXXXXXXXXXXXXX

SEQ ID NO: 719 - protein, 81 aa, synthetic construct
  VARIANT 1..20: note = X can be G, A, or deleted
  VARIANT 22: note = X can be any naturally-occurring amino acid or any unnatural amino acid
  VARIANT 46: note = X can be any naturally-occurring amino acid or any unnatural amino acid
  VARIANT 57: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDFEXEMATAASSSSXEKSYXXXXXXXXXXXXXXXXXXXX

SEQ ID NO: 720 - protein, 81 aa, synthetic construct
  VARIANT 1..20: note = X can be G, A, or deleted
  VARIANT 22: note = X can be any naturally-occurring amino acid or any unnatural amino acid
  VARIANT 46: note = X can be any naturally-occurring amino acid or any unnatural amino acid
  VARIANT 58: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDFEXEMATAASSSSLXKSYXXXXXXXXXXXXXXXXXXXX

SEQ ID NO: 721 - protein, 81 aa, synthetic construct
  VARIANT 1..20: note = X can be G, A, or deleted
  VARIANT 22: note = X can be any naturally-occurring amino acid or any unnatural amino acid
  VARIANT 46: note = X can be any naturally-occurring amino acid or any unnatural amino acid
  VARIANT 59: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDFEXEMATAASSSSLEXSYXXXXXXXXXXXXXXXXXXXX

SEQ ID NO: 722 - protein, 81 aa, synthetic construct
  VARIANT 1..20: note = X can be G, A, or deleted
  VARIANT 22: note = X can be any naturally-occurring amino acid or any unnatural amino acid
  VARIANT 46: note = X can be any naturally-occurring amino acid or any unnatural amino acid
  VARIANT 60: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDFEXEMATAASSSSLEKXYXXXXXXXXXXXXXXXXXXXX

SEQ ID NO: 723 - protein, 81 aa, synthetic construct
  VARIANT 1..20: note = X can be G, A, or deleted
  VARIANT 22: note = X can be any naturally-occurring amino acid or any unnatural amino acid
  VARIANT 46: note = X can be any naturally-occurring amino acid or any unnatural amino acid
  VARIANT 61: note = X can be any naturally-occurring amino acid or any unnatural amino acid
  VARIANT 62..81: note = X can be G, A, or deleted
XXXXXXXXXXXXXXXXXXXXFXTTAEREIVRDIKEKLCYVALDFEXEMATAASSSSLEKSXXXXXXXXXXXXXXXXXXXXX

SEQ ID NO: 724 - protein, 50 aa, synthetic construct
  VARIANT 1..20: note = X can be any naturally-occurring amino acid or any unnatural amino acid or deleted
  VARIANT 31..50: note = X can be any naturally-occurring amino acid or any unnatural amino acid or deleted
XXXXXXXXXXXXXXXXXXXXKYSVWIGGSIXXXXXXXXXXXXXXXXXXXX

SEQ ID NO: 725 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXYSVWIGGSIXXXXXXXXXXXXXXXXXXXX

SEQ ID NO: 726 - protein, 50 aa, synthetic construct
  VARIANT 1..20: note = X can be G, A, or deleted
  VARIANT 22: note = X can be any naturally-occurring amino acid or any unnatural amino acid
  VARIANT 31..50: note = X can be G, A, or deleted
XXXXXXXXXXXXXXXXXXXXKXSVWIGGSIXXXXXXXXXXXXXXXXXXXX

SEQ ID NO: 727 - protein, 50 aa, synthetic construct
  VARIANT 1..20: note = X can be G, A, or deleted
  VARIANT 23: note = X can be any naturally-occurring amino acid or any unnatural amino acid
  VARIANT 31..50: note = X can be G, A, or deleted
XXXXXXXXXXXXXXXXXXXXKYXVWIGGSIXXXXXXXXXXXXXXXXXXXX

SEQ ID NO: 728 - protein, 50 aa, synthetic construct
  VARIANT 1..20: note = X can be G, A, or deleted
  VARIANT 24: note = X can be any naturally-occurring amino acid or any unnatural amino acid
  VARIANT 31..50: note = X can be G, A, or deleted
XXXXXXXXXXXXXXXXXXXXKYSXWIGGSIXXXXXXXXXXXXXXXXXXXX

SEQ ID NO: 729 - protein, 50 aa, synthetic construct
  VARIANT 1..20: note = X can be G, A, or deleted
  VARIANT 25: note = X can be any naturally-occurring amino acid or any unnatural amino acid
  VARIANT 31..50: note = X can be G, A, or deleted
XXXXXXXXXXXXXXXXXXXXKYSVXIGGSIXXXXXXXXXXXXXXXXXXXX

SEQ ID NO: 730 - protein, 50 aa, synthetic construct
  VARIANT 1..20: note = X can be G, A, or deleted
  VARIANT 26: note = X can be any naturally-occurring amino acid or any unnatural amino acid
  VARIANT 31..50: note = X can be G, A, or deleted
XXXXXXXXXXXXXXXXXXXXKYSVWXGGSIXXXXXXXXXXXXXXXXXXXX

SEQ ID NO: 731 - protein, 50 aa, synthetic construct
  VARIANT 1..20: note = X can be G, A, or deleted
  VARIANT 27: note = X can be any naturally-occurring amino acid or any unnatural amino acid
  VARIANT 31..50: note = X can be G, A, or deleted
XXXXXXXXXXXXXXXXXXXXKYSVWIXGSIXXXXXXXXXXXXXXXXXXXX

SEQ ID NO: 732 - protein, 50 aa, synthetic construct
  VARIANT 1..20: note = X can be G, A, or deleted
  VARIANT 28: note = X can be any naturally-occurring amino acid or any unnatural amino acid
  VARIANT 31..50: note = X can be G, A, or deleted
XXXXXXXXXXXXXXXXXXXXKYSVWIGXSIXXXXXXXXXXXXXXXXXXXX

SEQ ID NO: 733 - protein, 50 aa, synthetic construct
  VARIANT 1..20: note = X can be G, A, or deleted
  VARIANT 29: note = X can be any naturally-occurring amino acid or any unnatural amino acid
  VARIANT 31..50: note = X can be G, A, or deleted
XXXXXXXXXXXXXXXXXXXXKYSVWIGGXIXXXXXXXXXXXXXXXXXXXX

SEQ ID NO: 734 - protein, 50 aa, synthetic construct
  VARIANT 1..20: note = X can be G, A, or deleted
  VARIANT 30: note = X can be any naturally-occurring amino acid or any unnatural amino acid
  VARIANT 31..50: note = X can be G, A, or deleted
XXXXXXXXXXXXXXXXXXXXKYSVWIGGSXXXXXXXXXXXXXXXXXXXXX

SEQ ID NO: 735 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXPSTMKIKIIXXXXXXXXXXXXXXXXXXXX

SEQ ID NO: 736 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXSTMKIKIIAXXXXXXXXXXXXXXXXXXXX

SEQ ID NO: 737 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXTMKIKIIAPXXXXXXXXXXXXXXXXXXXX

SEQ ID NO: 738 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXMKIKIIAPPXXXXXXXXXXXXXXXXXXXX

SEQ ID NO: 739 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXKIKIIAPPEXXXXXXXXXXXXXXXXXXXX

SEQ ID NO: 740 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXIKIIAPPERXXXXXXXXXXXXXXXXXXXX

SEQ ID NO: 741 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXKIIAPPERKXXXXXXXXXXXXXXXXXXXX

SEQ ID NO: 742 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXIIAPPERKYXXXXXXXXXXXXXXXXXXXX

SEQ ID NO: 743 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXIAPPERKYSXXXXXXXXXXXXXXXXXXXX

SEQ ID NO: 744 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXAPPERKYSVXXXXXXXXXXXXXXXXXXXX

SEQ ID NO: 745 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXPPERKYSVWXXXXXXXXXXXXXXXXXXXX

SEQ ID NO: 746 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXPERKYSVWIXXXXXXXXXXXXXXXXXXXX

SEQ ID NO: 747 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXERKYSVWIGXXXXXXXXXXXXXXXXXXXX

SEQ ID NO: 748 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXRKYSVWIGGXXXXXXXXXXXXXXXXXXXX

SEQ ID NO: 749 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXKYSVWIGGSXXXXXXXXXXXXXXXXXXXX

SEQ ID NO: 750 - protein, 50 aa, synthetic construct; identical to SEQ ID NO: 725
  VARIANT 1..20: note = X can be G, A, or deleted
  VARIANT 21: note = X can be any naturally-occurring amino acid or any unnatural amino acid
  VARIANT 31..50: note = X can be G, A, or deleted

SEQ ID NO: 751 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXSVWIGGSILXXXXXXXXXXXXXXXXXXXX

SEQ ID NO: 752 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXVWIGGSILAXXXXXXXXXXXXXXXXXXXX

SEQ ID NO: 753 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXWIGGSILASXXXXXXXXXXXXXXXXXXXX

SEQ ID NO: 754 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXIGGSILASLXXXXXXXXXXXXXXXXXXXX

SEQ ID NO: 755 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXGGSILASLSXXXXXXXXXXXXXXXXXXXX

SEQ ID NO: 756 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXGSILASLSTXXXXXXXXXXXXXXXXXXXX

SEQ ID NO: 757 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXSILASLSTFXXXXXXXXXXXXXXXXXXXX

SEQ ID NO: 758 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXILASLSTFQXXXXXXXXXXXXXXXXXXXX

SEQ ID NO: 759 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXLASLSTFQQXXXXXXXXXXXXXXXXXXXX

SEQ ID NO: 760 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXASLSTFQQMXXXXXXXXXXXXXXXXXXXX

SEQ ID NO: 761 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXSLSTFQQMWXXXXXXXXXXXXXXXXXXXX

SEQ ID NO: 762 - protein, 50 aa, synthetic construct
  VARIANT 1..20: note = X can be G, A, or deleted
  VARIANT 21: note = X can be any naturally-occurring amino acid or any unnatural amino acid
  VARIANT 31..50: note = X can be G, A, or deleted
XXXXXXXXXXXXXXXXXXXXXLSTFQQMWIXXXXXXXXXXXXXXXXXXXX

SEQ ID NO: 763 - protein, 50 aa, synthetic construct
  VARIANT 1..20: note = X can be G, A, or deleted
  VARIANT 30: note = X can be any naturally-occurring amino acid or any unnatural amino acid
  VARIANT 31..50: note = X can be G, A, or deleted
XXXXXXXXXXXXXXXXXXXXLSTFQQMWIXXXXXXXXXXXXXXXXXXXXX

SEQ ID NO: 764 - protein, 50 aa, synthetic construct
  VARIANT 1..20: note = X can be G, A, or deleted
  VARIANT 29: note = X can be any naturally-occurring amino acid or any unnatural amino acid
  VARIANT 31..50: note = X can be G, A, or deleted
XXXXXXXXXXXXXXXXXXXXSTFQQMWIXKXXXXXXXXXXXXXXXXXXXX

SEQ ID NO: 765 - protein, 50 aa, synthetic construct
  VARIANT 1..20: note = X can be G, A, or deleted
  VARIANT 28: note = X can be any naturally-occurring amino acid or any unnatural amino acid
  VARIANT 31..50: note = X can be G, A, or deleted
XXXXXXXXXXXXXXXXXXXXTFQQMWIXKQXXXXXXXXXXXXXXXXXXXX

SEQ ID NO: 766 - protein, 60 aa, synthetic construct
  VARIANT 1..20: note = X can be G, A, or deleted
  VARIANT 41..60: note = X can be G, A, or deleted
XXXXXXXXXXXXXXXXXXXXAPSTMKIKIIAPPERKYSVWXXXXXXXXXXXXXXXXXXXX

SEQ ID NO: 767 - protein, 60 aa, synthetic construct
  VARIANT 1..20: note = X can be G, A, or deleted
  VARIANT 41..60: note = X can be G, A, or deleted
XXXXXXXXXXXXXXXXXXXXPSTMKIKIIAPPERKYSVWIXXXXXXXXXXXXXXXXXXXX

SEQ ID NO: 768 - protein, 60 aa, synthetic construct
  VARIANT 1..20: note = X can be G, A, or deleted
  VARIANT 41..60: note = X can be G, A, or deleted
XXXXXXXXXXXXXXXXXXXXSTMKIKIIAPPERKYSVWIGXXXXXXXXXXXXXXXXXXXX

SEQ ID NO: 769 - protein, 60 aa, synthetic construct
  VARIANT 1..20: note = X can be G, A, or deleted
  VARIANT 41..60: note = X can be G, A, or deleted
XXXXXXXXXXXXXXXXXXXXTMKIKIIAPPERKYSVWIGGXXXXXXXXXXXXXXXXXXXX

SEQ ID NO: 770 - protein, 60 aa, synthetic construct
  VARIANT 1..20: note = X can be G, A, or deleted
  VARIANT 41..60: note = X can be G, A, or deleted
XXXXXXXXXXXXXXXXXXXXMKIKIIAPPERKYSVWIGGSXXXXXXXXXXXXXXXXXXXX

SEQ ID NO: 771 - protein, 60 aa, synthetic construct
  VARIANT 1..20: note = X can be G, A, or deleted
  VARIANT 41..60: note = X can be G, A, or deleted
XXXXXXXXXXXXXXXXXXXXKIKIIAPPERKYSVWIGGSIXXXXXXXXXXXXXXXXXXXX

SEQ ID NO: 772 - protein, 60 aa, synthetic construct
  VARIANT 1..20: note = X can be G, A, or deleted
  VARIANT 41..60: note = X can be G, A, or deleted
XXXXXXXXXXXXXXXXXXXXIKIIAPPERKYSVWIGGSILXXXXXXXXXXXXXXXXXXXX

SEQ ID NO: 773 - protein, 60 aa, synthetic construct
  VARIANT 1..20: note = X can be G, A, or deleted
  VARIANT 41..60: note = X can be G, A, or deleted
XXXXXXXXXXXXXXXXXXXXKIIAPPERKYSVWIGGSILAXXXXXXXXXXXXXXXXXXXX

SEQ ID NO: 774 - protein, 60 aa, synthetic construct
  VARIANT 1..20: note = X can be G, A, or deleted
  VARIANT 41..60: note = X can be G, A, or deleted
XXXXXXXXXXXXXXXXXXXXIIAPPERKYSVWIGGSILASXXXXXXXXXXXXXXXXXXXX

SEQ ID NO: 775 - protein, 60 aa, synthetic construct
  VARIANT 1..20: note = X can be G, A, or deleted
  VARIANT 41..60: note = X can be G, A, or deleted
XXXXXXXXXXXXXXXXXXXXIAPPERKYSVWIGGSILASLXXXXXXXXXXXXXXXXXXXX

SEQ ID NO: 776 - protein, 60 aa, synthetic construct
  VARIANT 1..20: note = X can be G, A, or deleted
  VARIANT 41..60: note = X can be G, A, or deleted
XXXXXXXXXXXXXXXXXXXXAPPERKYSVWIGGSILASLSXXXXXXXXXXXXXXXXXXXX

SEQ ID NO: 777 - protein, 60 aa, synthetic construct
  VARIANT 1..20: note = X can be G, A, or deleted
  VARIANT 41..60: note = X can be G, A, or deleted
XXXXXXXXXXXXXXXXXXXXPPERKYSVWIGGSILASLSTXXXXXXXXXXXXXXXXXXXX

SEQ ID NO: 778 - protein, 60 aa, synthetic construct
  VARIANT 1..20: note = X can be G, A, or deleted
  VARIANT 41..60: note = X can be G, A, or deleted
XXXXXXXXXXXXXXXXXXXXPERKYSVWIGGSILASLSTFXXXXXXXXXXXXXXXXXXXX

SEQ ID NO: 779 - protein, 60 aa, synthetic construct
  VARIANT 1..20: note = X can be G, A, or deleted
  VARIANT 41..60: note = X can be G, A, or deleted
XXXXXXXXXXXXXXXXXXXXERKYSVWIGGSILASLSTFQXXXXXXXXXXXXXXXXXXXX

SEQ ID NO: 780 - protein, 60 aa, synthetic construct
  VARIANT 1..20: note = X can be G, A, or deleted
  VARIANT 41..60: note = X can be G, A, or deleted
XXXXXXXXXXXXXXXXXXXXRKYSVWIGGSILASLSTFQQXXXXXXXXXXXXXXXXXXXX

SEQ ID NO: 781 - protein, 60 aa, synthetic construct
  VARIANT 1..20: note = X can be G, A, or deleted
  VARIANT 41..60: note = X can be G, A, or deleted
XXXXXXXXXXXXXXXXXXXXKYSVWIGGSILASLSTFQQMXXXXXXXXXXXXXXXXXXXX

SEQ ID NO: 782 - protein, 60 aa, synthetic construct
  VARIANT 1..20: note = X can be G, A, or deleted
  VARIANT 41..60: note = X can be G, A, or deleted
XXXXXXXXXXXXXXXXXXXXYSVWIGGSILASLSTFQQMWXXXXXXXXXXXXXXXXXXXX

SEQ ID NO: 783 - protein, 60 aa, synthetic construct
  VARIANT 1..20: note = X can be G, A, or deleted
  VARIANT 41..60: note = X can be G, A, or deleted
XXXXXXXXXXXXXXXXXXXXSVWIGGSILASLSTFQQMWIXXXXXXXXXXXXXXXXXXXX

SEQ ID NO: 784 - protein, 60 aa, synthetic construct
  VARIANT 1..20: note = X can be G, A, or deleted
  VARIANT 40: note = X can be any naturally-occurring amino acid or any unnatural amino acid
  VARIANT 41..60: note = X can be G, A, or deleted
XXXXXXXXXXXXXXXXXXXXVWIGGSILASLSTFQQMWIXXXXXXXXXXXXXXXXXXXXX

SEQ ID NO: 785 - protein, 60 aa, synthetic construct
  VARIANT 1..20: note = X can be G, A, or deleted
  VARIANT 39: note = X can be any naturally-occurring amino acid or any unnatural amino acid
  VARIANT 41..60: note = X can be G, A, or deleted
XXXXXXXXXXXXXXXXXXXXWIGGSILASLSTFQQMWIXKXXXXXXXXXXXXXXXXXXXX

SEQ ID NO: 786 - protein, 60 aa, synthetic construct
  VARIANT 1..20: note = X can be G, A, or deleted
  VARIANT 38: note = X can be any naturally-occurring amino acid or any unnatural amino acid
  VARIANT 41..60: note = X can be G, A, or deleted
XXXXXXXXXXXXXXXXXXXXIGGSILASLSTFQQMWIXKQXXXXXXXXXXXXXXXXXXXX

SEQ ID NO: 787 - protein, 60 aa, synthetic construct
  VARIANT 1..20: note = X can be G, A, or deleted
  VARIANT 21: note = X can be any naturally-occurring amino acid or any unnatural amino acid
  VARIANT 41..60: note = X can be G, A, or deleted
XXXXXXXXXXXXXXXXXXXXXPPERKYSVWIGGSILASLSXXXXXXXXXXXXXXXXXXXX

SEQ ID NO: 788 - protein, 60 aa, synthetic construct
  VARIANT 1..20: note = X can be G, A, or deleted
  VARIANT 22: note = X can be any naturally-occurring amino acid or any unnatural amino acid
  VARIANT 41..60: note = X can be G, A, or deleted
XXXXXXXXXXXXXXXXXXXXAXPERKYSVWIGGSILASLSXXXXXXXXXXXXXXXXXXXX

SEQ ID NO: 789 - protein, 60 aa, synthetic construct
  VARIANT 1..20: note = X can be G, A, or deleted
  VARIANT 23: note = X can be any naturally-occurring amino acid or any unnatural amino acid
  VARIANT 41..60: note = X can be G, A, or deleted
XXXXXXXXXXXXXXXXXXXXAPXERKYSVWIGGSILASLSXXXXXXXXXXXXXXXXXXXX

SEQ ID NO: 790 - protein, 60 aa, synthetic construct
  VARIANT 1..20: note = X can be G, A, or deleted
  VARIANT 24: note = X can be any naturally-occurring amino acid or any unnatural amino acid
  VARIANT 41..60: note = X can be G, A, or deleted
XXXXXXXXXXXXXXXXXXXXAPPXRKYSVWIGGSILASLSXXXXXXXXXXXXXXXXXXXX

SEQ ID NO: 791 - protein, 60 aa, synthetic construct
  VARIANT 1..20: note = X can be G, A, or deleted
  VARIANT 25: note = X can be any naturally-occurring amino acid or any unnatural amino acid
  VARIANT 41..60: note = X can be G, A, or deleted
XXXXXXXXXXXXXXXXXXXXAPPEXKYSVWIGGSILASLSXXXXXXXXXXXXXXXXXXXX

SEQ ID NO: 792 - protein, 60 aa, synthetic construct
  VARIANT 1..20: note = X can be G, A, or deleted
  VARIANT 26: note = X can be any naturally-occurring amino acid or any unnatural amino acid
  VARIANT 41..60: note = X can be G, A, or deleted
XXXXXXXXXXXXXXXXXXXXAPPERXYSVWIGGSILASLSXXXXXXXXXXXXXXXXXXXX

SEQ ID NO: 793 - protein, 60 aa, synthetic construct
  VARIANT 1..20: note = X can be G, A, or deleted
  VARIANT 27: note = X can be any naturally-occurring amino acid or any unnatural amino acid
  VARIANT 41..60: note = X can be G, A, or deleted
XXXXXXXXXXXXXXXXXXXXAPPERKXSVWIGGSILASLSXXXXXXXXXXXXXXXXXXXX

SEQ ID NO: 794 - protein, 60 aa, synthetic construct
  VARIANT 1..20: note = X can be G, A, or deleted
  VARIANT 28: note = X can be any naturally-occurring amino acid or any unnatural amino acid
  VARIANT 41..60: note = X can be G, A, or deleted
XXXXXXXXXXXXXXXXXXXXAPPERKYXVWIGGSILASLSXXXXXXXXXXXXXXXXXXXX

SEQ ID NO: 795 - protein, 60 aa, synthetic construct
  VARIANT 1..20: note = X can be G, A, or deleted
  VARIANT 29: note = X can be any naturally-occurring amino acid or any unnatural amino acid
  VARIANT 41..60: note = X can be G, A, or deleted
XXXXXXXXXXXXXXXXXXXXAPPERKYSXWIGGSILASLSXXXXXXXXXXXXXXXXXXXX

SEQ ID NO: 796 - protein, 60 aa, synthetic construct
  VARIANT 1..20: note = X can be G, A, or deleted
  VARIANT 30: note = X can be any naturally-occurring amino acid or any unnatural amino acid
  VARIANT 41..60: note = X can be G, A, or deleted
XXXXXXXXXXXXXXXXXXXXAPPERKYSVXIGGSILASLSXXXXXXXXXXXXXXXXXXXX

SEQ ID NO: 797 - protein, 60 aa, synthetic construct
  VARIANT 1..20: note = X can be G, A, or deleted
  VARIANT 31: note = X can be any naturally-occurring amino acid or any unnatural amino acid
  VARIANT 41..60: note = X can be G, A, or deleted
XXXXXXXXXXXXXXXXXXXXAPPERKYSVWXGGSILASLSXXXXXXXXXXXXXXXXXXXX

SEQ ID NO: 798 - protein, 60 aa, synthetic construct
  VARIANT 1..20: note = X can be G, A, or deleted
  VARIANT 32: note = X can be any naturally-occurring amino acid or any unnatural amino acid
  VARIANT 41..60: note = X can be G, A, or deleted
XXXXXXXXXXXXXXXXXXXXAPPERKYSVWIXGSILASLSXXXXXXXXXXXXXXXXXXXX

SEQ ID NO: 799 - protein, 60 aa, synthetic construct
  VARIANT 1..20: note = X can be G, A, or deleted
  VARIANT 33: note = X can be any naturally-occurring amino acid or any unnatural amino acid
  VARIANT 41..60: note = X can be G, A, or deleted
XXXXXXXXXXXXXXXXXXXXAPPERKYSVWIGXSILASLSXXXXXXXXXXXXXXXXXXXX

SEQ ID NO: 800 - protein, 60 aa, synthetic construct
  VARIANT 1..20: note = X can be G, A, or deleted
  VARIANT 34: note = X can be any naturally-occurring amino acid or any unnatural amino acid
  VARIANT 41..60: note = X can be G, A, or deleted
XXXXXXXXXXXXXXXXXXXXAPPERKYSVWIGGXILASLSXXXXXXXXXXXXXXXXXXXX

SEQ ID NO: 801 - protein, 60 aa, synthetic construct
  VARIANT 1..20: note = X can be G, A, or deleted
  VARIANT 35: note = X can be any naturally-occurring amino acid or any unnatural amino acid
  VARIANT 41..60: note = X can be G, A, or deleted
XXXXXXXXXXXXXXXXXXXXAPPERKYSVWIGGSXLASLSXXXXXXXXXXXXXXXXXXXX

SEQ ID NO: 802 - protein, 60 aa, synthetic construct
  VARIANT 1..20: note = X can be G, A, or deleted
  VARIANT 36: note = X can be any naturally-occurring amino acid or any unnatural amino acid
  VARIANT 41..60: note = X can be G, A, or deleted
XXXXXXXXXXXXXXXXXXXXAPPERKYSVWIGGSIXASLSXXXXXXXXXXXXXXXXXXXX

SEQ ID NO: 803 - protein, 60 aa, synthetic construct
  VARIANT 1..20: note = X can be G, A, or deleted
  VARIANT 37: note = X can be any naturally-occurring amino acid or any unnatural amino acid
  VARIANT 41..60: note = X can be G, A, or deleted
XXXXXXXXXXXXXXXXXXXXAPPERKYSVWIGGSILXSLSXXXXXXXXXXXXXXXXXXXX

SEQ ID NO: 804 - protein, 60 aa, synthetic construct
  VARIANT 1..20: note = X can be G, A, or deleted
  VARIANT 38: note = X can be any naturally-occurring amino acid or any unnatural amino acid
  VARIANT 41..60: note = X can be G, A, or deleted
XXXXXXXXXXXXXXXXXXXXAPPERKYSVWIGGSILAXLSXXXXXXXXXXXXXXXXXXXX

SEQ ID NO: 805 - protein, 60 aa, synthetic construct
  VARIANT 1..20: note = X can be G, A, or deleted
  VARIANT 39: note = X can be any naturally-occurring amino acid or any unnatural amino acid
  VARIANT 41..60: note = X can be G, A, or deleted
XXXXXXXXXXXXXXXXXXXXAPPERKYSVWIGGSILASXSXXXXXXXXXXXXXXXXXXXX

SEQ ID NO: 806 - protein, 60 aa, synthetic construct
  VARIANT 1..20: note = X can be G, A, or deleted
  VARIANT 40: note = X can be any naturally-occurring amino acid or any unnatural amino acid
  VARIANT 41..60: note = X can be G, A, or deleted
XXXXXXXXXXXXXXXXXXXXAPPERKYSVWIGGSILASLXXXXXXXXXXXXXXXXXXXXX

SEQ ID NO: 807 - protein, 80 aa, synthetic construct
  VARIANT 1..20: note = X can be G, A, or deleted
  VARIANT 21: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXXPSTMKIKIIAPPERKYSVWIGGSILASLSTFQQMWIXKQXXXXXXXXXXXXXXXXXXXX

SEQ ID NO: 808 - protein, 80 aa, synthetic construct
  VARIANT 1..20: note = X can be G, A, or deleted
  VARIANT 22: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAXSTMKIKIIAPPERKYSVWIGGSILASLSTFQQMWIXKQXXXXXXXXXXXXXXXXXXXX

SEQ ID NO: 809 - protein, 80 aa, synthetic construct
  VARIANT 1..20: note = X can be G, A, or deleted
  VARIANT 23: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPXTMKIKIIAPPERKYSVWIGGSILASLSTFQQMWIXKQXXXXXXXXXXXXXXXXXXXX

SEQ ID NO: 810 - protein, 80 aa, synthetic construct
  VARIANT 1..20: note = X can be G, A, or deleted
  VARIANT 24: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSXMKIKIIAPPERKYSVWIGGSILASLSTFQQMWIXKQXXXXXXXXXXXXXXXXXXXX

SEQ ID NO: 811 - protein, 80 aa, synthetic construct
  VARIANT 1..20: note = X can be G, A, or deleted
  VARIANT 25: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTXKIKIIAPPERKYSVWIGGSILASLSTFQQMWIXKQXXXXXXXXXXXXXXXXXXXX

SEQ ID NO: 812 - protein, 80 aa, synthetic construct
  VARIANT 1..20: note = X can be G, A, or deleted
  VARIANT 26: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMXIKIIAPPERKYSVWIGGSILASLSTFQQMWIXKQXXXXXXXXXXXXXXXXXXXX

SEQ ID NO: 813 - protein, 80 aa, synthetic construct
  VARIANT 1..20: note = X can be G, A, or deleted
  VARIANT 27: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKXKIIAPPERKYSVWIGGSILASLSTFQQMWIXKQXXXXXXXXXXXXXXXXXXXX

SEQ ID NO: 814 - protein, 80 aa, synthetic construct
  VARIANT 1..20: note = X can be G, A, or deleted
  VARIANT 28: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIXIIAPPERKYSVWIGGSILASLSTFQQMWIXKQXXXXXXXXXXXXXXXXXXXX

SEQ ID NO: 815 - protein, 80 aa, synthetic construct
  VARIANT 1..20: note = X can be G, A, or deleted
  VARIANT 29: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKXIAPPERKYSVWIGGSILASLSTFQQMWIXKQXXXXXXXXXXXXXXXXXXXX

SEQ ID NO: 816 - protein, 80 aa, synthetic construct
  VARIANT 1..20: note = X can be G, A, or deleted
  VARIANT 30: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIXAPPERKYSVWIGGSILASLSTFQQMWIXKQXXXXXXXXXXXXXXXXXXXX

SEQ ID NO: 817 - protein, 80 aa, synthetic construct
  VARIANT 1..20: note = X can be G, A, or deleted
  VARIANT 31: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIXPPERKYSVWIGGSILASLSTFQQMWIXKQXXXXXXXXXXXXXXXXXXXX

SEQ ID NO: 818 - protein, 80 aa, synthetic construct
  VARIANT 1..20: note = X can be G, A, or deleted
  VARIANT 32: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAXPERKYSVWIGGSILASLSTFQQMWIXKQXXXXXXXXXXXXXXXXXXXX

SEQ ID NO: 819 - protein, 80 aa, synthetic construct
  VARIANT 1..20: note = X can be G, A, or deleted
  VARIANT 33: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPXERKYSVWIGGSILASLSTFQQMWIXKQXXXXXXXXXXXXXXXXXXXX

SEQ ID NO: 820 - protein, 80 aa, synthetic construct
  VARIANT 1..20: note = X can be G, A, or deleted
  VARIANT 34: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPXRKYSVWIGGSILASLSTFQQMWIXKQXXXXXXXXXXXXXXXXXXXX

SEQ ID NO: 821 - protein, 80 aa, synthetic construct
  VARIANT 1..20: note = X can be G, A, or deleted
  VARIANT 35: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPEXKYSVWIGGSILASLSTFQQMWIXKQXXXXXXXXXXXXXXXXXXXX

SEQ ID NO: 822 - protein, 80 aa, synthetic construct
  VARIANT 1..20: note = X can be G, A, or deleted
  VARIANT 36: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERXYSVWIGGSILASLSTFQQMWIXKQXXXXXXXXXXXXXXXXXXXX

SEQ ID NO: 823 - protein, 80 aa, synthetic construct
  VARIANT 1..20: note = X can be G, A, or deleted
  VARIANT 37: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKXSVWIGGSILASLSTFQQMWIXKQXXXXXXXXXXXXXXXXXXXX

SEQ ID NO: 824 - protein, 80 aa, synthetic construct
  VARIANT 1..20: note = X can be G, A, or deleted
  VARIANT 38: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYXVWIGGSILASLSTFQQMWIXKQXXXXXXXXXXXXXXXXXXXX

SEQ ID NO: 825 - protein, 80 aa, synthetic construct
  VARIANT 1..20: note = X can be G, A, or deleted
  VARIANT 39: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSXWIGGSILASLSTFQQMWIXKQXXXXXXXXXXXXXXXXXXXX

SEQ ID NO: 826 - protein, 80 aa, synthetic construct
  VARIANT 1..20: note = X can be G, A, or deleted
  VARIANT 40: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVXIGGSILASLSTFQQMWIXKQXXXXXXXXXXXXXXXXXXXX

SEQ ID NO: 827 - protein, 80 aa, synthetic construct
  VARIANT 1..20: note = X can be G, A, or deleted
  VARIANT 41: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWXGGSILASLSTFQQMWIXKQXXXXXXXXXXXXXXXXXXXX

SEQ ID NO: 828 - protein, 80 aa, synthetic construct
  VARIANT 1..20: note = X can be G, A, or deleted
  VARIANT 42: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIXGSILASLSTFQQMWIXKQXXXXXXXXXXXXXXXXXXXX

SEQ ID NO: 829 - protein, 80 aa, synthetic construct
  VARIANT 1..20: note = X can be G, A, or deleted
  VARIANT 43: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGXSILASLSTFQQMWIXKQXXXXXXXXXXXXXXXXXXXX

SEQ ID NO: 830 - protein, 80 aa, synthetic construct
  VARIANT 1..20: note = X can be G, A, or deleted
  VARIANT 44: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGGXILASLSTFQQMWIXKQXXXXXXXXXXXXXXXXXXXX

SEQ ID NO: 831 - protein, 80 aa, synthetic construct
  VARIANT 1..20: note = X can be G, A, or deleted
  VARIANT 45: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGGSXLASLSTFQQMWIXKQXXXXXXXXXXXXXXXXXXXX

SEQ ID NO: 832 - protein, 80 aa, synthetic construct
  VARIANT 1..20: note = X can be G, A, or deleted
  VARIANT 46: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGGSIXASLSTFQQMWIXKQXXXXXXXXXXXXXXXXXXXX

SEQ ID NO: 833 - protein, 80 aa, synthetic construct
  VARIANT 1..20: note = X can be G, A, or deleted
  VARIANT 47: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGGSILXSLSTFQQMWIXKQXXXXXXXXXXXXXXXXXXXX

SEQ ID NO: 834 - protein, 80 aa, synthetic construct
  VARIANT 1..20: note = X can be G, A, or deleted
  VARIANT 48: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGGSILAXLSTFQQMWIXKQXXXXXXXXXXXXXXXXXXXX

SEQ ID NO: 835 - protein, 80 aa, synthetic construct
  VARIANT 1..20: note = X can be G, A, or deleted
  VARIANT 49: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGGSILASXSTFQQMWIXKQXXXXXXXXXXXXXXXXXXXX

SEQ ID NO: 836 - protein, 80 aa, synthetic construct
  VARIANT 1..20: note = X can be G, A, or deleted
  VARIANT 50: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGGSILASLXTFQQMWIXKQXXXXXXXXXXXXXXXXXXXX

SEQ ID NO: 837 - protein, 80 aa, synthetic construct
  VARIANT 1..20: note = X can be G, A, or deleted
  VARIANT 51: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGGSILASLSXFQQMWIXKQXXXXXXXXXXXXXXXXXXXX

SEQ ID NO: 838 - protein, 80 aa, synthetic construct
  VARIANT 1..20: note = X can be G, A, or deleted
  VARIANT 52: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGGSILASLSTXQQMWIXKQXXXXXXXXXXXXXXXXXXXX

SEQ ID NO: 839 - protein, 80 aa, synthetic construct
  VARIANT 1..20: note = X can be G, A, or deleted
  VARIANT 53: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGGSILASLSTFXQMWIXKQXXXXXXXXXXXXXXXXXXXX

SEQ ID NO: 840 - protein, 80 aa, synthetic construct
  VARIANT 1..20: note = X can be G, A, or deleted
  VARIANT 54: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGGSILASLSTFQXMWIXKQXXXXXXXXXXXXXXXXXXXX

SEQ ID NO: 841 - protein, 80 aa, synthetic construct
  VARIANT 1..20: note = X can be G, A, or deleted
  VARIANT 55: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGGSILASLSTFQQXWIXKQXXXXXXXXXXXXXXXXXXXX

SEQ ID NO: 842 - protein, 80 aa, synthetic construct
  VARIANT 1..20: note = X can be G, A, or deleted
  VARIANT 56: note = X can be any naturally-occurring amino acid or any unnatural amino acid
  VARIANT 58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGGSILASLSTFQQMXIXKQXXXXXXXXXXXXXXXXXXXX

SEQ ID NO: 843 - protein, 80 aa, synthetic construct
  VARIANT 1..20: note = X can be G, A, or deleted
  VARIANT 57..58: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGGSILASLSTFQQMWXXKQXXXXXXXXXXXXXXXXXXXX

SEQ ID NO: 844 - protein, 80 aa, synthetic construct
  VARIANT 1..20: note = X can be G, A, or deleted
  VARIANT 58..59: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGGSILASLSTFQQMWIXXQXXXXXXXXXXXXXXXXXXXX

SEQ ID NO: 845 - protein, 80 aa, synthetic construct
  VARIANT 1..20: note = X can be G, A, or deleted
  VARIANT 58: note = X can be any naturally-occurring amino acid or any unnatural amino acid
  VARIANT 60: note = X can be any naturally-occurring amino acid or any unnatural amino acid
  VARIANT 61..80: note = X can be G, A, or deleted
XXXXXXXXXXXXXXXXXXXXAPSTMKIKIIAPPERKYSVWIGGSILASLSTFQQMWIXKXXXXXXXXXXXXXXXXXXXXX

(19) United States
(12) Patent Application Publication
(10) Pub. No.: US 2025/0051411 A1
(43) Pub. Date: Feb. 13, 2025

(54) COMPOSITIONS AND METHODS FOR USING ACTIN-BASED PEPTIDES TO MODULATE CELLULAR BIOACTIVITY AND CELLULAR SUSCEPTIBILITY TO INTRACELLULAR PATHOGENS

(71) Applicant: Virongy L.L.C., Manassas, VA (US)

(72) Inventor: Yuntao Wu, Manassas, VA (US)

(21) Appl. No.: 18/602,390

(22) Filed: Mar. 12, 2024

Related U.S. Application Data
(62) Division of application No. 17/317,803, filed on May 11, 2021, now abandoned.
Application No. 17/317,803 is a division of application No. 15/446,240, filed on Mar. 1, 2017, now Pat. No. 11,028,138.
(60) Provisional application No. 62/357,991, filed on Jul. 2, 2016.

(51) Int. Cl. C07K 14/47; A61K 38/00; A61K 38/17; A61K 47/51; A61K 47/64

(52) U.S. Cl. CPC C07K 14/4716; A61K 38/1719; A61K 47/51; A61K 47/64; A61K 38/00; C07K 2319/10

(57) ABSTRACT

Compositions and methods for using actin-based peptides to modulate cellular bioactivity. including modulation of cellular susceptibility to intracellular pathogens, such as bacteria and viruses.

### CROSS-REFERENCE TO RELATED APPLICATION

This application is a Divisional of U.S. patent application Ser. No. 17/317,803, filed May 11, 2021, which is a Divisional of U.S. patent application Ser. No. 15/446,240, filed Mar. 1, 2017, now U.S. Pat. No. 11,028,138, which claims priority to U.S. Application No. 62/357,991, filed Jul. 2. 2016, the entire content of each application is incorporated by reference herein in its entirety.

### INCORPORATION BY REFERENCE STATEMENT

This statement, made under Rules 77 (b) (5) (ii) and any other applicable rule incorporates into the present specification of an XML file for a “Sequence Listing XML” (see Rule 831 (a)), submitted via the USPTO patent electronic filing system or on one or more read-only optical discs (see Rule 1.52 (e) (8)), identifying the names of each file, the date of creation of each file, and the size of each file in bytes as follows:
File name: 173-013A1_Sequence_Replacement
Creation date: Oct. 22, 2024
Byte size: 1,939,629

### FIELD

The present disclosure relates to modulating cellular bioactivity and cell susceptibility to viral infection through modulating actin dynamics. Provided herein are methodology, compositions, and the like for modulating actin-related cellular bioactivity and cellular susceptibility to viral infection.

### INTRODUCTION

Actin-related Cellular Bioactivity: The cytoskeleton of a cell consists of three main filaments: actin filaments (also called microfilaments), intermediate filaments, and microtubules. The cytoskeleton provides a cell with shape, structure, and an anchorage for various organelles. It also provides driving forces for cell migration and intracellular transport of macromolecules. In addition, actin regulates many key functions in cells, including:
The formation of various of actin networks in cells for providing cellular trafficking routes.
Modulation of cell surface receptors. Actin is heavily involved in receptor clustering and cycling.
Cytokinesis. Cell division involves a constracting ring that is composed of actin, myosin, and a-actinin.
Apoptosis. Actin is degraded into two fragments during apoptosis. Stress can induces the formation of stress fibers that is involved in apoptosis. The mitochondrial migration of the actin binding protein cofilin is also involved in triggering cell apopotosis.
Cell adhesion and development. Adhesion of epithelial cells with extracellular matrix or with adjacent cells involves the actin cytoskeleton as well as cadherins and catenins.
Gene expression modulation. Actin's state of polymerization can affect the pattern of gene expression.
Smooth and hear muscle contraction: both actin and myosin are involved in muscle contraction and relaxation, and they make up 90% of muscle protein. The interaction of actin-myosin results in the movement of the two proteins that produces contractions.
Nuclear actin function: actin can also enter the nucleus, and is involved in the architectural integrity of the nucleus, gene transcription, and translocation of the activated chromosome fragment from under membrane to euchromatin.

Actin Filaments: The major cytoskeletal protein of cells is actin, a 43-kd protein that polymerizes to form actin filaments-thin, flexible fibers approximately 7 nm in diameter and up to several micrometers in length. Within the cell, actin filaments are organized into higher-order structures, forming bundles or three-dimensional networks with the properties of semisolid gels. The assembly and disassembly of actin filaments, their crosslinking into bundles and networks, and their association with other cell structures such as the plasma membrane are regulated by a variety of actin-binding proteins which are critical components of the actin cytoskeleton. Actin filaments are particularly abundant beneath the plasma membrane, where they form a network that provides mechanical support, determines cell shape, and allows movement of the cell surface, thereby enabling cells to migrate, engulf particles, and divide. Cooper, G. M., The Cell: A Molecular Approach, 2^{nd} Ed., Sinauer Associates, Sunderland, M A (2000). ncbi.nlm.nih.gov Actin structure, function and sequences are described by Otterbein, L R, The Crystal Structure of Uncomplexed Actin in the ADP State, Science, Vol. 293, 27 Jul. 2001; Kabsch, W., The Actin Fold, FASEB J. 9, 167-174 (1995); Homes, K. C. et al., Atomic model of the actin filament, Nature, Vol. 347, 6 Sep. 1990; Mattila, P. K. et al., Dynamics of the actin cytoskeleton mediates receptor cross talk: An emerging concept in tuning receptor signaling, J. Cell Biol. Vol. 212 No. 3, 267-280 (2016); Sandiford, S. L. et al. (2015), Cytoplasmic Actin Is an Extracellular Insect, PLoS Pathog 11 (2): e1004631. doi: 10.1371/journal.ppat. 1004631; Hardin, J., Regulating cell-cell junctions from A to Z, J. Cell Biol. Vol. 213 No. 2 151-153 (2016); and Yoder, A. et al., HIV Envelope-CXCR4 Signaling Activates Cofilin to Overcome Cortical Actin Restriction in Resting CD4 T Cells, Cell 134, 782-792, Sep. 5, 2008.

There are three actin isoforms, alpha, beta, and gamma. Alpha actin is mainly found in contractile structures such as those of muscle cells. Beta actin is found at the leading edge of migrating cells to drive cell mobility. Gamma actin is found in the filaments of stress fibers. Actins exist either as a monomeric form (G-actin) or as assembled, double helical, filamentous polymers (filamentous actin, F-actin, or microfilaments).

In cells, the actin cytoskeleton provides mechanical support and is a major driving force for cell motility. Actin also participates in many cellular processes such as cell division, surface receptor cycling, vesicle and organelle movement, and signal transduction. In immune cells, actin is also involved in cell adhesion, cell migration, chemotaxis, and T cell activation (Wulfing and Davis, 1998).

Intracellular actin activity is regulated through actin polymerization and depolymerization, which are regulated by multiple actin modulators in cells. In test tubes, in the absence of other actin regulators, G-actin can automatically polymerize at high salt concentration. Polymerized actin filaments (F-actin) can also dissociate into G-actin. Structurally, polymerized actin filaments have polarity, with (+) and (−) ends (or barbed and pointed ends). When actin polymerization and depolymerization reactions reach a steady-state, G-actin is added to the (+) end and dissociated from the (−) end at equal rates, so the length of the actin filaments remains constant. This process is called actin treadmilling, which resembles a process of actin subunits “walking” from the (+) end to the (−) end (Pollard and Borisy, 2003).

In cells, actin dynamics are tightly regulated to ensure proper and speedy response to stimuli. G-actin is normally associated with cellular regulators such as profilin and thymosin β4. Profilin acts as a nucleotide-exchange factor, which allows the switch from ADP-actin to ATP-actin ready for actin polymerization. Thymosin β4 is an actin-sequestering protein, which serves as a buffering protein for the maintenance of the monomeric actin pool. F-actin is also associated with multiple cellular regulators such as ADF/cofilin and the Apr2/3 complex. ADF/Cofilin is a family of actin-severing proteins which depolymerize F-actin mainly at the (−) end. The Arp2/3 complex is a seven-subunit protein that promotes the growth of new actin filaments. In addition, actin capping proteins such as CapZ and gelsolin can cap the ends of actin filaments, preventing actin polymerization or depolymerization (Pollard and Borisy, 2003).

In cells, actin and microtubule cytoskeleton gives mechanical support and control of cell shape. The actin and microtubule cytoskeleton networks also provide trafficking routes through cytoplasm for intracellular macromolecules and, sometimes, invading bacteria and viruses. In non-muscle cells, actin polymerization and depolymerization provide a major driving force for cell migration through treadmilling, whereas in muscle cells, actin fiber is the scaffold on which myosin proteins generate force to produce muscle contraction. In addition, actin has a wide range of roles, including regulation of cell surface receptors, cell division, cell adhesion and differentiation, T cell/B cell activation, and gene expression.

Actin is also involved in the process of apoptosis, in which cellular stress induces the reorganization of the actin cytoskeleton, giving rise to actin stress fiber. During apoptosis, actin is fragmented into two fragments of 15 kD and 31 kD by the ICE/ced-3 family of proteases, which is one of the major characteristics of apoptotic cells.

The microtubule network also plays important roles in many cellular processes. Microtubules provide means of intracellular transport of organelles and secretory vesicles. They are also involved in the formation of mitotic spindles during cell division (mitosis and meiosis). Drugs targeting microtubules are known to induce apoptosis of cancer cells.

Actin and microtubule dynamics are also important for intracellular bacterial and viral infection of cells. Actin polymerization and depolymerization can modulate receptor dynamics, facilitating the entry of bacteria and viruses. Actin polymerization can produce essential driving forces and scaffolds for bacterial and viral intracellular migration, nucleic acid synthesis and transcription, or virion assembly and budding (Spear et al. 2012; Spear et al. 2013; Taylor et al 2011). The microtubule network is frequently used by intracellular bacteria and viruses for migrations inside and between cells (Kotsakis et al. 2001).

In general, the actin-derived peptides of the invention are modified in order to penetrate cells, e.g., by addition of an N-terminal polyarginine segment. Relevant reviews of cell-penetrating peptides are: C. Bechara et al., Cell-penetrating peptides: 20 years later, where do we stand?, FEBS Letters 587 (2013) 1693-1702; B. Gupta, et al., Intracellular delivery of large molecules and small particles by cell-penetrating proteins and peptides, Advanced Drug Delivery Reviews 57 (2005) 637-651; M. Lindgren, et al., cell-penetrating peptides, TiPS-March 2000 (Vol. 21) 99-103; M. Kristensen, et al., Applications and Challenges for Use of Cell-Penetrating Peptides as Delivery Vectors for Peptide and Protein Cargos, Int. J. Mol. Sci. 2016, 17, 185; and F. Milletti, Cell-penetrating peptides: classes, origin, and current landscape, Drug Discovery Today, Vol. 17, No 15-16, August 2012.

Actin is involved in: 1) supporting cell shape and cell growth; 2) driving cells to migrate; 3) cell signal transduction; 4) cell surface receptor cycling between the surface and the cytoplasm; 5) cell-cell junction and cell adhesion: 6) muscle cell contraction; 7) in the cardiovascular system, actin activity affects heart beat and blood vessel elasticity and capacitance; 8) the process of viral and bacterial infection. Actin can function as a barrier to block viral infection, while viruses and bacteria may also use actin polymerization to promote inter-and intracellular migration; 9) the intracellular delivery of chemical compounds (e.g. small-molecule drugs) and biological substances (e.g. DNA, peptides, and proteins et al). The cortical actin in a cell structure can also block the intracellular delivery of compounds and substances, affecting the delivery efficiency: 10) the process of cell growth, differentiation and apoptosis.

The crystal structure of G-actin coupled with DNase I was solved in 1990 by Kabsch and colleagues [5]. The crystal structure of uncomplexed actin was also solved by Otterbein and coauthors [2]. The near-atomic resolution structure of F-actin was deduced by Holmes and colleagues using a combination of the crystal structure of G-actin and X-ray fiber diffraction data [3]. Currently, there are also over 80 structures of actin in complex with various actin binding proteins such as gelsolin, cofilin, and profilin [4]. Structurally, actin is organized into two related domains, which can be further subdivided into 4 subdomains numbered 1 to 4 (FIG. 2D).

β-actin structure, showing subdomains 1 to 4 and the hydrophobic cleft between subdomains 1 and 3 (red circle). This hydrophobic cleft is a common binding pocket of most actin binding proteins (ABPs) such as gelsolin, profilin, and ADF/cofilin as shown. Two diametrically opposed clefts separate the two large domains of actin. The larger cleft, between subdomains 2 and 4, constitutes the nucleotide-binding site, whereas the smaller cleft, between subdomains 1 and 3, mediates the interactions of actin with most actin binding proteins (ABPs) (FIG. 2D). ABPs are extremely diverse, both structurally and functionally, but for some reason related to the filament structure, most ABPs seem to share a common binding area on the actin surface, which is the hydrophobic pocket between actin subdomains 1 and 3. Both gelsolin and cofilin bind to this cleft, and profilin also interacts with the back of this cleft. It is possible that our peptides interface with the binding of some ABPs, thereby disturbing actin dynamics, and indirectly the dynamics of surface receptors.

### SUMMARY

In one aspect, provided is an actin-based peptide, comprising an actin core domain and a cell penetration domain, operably linked together by a a spacer domain, wherein an N-terminus or C-terminus may have either the actin core domain or the cell penetration domain.

In one embodiment, the actin core domain is selected from the group consisting of NB5 (SEQ ID NO: 1, 7, 15-138), NB7 (SEQ ID NO: 2, 8, 9, 139-290), NB9 (SEQ ID NO: 3, 9, 10, 11, 291-470), NB10 (SEQ ID NO: 4, 12, 471-601), B11 (SEQ ID NO: 5, 13, 602-723), and B17 (SEQ ID NO: 6, 14, 724-845).

In another embodiment, the actin core domain is labeled with a traceable dye or marker.

In another aspect, provided is an actin-based peptide, comprising a B11 core selected from the sequences set forth in SEQ ID NO: 5, 13, and 602-723.

In another aspect, provided is a method for controlling viral or bacterial infection, comprising administering an actin-based peptide to a cell, virus, or bacteria, in an amount sufficient to inhibit or enhance infection of said virus or bacteria. In one embodiment, HIV infection is increased by administering said actin-based peptide to a cell. In another embodiment, the actin-based peptide comprises an actin core domain selected from NB7 (SEQ ID NO: 2, 8, 9, 139-290), and NB9 (SEQ ID NO: 3, 9, 10, 11, 291-470).

In another aspect, provided is a method for inhibiting or enhancing viral or bacterial infection, comprising: administering an agent to a cell or a virus or bacteria, with an amount of one or more peptide sequences selected from SEQ ID NOs: 1-845 effective to inhibit or enhance infection of said virus or bacteria.

In another aspect, provided is a method for inhibiting or enhancing viral or bacterial vector delivery into a cell, comprising: administering an agent to a cell or a viral or bacterial vector, with an amount of one or more peptide sequences selected from SEQ ID NOs: 1-845 effective to inhibit or enhance vector delivery to said virus or bacteria.

In another aspect, provided is a method for modulating actin dynamics in a cell, comprising: delivering into a cell one or more actin peptides selected from SEQ ID NOs: 1-845, in an amount effective to modulate actin dynamics.

In another aspect, provided is a method for modulating cellular bioactivity, comprising: delivering into a cell one or more actin-based peptides selected from SEQ ID NOs: 1-845, in an amount effective to modulate cellular bioactivity. In one embodiment, the cellular bioactivity is: cytoplasmic and nuclear actin dynamics, cell morphology, cell motility and mobility, cell surface receptor density, cell adhesion processes, gene expression, cell division, growth, differentiation, and apoptosis, the processes of endocytosis and exocytosis, smooth and heart muscle cell contraction and relaxation, blood vessel elasticity and capacitance, a process of inflammation, cellular susceptibility to intracellular pathogens, or cellular uptake of chemical compounds, including small and macro molecules, medical drugs, nucleic acids, and proteins.

In another embodiment, provided is an actin-based peptide conjugated to a cell-penetration peptide or membrane-permeable molecule. In one embodiment, the peptide fragment is selected from SEQ ID NO: 1-845. In another embodiment, the peptide fragment is labeled with a traceable dye or marker.

In another aspect, provided is a method of making a peptide composition, comprising:
a) synthesizing one or more actin-based peptides; and
b) covalently conjugating or non-covalently conjugating said actin-based peptide with a traceable dye, a small molecule, nucleic acid, peptide, or protein.

In another aspect, provided is a method of making a peptide composition, comprising:
a) synthesizing one or more actin-based peptides selected from SEQ ID NOs: 1-845; and
b) covalently conjugating or non-covalently conjugating said actin-based peptide with a traceable dye, a small molecule, nucleic acid, peptide, or protein.

In another aspect, provided is a method for intracellular delivery of an actin peptide fragment, comprising: conjugating said actin peptide fragment with a cell-penetration peptide or membrane-permeable molecule.

In another aspect, provided is a peptide having a formula set forth in SEQ ID NOs: 1-845. In one embodiment, the peptide is a cyclic peptide.

In another aspect, provide is a kit comprising one or more peptides having a formula set forth in SEQ ID NOs: 1-845, and an effective amount of a therapeutic compound, a diagnostic compound, or a compound for modulating cellular activity.

Objects of the instant actin-derived peptides:

1) To enhance or inhibit cell signal transduction; enhance or block the activation of signaling pathways related to regulating actin dynamics.

2) To up-regulate or down-regulate receptor density on the cell surface such as up-regulate the chemokine receptors, adhesion molecules, cancer related surface receptors on the cell surface.

3) To enhance or inhibit viral infection or viral vector transduction of cells; promote or inhibit viral spreading.

4) To affect the process of cell growth, differentiation and apoptosis.

Other objects include providing compositions and methods for enhancing or inhibiting viral infections. In one embodiment, actin-based peptide can be used to trigger actin dynamics to modulate pathogen infection.

Another object is to provide actin-based peptides which can be used to enhance viral vector entry into cells such as cancer cells, stem cells, or terminally-differentiated cells for gene delivery or gene therapy.

In another aspect, the actin-based peptides can be used to enhance viral amplification in primary culture of cells from biological samples, such as blood samples or tissue samples. The use of these peptides can facilitate the isolation and recovery of viruses from biological samples such as the recovery of HIV from blood CD4 T cells.

In another aspect, because modulating actin and cofilin activity can lead to apoptosis, the subject peptides can be used to trigger cell death for therapy, such as killing cancer cells.

Another object is to provide peptides and methods of modulating actin activity, to either inhibit or enhance cell migration and cell mobility. Such peptides can be used to inhibit or enhance cell migration in the body. They can be used to slow down cancer cell migration, or used as anti-inflammatory drugs, or used to modulate the movement of stem cells in the body, or used to attract progenitor cells for tissue damage repairs.

In another aspect, actin-based peptides can be used to up-regulate or down-regulate cell surface receptors, change cellular behaviors or responses to receptor-mediated signal transduction. This property can be used to facilitate the study of receptor dynamics and cell signaling, or to facilitate the efficacy of medical drugs targeting cell receptors and signaling pathways.

In another aspect, actin-based peptides can be labeled with a traceable dye or marker and be used to monitor intracellular actin and microtubule dynamics, and the behaviors of actin-binding proteins.

### BRIEF DESCRIPTION OF THE DRAWINGS

FIG. 1: (A) Schematic representation of the design and discovery of the actin-based peptides. The whole amino acid (AA) sequence of β-actin (L=375 AA) can be divided into a library of overlapping peptides with defined amino acid length (e.g. 10 to 40 AA), which is defined as the actin core domain. The actin-based peptides also contain an additional domain which allows them to penetrate cell membrane to enter cells. In addition, the actin-based peptides also contain a spacer domain which links the actin core domain with the cell penetration domain. (B) Screening of an actin-based peptide library for bioactivities in enhancing HIV infection. An HIV reporter T cells, Rev-CEM-GFP, were treated with each actin-based peptide, and then infected with HIV-1. Enhancement of HIV infection was quantified. For controls, HIV replication in peptide-untreated cells was quantified, and plotted as 100%. In this screening, peptide N5, N7, and N9 were found to enhance HIV replication from 350% to 400%.

FIG. 2: (A) Schematic representation of the actin-core domains discovered from screening. (B) The amino acid sequences of the core domains. (C) The location and AA position of the actin-core domains within the human β-actin protein (SEQ ID NO: 848). (D) β-actin structure, showing subdomains 1 to 4 and the hydrophobic cleft between subdomains 1 and 3 (red circle). This hydrophobic cleft is a common binding pocket of most actin binding proteins (ABPs) such as gelsolin, profilin, and ADF/cofilin as shown.

FIG. 3: Enhancement of HIV infection by actin-based peptides N7 and N9. (A) The HIV Rev-dependent indicator cell, Rev-CEM-GFP, was not infected (+Cell) or infected with HIV-1 (+HIV), or pre-treated with peptide N7, and then infected with HIV-1 (N7+HIV). Following infection for 2 hours, cells were washed to remove the virus and the peptide. Viral replication was measured at 2 days post infection by flow cytometry. N7 enhances HIV infection from 0.79% to 15.5%, as measured by the percentage of GFP+cells. PI, propidium iodide, was added before flow cytometry for cytotoxicity control. GFP+cells were counted only when the cells are viable (low PI staining). (B) Effects of peptide N9 were similarly measured. It enhanced HIV infection from 1.95% to 14.07%

FIG. 4: Up-regulation of cell surface receptors by N7. CEM-SS T cells or blood resting CD4 T cells were stimulated with peptide N7 for a time course. Effects on surface CD4, CXCR4 density were measured by anti-CD4 or anti-CXCR4, followed by flow cytometry.

FIG. 5: Peptide N7 and N9 modulate cell signal transduction and chemotactic cofilin and actin activity. (A) Resting memory CD4 T cells were treated with N7 for a time course, cofilin phosphorylation was measured by Western Blot. Total cofilin and GAPDH was also measured for loading control. (B) Resting CD4 T cells were treated with N9, and actin polmerization and depolymerization were measured by FITC-phalloidin staining.

FIG. 6: Peptide N7 and N9 enhance HIV DNA synthesis, nuclear migration, and integration. (A) CEM-SS T cells were treated with N7 during HV infection for 2 hours, washed, and then analyzed for viral DNA synthesis and 2-LTR circles, a marker for HIV nuclear migration. (B) Blood resting CD4 T cells were similar infected, washed, and cultured without T cell activation. Viral DNA synthesis and DNA integration were analyzed.

FIG. 7: Peptide N9 promotes HIV infection of resting CD4 T cells. Resting CD4 T cells were treated with N9, and then infected with HIV for 2 hours. Following infection, cells were washed, and culture in the absence of T cell activation and N9. Viral p24 release was measured. As controls, cells were culture with the addition of IL-7 (daily addition), or with a scrambled peptide, or only in the culture medium.

FIG. 8: Human T lymphoblast cell A3.01 was treated with N10 (10 uM). N10-mediated cell killing was monitored by staining of dead cells with Propidium iodide and FITC-labelled Annexin V. Cells were analyzed by flow cytometry.

### DETAILED DESCRIPTION

The present disclosure relates to composition and methodology for using actin-based peptide fragments to modulate cellular bioactivity, including but not limited to modulating cytoplasmic and nuclear actin dynamics, cell morphology, modulating cell motility and mobility, cell receptor dynamics, cell adhesion processes, gene expression, cell division, growth, differentiation, and apoptosis, the processes of endocytosis and exocytosis, the process of inflammation, and cellular susceptibility to intracellular pathogens such as viruses.

In one embodiment, and as described below, actin dynamics can be modulated by using actin-based peptides that are delivered intra-cellularly. These actin-based peptides, either in the L- or D-forms, can trigger or modulate actin dynamics, which lead to the modulation of cellular bioactivity such as modulation of cell surface receptors, inhibiting or promoting cell migration, increasing or decreasing cell death, or altering cellular susceptibility to intracellular bacterial or viral pathogens.

The intracellular delivery of actin-based peptides can be achieved through conjugation with cell-penetration peptide (CPPs) or membrane-permeable molecule. Currently, there are many documented CPPs that are derived from various sources (e. g. Milletti F. Cell-penetrating peptides: classes, origin, and current landscape. Drug Discovery Today, Vol. 17, Numbers 15/16, August 2012: ). CPPs are groups of diverse peptides around 5-30 amino acids. CPPs can cross the cellular membrane and act as vehicles for the intracellular delivery of proteins, peptides, small molecules, and nucleic acids. Currently, CPPs can be classified into 3 major classes: cationic, amphipathic, and hydrophobic (Table 1-3, Milletti F. Drug Discovery Today, Vol. 17, Numbers 15/16, August 2012). Cationic CPPs are mostly derived from heparin-, RNA-, and DNA-binding proteins, such as the HIV Tat peptide, arginine-based peptides, FHV coat peptide, Yeast PrP6, Penetratin, HoxA-13, PDX-1 et al. Many amphipathic CPPs are derived from antimicrobial peptides, such as LL-37, L-2, Crotamine, Buforin2, or from viral proteins, such as Ribotoxin 2 L3 loop, PreS2-TLM, VP22 et al. Many amphipathic CPPs are also derived from signal peptides [e.g. K-FGF+NLS, MPrPp (1-30)], or various natural proteins (pVEC, Azurin p18, hT18-32). There are only a few hydrophobic CPPs, compared with cathion and amphipathic peptides. Some of the hydrophobic CPPs are derived from signal peptide, such as K-FGF, or from various nature proteins, such as Bip, C105Y, FGF12 et al.

All technical terms in this description are commonly used in biochemistry, molecular biology and immunology, respectively, and can be understood by those skilled in the field of this invention. Those technical terms can be found in: MOLECULAR CLONING: A LABORATORY MANUAL, 3rd ed., vol. 1-3, ed. Sambrook and Russel, Cold Spring Harbor Laboratory Press, Cold Spring Harbor, N. Y., 2001; CURRENT PROTOCOLS IN MOLECULAR BIOLOGY, ed. Ausubel et al., Greene Publishing Associates and WileyInterscience, New York, 1988 (with periodic updates); SHORT PROTOCOLS IN MOLECULAR BIOLOGY: A COMPENDIUM OF METHODS FROM CURRENT PROTOCOLS IN MOLECULAR BIOLOGY, 5.sup.th ed., vol. 1-2, ed. Ausubel et al., John Wiley & Sons, Inc., 2002; GENOME ANALYSIS: A LABORATORY MANUAL, vol. 1-2, ed. Green et al., Cold Spring Harbor Laboratory Press, Cold Spring Harbor, N. Y., 1997; CELLULAR AND MOLECULAR IMMUNOLOGY, 4.sup.th ed. Abbas et al., W B Saunders, 1994.

As used herein, an actin-based peptide comprises an actin core domain and a cell penetration domain, operably linked together by a spacer domain. Unless indicated otherwise, the actin-based peptide N-terminus or C-terminus may have either the actin core domain or the cell penetration domain. Exemplary actin-based peptides include but are not limited to the peptides set forth in SEQ ID NOs: 1-845.

Actin core domain refers to a highly conserved domain from the actin protein sequences of human, mouse, rabbit, fly, etc. Exemplary actin core domains include NB5 (YNELRVAPEE) SEQ ID NO: 853, NB7 (QIMFETFNTP) SEQ ID NO: 854, NB9 (LPHAILRLDL) SEQ ID NO: 855, NB10 (AGRDLTDYLM) SEQ ID NO: 856, B11 (LCYVALDFEQ) SEQ ID NO: 857, and B17 (KYSVWIGGSI) SEQ ID NO: 858. Using these core domains, the present inventor developed the instant actin-based peptides set forth in SEQ ID NOs 1-845.

Cell penetration domain derive from cell-penetration peptide (CPPs) or membrane-permeable molecules.

Spacer domain refers to an amino acid sequence that links an actin core domain to a cell penetration domain. Exemplary spacer domain sequences include but are not limited to poly glycine or poly alanine.

As contemplated herein, a composition can be delivered into a cell in any form by any effective route, including but not limited to oral, parenteral, enteral, intraperitoneal, topical, transdermal (e.g., using any standard patch), ophthalmic, nasally, local, non-oral, such as aerosol, spray, inhalation, percutaneous (epidermal), subcutaneous, intravenous, intramuscular, buccal, sublingual, rectal, vaginal, intra-arterial, mucosal, and intrathecal. A composition can be administered alone, or in combination with any ingredient(s), active or inactive.

Without being bound by theory, it is thought that the actin-based peptides of the invention are likely to act through three different mechanisms: 1) the peptides disturb actin polymerization and actin depolymerization at the plus and minus ends. Thus, disturbing the process of actin treadmilling: 2) these peptides may compete directly with actin binding proteins such as cofilin, Arp2/3, thus, affecting actin dynamics; 3) these peptides may compete with actin binding adaptor proteins or actin binding motifs present on the intracellular domains of receptors.

Actin-peptides can inhibit or enhance viral infection processes at multiple steps, such as: 1) enhance or inhibit of viral entry: 2) enhance or inhibit viral intracellular migration, 3) enhance or inhibit viral nuclear migration, 4) enhance or inhibit viral assembly and budding; 5) enhance or inhibit viral cell-cell transmission.

Interaction of viruses with actin cytoskeleton: The actin cytoskeleton exists as a polarized array of filaments, termed F-actin, in dynamic equilibrium with a globular actin, or G-actin, pool. Polymerization of the F-actin filament occurs predominately at the barbed end (also known as the plus end), while depolymerization occurs predominantly at the pointed end (also known as the minus end). The precise spatiotemporal regulation of actin polymerization, mediated by actin-binding proteins (ABPs) and their upstream regulators, coordinates the force-generating and scaffolding properties of actin. These, in turn, regulate complex cellular processes including chemotaxis, cell adhesion, cytokinesis, the formation of cellular processes (microvilli, filopodia, lamellipodia, invadopodia, etc.), organelle movement, endocytosis, and the assembly of macromolecular domains during signal transduction and receptor internalization.

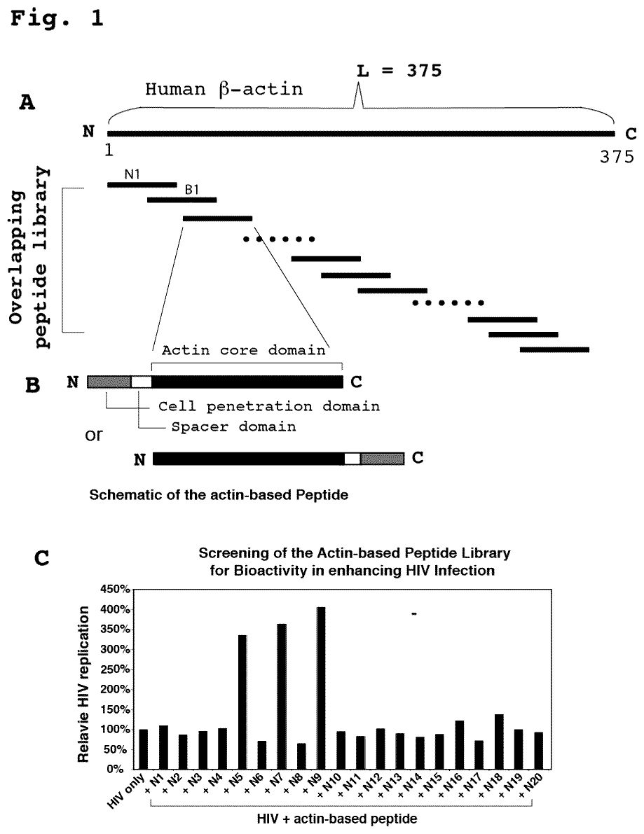

These primary functions of the actin cytoskeleton are themselves regulated by the actin-modulating ABPs, which exhibit various activities towards F-actin, including bundling and crosslinking (Fimbrin, Fascin, Filamin A, etc.), membrane and receptor association (ERM proteins, among others), capping (CapZ, tropomodulin, etc.), anti-capping (Ena/VASP proteins), nucleating and branching (the Arp2/3 complex), nucleation and polymerizing (Spire1/2 and formins), actin-associated motor activity (Myosins), and severing functions (cofilin, gelsolin, etc.). Directly upstream of these factors and their associated activities are the Rho-family monomeric GTPases, which are activated by nucleotide exchange of bound GDP to GTP. The Rho GTPases, which include 20 members, are divided into canonical signaling motifs that mediate particular cellular processes (FIG. 1). Among these are the Rac1-PAK-LIMK-Cofilin pathway, which terminates with LIMK phosphorylating and inactivating cofilin; the Rac1-WAVE-Arp2/3 pathway, which promotes actin nucleation and branching, and lamellipodium generation; the Cdc42-WASP-Arp2/3 and Cdc42-mDia pathways, which activate Arp2/3 and the formin, mDia, respectively, and promote actin nucleation, branching, polymerization, and the formation of filopodia; and the RhoA-ROCK-LIMK-Cofilin and RhoA-ROCK-MLC pathways, which are partially responsible for the assembly of the contractile uropod, stress fibers, and focal adhesions.

It is these force-generating and scaffolding properties exhibited by the actin cytoskeleton and its effectors that frequently become targets of necessity for viral replication, which also require these activities. As discussed in details below, functions such as Arp2/3 nucleation and branching and formin-mediated actin polymerization are co-opted to produce force for viral motility. Similarly, the normal mechanisms of actin rearrangement, such as cofilin activation and inactivation cycling, are dysregulated to create favorable microcompartments for genome replication and nuclear localization. As such, each stage in the viral life cycle, from entry to egress and dissemination, will be considered in turn, explicating the various motifs of viral subversion of normal cellular actin signaling functions.

As a fundamental component of the host cellular cytoskeleton, actin is routinely engaged by infecting bacterial and viruses. Bacteria and Viruses from diverse groups, and infecting diverse hosts, have convergently evolved an array of mechanisms for manipulating the actin cytoskeleton for efficacious infection. The actin cytoskeleton is critical for viral infection at many stages of the life cycle, including binding, cell entry, nuclear localization, genomic transcription and reverse transcription, assembly, and egress/dissemination. Specifically, viruses subvert the force-generating and macromolecular scaffolding properties of the actin cytoskeleton to propel viral surfing, internalization, and migration within the cell. Additionally, viruses utilize the actin cytoskeleton to support and organize assembly sites, and eject budding virions for cell-to-cell transmission.

Actin and Viral Entry: After engagement of the extracellular matrix (ECM) and the viral receptor, viruses must migrate to sites favorable for their particular mode of entry, whether it is direct plasma membrane fusion, or routes as divergent as macropinocytosis, phagocytosis, and the various forms of clathrin-mediated and clathrin-independent endocytosis. During this process, viruses frequently encounter actin-based processes, such as filopodia and microvilli, and utilize these structures to efficiently migrate to the cell membrane for entry.

Among the most common forms of this process is virus surfing, in which engagement of the receptor or ECM promotes movement of the attached virion towards the cell body in an actin-dependent mechanism. For instance, Lehmann et al. exhibited Murine leukemia virus (MLV), Avian leukosis virus (ALV), and Human immunodeficiency virus (HIV) surfing along filopodia in HEK293T cells transfected with their respective receptors (Lehmann M J, et al., 2005). MLV particles, in particular, migrated to the base of the filopodia and fused with the membrane, indicating that this was the active, and perhaps predominant, entry location (Lehmann M J, et al., 2005). Furthermore, this viral surfing was energy-dependent, being inhibited by sodium azide, and was dependent on functional myosin II and F-actin: treatment of cells with the myosin II inhibitor, blebbistatin, or cytochalasin D, which blocks F-actin barbed ends from polymerization, eliminated virus surfing and promoted random motility at the plasma membrane (Lehmann M J, et al., 2005). These treatments additionally inhibited MLV and ALV viral infection in rat XC cells, indicating that the viral surfing route contributed to productive infection (Lehmann M J, et al., 2005). The model developed around this process is centered around actin retrograde flow in which myosin II generates forces that pull the filopodia-associated actin filaments towards the cell body, which is commensurate with viral receptor-actin association and filopodial tip actin polymerization. The net result is that the virus-receptor complex is processively pushed and dragged towards the cell body where entry occurs.

Viral surfing on filopodia has also been described for Herpes simplex virus type 1 (HSV-1) (Clement C, et al., 2006; Dixit R, et al., 2008), vaccinia virus (Mercer J, et al., 2008), Human papilloma virus 16 (HPV-16) (Schelhaas M, et al., 2008), Hepatitis C virus (HCV) (Coller K E, et al., 2009), and HIV-1 (Lehmann M J, et al., 2005), indicating that utilization of actin retrograde flow in cellular processes, particularly filopodia, may be a general mechanism for extracellular viral migration towards the cell body. To some extent, the intracellular migration of viruses is a virus-dependent active process, requiring signal transduction from or clustering of the viral receptor. For instance, HSV-1 not only migrated upon filopodia, but dramatically induced their formation in P19 neuron-like cells (Dixit R, et al., 2008). This indicats that HSV-1 engagement of the viral receptor specifically introduces signaling events to mediate filopodia formation, implicating that Rho and PI3K-dependent pathways are critical for mediating cytoskeletal structures essential for viral infection (Clement C, et al., 2006; Zheng K, et al., 2014b).

As indicated above for HSV-1, another common theme preceding cell entry is that virus-LIMK-cofilin receptor engagements can actively signal to the actin cytoskeleton. This receptor-mediated signaling can dramatically affect entry events, promoting receptor clustering, receptor endocytosis, and fusion complex stabilization. Additionally, these signal transduction events often have important impacts on subsequent events in the viral life cycle. For instance, binding of HIV-1 to the chemokine coreceptor CXCR4/CCR5 triggers the activation of Rac1-PAK1/2-LIMK-cofilin (Yoder A, et al., 2008; Vorster P J, et al., 2011). This signaling pathway regulates actin dynamics and the surface cycling of CXCR4, facilitating viral entry and post entry DNA synthesis and nuclear migration (Vorster P J, et al., 2011). Clustering of HIV-1 viral receptor and coreceptor (CD4 and either CCR5 or CXCR4) has also been suggested to be dependent on viral receptor signaling: specifically, engagement of both CD4 and CXCR4 activates Filamin A (Jiménez-Baranda S, et al., 2007), which promots receptor motility and clustering. The ERM protein, Moesin, was also found to play a similar role in HIV-1 receptor clustering, in which the membrane receptor-actin crosslinking activity and receptor clustering occur after a viral receptor-mediated Moesin phosphorylation event (Barrero-Villar M, et al., 2009). It is probable that many other viruses utilize the actin cytoskeleton to induce receptor clustering and promote viral entry.

After engagement with the viral receptor, viral entry occurs either at the plasma membrane directly, or during/after internalization by one of the endocytic routes. Many, and perhaps all, of these routes depend on actin to one extent or another, particularly in primary, differentiated cells. For instance, macropinocytosis, which involves the Rac1-PAK-LIMK-Cofilin and, possibly, Rac1-WAVE-Arp2/3 pathways, has been shown to be required for HIV-1entry into macrophages (Carter GC, et al., 2011). Macropinocytosis is also required for the entry of African swine fever virus (ASFV) (Sánchez E G, et al., 2012) and HPV-16 (Schelhaas M, et al., 2012). It also serves as an alternate entry route for Influenza A virus (IAV) (de Vries E, et al., 2011). An interesting twist on this is found in vaccinia virus entry, where viral envelope-associated phosphatidylserine induces an apoptotic body-mimicking signaling pathway, inducing a Rac1, PAK, tyrosine kinase, and actin-dependent macropinocytic entry route. Although, as reviewed by Moss (Moss B, 2012), the poxvirus entry fusion complex-composed of 11-12glycosylated proteins-is abnormally complex, and other entry routes exist. Huttunem et al. (2014) also implicate a Rac1-dependent entry mechanism, involving neutral multivesicular bodies, in Coxsackievirus A9 entry (Huttunen M, et al., 2014). Similarly, HSV-1 has been shown to utilize a RhoA, tyrosine kinase, and actin-dependent phagocytosis-like route for entry (Clement C, et al., 2006; Zheng K, et al., 2014b). Poliovirus also utilizes an actin and tyrosine-kinase dependent route for entry, wherein genomic RNA release occurs in vesicles or membrane invaginations within 100-200 nm of the plasma membrane (Brandenburg B, et al., 2007). These examples, including many viruses from divergent groups, illuminate the need to co-opt the force generating and scaffolding functions of the cortical actin cytoskeleton, and the signaling pathways that modulate these functions, during viral entry.

Actin and nuclear migration: After accessing the cortisol, most viruses must traverse the gap between the entry site and the nucleus for expression, genome replication, and, for some viruses, assembly. It is to this end that viruses utilize cellular force-generating apparatuses, which, along with dynein and kinesin for microtubule-based transport, include the motor and polymerizing proteins for the actin cytoskeleton; specifically, spire, formins, Arp2/3, and myosins. For instance, HIV-1 migrates to the nucleus utilizing two downstream components of Rac signaling, the Rac1-PAK1/2-LIMK-Cofiln pathway (Yoder A, et al., 2008; Vorster P J, et al., 2011), and the Rac1-WAVE2-Arp2/3 signaling pathway (Spear M, et al., 2014). The initial stimulus for Rac activation was also shown to require coreceptor-CCR5 or CXCR4-engagement and signal transduction (Yoder A, et al., 2008; Vorster P J, et al., 2011; Spear M, et al., 2014). Interestingly, from a potential therapeutic standpoint, treatment of CD4 T lymphocytes with the Arp2/3 inhibitor, CK-548 (Nolen BJ, et al., 2009), dramatically reduced HIV-1 replication at dosages that did not affect CD4 T cell activation or induce cytotoxicity (Spear M, et al., 2014). For viruses with larger genomes, more elaborate mechanisms for mediating nuclear localization exist. This is the case with baculovirus, where the p78/83 capsid encodes a WASP/WAVE homology domain, the WASP homology-Cofilin homology/Connector-Acidic domain (WCA) (Machesky LM, et al., 2001). This domain is normally found in the Nuclear Promoting Factors (NPFs) of the WASP/WAVE family of proteins, whereupon NPF activation leads to WCA exposure and activation of the Arp2/3complex for actin polymerization (Higgs H N, et al., 1999, 2001; Pollard T D, et al., 2003). However, in the presence of baculovirus p78/83, the viral WCA domain activates Arp2/3 at the viral core surface, generating forces and an actin comet tail that propel the core to the nucleus for expression and replication (Goley E D, et al., 2006; Ohkawa T, et al., 2010). As will be discussed later, nuclear translocation of the Arp2/3 complex, nuclear F-actin polymerization, and the resultant perturbation of nuclear structure also play a critical role during viral assembly and egress (Ohkawa T, et al., 1999; Goley ED, et al., 2006). HSV-1 has also been shown to utilize actin for intracellular motility and nuclear localization in a process involving the cofilin phosphatase and activator, slingshot, and calcium-regulated protease, calpain (Zheng K, et al., 2014a). The siRNA knockdown of slingshot and calpain reduced viral infection and led to a perinuclear localization of the viral cores, whereas wild-type virus localized to the nucleus efficaciously. In a somewhat stunning display of the requirements for specificity in actin signaling at distinct stages of viral replication, at later time points (8 hours post-infection and beyond), HSV-1 promotes ubiquitin/proteasome-dependent degradation of slingshot, leading to cofilin activation and promoting viral replication (Xiang Y, et al., 2014).

Actin in assembly: Once localized to the nucleus, or cytoplasm for large dsDNA viruses, viruses must concentrate, organize, and assemble their structural proteins, early-acting accessory and catalytic proteins, and genomic nucleic acids prior to egress of the viral particle. Needless to say, this requirement for colocalization of viral proteins, viral nucleic acids, and cellular factors requires scaffolding functions, and filamentous actin is often subverted for this purpose. This was shown for baculovirus: specifically, that mutation of the baculoviral CA domain dramatically abrogated nuclear F-actin accumulation and viral replication; furthermore, that viral progeny produced by one such mutant were distorted, either lacking an envelope altogether or having capsids that did not align with their envelope (Goley E D, et al., 2006).

In a similar fashion, Potato virus X (PVX), radically rearranges the ER and Golgi membranes and host actin into enormous assembly sites called the X-body (Tilsner J, et al., 2012). This process is mediated by the triple-gene block proteins (TGB1, 2, and 3), which are involved in cell-to-cell transfer through plasmodesmata, with PVX TGB1 being able to produce X-like-bodies when expressed alone (Tilsner J, et al., 2012). Although the role of actin in the X-body, and its relation to assembly was not specifically addressed, it has been shown that actin is required for intercellular transfer of PVX, in addition to Tobacco mosaic virus (TMV) and Tomato bushy stunt virus (TBSV), through plasmodesmata (Harries PA, et al., 2009). Thus, it is tempting to speculate that, even for certain plant viruses, actin may contribute to both assembly and cell-to-cell transfer.

Assembly of Influenza A virus (IAV) filamentous particles has also been shown to partially require an intact cortical actin cytoskeleton, as disruption with cytochalasin D (CCD) dramatically abrogated the titers of the filamentous, but not spherical, virions (Roberts PC, et al., 1998). This was later corroborated by Simpson-Holley et al. (2002), showing that treatment of MDCK cells with CCD, jasplakinolide (Jas), or latrunculin A (LatA) dramatically redistributed plasma membrane hemagglutinin (HA), lipid rafts, and actin into annular structures; there was also a commensurate loss of cell surface HA-containing filamentous projections and filamentous virus production (Simpson-Holley M, et al., 2002a). The authors attributed the loss of filamentous viral assembly not to actin playing a specific role in assembly, per se, but rather mobilizing and recruiting lipid rafts to the assembly sites of filamentous particles, which, given their size, require a much larger pool of lipids than their spherical particle counterparts. As such, with lipid rafts, actin, and HA reorganized into small annuli, only spherical particle production occurs owing to the lack of sufficient lipid raft material for the larger filamentous particles (Simpson-Holley M, et al., 2002b).

Measles virus (MeV) has also been shown to utilize actin during assembly. For instance, disruption of the actin cytoskeleton rapidly reduced MeV particle release from the plasma membrane (Stallcup K C, et al., 1983). Furthermore, actin has been observed to colocalize with budding MeV, with meromyosin-labeled actin barbed ends protruding into the nascent virions in close juxtaposition with the viral nucleocapsid (Bohn W, et al., 1986). Additionally, the viral Matrix protein (M) was found to associate with F-actin, and this was shown to reduce the interaction with viral hemagglutinin (H) protein, as CCD treatment dramatically reduced actin co-immunoprecipitating with M, while enhancing H co-immunoprecipitation (Wakimoto H, et al., 2013). This study also indicated that there is a tradeoff between M-actin affinity and viral infectivity, wherein a higher affinity between M and H increases the amount of H, and hence the infectivity, of cell-free virus at the expense of cell-cell fusion. Furthermore, mutations in the M protein affecting M-actin affinity may arise in cell types where cell-cell fusion is more productive than free viral infection (Wakimoto H, et al., 2013). In another study, Dietzel et al. (2013) exhibited that, while CCD treatment enhanced the rate of cell-cell fusion, it also reduces co-transport of M-RNP to the cell surface (Dietzel E, et al., 2013). Additionally, Jas treatment blocked free-virus production, while not affecting M-RNP co-transport, indicating that a dynamic actin cytoskeleton is required for cell-free virus maturation and release (Dietzel E, et al., 2013).

ROCK, LIMK, and cofilin have also been implicated in HIV-1 and Mason-Pfizer monkey virus assembly, release, and cell-cell transmission (Wen X, et al., 2014). Knockdown of LIMK did not inhibit HIV-1 maturation, yet, fully enveloped particles remained associated with the plasma membrane as mature virion aggregates (Wen X, et al., 2014). LIMK was also shown to be recruited to assembly sites (Wen X, et al., 2014). Although the exact mechanism for ROCK-LIMK-Cofilin contribution to virion release remains incompletely resolved, the authors suggested a factor retains the viral particles when normal cytoskeletal dynamics are disrupted (Wen X, et al., 2014).

Actin and cell-cell transmission: Cell-to-cell transmission of animal viruses is a highly efficacious route of dissemination that minimizes interactions with the innate and adaptive immune system. Additionally, this process can render certain antivirals less effective, giving the issue increasing prominence in therapeutic research (Agosto L M, et al., 2014). It constitutes the infection of neighboring cells by an infected cell, typically mediated by the fusogenic surface protein or envelope glycoprotein. Perhaps the best-studied example of this is Vaccinia virus cell-cell transmission (reviewed in (Welch M D, et al., 2013)). In 1976, Vaccinia virions were found to project from CCB-sensitive microvilli-like structures (Stokes G V, 1976); later, it was discovered that these virus-associated projections contained, in addition to actin, a-actinin (Hiller G, et al., 1979), fimbrin, and filamin (Hiller G, et al., 1981; Krempien U, et al., 1981). Additionally, Intracellular Enveloped Virions (IEVs) were shown to induce CCD-sensitive actin comet tails, reminiscent of Listeria, Shigella and Rickettsia infections (Cudmore S, et al., 1995). The actin comet-inducing factor, as identified by phenotypic characterization of deletion mutant viruses, was viral A36, a type 1b membrane protein (Röttger S, et al., 1999) that becomes tyrosine phosphorylated (Frischknecht F, et al., 1999a) at Y112 and Y132 (Frischknecht F, et al., 1999b). Y112 phosphorylation recruits the adaptor protein, Nck, via its SH2 domain (Frischknecht F, et al., 1999b), while Y132 was shown to recruit another adaptor protein, Grb2 (Scaplehorn N, et al., 2002). These proteins, in turn, help to recruit the N-WASP NPF complex (Frischknecht F, et al., 1999b; Moreau V, et al., 2000), which mediates Arp2/3 activation via its WCA domain. The result of this signaling cascade is the production of actin comet tails. When IEVs fuse with the plasma membrane, becoming Cell-associated Enveloped Virions (CEVs), viral A36 remains in the plasma membrane below the virion, promoting the production of the long, microvilli-like structures identified in 1976, and promoting viral dissemination (Welch M D, et al., 2013). Other factors that promote and regulate vaccinia virus motility are continually being discovered, including the formin, FHOD1, its upstream regulator, Rac1 (Alvarez D E, et al., 2013), and casein kinase 2 (Alvarez D E, et al., 2012).

HIV has also been shown to utilize an actin-dependent (Jolly C, et al., 2004, 2007) mode of transmission between T cells, in which viral Env glycoprotein on the donor cell organizes a polarized Virological Synapse (VS) that mediates highly efficient cell-cell transmission. This process may be up to 18,000 times more efficacious that free viral infection (Chen P, et al., 2007). Morphologically, the VS and associated signaling complex, which contains the viral receptor, CD4, and viral coreceptor, CXCR4 or CCR5, resembles the Supramolecular Activation Complex (SMAC) observed in T cell activation (Vasiliver-Shamis G, et al., 2008), including the incorporation of the T cell Receptor (TCR) (Vasiliver-Shamis G, et al., 2009). Although the derived signals are insufficient to promote T cell activation, they do create an actin-depleted zone, which may facilitate viral infection at its earliest stages (Vasiliver-Shamis G, et al., 2009). Additionally, HIV has been shown to transfer between T cells using cellular nanotube processes, which may mimic aspects of the VS (Sowinski S, et al., 2008). An interesting twist on these forms of cell-to-cell transmission can be found in infected dendritic cells (DCs), wherein viral filopodia, containing immature virions at their tips, were observed (Aggarwal A, et al., 2012). These viral filopodia were found to be quite dynamic, allowing up to 800 CD4 T cell contacts per hour (Aggarwal A, et al., 2012). Additionally, these viral filopodia partially required the formin, Diaphanous-2 (Diaph2), and the viral Nef accessory protein (Aggarwal A, et al., 2012).

Other roles of actin: In addition to the more canonical modes of viral cooption of the actin cytoskeleton thus described, there is accumulating evidence that cytosolic and nuclear actin may play important roles in viral transcription, translation, and genome replication though somewhat unique modalities.

For instance, in HIV-1 infection, after entry into the host cell, the viral core is deposited on a dense meshwork of cortical actin that undergoes dynamics related to signal transduction mediated by the viral Env-CD4 and Env-CXCR4/CCR5 interactions (Jiménez-Baranda S, et al., 2007; Harmon B, et al., 2008, 2010; Yoder A, et al., 2008; Barrero-Villar M, et al., 2009; Vorster P J, et al., 2011; Spear M, et al., 2014). It is in this submembranous, dynamic actin cortex that the Reverse Transcriptase Complex (RTC) must convert the ssRNA genome into dsDNA. The first indication that the RTC may utilize the actin cytoskeleton came in 1995, when Hottiger et al. exhibited an interaction between beta-actin and the large subunit of Reverse Transcriptase (RT), or the Pol polyprotein precursor (Hottiger M, et al., 1995). Later, Bukrinskaya et al. (1998) corroborated these findings, showing that the RTC does indeed associate with the actin cytoskeleton in infected cells, and that pretreating cells with CCD, but not treatment 2 hours post-infection, reduced the accumulation of early and late reverse transcription products, with a more severe phenotype for late products (Bukrinskaya A, et al., 1998). These results indicated that actin disruption may directly impact the function of RT (Bukrinskaya A, et al., 1998). As was corroborated by later studies, CCD pretreatment reduced the nuclear accumulation of late RT products by 25-fold (Bukrinskaya A, et al., 1998). This process is presumably related to both the reliance on F-actin for both reverse transcription and nuclear migration of the Pre-Integration Complex (PIC) (Yoder A, et al., 2008; Cameron P U, et al., 2010; Vorster P J, et al., 2011; Spear M, et al., 2014). Although, the exact mechanism by which F-actin contributes to RTC activity remain unresolved. Nuclear actin bundles have also been implicated in late gene (gag) mRNA nuclear export and expression (Kimura T, et al., 2000): specifically, treatment with LatA, which disrupted viral Rev-RNP-induced nuclear actin bundles, caused retention of gag mRNA in the nucleus, decreasing cytosolic gag mRNA and, presumably, reducing Gag and Gag-Pol protein expression (Kimura T, et al., 2000).

Rac1 has also been shown to be important for Influenza A virus (IAV) polymerase complex activity (Dierkes R, et al., 2014). Treatment of cells with a Rac1 inhibitor, NSC23766(Gao Y, et al., 2004), reduced viral replication in A549 cells with an IC50 of 22 uM (Dierkes R, et al., 2014). Furthermore, knockdown of TIAM1, the Rac1-activating GEF, or Rac1 itself also reduced viral replication (Dierkes R, et al., 2014). Additionally, NSC23766 reduced viral protein expression, which was later linked to inhibition of the viral polymerase complex (Dierkes R, et al., 2014). Fascinatingly, mice infected with IAV and treated with NSC23766 showed reduce titer of IAV in lung tissue, increased body weights, and a higher survivability than solvent-treated control mice (Dierkes R, et al., 2014). Although there was no specific study of whether NSC23766-mediated inhibition of the viral polymerase complex required actin, it is likely that actin played some role.

### REFERENCES

Aggarwal A, Iemma T L, Shih I, Newsome T P, McAllery S, Cunningham A L, Turville S G. 2012. Mobilization of HIV spread by diaphanous 2 dependent filopodia in infected dendritic cells. PLOS Pathog, 8: e1002762.

Agosto L M, Zhong P. Munro J, Mothes W. 2014. Highly Active Antiretroviral Therapies Are Effective against HIV-1 Cell-to-Cell Transmission. PLOS Pathog, 10: e1003982.

Alvarez D E, Agaisse H. 2012. Casein kinase 2 regulates vaccinia virus actin tail formation. Virology, 423:143-151.

Alvarez D E, Agaisse H. 2013. The formin FHOD1 and the small GTPase Rac1 promote vaccinia virus actin-based motility. J Cell Biol, 202:1075-1090.

Barrero-Villar M, Cabrero J R, Gordón-Alonso M, Barroso-González J, Alvarez-Losada S, Muñoz-Fernández M A, Sánchez-Madrid F, Valenzuela-Fernández A. 2009. Moesin is required for HIV-1-induced CD4-CXCR4 interaction, F-actin redistribution, membrane fusion and viral infection in lymphocytes. J Cell Sci, 122:103-113.

Bohn W, Rutter G, Hohenberg H, Mannweiler K, Nobis P. 1986. Involvement of actin filaments in budding of measles virus: studies on cytoskeletons of infected cells. Virology, 149:91-106.

Brandenburg B, Lee L Y, Lakadamyali M, Rust M J, Zhuang X, Hogle J M. 2007. Imaging poliovirus entry in live cells. PLOS Biol, 5: e183.

Bukrinskaya A, Brichacek B, Mann A, Stevenson M. 1998. Establishment of a functional human immunodeficiency virus type 1 (HIV-1) reverse transcription complex involves the cytoskeleton. J Exp Med, 188:2113-2125.

Cameron P U, Saleh S, Sallmann G, Solomon A, Wightman F, Evans V A, Boucher G, Haddad E K, Sekaly R-P, Harman A N, Anderson J L, Jones K L, Mak J, Cunningham A L, Jaworowski A, Lewin S R. 2010. Establishment of HIV-1 latency in resting CD4+ T cells depends on chemokine-induced changes in the actin cytoskeleton. Proc Natl Acad Sci U S A, 107:16934-16939.

Carter G C, Bernstone L, Baskaran D, James W. 2011. HIV-1 infects macrophages by exploiting an endocytic route dependent on dynamin, Rac1 and Pak1. Virology, 409:234-250.

Chen P, Hübner W, Spinelli M A, Chen B K. 2007. Predominant mode of human immunodeficiency virus transfer between T cells is mediated by sustained Env-dependent neutralization-resistant virological synapses. J Virol, 81:12582-12595.

Clement C, Tiwari V, Scanlan P M, Valyi-Nagy T, Yue B Y J T, Shukla D. 2006. A novel role for phagocytosis-like uptake in herpes simplex virus entry. J Cell Biol, 174:1009-1021.

Coller K E, Berger K L, Heaton N S, Cooper J D, Yoon R, Randall G. 2009. RNA interference and single particle tracking analysis of hepatitis C virus endocytosis. PLOS Pathog, 5: e1000702.

Cudmore S, Cossart P, Griffiths G, Way M. 1995. Actin-based motility of vaccinia virus. Nature, 378:636-638.

Dierkes R, Warnking K, Liedmann S, Seyer R, Ludwig S, Ehrhardt C. 2014. The Rac1 inhibitor NSC23766 exerts anti-influenza virus properties by affecting the viral polymerase complex activity. PloS One, 9: e88520.

Dietzel E, Kolesnikova L, Maisner A. 2013. Actin filaments disruption and stabilization affect measles virus maturation by different mechanisms. Virol J, 10:249.

Dixit R, Tiwari V, Shukla D. 2008. Herpes simplex virus type 1 induces filopodia in differentiated P19 neural cells to facilitate viral spread. Neurosci Lett, 440:113-118.

Frischknecht F, Cudmore S, Moreau V, Reckmann I, Röttger S, Way M. 1999a. Tyrosine phosphorylation is required for actin-based motility of vaccinia but not Listeria or Shigella. Curr Biol CB, 9:89-92.

Frischknecht F, Moreau V, Röttger S, Gonfloni S, Reckmann I, Superti-Furga G, Way M. 1999b. Actin-based motility of vaccinia virus mimics receptor tyrosine kinase signalling. Nature, 401:926-929.

Gao Y, Dickerson J B, Guo F, Zheng J, Zheng Y. 2004. Rational design and characterization of a Rac GTPase-specific small molecule inhibitor. Proc Natl Acad Sci U S A, 101:7618-7623.

Goley E D, Ohkawa T, Mancuso J, Woodruff J B, D'Alessio J A, Cande W Z, Volkman L E, Welch M D. 2006. Dynamic Nuclear Actin Assembly by Arp2/3 Complex and a Baculovirus WASP-Like Protein. Science, 314:464 467.

Harmon B, Campbell N, Ratner L. 2010. Role of Abl kinase and the Wave2 signaling complex in HIV-1 entry at a post-hemifusion step. PLOS Pathog, 6: e1000956.

Harmon B, Ratner L. 2008. Induction of the Galpha (q) signaling cascade by the human immunodeficiency virus envelope is required for virus entry. J Virol, 82:9191-9205.

Harries P A, Park J-W, Sasaki N, Ballard K D, Maule A J, Nelson R S. 2009. Differing requirements for actin and myosin by plant viruses for sustained intercellular movement. Proc Natl Acad Sci U S A, 106:17594-17599.

Higgs HN, Pollard TD. 1999. Regulation of actin polymerization by Arp2/3 complex and WASp/Scar proteins. J Biol Chem, 274:32531-32534.

Higgs H N, Pollard T D. 2001. Regulation of actin filament network formation through ARP2/3 complex: activation by a diverse array of proteins. Annu Rev Biochem, 70:649-676.

Hiller G, Jungwirth C, Weber K. 1981. Fluorescence microscopical analysis of the life cycle of vaccinia virus in chick embryo fibroblasts. Virus-cytoskeleton interactions. Exp Cell Res, 132:81-87.

Hiller G, Weber K, Schneider L, Parajsz C, Jungwirth C. 1979. Interaction of assembled progeny pox viruses with the cellular cytoskeleton. Virology, 98:142-153.

Hottiger M, Gramatikoff K, Georgiev O, Chaponnier C, Schaffner W, Hübscher U. 1995. The large subunit of HIV-1 reverse transcriptase interacts with beta-actin. Nucleic Acids Res, 23:736-741.

Huttunen M, Waris M, Kajander R, Hyypiä T, Marjomäki V. 2014. Coxsackievirus A9 infects cells via nonacidic multivesicular bodies. J Virol, 88:5138-5151.

Jiménez-Baranda S, Gómez-Moutón C, Rojas A, Martínez-Prats L, Mira E, Ana Lacalle R, Valencia A, Dimitrov D S, Viola A, Delgado R, Martínez-A C, Mañes S. 2007. Filamin-A regulates actin-dependent clustering of HIV receptors. Nat Cell Biol, 9:838-846.

Jolly C, Kashefi K, Hollinshead M, Sattentau Q J. 2004. HIV-1 cell to cell transfer across an Env-induced, actin-dependent synapse. J Exp Med, 199:283-293.

Jolly C, Mitar I, Sattentau Q J. 2007. Requirement for an intact T-cell actin and tubulin cytoskeleton for efficient assembly and spread of human immunodeficiency virus type 1. J Virol, 81:5547-5560.

Kimura T, Hashimoto I, Yamamoto A, Nishikawa M, Fujisawa J I. 2000. Rev-dependent association of the intron-containing HIV-1 gag mRNA with the nuclear actin bundles and the inhibition of its nucleocytoplasmic transport by latrunculin-B. Genes Cells Devoted Mol Cell Mech, 5:289-307.

Krempien U, Schneider L, Hiller G, Weber K, Katz E, Jungwirth C. 1981. Conditions for pox virus-specific microvilli formation studied during synchronized virus assembly. Virology, 113:556-564.

Lehmann M J, Sherer N M, Marks C B, Pypaert M, Mothes W. 2005. Actin-and myosin-driven movement of viruses along filopodia precedes their entry into cells. J Cell Biol, 170:317-325.

Machesky LM, Insall RH. 2001. WASP homology sequences in baculoviruses. Trends Cell Biol, 11:286-287.

Mercer J, Helenius A. 2008. Vaccinia virus uses macropinocytosis and apoptotic mimicry to enter host cells. Science, 320:531-535.

Moss B. 2012. Poxvirus cell entry: how many proteins does it take? Viruses, 4:688-707.

Moreau V, Frischknecht F, Reckmann I, Vincentelli R, Rabut G, Stewart D, Way M. 2000. A complex of N-WASP and WIP integrates signalling cascades that lead to actin polymerization. Nat Cell Biol, 2:441-448.

Nolen B J, Tomasevic N, Russell A, Pierce D W, Jia Z, McCormick C D, Hartman J, Sakowicz R, Pollard T D. 2009. Characterization of two classes of small molecule inhibitors of Arp2/3 complex. Nature, 460:1031-1034.

Ohkawa T, Volkman LE, Welch M D. 2010. Actin-based motility drives baculovirus transit to the nucleus and cell surface. J Cell Biol, 190:187-195.

Ohkawa T, Volkman L E. 1999. Nuclear F-Actin Is Required for AcMNPV Nucleocapsid Morphogenesis. Virology, 264:1-4.

Pollard T D, Borisy G G. 2003. Cellular motility driven by assembly and disassembly of actin filaments. Cell, 112:453-465.

Roberts P C, Compans R W. 1998. Host cell dependence of viral morphology. Proc Natl Acad Sci USA, 95:5746-5751.

Röttger S, Frischknecht F, Reckmann I, Smith G L, Way M. 1999. Interactions between vaccinia virus IEV membrane proteins and their roles in IEV assembly and actin tail formation. J Virol, 73:2863-2875.

Sánchez E G, Quintas A, Pérez-Núñez D, Nogal M, Barroso S, Carrascosa Á L, Revilla Y. 2012. African swine fever virus uses macropinocytosis to enter host cells. PLOS Pathog, 8: e1002754.

Scaplehorn N, Holmström A, Moreau V, Frischknecht F, Reckmann I, Way M. 2002. Grb2 and Nck act cooperatively to promote actin-based motility of vaccinia virus. Curr Biol CB, 12:740-745.

Schelhaas M, Ewers H, Rajamäki M-L, Day P M, Schiller J T, Helenius A. 2008. Human papillomavirus type 16 entry: retrograde cell surface transport along actin-rich protrusions. PLOS Pathog, 4: e1000148.

Schelhaas M, Shah B, Holzer M, Blattmann P, Kühling L, Day P M, Schiller J T, Helenius A. 2012. Entry of Human Papillomavirus Type 16 by Actin-Dependent, Clathrin-and Lipid Raft-Independent Endocytosis. PLOS Pathog, 8: e1002657.

Simpson-Holley M, Ellis D, Fisher D, Elton D, McCauley J, Digard P. 2002a. A functional link between the actin cytoskeleton and lipid rafts during budding of filamentous influenza virions. Virology, 301:212-225.

Simpson-Holley M, Ellis D, Fisher D, Elton D, McCauley J, Digard P. 2002b. A Functional Link between the Actin Cytoskeleton and Lipid Rafts during Budding of Filamentous Influenza Virions. Virology, 301:212-225.

Sowinski S, Jolly C, Berninghausen O, Purbhoo M A, Chauveau A, Köhler K, Oddos S, Eissmann P, Brodsky F M, Hopkins C, Onfelt B, Sattentau Q, Davis D M. 2008. Membrane nanotubes physically connect T cells over long distances presenting a novel route for HIV-1 transmission. Nat Cell Biol, 10:211-219.

Spear M, Guo J, Turner A, Yu D, Wang W, Meltzer B, He S, Hu X, Shang H, Kuhn J, Wu Y. 2014. HIV-1 Triggers WAVE2 Phosphorylation in Primary CD4 T Cells and Macrophages, Mediating Arp2/3-dependent Nuclear Migration. J Biol Chem, 289:6949-6959.

Stallcup K C, Raine C S, Fields B N. 1983. Cytochalasin B inhibits the maturation of measles virus. Virology, 124:59-74.

Stokes G V. 1976. High-voltage electron microscope study of the release of vaccinia virus from whole cells. J Virol, 18:636-643.

Tilsner J, Linnik O, Wright K M, Bell K, Roberts A G, Lacomme C, Santa Cruz S, Oparka K J. 2012. The TGB 1 movement protein of Potato virus X reorganizes actin and endomembranes into the X-body, a viral replication factory. Plant Physiol, 158:1359-1370.

Vasiliver-Shamis G, Cho M W, Hioe C E, Dustin M L. 2009. Human Immunodeficiency Virus Type 1 Envelope gp120-Induced Partial T-Cell Receptor Signaling Creates an F-Actin-Depleted Zone in the Virological Synapse. J Virol, 83:11341-11355.

Vasiliver-Shamis G, Tuen M, Wu T W, Starr T, Cameron T O, Thomson R, Kaur G, Liu J, Visciano M L, Li H, Kumar R, Ansari R, Han D P, Cho M W, Dustin M L, Hioe C E. 2008. Human immunodeficiency virus type 1 envelope gp120 induces a stop signal and virological synapse formation in noninfected CD4+ T cells. J Virol, 82:9445-9457.

Vorster P J, Guo J, Yoder A, Wang W, Zheng Y, Xu X, Yu D, Spear M, Wu Y. 2011. LIM kinase 1 modulates cortical actin and CXCR4 cycling and is activated by HIV-1 to initiate viral infection. J Biol Chem, 286:12554-12564.

De Vries E, Tscherne D M, Wienholts M J, Cobos-Jiménez V, Scholte F, García-Sastre A, Rottier P J M, de Haan CAM. 2011. Dissection of the Influenza A Virus Endocytic Routes Reveals Macropinocytosis as an Alternative Entry Pathway. PLOS Pathog, 7: e1001329.

Wakimoto H, Shimodo M, Satoh Y, Kitagawa Y, Takeuchi K, Gotoh B, Itoh M. 2013. F-Actin Modulates Measles Virus Cell-Cell Fusion and Assembly by Altering the Interaction between the Matrix Protein and the Cytoplasmic Tail of Hemagglutinin. J Virol, 87:1974-1984.

Welch M D, Way M. 2013. Arp2/3-Mediated Actin-Based Motility: A Tail of Pathogen Abuse. Cell Host Microbe, 14:242-255.

Wen X, Ding L, Wang J-J, Qi M, Hammonds J, Chu H, Chen X, Hunter E, Spearman P. 2014. ROCK1 and LIM Kinase Modulate Retrovirus Particle Release and Cell-Cell Transmission Events. J Virol; DOI: 10.1128/JVI.00023-14.

Xiang Y, Zheng K, Zhong M, Chen J, Wang X, Wang Q, Wang S, Ren Z, Fan J, Wang Y. 2014. Ubiquitin-proteasome-dependent slingshot 1 downregulation in neuronal cells inactivates cofilin to facilitate HSV-1 replication. Virology, 449:88-95.

Yoder A, Yu D. Dong L, Iyer S R, Xu X, Kelly J, Liu J, Wang W. Vorster P J, Agulto L. Stephany D A, Cooper J N, Marsh J W, Wu Y. 2008. HIV envelope-CXCR4 signaling activates cofilin to overcome cortical actin restriction in resting CD4 T cells. Cell, 134:782-792.

Zheng K. Xiang Y, Wang Q. Jin F. Chen M, Ma K, Ren Z, Wang Y. 2014a. Calcium-signal facilitates herpes simplex virus type 1 nuclear transport through slingshot 1 and calpain-1 activation. Virus Res, 188C: 32-37.

Zheng K. Xiang Y, Wang X, Wang Q. Zhong M. Wang S, Wang X, Fan J, Kitazato K, Wang Y. 2014b. Epidermal growth factor receptor-PI3K signaling controls cofilin activity to facilitate herpes simplex virus 1 entry into neuronal cells. mBio, 5: e00958-00913.

### Actin-Based Peptides can Inhibit or Enhance Bacterial Infection Through Inhibiting Bacterial Motility and Mobility

Actin and bacterial mobility: The cytoskeleton is a dynamic structure composed of microfilaments (filamentous actin or F-actin), intermediate filaments, and microtubules that are mainly responsible for defining cell shape, mediating motility, and transporting macromolecules and organelles. The actin cytoskeleton, in particular, has been shown to be the driving force for cell motility and migration [1]. As an integral component of intracellular molecular networks, the actin cytoskeleton is also a target for pathogens. It has been known that the human bacterial pathogens Listeria monocytogenes [2, 3] effectively uses the driving force generated from actin polymerization for mobility.

Actin-Based Peptides cause Receptor Up/Down Regulation

Mattila and co-authors have suggested that actin regulates receptors through controlling receptor compartmentalization, receptor dynamics, and receptor clustering [4]. Actin cytoskeleton is also a point of integration for receptor cross talk through modulation of protein dynamics and clustering.

Gowriskankar and coauthors [5] have also proposed a model in which active remodeling of cortical actin regulates spatiotemporal organization of cell surface molecules such as surface receptors. The authors classify surface molecules into 3 classes based on their relationship with the cortical actin-namely, inert surface molecules, passive surface molecules, and active surface molecules. The inert (e.g. short-chain lipids) and passive (e. g. transmembrane proteins with actin-binding domains) molecules on the cell surface can be driven to form transient nanoclusters by actin dynamics and local actin architecture. Many cell surface proteins can directly connect to the cytoplasmic actin using actin-binding motifs such as PDZ, SH3 (Src- or PRD (proline-rich domain). Active cell surface molecules (e.g. integrin and T cell receptors) are capable of interacting with actin and modifying its dynamics. These molecules can recruit molecules involved in actin remodulation. They can also be organized in microclusters by actin-dependent processes.

The instant actin-based peptides can modulate cell surface molecules and receptors through promoting or inhibiting actin dynamics.

For example, and in no way limiting, the instant actin-based peptides can modulate cancer receptors. A review article describing the most important cancer receptors is A. M. Scott et al., Antibody therapy of cancer, Nature Reviews Cancer, Vol. 12, APRIL 2012, 278-287. Table 2 in the article lists important receptors that can be targeted for therapy (e.g, CD20, CD30, CD33, CD52, EpCAM, CEA, gpA33, Mucins, TAG-72, CAIX, PSMA, VEGF, VEGFR, EGFR, Integrin αVB3, Integrin α5B3, ERBB2, ERBB3, EPHA3, TRAILR1, RANKL, FAP, Tenascin)

### REFERENCES

[1]. Pollard T D, Borisy G G. Cellular motility driven by assembly and disassembly of actin filaments. Cell. 2003; 112 (4): 453-65. PubMed PMID: 12600310.
[2]. Loisel T P, Boujemaa R, Pantaloni D, Carlier M F. Reconstitution of actin-based motility of Listeria and Shigella using pure proteins. Nature. 1999;401 (6753): 613-6. PubMed PMID: 10524632.
[3]. Cameron L A, Giardini P A, Soo F S, Theriot J A. Secrets of actin-based motility revealed by a bacterial pathogen. Nat Rev Mol Cell Biol. 2000; 1 (2): 110-9. PubMed PMID: 11253363.
[4]. Mattila P K, Batista F D, Treanor B. Dynamics of the actin cytoskeleton mediates receptor cross talk: An emerging concept in tuning receptor signaling. J Cell Biol. 2016;212 (3): 267-80. doi: 10.1083/jcb.201504137. PubMed PMID: 26833785; PMCID: PMC4748574.
[5]. Gowrishankar K, Ghosh S. Saha S. C R, Mayor S, Rao M. Active remodeling of cortical actin regulates spatiotemporal organization of cell surface molecules. Cell. 2012; 149 (6): 1353-67. doi: 10.1016/j.cell.2012.05.008. PubMed PMID: 22682254.
The Instant Actin-Based Peptides Provide Anti-Inflammatory/Inhibit Cheomotaxis: Actin polymerization is the major driving force for the chemotaxis of immune cells. It is known that inhibiting actin polymerization inhibits chemotaxis, which is required for the inflammatory responses of immune cells. The instant actin-based peptides can interfere with actin polymerization process, and may possess anti-inflammatory properties.

### REFERENCE

1. Felgner P L, Gadek T R, Holm M, Roman R, Chan H W, Wenz M, Northrop J P, Ringold G M, Danielsen M. Lipofection: a highly efficient, lipid-mediated DNA-transfection procedure. Proc Natl Acad Sci U S A. 1987: 84 (21): 7413-7. PubMed PMID: 2823261; PMCID: PMC299306.

### EXAMPLES

Illustrative Examples are presented below. They are exemplary and non-limiting.

### EXAMPLE 1: Human and Related Composition

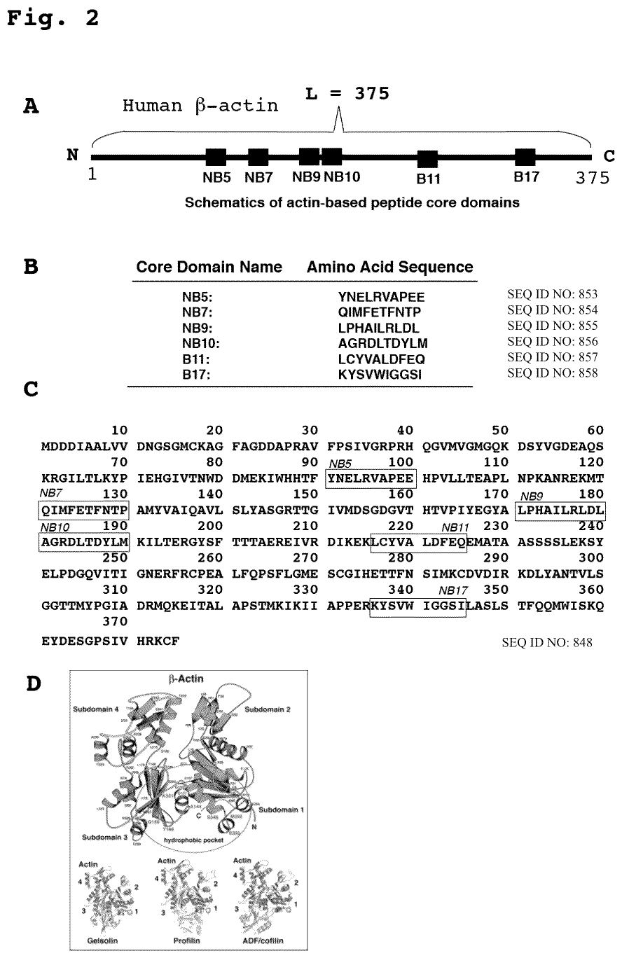

As exemplified in FIG. 1 and FIG. 2, the amino acid (AA) sequence of human beta-actin, which has 375 amino acids (L=375), can be divided into multiple overlapping fragments (in L- or D-form peptides) with various lengths (P). The total number of peptides (N) can be generated will be: N=L−P, where N=total number of peptides that could be generated, L=the number of amino acids in an actin protein isoform. P=the number of amino acids in an actin peptide, P can be from 2 amino acids to (L-1) amino acids. As exemplified in FIG. 1A, human beta-actin can be divided into multiple peptides (N=375-20 =350) of 20 AA in length. These peptides can be chemically conjugated to a cell penetration peptide or member-permeable molecules (either in the N terminus or the C terminus) for intracellular delivery (FIG. 1B). Conjugation is well known in the chemical arts, and can occur by any art-accepted method.

### EXAMPLE 2: Enhancement of HIV Infection by Actin-based Peptide N7 and N9

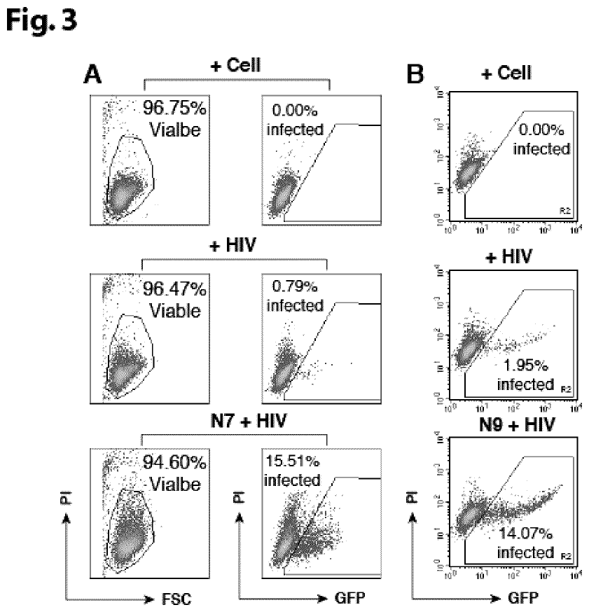

In screening a peptide library (FIG. 1C), multiple peptides including Peptide N7 and N9 were identified that can drastically enhance HIV infection of human T cells (FIG. 1C). As shown in FIG. 3, the actin-based peptides N7 and N9 enhance HIV infection. The HIV Rev-dependent indicator cell, Rev-CEM-GFP, was not infected (+Cell) or infected with HIV-1 (+HIV), or pre-treated with peptide N7, and then infected with HIV-1 (N7+HIV). Following infection for 2 hours, cells were washed to remove the virus and the peptide. Viral replication was measured at 2 days post infection by flow cytometry.

N7 enhances HIV infection from 0.79% to 15.5%, as measured by the percentage of GFP+ cells. PI, propidium iodide, was added before flow cytometry for cytotoxicity control. GFP+ cells were counted only when the cells are viable (low PI staining).

Similar effects were observed and measured for peptide N9. N9 enhanced HIV infection from 1.95% to 14.07%.

### EXAMPLE 3: Peptide N7 Up-Regulates Surface Receptors

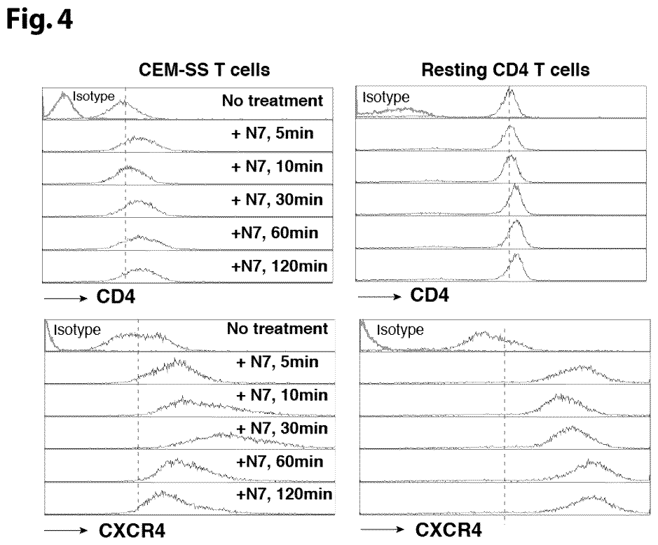

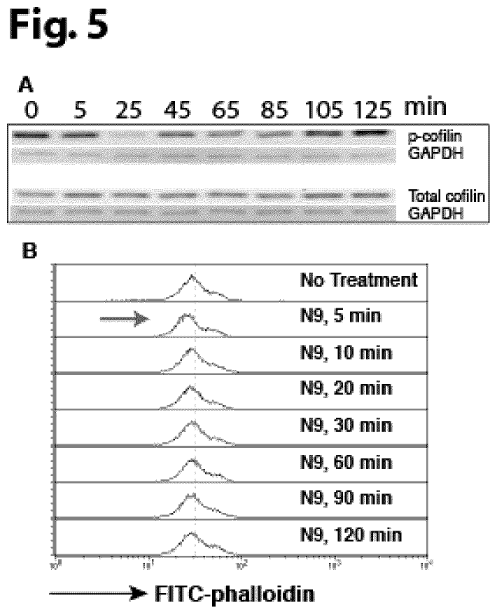

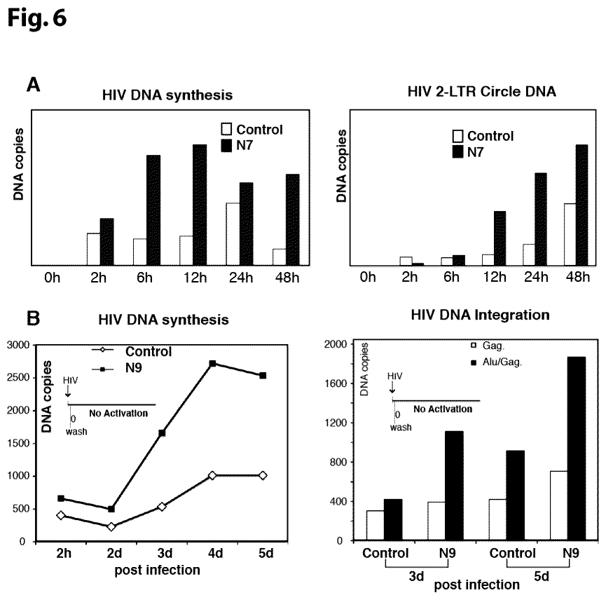

As shown in FIG. 4, exposure of T cells to Peptide N7 was also found to drastically up-regulate the surface CXCR4 receptors, while there were a moderate up-regulation of the surface CD4 receptor (FIG. 4). Mechanistic studies revealed that Peptide N7 briefly triggers cofilin activation and actin depolymerization (FIG. 5), likely causing a transient breakage of the cortical actin layer, leading to higher levels of surface receptors, HIV viral entry, viral DNA synthesis, and nuclear migration (FIG. 6).

### Experimental Procedure of N7 Up-Regulation of Surface Receptors:

1) Culture human CEM-SS T cells at 5×10⁵ cells/ml

2) Add peptide N7 to the cell to a final concentration of 10 uM at various times from 5 min to 120 min (e. g. 5, 10, 15, 30, 45, 60, 120 min).

3) Take out 1−2×10⁵ cells, put on ice, perform routine surface staining using FITC-, PE- or PE-cy5-labelled specific antibodies against cell receptors as recommended by the reagent manufacturer for cell surface staining.

4) Analyzed the staining by flow cytometer.

### EXAMPLE 4: Peptide N7 and N9 Modulate Cofilin and Actin Activity

Resting memory CD4 T cells were treated with N7 for a time course, and cofilin phosphorylation was measured by Western Blot. Total cofilin and GAPDH was also measured for loading control (FIG. 5A). Resting CD4 T cells were treated with N9, and actin polmerization and depolymerization were measured by FITC-phalloidin staining (FIG. 5B). See FIG. 5.

### EXAMPLE 5: Peptide N7 and N9 Enhance HIV DNA Synthesis, Nuclear Migration, and Integration

As shown in FIG. 6, peptide N7 and N9 enhance HIV DNA synthesis, nuclear migration, and integration. CEM-SS T cells were treated with N7 during HV infection for 2 hours, washed, and then analyzed for viral DNA synthesis and 2-LTR circles, a marker for HIV nuclear migration. See FIG. 6 (A). Blood resting CD4 T cells were similar infected, washed, and cultured without T cell activation. Viral DNA synthesis and DNA integration were analyzed. See FIG. 6 (B).

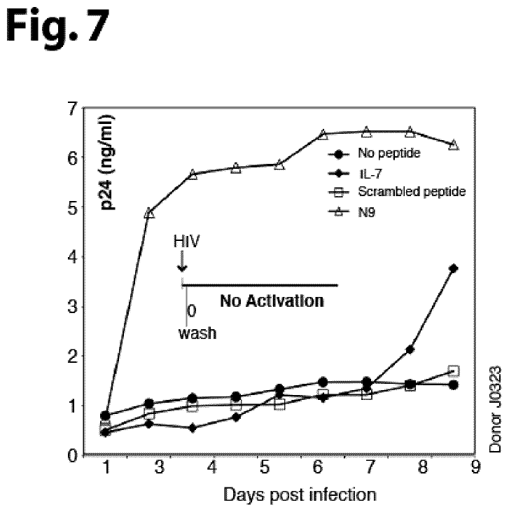

The ability of N9 to stimulate HIV infection of blood resting CD4 T cells was also tested. Resting CD4 T cells are relatively resistant to HIV infection. The lack of activity in the cortical actin limits viral early steps in the infection of resting T cells (Yoder et al., 2008). As shown in FIG. 7, a single-dosage N9 treatment prior to HIV infection drastically stimulated HIV infection of resting T cells. Importantly, this enhancement occurs in the absence of T cell activation or cytokine stimulation, and N9 is more potent at early time in stimulating viral infection than certain cytokines such as IL-7 (administered daily).

Assay protocols for using peptides of the invention

### Example 6: Protocol for Using Actin-Based Peptides to Enhance Viral Infection

1) Culture human T cell in growth medium

2) Take 1−2×10⁶ cells, resuspend into 2×10⁶ per ml

3) Add actin-peptides to cell to a final concentration of 10 uM

4) Incubate for 1-2 hours

5) Add viruses (e. g. 100-500 ul), and then add additional amount of actin-peptide to maintain the final concentration to 10 uM

6) Infect cells for 1-4 hours

7) Wash cells to remove the virus and the peptide

8) Culture for 1-7 days post infection to monitor viral replication.

### Example 7: Protocol for Using Actin-Based Peptides to Block Cell Migration (Chemotaxis)

1) Take 0.5×10⁵ cells, resuspend into 100 ul medium

2) Treat cells with actin-peptide for 1-2 hours

3) Add cells to the upper chamber of a 24-well transwell plate (corning)

4) The lower chamber is filled with 600 ul medium premixed with SDF-1 (40 ng/ml).

5) Incubate the plate at 37° C. for 2 hours 6) The upper chamber was removed, and cells in the lower chamber was counted.

### Example 8: Results and protocol for using peptide N10 to kill cancer cells

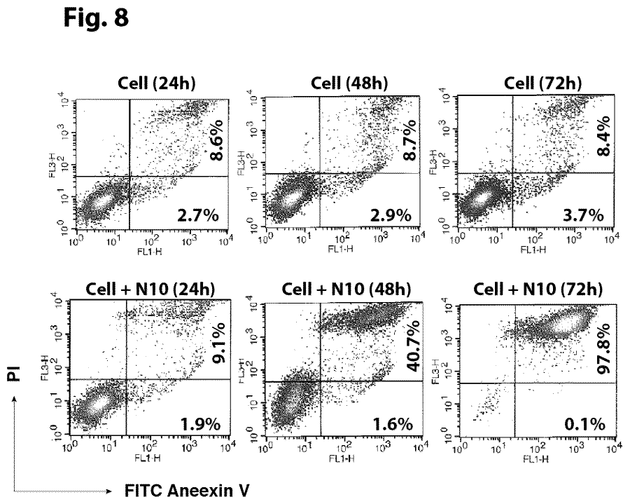

We found that treatment of human T lymphoma cells with N10 led to the killing of T cells (FIG. 8). FIG. 8 shows human T lymphoblast cell A3.01 was treated with N10 (10 uM). N10-mediated cell killing was monitored by staining of dead cells with Propidium iodide and FITC-labelled Annexin V. Cells were analyzed by flow cytometry.

### Experimental Procedure:

5) Culture human A3.01 cells at 5×10⁵ cells/ml

6) Add peptide N10 to the cell to a final concentration of 10 uM

7) Continue to culture cell at 37° C., maintain cell density below 1×10⁶ cells/ml

8) Take out 1-2×10⁵ cells, put on ice, add P.I., and FITC-Annexin V as recommended by the reagent manufacturer for dead/apoptotic cell staining.

9) Analyzed the staining by flow cytometer.

### REFERENCES

Kotsakis, A., Pomeranz, L. E., Blouin, A., and Blaho, J. A. (2001). Microtubule reorganization during herpes simplex virus type 1 infection facilitates the nuclear localization of VP22, a major virion tegument protein. J Virol75, 8697-8711.
Pollard, T. D., and Borisy, G. G. (2003). Cellular motility driven by assembly and disassembly of actin filaments. Cell 112, 453-465.
Spear, M., Guo, J., and Wu, Y. (2012). The trinity of the cortical actin in the initiation of HIV-1 infection. Retrovirology 9, 45.
Spear, M., Guo, J., and Wu, Y. (2013). Novel anti-HIV therapeutics targeting chemokine receptors and actin regulatory pathways. Immunological Reviews DOI: 10.1111imr.12106.
Taylor, M. P., Koyuncu, 0.0., and Enquist, L. W. (2011). Subversion of the actin cytoskeleton during viral infection. Nat Rev Microbiol9, 427-439.
Wulfing, C., and Davis, M. M. (1998). A receptor/cytoskeletal movement triggered by costimulation during T cell activation. Science 282, 2266-2269.
Yoder, A., Yu, D., Dong, L., Iyer, S.R., Xu, X., Kelly, J., Liu, J., Wang, W., Vorster, P. J., Agulto, L., et al. (2008). HIV envelope-CXCR4 signaling activates cofilin to overcome cortical actin restriction in resting CD4 T cells. Cell 134, 782-792.

The following sequences are essentially derived from the core domains of the actin-based bioactive peptides: NB5 (SEQ ID NO: 853), NB7 (SEQ ID NO: 854), NB9 (SEQ ID NO: 855), NB10 (SEQ ID NO: 856), B11 (SEQ ID NO: 857), B17 (SEQ ID NO: 858) (FIG. 2). These include 40 AA centered around each core domain, also all the possible 20 AA that can be derived around the core domain, and all the possible 10 AA that can be derived around the core domain. Also listed are multiple mutations on the 40AA, 20 AA, and 10 AA (X, X0 equal all possible AA).

(G/A)₀₋₂₀=0 to 20 amino acids of either G or A in each position.
x0=any one of the natural and un-natural amino acids.
X=any one of the natural and un-natural amino acids.
   The amino acid in any of the position in all of the sequences can be replaced by an amino acid that belongs to the same amino acid group as listed in Table 1. (Table 1, Group 1=R, H, K; Group 2=D, E; Group 3=S, T, N, Q; Group 4=A, I, L, M, F, W, Y, V; Group 5=C, U, G, P)

TABLE 1
Twenty-One Amino Acids

| | | |
|---|---|---|
| Group 1 | Amino acids with positively charged | R = Arginine (Arg) |
| | side chain | H = Histidine (His) |
| | | K = Lysine (Lys) |
| Group 2 | Amino acids with negatively charged | D = Aspartic acid (Asp) |
| | side chain | E = Glutamic acid (Glu) |
| Group 3 | Amino acids with polar | S = Serine (Ser) |
| | uncharged side chain | T = Threonine (Thr) |
| | | N = Asparagine (Asn) |
| | | Q = Glutamine (Gln) |
| Group 4 | Amino acids with hydrophobic | A = Alanine (Ala) |
| | side chain | I = Isoleucine (Ile) |
| | | L = Leucine (Leu) |
| | | M = Methionine (Met) |
| | | F = Phenylalanine (Phe) |
| | | W = Tryptophan (Trp) |
| | | Y = Tyrosine (Tyr) |
| | | V = Valine (Val) |
| Group 5 | Amino acids with special cases | C = Cysteine (Cys) |
| | | U = Selenocysteine (Sec) |
| | | G = Glycine (Gly) |
| | | P = Proline (Pro) |

A Peptide Sequence of the Invention includes One or More of the Following:

| ID (Name) | Peptide Sequence (N to C) |
|---|---|
| SEQ ID NO: 1(NB5-core): | RRRRRRRR(G/A)₀₋₂₀YNELRVAPEE |
| SEQ ID NO: 2(NB7-core): | RRRRRRRR(G/A)₀₋₂₀QIMFETFNTP |
| SEQ ID NO: 3(NB9-core): | RRRRRRRR(G/A)₀₋₂₀LPHAILRLDL |
| SEQ ID NO: 4(NB10-core): | RRRRRRRR(G/A)₀₋₂₀AGRDLTDYLM |
| SEQ ID NO: 5(B11-core): | RRRRRRRR(G/A)₀₋₂₀LCYVALDFEQ |
| SEQ ID NO: 6(B17-core): | RRRRRRRR(G/A)₀₋₂₀KYSVWIGGSI |
| SEQ ID NO: 7(B5-20AA): | RRRRRRRR(G/A)₀₋₂₀YNELRVAPEEHPVLLTEAPL |
| SEQ ID NO: 8(B6-20AA): | RRRRRRRR(G/A)₀₋₂₀NPKANREKMTQIMFETFNTP |
| SEQ ID NO: 9(N7-20AA): | RRRRRRRR(G/A)₀₋₂₀QIMFETFNTPAMYVAIQAVL |
| SEQ ID NO: 10(N9-20AA): | RRRRRRRR(G/A)₀₋₂₀HTVRIYEGYALRHAILRLDL |
| SEQ ID NO: 11(B9-20AA): | RRRRRRRR(G/A)₀₋₂₀LPHAILRLDLAGRDLTDYLM |
| SEQ ID NO: 12(N10-20AA): | RRRRRRRR(G/A)₀₋₂₀AGRDLTDYLMKILTERGYSF |
| SEQ ID NO: 13(B11-20AA): | RRRRRRRR(G/A)₀₋₂₀DIKEKLCYVALDFEQEMATA |
| SEQ ID NO: 14(B17-20AA): | RRRRRRRR(G/A)₀₋₂₀APPERKYSVWIGGSILASLS |
| SEQ ID NO: 15(NB5-V1): | (X)₀₋₂₀(X)NELRVAPEE(X)₀₋₂₀ |
| SEQ ID NO: 16(NB5-V2): | (G/A)₀₋₂₀Y(X)ELRVAPEE(G/A)₀₋₂₀ |
| SEQ ID NO: 17(NB5-V3): | (G/A)₀₋₂₀YN(X)LRVAPEE(G/A)₀₋₂₀ |
| SEQ ID NO: 18(NB5-V4): | (G/A)₀₋₂₀YNE(X)RVAREE(G/A)₀₋₂₀ |
| SEQ ID NO: 19(NB5-V5): | (G/A)₀₋₂₀YNEL(X)VAPEE(G/A)₀₋₂₀ |
| SEQ ID NO: 20(NB5-V6): | (G/A)₀₋₂₀YNELR(X)APEE(G/A)₀₋₂₀ |
| SEQ ID NO: 21(NB5-V7): | (G/A)₀₋₂₀YNELRV(X)PEE(G/A)₀₋₂₀ |
| SEQ ID NO: 22(NB5-V8): | (G/A)₀₋₂₀YNELRVA(X)EE(G/A)₀₋₂₀ |
| SEQ ID NO: 23(NB5-V9): | (G/A)₀₋₂₀YNELRVAP(X)E(G/A)₀₋₂₀ |
| SEQ ID NO: 24(NB5-V10): | (G/A)₀₋₂₀YNELRVAPE(X)(G/A)₀₋₂₀ |
| SEQ ID NO: 25(B5-NV2): | (X)₀₋₂₀WHHTFYNELR(X)₀₋₂₀ |
| SEQ ID NO: 26(B5-NV2): | (G/A)₀₋₂₀W(X)HTFYNELR(G/A)₀₋₂₀ |
| SEQ ID NO: 27(B5-NV3): | (G/A)₀₋₂₀WH(X)TFYNELR(G/A)₀₋₂₀ |
| SEQ ID NO: 28(B5-NV4): | (G/A)₀₋₂₀WHH(X)FYNELR(G/A)₀₋₂₀ |
| SEQ ID NO: 29(B5-NV5): | (G/A)₀₋₂₀WHHT(X)YNELR(G/A)₀₋₂₀ |
| SEQ ID NO: 30(B5-NV6): | (G/A)₀₋₂₀WHHTF(X)NELR(G/A)₀₋₂₀ |
| SEQ ID NO: 31(B5-NV7): | (G/A)₀₋₂₀WHHTFY(X)ELR(G/A)₀₋₂₀ |
| SEQ ID NO: 32(B5-NV8): | (G/A)₀₋₂₀WHHTFYN(X)LR(G/A)₀₋₂₀ |
| SEQ ID NO: 33(B5-NV9): | (G/A)₀₋₂₀WHHTFYNE(X)R(G/A)₀₋₂₀ |
| SEQ ID NO: 34(B5-NV10): | (G/A)₀₋₂₀WHHTFYNEL(X)(G/A)₀₋₂₀ |
| SEQ ID NO: 35(B5-N1): | (G/A)₀₋₂₀VTNWDDMEKI(G/A)₀₋₂₀ |
| SEQ ID NO: 36(B5-N2): | (G/A)₀₋₂₀TNWDDMEKIW(G/A)₀₋₂₀ |
| SEQ ID NO: 37(B5-N3): | (G/A)₀₋₂₀NWDDMEKIWH(G/A)₀₋₂₀ |
| SEQ ID NO: 38(B5-N4): | (G/A)₀₋₂₀WDDMEKIWHH(G/A)₀₋₂₀ |
| SEQ ID NO: 39(B5-N5): | (G/A)₀₋₂₀DDMEKIWHHT(G/A)₀₋₂₀ |
| SEQ ID NO: 40(B5-N6): | (G/A)₀₋₂₀DMEKIWHHTF(G/A)₀₋₂₀ |
| SEQ ID NO: 41(B5-N7): | (G/A)₀₋₂₀MEKIWHHTFY(G/A)₀₋₂₀ |
| SEQ ID NO: 42(B5-N8): | (G/A)₀₋₂₀EKIWHHTFYN(G/A)₀₋₂₀ |
| SEQ ID NO: 43(B5-N9): | (G/A)₀₋₂₀KIWHHTFYNE(G/A)₀₋₂₀ |
| SEQ ID NO: 44(B5-N10): | (G/A)₀₋₂₀IWHHTFYNEL(G/A)₀₋₂₀ |
| SEQ ID NO: 45(B5-N11): | (G/A)₀₋₂₀WHHTFYNELR(G/A)₀₋₂₀ |
| SEQ ID NO: 46(B5-N12): | (G/A)₀₋₂₀HHTFYNELRV(G/A)₀₋₂₀ |
| SEQ ID NO: 47(B5-N13): | (G/A)₀₋₂₀HTFYNELRVA(G/A)₀₋₂₀ |
| SEQ ID NO: 48(B5-N14): | (G/A)₀₋₂₀TFYNELRVAP(G/A)₀₋₂₀ |
| SEQ ID NO: 49(B5-N15): | (G/A)₀₋₂₀FYNELRVAPE(G/A)₀₋₂₀ |
| SEQ ID NO: 50(B5-N16): | (G/A)₀₋₂₀YNELRVAPEE(G/A)₀₋₂₀ |
| SEQ ID NO: 51(B5-N17): | (G/A)₀₋₂₀NELRVAPEEH(G/A)₀₋₂₀ |
| SEQ ID NO: 52(B5-N18): | (G/A)₀₋₂₀ELRVAPEEHP(G/A)₀₋₂₀ |
| SEQ ID NO: 53(B5-N19): | (G/A)₀₋₂₀LRVAPEEHPV(G/A)₀₋₂₀ |
| SEQ ID NO: 54(B5-N20): | (G/A)₀₋₂₀RVAPEEHPVL(G/A)₀₋₂₀ |
| SEQ ID NO: 55(B5-N21): | (G/A)₀₋₂₀VAPEEHPVLL(G/A)₀₋₂₀ |
| SEQ ID NO: 56(B5-N22): | (G/A)₀₋₂₀APEEHPVLLT(G/A)₀₋₂₀ |
| SEQ ID NO: 57(B5-N23): | (G/A)₀₋₂₀PEEHPVLLTE(G/A)₀₋₂₀ |
| SEQ ID NO: 58(B5-N24): | (G/A)₀₋₂₀EEHPVLLTEA(G/A)₀₋₂₀ |
| SEQ ID NO: 59(B5-N25): | (G/A)₀₋₂₀EHPVLLTEAP(G/A)₀₋₂₀ |
| SEQ ID NO: 60(B5-N26): | (G/A)₀₋₂₀HPVLLTEAPL(G/A)₀₋₂₀ |
| SEQ ID NO: 61(B5-N27): | (G/A)₀₋₂₀PVLLTEAPLN(G/A)₀₋₂₀ |
| SEQ ID NO: 62(B5-N28): | (G/A)₀₋₂₀VILTEAPLNP(G/A)₀₋₂₀ |
| SEQ ID NO: 63(B5-N29): | (G/A)₀₋₂₀LLTEAPLNPK(G/A)₀₋₂₀ |
| SEQ ID NO: 64(B5-N30): | (G/A)₀₋₂₀LTEAPLNPKA(G/A)₀₋₂₀ |
| SEQ ID NO: 65(B5-N31): | (G/A)₀₋₂₀TEAPLNPKAN(G/A)₀₋₂₀ |
| SEQ ID NO: 66(B5-M2): | (G/A)₀₋₂₀EHGIVTNWDDMEKIWHHTFY(G/A)₀₋₂₀ |
| SEQ ID NO: 67(B5-M3): | (G/A)₀₋₂₀HGIVTNWDDMEKIWHHTFYN(G/A)₀₋₂₀ |
| SEQ ID NO: 68(B5-M4): | (G/A)₀₋₂₀GIVTNWDDMEKIWHHTFYNE(G/A)₀₋₂₀ |
| SEQ ID NO: 69(B5-M5): | (G/A)₀₋₂₀IVTNWDDMEKIWHHTFYNEL(G/A)₀₋₂₀ |
| SEQ ID NO: 70(B5-M6): | (G/A)₀₋₂₀VTNWDDMEKIWHHTFYNELR(G/A)₀₋₂₀ |
| SEQ ID NO: 71(B5-M7): | (G/A)₀₋₂₀TNWDDMEKIWHHTFYNELRV(G/A)₀₋₂₀ |
| SEQ ID NO: 72(B5-M8): | (G/A)₀₋₂₀NWDDMEKIWHHTFYNELRVA(G/A)₀₋₂₀ |
| SEQ ID NO: 73(B5-M9): | (G/A)₀₋₂₀WDDMEKIWHHTFYNELRVAP(G/A)₀₋₂₀ |
| SEQ ID NO: 74(B5-M10): | (G/A)₀₋₂₀DDMEKIWHHTFYNELRVAPE(G/A)₀₋₂₀ |
| SEQ ID NO: 75(B5 ): | (G/A)₀₋₂₀DMEKIWHHTFYNELRVAPEE(G/A)₀₋₂₀ |
| SEQ ID NO: 76(B5-P1): | (G/A)₀₋₂₀MEKIWHHTFYNELRVAPEEH(G/A)₀₋₂₀ |
| SEQ ID NO: 77(B5-P2): | (G/A)₀₋₂₀EKIWHHTFYNELRVAPEEHP(G/A)₀₋₂₀ |
| SEQ ID NO: 78(B5-P3): | (G/A)₀₋₂₀KIWHHTFYNELRVAPEEHPV(G/A)₀₋₂₀ |
| SEQ ID NO: 79(B5-P4): | (G/A)₀₋₂₀IWHHTFYNELRVAPEEHPVL(G/A)₀₋₂₀ |
| SEQ ID NO: 80(B5-P5): | (G/A)₀₋₂₀WHHTFYNELRVAPEEHPVLL(G/A)₀₋₂₀ |
| SEQ ID NO: 81(B5-P6): | (G/A)₀₋₂₀HHTFYNELRVAPEEHPVLLT(G/A)₀₋₂₀ |
| SEQ ID NO: 82(B5-P7): | (G/A)₀₋₂₀HTFYNELRVAPEEHPVLLTE(G/A)₀₋₂₀ |
| SEQ ID NO: 83(B5-P8): | (G/A)₀₋₂₀TFYNELRVAPEEHPVLLTEA(G/A)₀₋₂₀ |
| SEQ ID NO: 84(B5-P9): | (G/A)₀₋₂₀FYNELRVAPEEHPVLLTEAP(G/A)₀₋₂₀ |
| SEQ ID NO: 85(B5-P10): | (G/A)₀₋₂₀YNELRVAPEEHPVLLTEAPL(G/A)₀₋₂₀ |
| SEQ ID NO: 86(B5-V1): | (G/A)₀₋₂₀(X)MEKIWHHTFYNELRVAPEE(G/A)₀₋₂₀ |
| SEQ ID NO: 87(B5-V2): | (G/A)₀₋₂₀D(X)EKIWHHTFYNELRVAPEE(G/A)₀₋₂₀ |
| SEQ ID NO: 88(B5-V3): | (G/A)₀₋₂₀DM(X)KIWHHTFYNELRVAPEE(G/A)₀₋₂₀ |
| SEQ ID NO: 89(B5-V4): | (G/A)₀₋₂₀DME(X)IWHHTFYNELRVAPEE(G/A)₀₋₂₀ |
| SEQ ID NO: 90(B5-V5): | (G/A)₀₋₂₀DMEK(X)WHHTFYNELRVAPEE(G/A)₀₋₂₀ |
| SEQ ID NO: 91(B5-V6): | (G/A)₀₋₂₀DMEKI(X)HHTFYNELRVAPEE(G/A)₀₋₂₀ |
| SEQ ID NO: 92(B5-V7): | (G/A)₀₋₂₀DMEKIW(X)HTFYNELRVAPEE(G/A)₀₋₂₀ |
| SEQ ID NO: 93(B5-V8): | (G/A)₀₋₂₀DMEKIWH(X)TFYNELRVAPEE(G/A)₀₋₂₀ |
| SEQ ID NO: 94(B5-V9): | (G/A)₀₋₂₀DMEKIWHH(X)FYNELRVAPEE(G/A)₀₋₂₀ |
| SEQ ID NO: 95(B5-V10): | (G/A)₀₋₂₀DMEKIWHHT(X)YNELRVAPEE(G/A)₀₋₂₀ |
| SEQ ID NO: 96(B5-V11): | (G/A)₀₋₂₀DMEKIWHHTF(X)NELRVAPEE(G/A)₀₋₂₀ |
| SEQ ID NO: 97(B5-V12): | (G/A)₀₋₂₀DMEKIWHHTFY(X)ELRVAPEE(G/A)₀₋₂₀ |
| SEQ ID NO: 98(B5-V13): | (G/A)₀₋₂₀DMEKIWHHTFYN(X)LRVAPEE(G/A)₀₋₂₀ |
| SEQ ID NO: 99(B5-X1): | (G/A)₀₋₂₀(X)EHGIVTNWDDMEKIWHHTFYNELRVAPEEHPVLLTEAPL(G/A)₀₋₂₀ |
| SEQ ID NO: 100(B5-X2): | (G/A)₀₋₂₀I(X)HGIVTNWDDMEKIWHHTFYNELRVAPEEHPVLLTEAPL(G/A)₀₋₂₀ |
| SEQ ID NO: 101(B5-X3): | (G/A)₀₋₂₀IE(X)GIVTNWDDMEKIWHHTFYNELRVAPEEHPVLLTEAPL(G/A)₀₋₂₀ |
| SEQ ID NO: 102(B5-X4): | (G/A)₀₋₂₀IEH(X)IVTNWDDMEKIWHHTFYNELRVAPEEHPVLLTEAPL(G/A)₀₋₂₀ |
| SEQ ID NO: 103(B5-X5): | (G/A)₀₋₂₀IEHG(X)VTNWDDMEKIWHHTFYNELRVAPEEHPVLLTEAPL(G/A)₀₋₂₀ |
| SEQ ID NO: 104(B5-X6): | (G/A)₀₋₂₀IEHGI(X)TNWDDMEKIWHHTFYNELRVAPEEHPVLLTEAPL(G/A)₀₋₂₀ |
| SEQ ID NO: 105(B5-X7): | (G/A)₀₋₂₀IEHGIV(X)NWDDMEKIWHHTFYNELRVAPEEHPVLLTEAPL(G/A)₀₋₂₀ |
| SEQ ID NO: 106(B5-X8): | (G/A)₀₋₂₀IEHGIVT(X)WDDMEKIWHHTFYNELRVAPEEHPVLLTEAPL(G/A)₀₋₂₀ |
| SEQ ID NO: 107(B5-X9): | (G/A)₀₋₂₀IEHGIVTN(X)DDMEKIWHHTFYNELRVAPEEHPVLLTEAPL(G/A)₀₋₂₀ |
| SEQ ID NO: 108(B5-X10): | (G/A)₀₋₂₀IEHGIVTNW(X)DMEKIWHHTFYNELRVAPEEHPVLLTEAPL(G/A)₀₋₂₀ |
| SEQ ID NO: 109(B5-X11): | (G/A)₀₋₂₀IEHGIVTNWD(X)MEKIWHHTFYNELRVAPEEHPVLLTEAPL(G/A)₀₋₂₀ |
| SEQ ID NO: 110(B5-X12): | (G/A)₀₋₂₀IEHGIVTNWDD(X)EKIWHHTFYNELRVAPEEHPVLLTEAPL(G/A)₀₋₂₀ |
| SEQ ID NO: 111(B5-X13): | (G/A)₀₋₂₀IEHGIVTNWDDM(X)KIWHHTFYNELRVAPEEHPVLLTEAPL(G/A)₀₋₂₀ |
| SEQ ID NO: 112(B5-X14): | (G/A)₀₋₂₀IEHGIVTNWDDME(X)IWHHTFYNELRVAPEEHPVLLTEAPL(G/A)₀₋₂₀ |
| SEQ ID NO: 113(B5-X15): | (G/A)₀₋₂₀IEHGIVTNWDDMEK(X)WHHTFYNELRVAPEEHPVLLTEAPL(G/A)₀₋₂₀ |
| SEQ ID NO: 114(B5-X16): | (G/A)₀₋₂₀IEHGIVTNWDDMEKI(X)HHTFYNELRVAPEEHPVLLTEAPL(G/A)₀₋₂₀ |
| SEQ ID NO: 115(B5-X17): | (G/A)₀₋₂₀IEHGIVTNWDDMEKIW(X)HTFYNELRVAPEEHPVLLTEAPL(G/A)₀₋₂₀ |
| SEQ ID NO: 116(B5-X18): | (G/A)₀₋₂₀IEHGIVTNWDDMEKIWH(X)TFYNELRVAPEEHPVLLTEAPL(G/A)₀₋₂₀ |
| SEQ ID NO: 117(B5-X19): | (G/A)₀₋₂₀IEHGIVTNWDDMEKIWHH(X)FYNELRVAPEEHPVLLTEAPL(G/A)₀₋₂₀ |
| SEQ ID NO: 118(B5-X20): | (G/A)₀₋₂₀IEHGIVTNWDDMEKIWHHT(X)YNELRVAPEEHPVLLTEAPL(G/A)₀₋₂₀ |
| SEQ ID NO: 119(B5-X21): | (G/A)₀₋₂₀IEHGIVTNWDDMEKIWHHTF(X)NELRVAPEEHPVLLTEAPL(G/A)₀₋₂₀ |
| SEQ ID NO: 120(B5-X22): | (G/A)₀₋₂₀IEHGIVTNWDDMEKIWHHTFY(X)ELRVAPEEHPVLLTEAPL(G/A)₀₋₂₀ |
| SEQ ID NO: 121(B5-X23): | (G/A)₀₋₂₀IEHGIVTNWDDMEKIWHHTFYN(X)LRVAPEEHPVLLTEAPL(G/A)₀₋₂₀ |
| SEQ ID NO: 122(B5-X24): | (G/A)₀₋₂₀IEHGIVTNWDDMEKIWHHTFYNE(X)RVAPEEHPVLLTEAPL(G/A)₀₋₂₀ |
| SEQ ID NO: 123(B5-X25): | (G/A)₀₋₂₀IEHGIVTNWDDMEKIWHHTFYNEL(X)VAPEEHPVLLTEAPL(G/A)₀₋₂₀ |
| SEQ ID NO: 124(B5-X26): | (G/A)₀₋₂₀IEHGIVTNWDDMEKIWHHTFYNELR(X)APEEHPVLLTEAPL(G/A)₀₋₂₀ |
| SEQ ID NO: 125(B5-X27): | (G/A)₀₋₂₀IEHGIVTNWDDMEKIWHHTFYNELRV(X)PEEHPVLLTEAPL(G/A)₀₋₂₀ |
| SEQ ID NO: 126(B5-X28): | (G/A)₀₋₂₀IEHGIVTNWDDMEKIWHHTFYNELRVA(X)EEHPVLLTEAPL(G/A)₀₋₂₀ |
| SEQ ID NO: 127(B5-X29): | (G/A)₀₋₂₀IEHGIVTNWDDMEKIWHHTFYNELRVAP(X)EHPVLLTEAPL(G/A)₀₋₂₀ |
| SEQ ID NO: 128(B5-X30): | (G/A)₀₋₂₀IEHGIVTNWDDMEKIWHHTFYNELRVAPE(X)HPVLLTEAPL(G/A)₀₋₂₀ |
| SEQ ID NO: 129(B5-X31): | (G/A)₀₋₂₀IEHGIVTNWDDMEKIWHHTFYNELRVAPEE(X)PVLLTEAPL(G/A)₀₋₂₀ |
| SEQ ID NO: 130(B5-X32): | (G/A)₀₋₂₀IEHGIVTNWDDMEKIWHHTFYNELRVAPEEH(X)VLLTEAPL(G/A)₀₋₂₀ |
| SEQ ID NO: 131(B5-X33): | (G/A)₀₋₂₀IEHGIVTNWDDMEKIWHHTFYNELRVAPEEHP(X)LLTEAPL(G/A)₀₋₂₀ |
| SEQ ID NO: 132(B5-X34): | (G/A)₀₋₂₀IEHGIVTNWDDMEKIWHHTFYNELRVAPEEHPV(X)LTEAPL(G/A)₀₋₂₀ |
| SEQ ID NO: 133(B5-X35): | (G/A)₀₋₂₀IEHGIVTNWDDMEKIWHHTFYNELRVAPEEHPVL(X)TEAPL(G/A)₀₋₂₀ |
| SEQ ID NO: 134(B5-X36): | (G/A)₀₋₂₀IEHGIVTNWDDMEKIWHHTFYNELRVAPEEHPVLL(X)EAPL(G/A)₀₋₂₀ |
| SEQ ID NO: 135(B5-X37): | (G/A)₀₋₂₀IEHGIVTNWDDMEKIWHHTFYNELRVAPEEHPVLLT(X)APL(G/A)₀₋₂₀ |
| SEQ ID NO: 136(B5-X38): | (G/A)₀₋₂₀IEHGIVTNWDDMEKIWHHTFYNELRVAPEEHPVLLTE(X)PL(G/A)₀₋₂₀ |
| SEQ ID NO: 137(B5-X39): | (G/A)₀₋₂₀IEHGIVTNWDDMEKIWHHTFYNELRVAPEEHPVLLTEA(X)L(G/A)₀₋₂₀ |
| SEQ ID NO: 138(B5-X40): | (G/A)₀₋₂₀IEHGIVTNWDDMEKIWHHTFYNELRVAPEEHPVLLTEAP(X)(G/A)₀₋₂₀ |
| SEQ ID NO: 139(NB7-V1): | (X)₀₋₂₀(X)IMFETFNTP(X)₀₋₂₀ |
| SEQ ID NO: 140(NB7-V2): | (G/A)₀₋₂₀Q(X)MFETFNTP(G/A)₀₋₂₀ |
| SEQ ID NO: 141(NB7-V3): | (G/A)₀₋₂₀QI(X)FETFNTP(G/A)₀₋₂₀ |
| SEQ ID NO: 142(NB7-V4): | (G/A)₀₋₂₀QIM(X)ETFNTP(G/A)₀₋₂₀ |
| SEQ ID NO: 143(NB7-V5): | (G/A)₀₋₂₀QIMF(X)TFNTP(G/A)₀₋₂₀ |
| SEQ ID NO: 144(NB7-V6): | (G/A)₀₋₂₀QIMFE(X)FNTP(G/A)₀₋₂₀ |
| SEQ ID NO: 145(NB7-V7): | (G/A)₀₋₂₀QIMFET(X)NTP(G/A)₀₋₂₀ |
| SEQ ID NO: 146(NB7-V8): | (G/A)₀₋₂₀QIMFETF(X)TP(G/A)₀₋₂₀ |
| SEQ ID NO: 147(NB7-V9): | (G/A)₀₋₂₀QIMFETFN(X)P(G/A)₀₋₂₀ |
| SEQ ID NO: 148(NB7-V10): | (G/A)₀₋₂₀QIMFETFNT(X)(G/A)₀₋₂₀ |
| SEQ ID NO: 149(N7-NX1): | (X)₀₋₂₀TFN(X0)PAMYVA(X)₀₋₂₀ |
| SEQ ID NO: 150(N7-NX1): | (G/A)₀₋₂₀(X)FN(X0)PAMYVA(G/A)₀₋₂₀ |
| SEQ ID NO: 151(N7-NX2): | (G/A)₀₋₂₀T(X)N(X0)PAMYVA(G/A)₀₋₂₀ |
| SEQ ID NO: 152(N7-NX3): | (G/A)₀₋₂₀TF(X)(X0)PAMYVA(G/A)₀₋₂₀ |
| SEQ ID NO: 153(N7-NX4): | (G/A)₀₋₂₀TFN(X0)(X)AMYVA(G/A)₀₋₂₀ |
| SEQ ID NO: 154(N7-NX5): | (G/A)₀₋₂₀TFN(X0)P(X)MYVA(G/A)₀₋₂₀ |
| SEQ ID NO: 155(N7-NX6): | (G/A)₀₋₂₀TFN(X0)PA(X)YVA(G/A)₀₋₂₀ |
| SEQ ID NO: 156(N7-NX7): | (G/A)₀₋₂₀TFN(X0)PAM(X)VA(G/A)₀₋₂₀ |
| SEQ ID NO: 157(N7-NX8): | (G/A)₀₋₂₀TFN(X0)PAMY(X)A(G/A)₀₋₂₀ |
| SEQ ID NO: 158(N7-NX9): | (G/A)₀₋₂₀TFN(X0)PAMYV(X)(G/A)₀₋₂₀ |
| SEQ ID NO: 159(N7-T1): | (G/A)₀₋₂₀TEAPLNPKAN(G/A)₀₋₂₀ |
| SEQ ID NO: 160(N7-T2): | (G/A)₀₋₂₀EAPLNPKANR(G/A)₀₋₂₀ |
| SEQ ID NO: 161(N7-T3): | (G/A)₀₋₂₀APLNPKANRE(G/A)₀₋₂₀ |
| SEQ ID NO: 162(N7-T4): | (G/A)₀₋₂₀PLNPKANREK(G/A)₀₋₂₀ |
| SEQ ID NO: 163(N7-T5): | (G/A)₀₋₂₀LNPKANREKM(G/A)₀₋₂₀ |
| SEQ ID NO: 164(N7-T6): | (G/A)₀₋₂₀NPKANREKMT(G/A)₀₋₂₀ |
| SEQ ID NO: 165(N7-T7): | (G/A)₀₋₂₀PKANREKMTQ(G/A)₀₋₂₀ |
| SEQ ID NO: 166(N7-T8): | (G/A)₀₋₂₀KANREKMTQI(G/A)₀₋₂₀ |
| SEQ ID NO: 167(N7-T9): | (G/A)₀₋₂₀ANREKMTQIM(G/A)₀₋₂₀ |
| SEQ ID NO: 168(N7-T10): | (G/A)₀₋₂₀NREKMTQIMF(G/A)₀₋₂₀ |
| SEQ ID NO: 169(N7-T11): | (G/A)₀₋₂₀REKMTQIMFE(G/A)₀₋₂₀ |
| SEQ ID NO: 170(N7-T12): | (G/A)₀₋₂₀EKMTQIMFET(G/A)₀₋₂₀ |
| SEQ ID NO: 171(N7-T13): | (G/A)₀₋₂₀KMTQIMFETF(G/A)₀₋₂₀ |
| SEQ ID NO: 172(N7-T14): | (G/A)₀₋₂₀MTQIMFETFN(G/A)₀₋₂₀ |
| SEQ ID NO: 173(N7-T15): | (G/A)₀₋₂₀TQIMFETFN(X0)(G/A)₀₋₂₀ |
| SEQ ID NO: 174(N7-T16): | (G/A)₀₋₂₀QIMFETFN(X0)P(G/A)₀₋₂₀ |
| SEQ ID NO: 175(N7-T17): | (G/A)₀₋₂₀IMFETFN(X0)PA(G/A)₀₋₂₀ |
| SEQ ID NO: 176(N7-T18): | (G/A)₀₋₂₀MFETFN(X0)PAM(G/A)₀₋₂₀ |
| SEQ ID NO: 177(N7-T19): | (G/A)₀₋₂₀FETFN(X0)PAMY(G/A)₀₋₂₀ |
| SEQ ID NO: 178(N7-T20): | (G/A)₀₋₂₀ETFN(X0)PAMYV(G/A)₀₋₂₀ |
| SEQ ID NO: 179(N7-T21): | (G/A)₀₋₂₀TFN(X0)PAMYVA(G/A)₀₋₂₀ |
| SEQ ID NO: 180(N7-T22): | (G/A)₀₋₂₀FN(X0)PAMYVAI(G/A)₀₋₂₀ |
| SEQ ID NO: 181(N7-T23): | (G/A)₀₋₂₀N(X0)PAMYVAIQ(G/A)₀₋₂₀ |
| SEQ ID NO: 182(N7-T24): | (G/A)₀₋₂₀(X0)PAMYVAIQA(G/A)₀₋₂₀ |
| SEQ ID NO: 183(N7-T25): | (G/A)₀₋₂₀PAMYVAIQAV(G/A)₀₋₂₀ |
| SEQ ID NO: 184(N7-T26): | (G/A)₀₋₂₀AMYVAIQAVL(G/A)₀₋₂₀ |
| SEQ ID NO: 185(N7-T27): | (G/A)₀₋₂₀MYVAIQAVLS(G/A)₀₋₂₀ |
| SEQ ID NO: 186(N7-T28): | (G/A)₀₋₂₀YVAIQAVLSL(G/A)₀₋₂₀ |
| SEQ ID NO: 187(N7-T29): | (G/A)₀₋₂₀VAIQAVLSLY(G/A)₀₋₂₀ |
| SEQ ID NO: 188(N7-T30): | (G/A)₀₋₂₀AIQAVLSLYA(G/A)₀₋₂₀ |
| SEQ ID NO: 189(N7-T31): | (G/A)₀₋₂₀IQAVLSLYAS(G/A)₀₋₂₀ |
| SEQ ID NO: 190(N7-T32): | (G/A)₀₋₂₀QAVLSLYASG(G/A)₀₋₂₀ |
| SEQ ID NO: 191(N7-M1): | (G/A)₀₋₂₀TEAPLNPKANREKMTQIMFE(G/A)₀₋₂₀ |
| SEQ ID NO: 192(N7-M2): | (G/A)₀₋₂₀EAPLNPKANREKMTQIMFET(G/A)₀₋₂₀ |
| SEQ ID NO: 193(N7-M3): | (G/A)₀₋₂₀APLNPKANREKMTQIMFETF(G/A)₀₋₂₀ |
| SEQ ID NO: 194(N7-M4): | (G/A)₀₋₂₀PLNPKANREKMTQIMFETFN(G/A)₀₋₂₀ |
| SEQ ID NO: 195(N7-M5): | (G/A)₀₋₂₀LNPKANREKMTQIMFETFN(X0)(G/A)₀₋₂₀ |
| SEQ ID NO: 196(N7-M6): | (G/A)₀₋₂₀NPKANREKMTQIMFETFN(X0)P(G/A)₀₋₂₀ |
| SEQ ID NO: 197(N7-M7): | (G/A)₀₋₂₀PKANREKMTQIMFETFN(X0)PA(G/A)₀₋₂₀ |
| SEQ ID NO: 198(N7-M8): | (G/A)₀₋₂₀KANREKMTQIMFETFN(X0)PAM(G/A)₀₋₂₀ |
| SEQ ID NO: 199(N7-M9): | (G/A)₀₋₂₀ANREKMTQIMFETFN(X0)PAMY(G/A)₀₋₂₀ |
| SEQ ID NO: 200(N7-M10): | (G/A)₀₋₂₀NREKMTQIMFETFN(X0)PAMYV(G/A)₀₋₂₀ |
| SEQ ID NO: 201(N7-M11): | (G/A)₀₋₂₀REKMTQIMFETFN(X0)PAMYVA(G/A)₀₋₂₀ |
| SEQ ID NO: 202(N7-M12): | (G/A)₀₋₂₀EKMTQIMFETFN(X0)PAMYVAI(G/A)₀₋₂₀ |
| SEQ ID NO: 203(N7-M13): | (G/A)₀₋₂₀KMTQIMFETFN(X0)PAMYVAIQ(G/A)₀₋₂₀ |
| SEQ ID NO: 204(N7-M14): | (G/A)₀₋₂₀MTQIMFETFN(X0)PAMYVAIQA(G/A)₀₋₂₀ |
| SEQ ID NO: 205(N7-M15): | (G/A)₀₋₂₀TQIMFETFN(X0)PAMYVAIQAV(G/A)₀₋₂₀ |
| SEQ ID NO: 206(N7 ): | (G/A)₀₋₂₀QIMFETFN(X0)PAMYVAIQAVL(G/A)₀₋₂₀ |
| SEQ ID NO: 207(N7-P1): | (G/A)₀₋₂₀IMFETFN(X0)PAMYVAIQAVLS(G/A)₀₋₂₀ |
| SEQ ID NO: 208(N7-P2): | (G/A)₀₋₂₀MFETFN(X0)PAMYVAIQAVLSL(G/A)₀₋₂₀ |
| SEQ ID NO: 209(N7-P3): | (G/A)₀₋₂₀FETFN(X0)PAMYVAIQAVLSLY(G/A)₀₋₂₀ |
| SEQ ID NO: 210(N7-P4): | (G/A)₀₋₂₀ETFN(X0)PAMYVAIQAVLSLYA(G/A)₀₋₂₀ |
| SEQ ID NO: 211(N7-P5): | (G/A)₀₋₂₀TFN(X0)PAMYVAIQAVLSLYAS(G/A)₀₋₂₀ |
| SEQ ID NO: 212(N7-V1): | (G/A)₀₋₂₀(X)QIMFETFN(X0)PAMYVAIQAVL(G/A)₀₋₂₀ |
| SEQ ID NO: 213(N7-V2): | (G/A)₀₋₂₀Q(X)IMFETFN(X0)PAMYVAIQAVL(G/A)₀₋₂₀ |
| SEQ ID NO: 214(N7-V3): | (G/A)₀₋₂₀QI(X)MFETFN(X0)PAMYVAIQAVL(G/A)₀₋₂₀ |
| SEQ ID NO: 215(N7-V4): | (G/A)₀₋₂₀QIM(X)FETFN(X0)PAMYVAIQAVL(G/A)₀₋₂₀ |
| SEQ ID NO: 216(N7-V5): | (G/A)₀₋₂₀QIMF(X)ETFN(X0)PAMYVAIQAVL(G/A)₀₋₂₀ |
| SEQ ID NO: 217(N7-V6): | (G/A)₀₋₂₀QIMFE(X)TFN(X0)PAMYVAIQAVL(G/A)₀₋₂₀ |
| SEQ ID NO: 218(N7-V7): | (G/A)₀₋₂₀QIMFET(X)FN(X0)PAMYVAIQAVL(G/A)₀₋₂₀ |
| SEQ ID NO: 219(N7-V8): | (G/A)₀₋₂₀QIMFETF(X)N(X0)PAMYVAIQAVL(G/A)₀₋₂₀ |
| SEQ ID NO: 220(N7-V9): | (G/A)₀₋₂₀QIMFETFN(X)(X0)PAMYVAIQAVL(G/A)₀₋₂₀ |
| SEQ ID NO: 221(N7-V10): | (G/A)₀₋₂₀QIMFETFN(X0)P(X)AMYVAIQAVL(G/A)₀₋₂₀ |
| SEQ ID NO: 222(N7-V11): | (G/A)₀₋₂₀QIMFETFN(X0)PA(X)MYVAIQAVL(G/A)₀₋₂₀ |
| SEQ ID NO: 223(N7-V12): | (G/A)₀₋₂₀QIMFETFN(X0)PAM(X)YVAIQAVL(G/A)₀₋₂₀ |
| SEQ ID NO: 224(N7-V13): | (G/A)₀₋₂₀QIMFETFN(X0)PAMY(X)VAIQAVL(G/A)₀₋₂₀ |
| SEQ ID NO: 225(N7-V14): | (G/A)₀₋₂₀QIMFETFN(X0)PAMYV(X)AIQAVL(G/A)₀₋₂₀ |
| SEQ ID NO: 226(N7-V15): | (G/A)₀₋₂₀QIMFETFN(X0)PAMYVA(X)IQAVL(G/A)₀₋₂₀ |
| SEQ ID NO: 227(N7-V16): | (G/A)₀₋₂₀QIMFETFN(X0)PAMYVAI(X)QAVL(G/A)₀₋₂₀ |
| SEQ ID NO: 228(N7-V17): | (G/A)₀₋₂₀QIMFETFN(X0)PAMYVAIQ(X)AVL(G/A)₀₋₂₀ |
| SEQ ID NO: 229(N7-V18): | (G/A)₀₋₂₀QIMFETFN(X0)PAMYVAIQA(X)VL(G/A)₀₋₂₀ |
| SEQ ID NO: 230(N7-V19): | (G/A)₀₋₂₀QIMFETFN(X0)PAMYVAIQAV(X)L(G/A)₀₋₂₀ |
| SEQ ID NO: 231(N7-V20): | (G/A)₀₋₂₀QIMFETFN(X0)PAMYVAIQAVL(X)(G/A)₀₋₂₀ |
| SEQ ID NO: 232(N7-X1): | (G/A)₀₋₂₀(X)PKANREKMTQIMFETFN(X0)PAMYVAIQAVLSLYASGRTTGI(G/A)₀₋₂₀ |
| SEQ ID NO: 233(N7-X2): | (G/A)₀₋₂₀N(X)KANREKMTQIMFETFN(X0)PAMYVAIQAVLSLYASGRTTGI(G/A)₀₋₂₀ |
| SEQ ID NO: 234(N7-X3): | (G/A)₀₋₂₀NP(X)ANREKMTQIMFETFN(X0)PAMYVAIQAVLSLYASGRTTGI(G/A)₀₋₂₀ |
| SEQ ID NO: 235(N7-X4): | (G/A)₀₋₂₀NPK(X)NREKMTQIMFETFN(X0)PAMYVAIQAVLSLYASGRTTGI(G/A)₀₋₂₀ |
| SEQ ID NO: 236(N7-X5): | (G/A)₀₋₂₀NPKA(X)REKMTQIMFETFN(X0)PAMYVAIQAVLSLYASGRTTGI(G/A)₀₋₂₀ |
| SEQ ID NO: 237(N7-X6): | (G/A)₀₋₂₀NPKAN(X)EKMTQIMFETFN(X0)PAMYVAIQAVLSLYASGRTTGI(G/A)₀₋₂₀ |
| SEQ ID NO: 238(N7-X7): | (G/A)₀₋₂₀NPKANR(X)KMTQIMFETFN(X0)PAMYVAIQAVLSLYASGRTTGI(G/A)₀₋₂₀ |
| SEQ ID NO: 239(N7-X8): | (G/A)₀₋₂₀NPKANRE(X)MTQIMFETFN(X0)PAMYVAIQAVLSLYASGRTTGI(G/A)₀₋₂₀ |
| SEQ ID NO: 240(N7-X9): | (G/A)₀₋₂₀NPKANREK(X)TQIMFETFN(X0)PAMYVAIQAVLSLYASGRTTGI(G/A)₀₋₂₀ |
| SEQ ID NO: 241(N7-X10): | (G/A)₀₋₂₀NPKANREKM(X)QIMFETFN(X0)PAMYVAIQAVLSLYASGRTTGI(G/A)₀₋₂₀ |
| SEQ ID NO: 242(N7-X11): | (G/A)₀₋₂₀NPKANREKMT(X)IMFETFN(X0)PAMYVAIQAVLSLYASGRTTGI(G/A)₀₋₂₀ |
| SEQ ID NO: 243(N7-X12): | (G/A)₀₋₂₀NPKANREKMTQ(X)MFETFN(X0)PAMYVAIQAVLSLYASGRTTGI(G/A)₀₋₂₀ |
| SEQ ID NO: 244(N7-X13): | (G/A)₀₋₂₀NPKANREKMTQI(X)FETFN(X0)PAMYVAIQAVLSLYASGRTTGI(G/A)₀₋₂₀ |
| SEQ ID NO: 245(N7-X14): | (G/A)₀₋₂₀NPKANREKMTQIM(X)ETFN(X0)PAMYVAIQAVLSLYASGRTTGI(G/A)₀₋₂₀ |
| SEQ ID NO: 246(N7-X15): | (G/A)₀₋₂₀NPKANREKMTQIMF(X)TFN(X0)PAMYVAIQAVLSLYASGRTTGI(G/A)₀₋₂₀ |
| SEQ ID NO: 247(N7-X16): | (G/A)₀₋₂₀NPKANREKMTQIMFE(X)FN(X0)PAMYVAIQAVLSLYASGRTTGI(G/A)₀₋₂₀ |
| SEQ ID NO: 248(N7-X17): | (G/A)₀₋₂₀NPKANREKMTQIMFET(X)N(X0)PAMYVAIQAVLSLYASGRTTGI(G/A)₀₋₂₀ |
| SEQ ID NO: 249(N7-X18): | (G/A)₀₋₂₀NPKANREKMTQIMFETF(X)(X0)PAMYVAIQAVLSLYASGRTTGI(G/A)₀₋₂₀ |
| SEQ ID NO: 250(N7-X19): | (G/A)₀₋₂₀NPKANREKMTQIMFETFN(X)(X0)AMYVAIQAVLSLYASGRTTGI(G/A)₀₋₂₀ |
| SEQ ID NO: 251(N7-X20): | (G/A)₀₋₂₀NPKANREKMTQIMFETFN(X)P(X0)MYVAIQAVLSLYASGRTTGI(G/A)₀₋₂₀ |
| SEQ ID NO: 252(N7-X21): | (G/A)₀₋₂₀NPKANREKMTQIMFETFN(X)PA(X0)YVAIQAVLSLYASGRTTGI(G/A)₀₋₂₀ |
| SEQ ID NO: 253(N7-X22): | (G/A)₀₋₂₀NPKANREKMTQIMFETFN(X)PAM(X0)VAIQAVLSLYASGRTTGI(G/A)₀₋₂₀ |
| SEQ ID NO: 254(N7-X23): | (G/A)₀₋₂₀NPKANREKMTQIMFETFN(X)PAMY(X0)AIQAVLSLYASGRTTGI(G/A)₀₋₂₀ |
| SEQ ID NO: 255(N7-X24): | (G/A)₀₋₂₀NPKANREKMTQIMFETFN(X)PAMYV(X0)IQAVLSLYASGRTTGI(G/A)₀₋₂₀ |
| SEQ ID NO: 256(N7-X25): | (G/A)₀₋₂₀NPKANREKMTQIMFETFN(X)PAMYVA(X0)QAVLSLYASGRTTGI(G/A)₀₋₂₀ |
| SEQ ID NO: 257(N7-X26): | (G/A)₀₋₂₀NPKANREKMTQIMFETFN(X)PAMYVAI(X0)AVLSLYASGRTTGI(G/A)₀₋₂₀ |
| SEQ ID NO: 258(N7-X27): | (G/A)₀₋₂₀NPKANREKMTQIMFETFN(X)PAMYVAIQ(X0)VLSLYASGRTTGI(G/A)₀₋₂₀ |
| SEQ ID NO: 259(N7-X28): | (G/A)₀₋₂₀NPKANREKMTQIMFETFN(X)PAVYVAIQA(X0)LSLYASGRTTGI(G/A)₀₋₂₀ |
| SEQ ID NO: 260(N7-X29): | (G/A)₀₋₂₀NPKANREKMTQIMFETFN(X)PAMYVAIQAV(X0)SLYASGRTTGI(G/A)₀₋₂₀ |
| SEQ ID NO: 261(N7-X30): | (G/A)₀₋₂₀NPKANREKMTQIMFETFN(X)PAMYVAIQAVL(X0)LYASGRTTGI(G/A)₀₋₂₀ |
| SEQ ID NO: 262(N7-X31): | (G/A)₀₋₂₀NPKANREKMTQIMFETFN(X)PAMYVAIQAVLS(X0)YASGRTTGI(G/A)₀₋₂₀ |
| SEQ ID NO: 263(N7-X32): | (G/A)₀₋₂₀NPKANREKMTQIMFETFN(X)PAMYVAIQAVLSL(X0)ASGRTTGI(G/A)₀₋₂₀ |
| SEQ ID NO: 264(N7-X33): | (G/A)₀₋₂₀NPKANREKMTQIMFETFN(X)PAMYVAIQAVLSLY(X0)SGRTTGI(G/A)₀₋₂₀ |
| SEQ ID NO: 265(N7-X34): | (G/A)₀₋₂₀NPKANREKMTQIMFETFN(X)PAMYVAIQAVLSLYA(X0)GRTTGI(G/A)₀₋₂₀ |
| SEQ ID NO: 266(N7-X35): | (G/A)₀₋₂₀NPKANREKMTQIMFETFN(X)PAMYVAIQAVLSLYAS(X0)RTTGI(G/A)₀₋₂₀ |
| SEQ ID NO: 267(N7-X36): | (G/A)₀₋₂₀NPKANREKMTQIMFETFN(X)PAMYVAIQAVLSLYASG(X0)TTGI(G/A)₀₋₂₀ |
| SEQ ID NO: 268(N7-X37): | (G/A)₀₋₂₀NPKANREKMTQIMFETFN(X)PAMYVAIQAVLSLYASGR(X0)TGI(G/A)₀₋₂₀ |
| SEQ ID NO: 269(N7-X38): | (G/A)₀₋₂₀NPKANREKMTQIMFETFN(X)PAMYVAIQAVLSLYASGRT(X0)GI(G/A)₀₋₂₀ |
| SEQ ID NO: 270(N7-X39): | (G/A)₀₋₂₀NPKANREKMTQIMFETFN(X)PAMYVAIQAVLSLYASGRTT(X0)I(G/A)₀₋₂₀ |
| SEQ ID NO: 271(N7-X40): | (G/A)₀₋₂₀NPKANREKMTQIMFETFN(X)PAMYVAIQAVLSLYASGRTTG(X0)(G/A)₀₋₂₀ |
| SEQ ID NO: 272(B6-V1): | (G/A)₀₋₂₀(X)PKANREKMTQIMFETFN(X0)P(G/A)₀₋₂₀ |
| SEQ ID NO: 273(B6-V1): | (G/A)₀₋₂₀N(X)KANREKMTQIMFETFN(X0)P(G/A)₀₋₂₀ |
| SEQ ID NO: 274(B6-V1): | (G/A)₀₋₂₀NP(X)ANREKMTQIMFETFN(X0)P(G/A)₀₋₂₀ |
| SEQ ID NO: 275(B6-V1): | (G/A)₀₋₂₀NPK(X)NREKMTQIMFETFN(X0)P(G/A)₀₋₂₀ |
| SEQ ID NO: 276(B6-V1): | (G/A)₀₋₂₀NPKA(X)REKMTQIMFETFN(X0)P(G/A)₀₋₂₀ |
| SEQ ID NO: 277(B6-V1): | (G/A)₀₋₂₀NPKAN(X)EKMTQIMFETFN(X0)P(G/A)₀₋₂₀ |
| SEQ ID NO: 278(B6-V1): | (G/A)₀₋₂₀NPKANR(X)KMTQIMFETFN(X0)P(G/A)₀₋₂₀ |
| SEQ ID NO: 279(B6-V1): | (G/A)₀₋₂₀NPKANRE(X)MTQIMFETFN(X0)P(G/A)₀₋₂₀ |
| SEQ ID NO: 280(B6-V1): | (G/A)₀₋₂₀NPKANREK(X)TQIMFETFN(X0)P(G/A)₀₋₂₀ |
| SEQ ID NO: 281(B6-V1): | (G/A)₀₋₂₀NPKANREKM(X)QIMFETFN(X0)P(G/A)₀₋₂₀ |
| SEQ ID NO: 282(B6-V1): | (G/A)₀₋₂₀NPKANREKMT(X)IMFETFN(X0)P(G/A)₀₋₂₀ |
| SEQ ID NO: 283(B6-V1): | (G/A)₀₋₂₀NPKANREKMTQ(X)MFETFN(X0)P(G/A)₀₋₂₀ |
| SEQ ID NO: 284(B6-V1): | (G/A)₀₋₂₀NPKANREKMTQI(X)FETFN(X0)P(G/A)₀₋₂₀ |
| SEQ ID NO: 285(B6-V1): | (G/A)₀₋₂₀NPKANREKMTQIM(X)ETFN(X0)P(G/A)₀₋₂₀ |
| SEQ ID NO: 286(B6-V1): | (G/A)₀₋₂₀NPKANREKMTQIMF(X)TFN(X0)P(G/A)₀₋₂₀ |
| SEQ ID NO: 287(B6-V1): | (G/A)₀₋₂₀NPKANREKMTQIMFE(X)FN(X0)P(G/A)₀₋₂₀ |
| SEQ ID NO: 288(B6-V1): | (G/A)₀₋₂₀NPKANREKMTQIMFET(X)N(X0)P(G/A)₀₋₂₀ |
| SEQ ID NO: 289(B6-V1): | (G/A)₀₋₂₀NPKANREKMTQIMFETF(X)(X0)P(G/A)₀₋₂₀ |
| SEQ ID NO: 290(B6-V1): | (G/A)₀₋₂₀NPKANREKMTQIMFETFN(X0)(X)(G/A)₀₋₂₀ |
| SEQ ID NO: 291(NB9-core): | (X)₀₋₂₀(X)PHAILRLDL(X)₀₋₂₀ |
| SEQ ID NO: 292(NB9-core): | (G/A)₀₋₂₀L(X)HAILRLDL(G/A)₀₋₂₀ |
| SEQ ID NO: 293(NB9-core): | (G/A)₀₋₂₀LP(X)AILRLDL(G/A)₀₋₂₀ |
| SEQ ID NO: 294(NB9-core): | (G/A)₀₋₂₀LPH(X)ILRLDL(G/A)₀₋₂₀ |
| SEQ ID NO: 295(NB9-core): | (G/A)₀₋₂₀LPHA(X)LRLDL(G/A)₀₋₂₀ |
| SEQ ID NO: 296(NB9-core): | (G/A)₀₋₂₀LPHAI(X)RLDL(G/A)₀₋₂₀ |
| SEQ ID NO: 297(NB9-core): | (G/A)₀₋₂₀LPHAIL(X)LDL(G/A)₀₋₂₀ |
| SEQ ID NO: 298(NB9-core): | (G/A)₀₋₂₀LPHAILRL(X)L(G/A)₀₋₂₀ |
| SEQ ID NO: 299(NB9-core): | (G/A)₀₋₂₀LPHAILRLD(X)(G/A)₀₋₂₀ |
| SEQ ID NO: 300(N9-NV1): | (X)₀₋₂₀(X)YALPHAI(X0)R(X)₀₋₂₀ |
| SEQ ID NO: 301(N9-NV2): | (G/A)₀₋₂₀G(X)ALPHAI(X0)R(G/A)₀₋₂₀ |
| SEQ ID NO: 302(N9-NV3): | (G/A)₀₋₂₀GY(X)LPHAI(X0)R(G/A)₀₋₂₀ |
| SEQ ID NO: 303(N9-NV4): | (G/A)₀₋₂₀GYA(X)PHAI(X0)R(G/A)₀₋₂₀ |
| SEQ ID NO: 304(N9-NV5): | (G/A)₀₋₂₀GYAL(X)HAI(X0)R(G/A)₀₋₂₀ |
| SEQ ID NO: 305(N9-NV6): | (G/A)₀₋₂₀GYALP(X)AI(X0)R(G/A)₀₋₂₀ |
| SEQ ID NO: 306(N9-NV7): | (G/A)₀₋₂₀GYALPH(X)I(X0)R(G/A)₀₋₂₀ |
| SEQ ID NO: 307(N9-NV8): | (G/A)₀₋₂₀GYALPHA(X)(X0)R(G/A)₀₋₂₀ |
| SEQ ID NO: 308(N9-NV9): | (G/A)₀₋₂₀GYALPHAI(X0)(X)(G/A)₀₋₂₀ |
| SEQ ID NO: 309(N9-N5): | (G/A)₀₋₂₀GDGVTH(X0)VPI(G/A)₀₋₂₀ |
| SEQ ID NO: 310(N9-N6): | (G/A)₀₋₂₀DGVTH(X0)VPIY(G/A)₀₋₂₀ |
| SEQ ID NO: 311(N9-N7): | (G/A)₀₋₂₀GVTH(X0)VPIYE(G/A)₀₋₂₀ |
| SEQ ID NO: 312(N9-N8): | (G/A)₀₋₂₀VDH(X0)VPIYEG(G/A)₀₋₂₀ |
| SEQ ID NO: 313(N9-N9): | (G/A)₀₋₂₀TH(X0)VPIYEGY(G/A)₀₋₂₀ |
| SEQ ID NO: 314(N9-N10): | (G/A)₀₋₂₀H(X0)VPIYEGYA(G/A)₀₋₂₀ |
| SEQ ID NO: 315(N9-N11): | (G/A)₀₋₂₀(X0)VPIYEGYAL(G/A)₀₋₂₀ |
| SEQ ID NO: 316(N9-N12): | (G/A)₀₋₂₀(X0)PIYEGYALP(G/A)₀₋₂₀ |
| SEQ ID NO: 317(N9-N13): | (G/A)₀₋₂₀(X0)IYEGYALPH(G/A)₀₋₂₀ |
| SEQ ID NO: 318(N9-N14): | (G/A)₀₋₂₀(X0)YEGYALPHA(G/A)₀₋₂₀ |
| SEQ ID NO: 319(N9-N15): | (G/A)₀₋₂₀(X0)EGYALPHAI(G/A)₀₋₂₀ |
| SEQ ID NO: 320(N9-N16): | (G/A)₀₋₂₀EGYALPHAI(X0)(G/A)₀₋₂₀ |
| SEQ ID NO: 321(N9-N17): | (G/A)₀₋₂₀GYALPHAI(X0)R(G/A)₀₋₂₀ |
| SEQ ID NO: 322(N9-N18): | (G/A)₀₋₂₀YALPHAI(X0)RL(G/A)₀₋₂₀ |
| SEQ ID NO: 323(N9-N19): | (G/A)₀₋₂₀ALPHAI(X0)RLD(G/A)₀₋₂₀ |
| SEQ ID NO: 324(N9-N20): | (G/A)₀₋₂₀LPHAI(X0)RLDL(G/A)₀₋₂₀ |
| SEQ ID NO: 325(N9-N21): | (G/A)₀₋₂₀PHAI(X0)RLDLA(G/A)₀₋₂₀ |
| SEQ ID NO: 326(N9-N22): | (G/A)₀₋₂₀HAI(X0)RLDLAG(G/A)₀₋₂₀ |
| SEQ ID NO: 327(N9-N23): | (G/A)₀₋₂₀AI(X0)RLDLAGR(G/A)₀₋₂₀ |
| SEQ ID NO: 328(N9-N24): | (G/A)₀₋₂₀I(X0)RLDLAGRD(G/A)₀₋₂₀ |
| SEQ ID NO: 329(N9-N25): | (G/A)₀₋₂₀(X0)RLDLAGRDL(G/A)₀₋₂₀ |
| SEQ ID NO: 330(N9-N26): | (G/A)₀₋₂₀LDLAGRDLTD(G/A)₀₋₂₀ |
| SEQ ID NO: 331(N9-N27): | (G/A)₀₋₂₀DLAGRDLTDY(G/A)₀₋₂₀ |
| SEQ ID NO: 332(N9-N28): | (G/A)₀₋₂₀LAGRDLTDYL(G/A)₀₋₂₀ |
| SEQ ID NO: 333(N9-N29): | (G/A)₀₋₂₀AGRDLTDYLM(G/A)₀₋₂₀ |
| SEQ ID NO: 334(N9-N30): | (G/A)₀₋₂₀GRDLTDYLMK(G/A)₀₋₂₀ |
| SEQ ID NO: 335(N9-N31): | (G/A)₀₋₂₀RDLTDYLMKI(G/A)₀₋₂₀ |
| SEQ ID NO: 336(N9-N32): | (G/A)₀₋₂₀DLTDYLMKIL(G/A)₀₋₂₀ |
| SEQ ID NO: 337(N9-N32): | (G/A)₀₋₂₀LTDYLMKILT(G/A)₀₋₂₀ |
| SEQ ID NO: 338(N9-N32): | (G/A)₀₋₂₀TDYLMKILTE(G/A)₀₋₂₀ |
| SEQ ID NO: 339(N9-N32): | (G/A)₀₋₂₀DYLMKILTER(G/A)₀₋₂₀ |
| SEQ ID NO: 340(B9-T28): | (G/A)₀₋₂₀YLMKILTERG(G/A)₀₋₂₀ |
| SEQ ID NO: 341(B9-T29): | (G/A)₀₋₂₀LMKILTERGY(G/A)₀₋₂₀ |
| SEQ ID NO: 342(B9-T30): | (G/A)₀₋₂₀MKILTERGYS(G/A)₀₋₂₀ |
| SEQ ID NO: 343(B9-T31): | (G/A)₀₋₂₀KILTERGYSF(G/A)₀₋₂₀ |
| SEQ ID NO: 344(N9-V1): | (G/A)₀₋₂₀(X)VPIYEGYALPHAI(X0)RLDLAG(G/A)₀₋₂₀ |
| SEQ ID NO: 345(N9-V2): | (G/A)₀₋₂₀(X)PIYEGYALPHAI(X0)RLDLAG(G/A)₀₋₂₀ |
| SEQ ID NO: 346(N9-V3): | (G/A)₀₋₂₀V(X)IYEGYALPHAI(X0)RLDLAG(G/A)₀₋₂₀ |
| SEQ ID NO: 347(N9-V4): | (G/A)₀₋₂₀VP(X)YEGYALPHAI(X0)RLDLAG(G/A)₀₋₂₀ |
| SEQ ID NO: 348(N9-V5): | (G/A)₀₋₂₀VPI(X)EGYALPHAI(X0)RLDLAG(G/A)₀₋₂₀ |
| SEQ ID NO: 349(N9-V6): | (G/A)₀₋₂₀VPIY(X)GYALPHAI(X0)RLDLAG(G/A)₀₋₂₀ |
| SEQ ID NO: 350(N9-V7): | (G/A)₀₋₂₀VPIYE(X)YALPHAI(X0)RLDLAG(G/A)₀₋₂₀ |
| SEQ ID NO: 351(N9-V8): | (G/A)₀₋₂₀VPIYEG(X)ALPHAI(X0)RLDLAG(G/A)₀₋₂₀ |
| SEQ ID NO: 352(N9-V9): | (G/A)₀₋₂₀VPIYEGY(X)LPHAI(X0)RLDLAG(G/A)₀₋₂₀ |
| SEQ ID NO: 353(N9-V10): | (G/A)₀₋₂₀VPIYEGYA(X)PHAI(X0)RLDLAG(G/A)₀₋₂₀ |
| SEQ ID NO: 354(N9-V11): | (G/A)₀₋₂₀VPIYEGYAL(X)HAI(X0)RLDLAG(G/A)₀₋₂₀ |
| SEQ ID NO: 355(N9-V12): | (G/A)₀₋₂₀VPIYEGYALP(X)AI(X0)RLDLAG(G/A)₀₋₂₀ |
| SEQ ID NO: 356(N9-V13): | (G/A)₀₋₂₀VPIYEGYALPH(X)I(X0)RLDLAG(G/A)₀₋₂₀ |
| SEQ ID NO: 357(N9-V14): | (G/A)₀₋₂₀VPIYEGYALPHA(X)(X0)RLDLAG(G/A)₀₋₂₀ |
| SEQ ID NO: 358(N9-V15): | (G/A)₀₋₂₀VPIYEGYALPHAI(X0)(X)LDLAG(G/A)₀₋₂₀ |
| SEQ ID NO: 359(N9-V16): | (G/A)₀₋₂₀VPIYEGYALPHAI(X0)R(X)DLAG(G/A)₀₋₂₀ |
| SEQ ID NO: 360(N9-V17): | (G/A)₀₋₂₀VPIYEGYALPHAI(X0)RL(X)LAG(G/A)₀₋₂₀ |
| SEQ ID NO: 361(N9-V18): | (G/A)₀₋₂₀VPIYEGYALPHAI(X0)RLD(X)AG(G/A)₀₋₂₀ |
| SEQ ID NO: 362(N9-V19): | (G/A)₀₋₂₀VPIYEGYALPHAI(X0)RLDL(X)G(G/A)₀₋₂₀ |
| SEQ ID NO: 363(N9-V20): | (G/A)₀₋₂₀VPIYEGYALPHAI(X0)RLDLA(X)(G/A)₀₋₂₀ |
| SEQ ID NO: 364(B9-V1): | (G/A)₀₋₂₀(X)PHAI(X0)RLDLAGRDLTDYLM(G/A)₀₋₂₀ |
| SEQ ID NO: 365(B9-V2): | (G/A)₀₋₂₀L(X)HAI(X0)RLDLAGRDLTDYLM(G/A)₀₋₂₀ |
| SEQ ID NO: 366(B9-V3): | (G/A)₀₋₂₀LP(X)AI(X0)RLDLAGRDLTDYLM(G/A)₀₋₂₀ |
| SEQ ID NO: 367(B9-V4): | (G/A)₀₋₂₀LPH(X)I(X0)RLDLAGRDLTDYLM(G/A)₀₋₂₀ |
| SEQ ID NO: 368(B9-V5): | (G/A)₀₋₂₀LPHA(X)(X0)RLDLAGRDLTDYLM(G/A)₀₋₂₀ |
| SEQ ID NO: 369(B9-V6): | (G/A)₀₋₂₀LPHAI(X0)(X)LDLAGRDLTDYLM(G/A)₀₋₂₀ |
| SEQ ID NO: 370(B9-V7): | (G/A)₀₋₂₀LPHAI(X0)R(X)DLAGRDLTDYLM(G/A)₀₋₂₀ |
| SEQ ID NO: 371(B9-V8): | (G/A)₀₋₂₀LPHAI(X0)RL(X)LAGRDLTDYLM(G/A)₀₋₂₀ |
| SEQ ID NO: 372(B9-V9): | (G/A)₀₋₂₀LPHAI(X0)RLD(X)AGRDLTDYLM(G/A)₀₋₂₀ |
| SEQ ID NO: 373(B9-V10): | (G/A)₀₋₂₀LPHAI(X0)RLDL(X)GRDLTDYLM(G/A)₀₋₂₀ |
| SEQ ID NO: 374(B9-V11): | (G/A)₀₋₂₀LPHAI(X0)RLDLA(X)RDLTDYLM(G/A)₀₋₂₀ |
| SEQ ID NO: 375(B9-V12): | (G/A)₀₋₂₀LPHAI(X0)RLDLAG(X)DLTDYLM(G/A)₀₋₂₀ |
| SEQ ID NO: 376(B9-V13): | (G/A)₀₋₂₀LPHAI(X0)RLDLAGR(X)LTDYLM(G/A)₀₋₂₀ |
| SEQ ID NO: 377(B9-V14): | (G/A)₀₋₂₀LPHAI(X0)RLDLAGRD(X)TDYLM(G/A)₀₋₂₀ |
| SEQ ID NO: 378(B9-V15): | (G/A)₀₋₂₀LPHAI(X0)RLDLAGRDL(X)DYLM(G/A)₀₋₂₀ |
| SEQ ID NO: 379(B9-V16): | (G/A)₀₋₂₀LPHAI(X0)RLDLAGRDLT(X)YLM(G/A)₀₋₂₀ |
| SEQ ID NO: 380(B9-V17): | (G/A)₀₋₂₀LPHAI(X0)RLDLAGRDLTD(X)LM(G/A)₀₋₂₀ |
| SEQ ID NO: 381(B9-V18): | (G/A)₀₋₂₀LPHAI(X0)RLDLAGRDLTDY(X)M(G/A)₀₋₂₀ |
| SEQ ID NO: 382(B9-V19): | (G/A)₀₋₂₀LPHAI(X0)RLDLAGRDLTDYL(X)(G/A)₀₋₂₀ |
| SEQ ID NO: 383 N9-M1): | (G/A)₀₋₂₀V(X0)DSGDGVTH(X0)VPIYEGYAL(G/A)₀₋₂₀ |
| SEQ ID NO: 384(N9-M2): | (G/A)₀₋₂₀(X0)DSGDGVTH(X0)VPIYEGYALP(G/A)₀₋₂₀ |
| SEQ ID NO: 385(N9-M3): | (G/A)₀₋₂₀DSGDGVTH(X0)VPIYEGYALPH(G/A)₀₋₂₀ |
| SEQ ID NO: 386(N9-M4): | (G/A)₀₋₂₀SGDGVTH(X0)VPIYEGYALPHA(G/A)₀₋₂₀ |
| SEQ ID NO: 387(N9-M5): | (G/A)₀₋₂₀GDGVTH(X0)VPIYEGYALPHAI(G/A)₀₋₂₀ |
| SEQ ID NO: 388(N9-M6): | (G/A)₀₋₂₀DGVTH(X0)VPIYEGYALPHAI(X0)(G/A)₀₋₂₀ |
| SEQ ID NO: 389(N9-M7): | (G/A)₀₋₂₀GVTH(X0)VPIYEGYALPHAI(X0)R(G/A)₀₋₂₀ |
| SEQ ID NO: 390(N9-M8): | (G/A)₀₋₂₀VTH(X0)VPIYEGYALPHAI(X0)RL(G/A)₀₋₂₀ |
| SEQ ID NO: 391(N9-M9): | (G/A)₀₋₂₀TH(X0)VPIYEGYALPHAI(X0)RLD(G/A)₀₋₂₀ |
| SEQ ID NO: 392(N9 ): | (G/A)₀₋₂₀H(X0)VPIYEGYALPHAI(X0)RLDL(G/A)₀₋₂₀ |
| SEQ ID NO: 393(N9-P1): | (G/A)₀₋₂₀(X0)VPIYEGYALPHAI(X0)RLDLA(G/A)₀₋₂₀ |
| SEQ ID NO: 394(N9-P2): | (G/A)₀₋₂₀VPIYEGYALPHAI(X0)RLDLAG(G/A)₀₋₂₀ |
| SEQ ID NO: 395(N9-P3): | (G/A)₀₋₂₀PIYEGYALPHAI(X0)RLDLAGR(G/A)₀₋₂₀ |
| SEQ ID NO: 396(N9-P4): | (G/A)₀₋₂₀IYEGYALPHAI(X0)RLDLAGRD(G/A)₀₋₂₀ |
| SEQ ID NO: 397(N9-P5): | (G/A)₀₋₂₀YEGYALPHAI(X0)RLDLAGRDL(G/A)₀₋₂₀ |
| SEQ ID NO: 398(N9-P6): | (G/A)₀₋₂₀EGYALPHAI(X0)RLDLAGRDLT(G/A)₀₋₂₀ |
| SEQ ID NO: 399(N9-P7): | (G/A)₀₋₂₀GYALPHAI(X0)RLDLAGRDLTD(G/A)₀₋₂₀ |
| SEQ ID NO: 400(N9-P8): | (G/A)₀₋₂₀YALPHAI(X0)RLDLAGRDLTDY(G/A)₀₋₂₀ |
| SEQ ID NO: 401(N9-P9): | (G/A)₀₋₂₀ALPHAI(X0)RLDLAGRDLTDYL(G/A)₀₋₂₀ |
| SEQ ID NO: 402(N9-P10): | (G/A)₀₋₂₀LPHAI(X0)RLDLAGRDLTDYLM(G/A)₀₋₂₀ |
| SEQ ID NO: 403(N9-P11): | (G/A)₀₋₂₀RHAI(X0)RLDLAGRDLTDYLMK(G/A)₀₋₂₀ |
| SEQ ID NO: 404(N9-P12): | (G/A)₀₋₂₀HAI(X0)RLDLAGRDLTDYLMKI(G/A)₀₋₂₀ |
| SEQ ID NO: 405(N9-P13): | (G/A)₀₋₂₀AI(X0)RLDLAGRDLTDYLMKIL(G/A)₀₋₂₀ |
| SEQ ID NO: 406(B9-P04): | (G/A)₀₋₂₀I(X0)RLDLAGRDLTDYLMKILT(G/A)₀₋₂₀ |
| SEQ ID NO: 407(B9-P05): | (G/A)₀₋₂₀(X0)RLDLAGRDLTDYLMKILTE(G/A)₀₋₂₀ |
| SEQ ID NO: 408(B9-P06): | (G/A)₀₋₂₀(X0)LDLAGRDLTDYLMKILTER(G/A)₀₋₂₀ |
| SEQ ID NO: 409(B9-P07): | (G/A)₀₋₂₀(X0)DLAGRDLTDYLMKILTERG(G/A)₀₋₂₀ |
| SEQ ID NO: 410(B9-P08): | (G/A)₀₋₂₀(X0)LAGRDLTDYLMKILTERGY(G/A)₀₋₂₀ |
| SEQ ID NO: 411(B9-P09): | (G/A)₀₋₂₀(X0)AGRDLTDYLMKILTERGYS(G/A)₀₋₂₀ |
| SEQ ID NO: 412(B9-P10): | (G/A)₀₋₂₀(X0)GRDLTDYLMKILTERGYSF(G/A)₀₋₂₀ |
| SEQ ID NO: 413(B9-NV01): | (G/A)₀₋₂₀(X)PHAI(X0)RLDLAGRDLTDYLM(G/A)₀₋₂₀ |
| SEQ ID NO: 414(B9-NV02): | (G/A)₀₋₂₀L(X)HAI(X0)RLDLAGRDLTDYLM(G/A)₀₋₂₀ |
| SEQ ID NO: 415(B9-NV03): | (G/A)₀₋₂₀LP(X)AI(X0)RLDLAGRDLTDYLM(G/A)₀₋₂₀ |
| SEQ ID NO: 416(B9-NV04): | (G/A)₀₋₂₀LPH(X)I(X0)RLDLAGRDLTDYLM(G/A)₀₋₂₀ |
| SEQ ID NO: 417(B9-NV05): | (G/A)₀₋₂₀LPHA(X)(X0)RLDLAGRDLTDYLM(G/A)₀₋₂₀ |
| SEQ ID NO: 418(B9-NV06): | (G/A)₀₋₂₀LPHA(X)(X0)RLDLAGRDLTDYLM(G/A)₀₋₂₀ |
| SEQ ID NO: 419(B9-NV07): | (G/A)₀₋₂₀LPHAI(X0)(X)LDLAGRDLTDYLM(G/A)₀₋₂₀ |
| SEQ ID NO: 420(B9-NV08): | (G/A)₀₋₂₀LPHAI(X0)R(X)DLAGRDLTDYLM(G/A)₀₋₂₀ |
| SEQ ID NO: 421(B9-NV09): | (G/A)₀₋₂₀LPHAI(X0)RL(X)LAGRDLTDYLM(G/A)₀₋₂₀ |
| SEQ ID NO: 422(B9-NV10): | (G/A)₀₋₂₀LPHAI(X0)RLD(X)AGRDLTDYLM(G/A)₀₋₂₀ |
| SEQ ID NO: 423(B9-NV11): | (G/A)₀₋₂₀LPHAI(X0)RLDL(X)GRDLTDYLM(G/A)₀₋₂₀ |
| SEQ ID NO: 424(B9-NV12): | (G/A)₀₋₂₀LPHAI(X0)RLDLA(X)RDLTDYLM(G/A)₀₋₂₀ |
| SEQ ID NO: 425(B9-NV13): | (G/A)₀₋₂₀LPHAI(X0)RLDLAG(X)DLTDYLM(G/A)₀₋₂₀ |
| SEQ ID NO: 426(B9-NV14): | (G/A)₀₋₂₀LPHAI(X0)RLDLAGR(X)LTDYLM(G/A)₀₋₂₀ |
| SEQ ID NO: 427(B9-NV15): | (G/A)₀₋₂₀LPHAI(X0)RLDLAGRD(X)TDYLM(G/A)₀₋₂₀ |
| SEQ ID NO: 428(B9-NV16): | (G/A)₀₋₂₀LPHAI(X0)RLDLAGRDL(X)DYLM(G/A)₀₋₂₀ |
| SEQ ID NO: 429(B9-NV17): | (G/A)₀₋₂₀LPHAI(X0)RLDLAGRDLT(X)YLM(G/A)₀₋₂₀ |
| SEQ ID NO: 430(B9-NV18): | (G/A)₀₋₂₀LPHAI(X0)RLDLAGRDLTD(X)LM(G/A)₀₋₂₀ |
| SEQ ID NO: 431(B9-NV19): | (G/A)₀₋₂₀LPHAI(X0)RLDLAGRDLTDY(X)M(G/A)₀₋₂₀ |
| SEQ ID NO: 432(B9-NV20): | (G/A)₀₋₂₀LPHAI(X0)RLDLAGRDLTDYL(X)(G/A)₀₋₂₀ |
| SEQ ID NO: 433(N9-X1): | (G/A)₀₋₂₀(X)(X0)DSGDGVTH(X0)VPIYEGYALPHAI(X0)RLDLAGRDLTDYLMKI(G/A)₀₋₂₀ |
| SEQ ID NO: 434(N9-X2): | (G/A)₀₋₂₀V(X0)(X)SGDGVTH(X0)VPIYEGYALPHAI(X0)RLDLAGRDLTDYLMKI(G/A)₀₋₂₀ |
| SEQ ID NO: 435(N9-X3): | (G/A)₀₋₂₀V(X0)D(X)GDGVTH(X0)VPIYEGYALPHAI(X0)RLDLAGRDLTDYLMKI(G/A)₀₋₂₀ |
| SEQ ID NO: 436(N9-X4): | (G/A)₀₋₂₀V(X0)DS(X)DGVTH(X0)VPIYEGYALPHAI(X0)RLDLAGRDLTDYLMKI(G/A)₀₋₂₀ |
| SEQ ID NO: 437(N9-X5): | (G/A)₀₋₂₀V(X0)DSG(X)GVTH(X0)VPIYEGYALPHAI(X0)RLDLAGRDLTDYLMKI(G/A)₀₋₂₀ |
| SEQ ID NO: 438(N9-X6): | (G/A)₀₋₂₀V(X0)DSGD(X)VTH(X0)VPIYEGYALPHAI(X0)RLDLAGRDLTDYLMKI(G/A)₀₋₂₀ |
| SEQ ID NO: 439(N9-X7): | (G/A)₀₋₂₀V(X0)DSGDG(X)TH(X0)VPIYEGYALPHAI(X0)RLDLAGRDLTDYLMKI(G/A)₀₋₂₀ |
| SEQ ID NO: 440(N9-X8): | (G/A)₀₋₂₀V(X0)DSGDGV(X)H(X0)VPIYEGYALPHAI(X0)RLDLAGRDLTDYLMKI(G/A)₀₋₂₀ |
| SEQ ID NO: 441(N9-X9): | (G/A)₀₋₂₀V(X0)DSGDGVT(X)(X0)VPIYEGYALPHAI(X0)RLDLAGRDLTDYLMKI(G/A)₀₋₂₀ |
| SEQ ID NO: 442(N9-X10): | (G/A)₀₋₂₀V(X0)DSGDGVTH(X0)(X)PIYEGYALPHAI(X0)RLDLAGRDLTDYLMKI(G/A)₀₋₂₀ |
| SEQ ID NO: 443(N9-X11): | (G/A)₀₋₂₀V(X0)DSGDGVTH(X0)V(X)IYEGYALPHAI(X0)RLDLAGRDLTDYLMKI(G/A)₀₋₂₀ |
| SEQ ID NO: 444(N9-X12): | (G/A)₀₋₂₀V(X0)DSGDGVTH(X0)VP(X)YEGYALPHAI(X0)RLDLAGRDLTDYLMKI(G/A)₀₋₂₀ |
| SEQ ID NO: 445(N9-X13): | (G/A)₀₋₂₀V(X0)DSGDGVTH(X0)VPI(X)EGYALPHAI(X0)RLDLAGRDLTDYLMKI(G/A)₀₋₂₀ |
| SEQ ID NO: 446(N9-X14): | (G/A)₀₋₂₀V(X0)DSGDGVTH(X0)VPIY(X)GYALPHAI(X0)RLDLAGRDLTDYLMKI(G/A)₀₋₂₀ |
| SEQ ID NO: 447(N9-X15): | (G/A)₀₋₂₀V(X0)DSGDGVTH(X0)VPIYE(X)YALPHAI(X0)RLDLAGRDLTDYLMKI(G/A)₀₋₂₀ |
| SEQ ID NO: 448(N9-X16): | (G/A)₀₋₂₀V(X0)DSGDGVTH(X0)VPIYEG(X)ALPHAI(X0)RLDLAGRDLTDYLMKI(G/A)₀₋₂₀ |
| SEQ ID NO: 449(N9-X17): | (G/A)₀₋₂₀V(X0)DSGDGVTH(X0)VPIYEGY(X)LPHAI(X0)RLDLAGRDLTDYLMKI(G/A)₀₋₂₀ |
| SEQ ID NO: 450(N9-X18): | (G/A)₀₋₂₀V(X0)DSGDGVTH(X0)VPIYEGYA(X)PHAI(X0)RLDLAGRDLTDYLMKI(G/A)₀₋₂₀ |
| SEQ ID NO: 451(N9-X19): | (G/A)₀₋₂₀V(X0)DSGDGVTH(X0)VPIYEGYAL(X)HAI(X0)RLDLAGRDLTDYLMKI(G/A)₀₋₂₀ |
| SEQ ID NO: 452(N9-X20): | (G/A)₀₋₂₀V(X0)DSGDGVTH(X0)VPIYEGYALP(X)AI(X0)RLDLAGRDLTDYLMKI(G/A)₀₋₂₀ |
| SEQ ID NO: 453(N9-X21): | (G/A)₀₋₂₀V(X0)DSGDGVTH(X0)VPIYEGYALPH(X)I(X0)RLDLAGRDLTDYLMKI(G/A)₀₋₂₀ |
| SEQ ID NO: 454(N9-X22): | (G/A)₀₋₂₀V(X0)DSGDGVTH(X0)VPIYEGYALPHA(X)(X0)RLDLAGRDLTDYLMKI(G/A)₀₋₂₀ |
| SEQ ID NO: 455(N9-X23): | (G/A)₀₋₂₀V(X0)DSGDGVTH(X0)VPIYEGYALPHAI(X0)(X)LDLAGRDLTDYLMKI(G/A)₀₋₂₀ |
| SEQ ID NO: 456(N9-X24): | (G/A)₀₋₂₀V(X0)DSGDGVTH(X0)VPIYEGYALPHAI(X0)R(X)DLAGRDLTDYLMKI(G/A)₀₋₂₀ |
| SEQ ID NO: 457(N9-X25): | (G/A)₀₋₂₀V(X0)DSGDGVTH(X0)VPIYEGYALPHAI(X0)RL(X)LAGRDLTDYLMKI(G/A)₀₋₂₀ |
| SEQ ID NO: 458(N9-X26): | (G/A)₀₋₂₀V(X0)DSGDGVTH(X0)VPIYEGYALPHAI(X0)RLD(X)AGRDLTDYLMKI(G/A)₀₋₂₀ |
| SEQ ID NO: 459(N9-X27): | (G/A)₀₋₂₀V(X0)DSGDGVTH(X0)VPIYEGYALPHAI(X0)RLDL(X)GRDLTDYLMKI(G/A)₀₋₂₀ |
| SEQ ID NO: 460(N9-X28): | (G/A)₀₋₂₀V(X0)DSGDGVTH(X0)VPIYEGYALPHAI(X0)RLDLA(X)RDLTDYLMKI(G/A)₀₋₂₀ |
| SEQ ID NO: 461(N9-X29): | (G/A)₀₋₂₀V(X0)DSGDGVTH(X0)VPIYEGYALPHAI(X0)RLDLAG(X)DLTDYLMKI(G/A)₀₋₂₀ |
| SEQ ID NO: 462(N9-X30): | (G/A)₀₋₂₀V(X0)DSGDGVTH(X0)VPIYEGYALPHAI(X0)RLDLAGR(X)LTDYLMKI(G/A)₀₋₂₀ |
| SEQ ID NO: 463(N9-X31): | (G/A)₀₋₂₀V(X0)DSGDGVTH(X0)VPIYEGYALPHAI(X0)RLDLAGRD(X)TDYLMKI(G/A)₀₋₂₀ |
| SEQ ID NO: 464(N9-X32): | (G/A)₀₋₂₀V(X0)DSGDGVTH(X0)VPIYEGYALPHAI(X0)RLDLAGRDL(X)DYLMKI(G/A)₀₋₂₀ |
| SEQ ID NO: 465(N9-X33): | (G/A)₀₋₂₀V(X0)DSGDGVTH(X0)VPIYEGYALPHAI(X0)RLDLAGRDLT(X)YLMKI(G/A)₀₋₂₀ |
| SEQ ID NO: 466(N9-X34): | (G/A)₀₋₂₀V(X0)DSGDGVTH(X0)VPIYEGYALPHAI(X0)RLDLAGRDLTD(X)LMKI(G/A)₀₋₂₀ |
| SEQ ID NO: 467(N9-X35): | (G/A)₀₋₂₀V(X0)DSGDGVTH(X0)VPIYEGYALPHAI(X0)RLDLAGRDLTDY(X)MKI(G/A)₀₋₂₀ |
| SEQ ID NO: 468(N9-X36): | (G/A)₀₋₂₀V(X0)DSGDGVTH(X0)VPIYEGYALPHAI(X0)RLDLAGRDLTDYL(X)KI(G/A)₀₋₂₀ |
| SEQ ID NO: 469(N9-X37): | (G/A)₀₋₂₀V(X0)DSGDGVTH(X0)VPIYEGYALPHAI(X0)RLDLAGRDLTDYLM(X)I(G/A)₀₋₂₀ |
| SEQ ID NO: 470(N9-X38): | (G/A)₀₋₂₀V(X0)DSGDGVTH(X0)VPIYEGYALPHAI(X0)RLDLAGRDLTDYLMK(X)(G/A)₀₋₂₀ |
| SEQ ID NO: 471(NB10-core): | (X)₀₋₂₀AGRDLTDYLM(X)₀₋₂₀ |
| SEQ ID NO: 472(NB10-core): | (G/A)₀₋₂₀(X)GRDLTDYLM(G/A)₀₋₂₀ |
| SEQ ID NO: 473(NB10-core): | (G/A)₀₋₂₀A(X)RDLTDYLM(G/A)₀₋₂₀ |
| SEQ ID NO: 474(NB10-core): | (G/A)₀₋₂₀AG(X)DLTDYLM(G/A)₀₋₂₀ |
| SEQ ID NO: 475(NB10-core): | (G/A)₀₋₂₀AGR(X)LTDYLM(G/A)₀₋₂₀ |
| SEQ ID NO: 476(NB10-core): | (G/A)₀₋₂₀AGRD(X)TDYLM(G/A)₀₋₂₀ |
| SEQ ID NO: 477(NB10-core): | (G/A)₀₋₂₀AGRDL(X)DYLM(G/A)₀₋₂₀ |
| SEQ ID NO: 478(NB10-core): | (G/A)₀₋₂₀AGRDLT(X)YLM(G/A)₀₋₂₀ |
| SEQ ID NO: 479(NB10-core): | (G/A)₀₋₂₀AGRDLTD(X)LM(G/A)₀₋₂₀ |
| SEQ ID NO: 480(NB10-core): | (G/A)₀₋₂₀AGRDLTDY(X)M(G/A)₀₋₂₀ |
| SEQ ID NO: 481(NB10-core): | (G/A)₀₋₂₀AGRDLTDYL(X)(G/A)₀₋₂₀ |
| SEQ ID NO: 482(N10-NV1): | (G/A)₀₋₂₀(X)DYLMKILTE(G/A)₀₋₂₀ |
| SEQ ID NO: 483(N10-NV2): | (G/A)₀₋₂₀T(X)YLMKILTE(G/A)₀₋₂₀ |
| SEQ ID NO: 484(N10-NV3): | (G/A)₀₋₂₀TD(X)LMKILTE(G/A)₀₋₂₀ |
| SEQ ID NO: 485(N10-NV4): | (G/A)₀₋₂₀TDY(X)MKILTE(G/A)₀₋₂₀ |
| SEQ ID NO: 486(N10-NV5): | (G/A)₀₋₂₀TDYL(X)KILTE(G/A)₀₋₂₀ |
| SEQ ID NO: 487(N10-NV6): | (G/A)₀₋₂₀TDYLM(X)ILTE(G/A)₀₋₂₀ |
| SEQ ID NO: 488(N10-NV7): | (G/A)₀₋₂₀TDYLMK(X)LTE(G/A)₀₋₂₀ |
| SEQ ID NO: 489(N10-NV8): | (G/A)₀₋₂₀TDYLMKI(X)TE(G/A)₀₋₂₀ |
| SEQ ID NO: 490(N10-NV9): | (G/A)₀₋₂₀TDYLMKIL(X)E(G/A)₀₋₂₀ |
| SEQ ID NO: 491(N10-NV10): | (G/A)₀₋₂₀TDYLMKILT(X)(G/A)₀₋₂₀ |
| SEQ ID NO: 492(N10-N1): | (G/A)₀₋₂₀LPHAI(X0)RLDL(G/A)₀₋₂₀ |
| SEQ ID NO: 493(N10-N2): | (G/A)₀₋₂₀PHAI(X0)RLDLA(G/A)₀₋₂₀ |
| SEQ ID NO: 494(N10-N3): | (G/A)₀₋₂₀HAI(X0)RLDLAG(G/A)₀₋₂₀ |
| SEQ ID NO: 495(N10-N4): | (G/A)₀₋₂₀AI(X0)RLDLAGR(G/A)₀₋₂₀ |
| SEQ ID NO: 496(N10-N5): | (G/A)₀₋₂₀I(X0)RLDLAGRD(G/A)₀₋₂₀ |
| SEQ ID NO: 497(N10-N6): | (G/A)₀₋₂₀(X0)RLDLAGRDL(G/A)₀₋₂₀ |
| SEQ ID NO: 498(N10-N7): | (G/A)₀₋₂₀(X0)LDLAGRDLT(G/A)₀₋₂₀ |
| SEQ ID NO: 499(N10-N8): | (G/A)₀₋₂₀(X0)DLAGRDLTD(G/A)₀₋₂₀ |
| SEQ ID NO: 500(N10-N9): | (G/A)₀₋₂₀(X0)LAGRDLTDY(G/A)₀₋₂₀ |
| SEQ ID NO: 501(N10-N10): | (G/A)₀₋₂₀(X0)AGRDLTDYL(G/A)₀₋₂₀ |
| SEQ ID NO: 502(N10-N11): | (G/A)₀₋₂₀(X0)GRDLTDYLM(G/A)₀₋₂₀ |
| SEQ ID NO: 503(N10-N12): | (G/A)₀₋₂₀(X0)RDLTDYLMK(G/A)₀₋₂₀ |
| SEQ ID NO: 504(N10-N13): | (G/A)₀₋₂₀(X0)DLTDYLMKI(G/A)₀₋₂₀ |
| SEQ ID NO: 505(N10-N14): | (G/A)₀₋₂₀(X0)LTDYLMKIL(G/A)₀₋₂₀ |
| SEQ ID NO: 506(N10-N15): | (G/A)₀₋₂₀(X0)TDYLMKILT(G/A)₀₋₂₀ |
| SEQ ID NO: 507(N10-N16): | (G/A)₀₋₂₀(X0)DYLMKILTE(G/A)₀₋₂₀ |
| SEQ ID NO: 508(N10-N17): | (G/A)₀₋₂₀(X0)YLMKILTER(G/A)₀₋₂₀ |
| SEQ ID NO: 509(N10-N18): | (G/A)₀₋₂₀(X0)LMKILTERG(G/A)₀₋₂₀ |
| SEQ ID NO: 510(N10-N19): | (G/A)₀₋₂₀(X0)MKILTERGY(G/A)₀₋₂₀ |
| SEQ ID NO: 511(N10-N20): | (G/A)₀₋₂₀(X0)KILTERGYS(G/A)₀₋₂₀ |
| SEQ ID NO: 512(N10-N21): | (G/A)₀₋₂₀(X0)ILTERGYSF(X0)TTAEREIVR(G/A)₀₋₂₀ |
| SEQ ID NO: 513(N10-N22): | (G/A)₀₋₂₀ILTERGYSF(X0)(G/A)₀₋₂₀ |
| SEQ ID NO: 514(N10-N23): | (G/A)₀₋₂₀LTERGYSF(X0)T(G/A)₀₋₂₀ |
| SEQ ID NO: 515(N10-N24): | (G/A)₀₋₂₀TERGYSF(X0)TT(G/A)₀₋₂₀ |
| SEQ ID NO: 516(N10-N25): | (G/A)₀₋₂₀ERGYSF(X0)TTA(G/A)₀₋₂₀ |
| SEQ ID NO: 517(N10-N26): | (G/A)₀₋₂₀RGYSF(X0)TTAE(G/A)₀₋₂₀ |
| SEQ ID NO: 518(N10-N27): | (G/A)₀₋₂₀GYSF(X0)TTAER(G/A)₀₋₂₀ |
| SEQ ID NO: 519(N10-N28): | (G/A)₀₋₂₀YSF(X0)TTAERE(G/A)₀₋₂₀ |
| SEQ ID NO: 520(N10-N29): | (G/A)₀₋₂₀SF(X0)TTAEREI(G/A)₀₋₂₀ |
| SEQ ID NO: 521(N10-N30): | (G/A)₀₋₂₀F(X0)TTAEREIV(G/A)₀₋₂₀ |
| SEQ ID NO: 522(N10-N31): | (G/A)₀₋₂₀(X0)TTAEREIVR(G/A)₀₋₂₀ |
| SEQ ID NO: 523(N10-V1): | (G/A)₀₋₂₀(X)GRDLTDYLMKILTERGYSF(G/A)₀₋₂₀ |
| SEQ ID NO: 524(N10-V2): | (G/A)₀₋₂₀A(X)RDLTDYLMKILTERGYSF(G/A)₀₋₂₀ |
| SEQ ID NO: 525(N10-V3): | (G/A)₀₋₂₀AG(X)DLTDYLMKILTERGYSF(G/A)₀₋₂₀ |
| SEQ ID NO: 526(N10-V4): | (G/A)₀₋₂₀AGR(X)LTDYLMKILTERGYSF(G/A)₀₋₂₀ |
| SEQ ID NO: 527(N10-V5): | (G/A)₀₋₂₀AGRD(X)TDYLMKILTERGYSF(G/A)₀₋₂₀ |
| SEQ ID NO: 528(N10-V6): | (G/A)₀₋₂₀AGRDL(X)DYLMKILTERGYSF(G/A)₀₋₂₀ |
| SEQ ID NO: 529(N10-V7): | (G/A)₀₋₂₀AGRDLT(X)YLMKILTERGYSF(G/A)₀₋₂₀ |
| SEQ ID NO: 530(N10-V8): | (G/A)₀₋₂₀AGRDLTD(X)LMKILTERGYSF(G/A)₀₋₂₀ |
| SEQ ID NO: 531(N10-V9): | (G/A)₀₋₂₀AGRDLTDY(X)MKILTERGYSF(G/A)₀₋₂₀ |
| SEQ ID NO: 532(N10-V10): | (G/A)₀₋₂₀AGRDLTDYL(X)KILTERGYSF(G/A)₀₋₂₀ |
| SEQ ID NO: 533(N10-V11): | (G/A)₀₋₂₀AGRDLTDYLM(X)ILTERGYSF(G/A)₀₋₂₀ |
| SEQ ID NO: 534(N10-V12): | (G/A)₀₋₂₀AGRDLTDYLMK(X)LTERGYSF(G/A)₀₋₂₀ |
| SEQ ID NO: 535(N10-V13): | (G/A)₀₋₂₀AGRDLTDYLMKI(X)TERGYSF(G/A)₀₋₂₀ |
| SEQ ID NO: 536(N10-V14): | (G/A)₀₋₂₀AGRDLTDYLMKIL(X)ERGYSF(G/A)₀₋₂₀ |
| SEQ ID NO: 537(N10-V15): | (G/A)₀₋₂₀AGRDLTDYLMKILT(X)RGYSF(G/A)₀₋₂₀ |
| SEQ ID NO: 538(N10-V16): | (G/A)₀₋₂₀AGRDLTDYLMKILTE(X)GYSF(G/A)₀₋₂₀ |
| SEQ ID NO: 539(N10-V17): | (G/A)₀₋₂₀AGRDLTDYLMKILTER(X)YSF(G/A)₀₋₂₀ |
| SEQ ID NO: 540(N10-V18): | (G/A)₀₋₂₀AGRDLTDYLMKILTERG(X)SF(G/A)₀₋₂₀ |
| SEQ ID NO: 541(N10-V19): | (G/A)₀₋₂₀AGRDLTDYLMKILTERGY(X)F(G/A)₀₋₂₀ |
| SEQ ID NO: 542(N10-V20): | (G/A)₀₋₂₀AGRDLTDYLMKILTERGYS(X)(G/A)₀₋₂₀ |
| SEQ ID NO: 543(N10-M1): | (G/A)₀₋₂₀LPHAI(X0)RLDLAGRDLTDYLM(G/A)₀₋₂₀ |
| SEQ ID NO: 544(N10-M2): | (G/A)₀₋₂₀PHAI(X0)RLDLAGRDLTDYLMK(G/A)₀₋₂₀ |
| SEQ ID NO: 545(N10-M3): | (G/A)₀₋₂₀HAI(X0)RLDLAGRDLTDYLMKI(G/A)₀₋₂₀ |
| SEQ ID NO: 546(N10-M4): | (G/A)₀₋₂₀AI(X0)RLDLAGRDLTDYLMKIL(G/A)₀₋₂₀ |
| SEQ ID NO: 547(N10-M5): | (G/A)₀₋₂₀I(X0)RLDLAGRDLTDYLMKILT(G/A)₀₋₂₀ |
| SEQ ID NO: 548(N10-M6): | (G/A)₀₋₂₀(X0)RLDLAGRDLTDYLMKILTE(G/A)₀₋₂₀ |
| SEQ ID NO: 549(N10-M7): | (G/A)₀₋₂₀RLDLAGRDLTDYLMKILTER(G/A)₀₋₂₀ |
| SEQ ID NO: 550(N10-M8): | (G/A)₀₋₂₀LDLAGRDLTDYLMKILTERG(G/A)₀₋₂₀ |
| SEQ ID NO: 551(N10-M9): | (G/A)₀₋₂₀DLAGRDLTDYLMKILTERGY(G/A)₀₋₂₀ |
| SEQ ID NO: 552(N10-M10): | (G/A)₀₋₂₀LAGRDLTDYLMKILTERGYS(G/A)₀₋₂₀ |
| SEQ ID NO: 553(N10 ): | (G/A)₀₋₂₀AGRDLTDYLMKILTERGYSF(G/A)₀₋₂₀ |
| SEQ ID NO: 554(N10-P1): | (G/A)₀₋₂₀GRDLTDYLMKILTERGYSF(X0)(G/A)₀₋₂₀ |
| SEQ ID NO: 555(N10-P2): | (G/A)₀₋₂₀RDLTDYLMKILTERGYSF(X0)T(G/A)₀₋₂₀ |
| SEQ ID NO: 556(N10-P3): | (G/A)₀₋₂₀DLTDYLMKILTERGYSF(X0)TT(G/A)₀₋₂₀ |
| SEQ ID NO: 557(N10-P4): | (G/A)₀₋₂₀LTDYLMKILTERGYSF(X0)TTA(G/A)₀₋₂₀ |
| SEQ ID NO: 558(N10-P5): | (G/A)₀₋₂₀TDYLMKILTERGYSF(X0)TTAE(G/A)₀₋₂₀ |
| SEQ ID NO: 559(N10-P6): | (G/A)₀₋₂₀DYLMKILTERGYSF(X0)TTAER(G/A)₀₋₂₀ |
| SEQ ID NO: 560(N10-P7): | (G/A)₀₋₂₀YLMKILTERGYSF(X0)TTAERE(G/A)₀₋₂₀ |
| SEQ ID NO: 561(N10-P8): | (G/A)₀₋₂₀LMKILTERGYSF(X0)TTAEREI(G/A)₀₋₂₀ |
| SEQ ID NO: 562(N10-P9): | (G/A)₀₋₂₀MKILTERGYSF(X0)TTAEREIV(G/A)₀₋₂₀ |
| SEQ ID NO: 563(N10-P10): | (G/A)₀₋₂₀KILTERGYSF(X0)TTAEREIVR(G/A)₀₋₂₀ |
| SEQ ID NO: 564(N10-X1): | (G/A)₀₋₂₀(X)PHAI(X0)RLDLAGRDLTDYLMKILTERGYSF(X0)TTAEREIVR(G/A)₀₋₂₀ |
| SEQ ID NO: 565(N10-X2): | (G/A)₀₋₂₀L(X)HAI(X0)RLDLAGRDLTDYLMKILTERGYSF(X0)TTAEREIVR(G/A)₀₋₂₀ |
| SEQ ID NO: 566(N10-X3): | (G/A)₀₋₂₀LP(X)AI(X0)RLDLAGRDLTDYLMKILTERGYSF(X0)TTAEREIVR(G/A)₀₋₂₀ |
| SEQ ID NO: 567(N10-X4): | (G/A)₀₋₂₀LPH(X)I(X0)RLDLAGRDLTDYLMKILTERGYSF(X0)TTAEREIVR(G/A)₀₋₂₀ |
| SEQ ID NO: 568(N10-X5): | (G/A)₀₋₂₀LPHA(X)(X0)RLDLAGRDLTDYLMKILTERGYSF(X0)TTAEREIVR(G/A)₀₋₂₀ |
| SEQ ID NO: 569(N10-X6): | (G/A)₀₋₂₀LPHAI(X0)(X)LDLAGRDLTDYLMKILTERGYSF(X0)TTAEREIVR(G/A)₀₋₂₀ |
| SEQ ID NO: 570(N10-X7): | (G/A)₀₋₂₀LPHAI(X0)R(X)DLAGRDLTDYLMKILTERGYSF(X0)TTAEREIVR(G/A)₀₋₂₀ |
| SEQ ID NO: 571(N10-X8): | (G/A)₀₋₂₀LPHAI(X0)RL(X)LAGRDLTDYLMKILTERGYSF(X0)TTAEREIVR(G/A)₀₋₂₀ |
| SEQ ID NO: 572(N10-X9): | (G/A)₀₋₂₀LPHAI(X0)RLD(X)AGRDLTDYLMKILTERGYSF(X0)TTAEREIVR(G/A)₀₋₂₀ |
| SEQ ID NO: 573(N10-X10): | (G/A)₀₋₂₀LPHAI(X0)RLDL(X)GRDLTDYLMKILTERGYSF(X0)TTAEREIVR(G/A)₀₋₂₀ |
| SEQ ID NO: 574(N10-X11): | (G/A)₀₋₂₀LPHAI(X0)RLDLA(X)RDLTDYLMKILTERGYSF(X0)TTAEREIVR(G/A)₀₋₂₀ |
| SEQ ID NO: 575(N10-X12): | (G/A)₀₋₂₀LPHAI(X0)RLDLAG(X)DLTDYLMKILTERGYSF(X0)TTAEREIVR(G/A)₀₋₂₀ |
| SEQ ID NO: 576(N10-X13): | (G/A)₀₋₂₀LPHAI(X0)RLDLAGR(X)LTDYLMKILTERGYSF(X0)TTAEREIVR(G/A)₀₋₂₀ |
| SEQ ID NO: 577(N10-X14): | (G/A)₀₋₂₀LPHAI(X0)RLDLAGRD(X)TDYLMKILTERGYSF(X0)TTAEREIVR(G/A)₀₋₂₀ |
| SEQ ID NO: 578(N10-X15): | (G/A)₀₋₂₀LPHAI(X0)RLDLAGRDL(X)DYLMKILTERGYSF(X0)TTAEREIVR(G/A)₀₋₂₀ |
| SEQ ID NO: 579(N10-X16): | (G/A)₀₋₂₀LPHAI(X0)RLDLAGRDLT(X)YLMKILTERGYSF(X0)TTAEREIVR(G/A)₀₋₂₀ |
| SEQ ID NO: 580(N10-X17): | (G/A)₀₋₂₀LPHAI(X0)RLDLAGRDLTD(X)LMKILTERGYSF(X0)TTAEREIVR(G/A)₀₋₂₀ |
| SEQ ID NO: 581(N10-X18): | (G/A)₀₋₂₀LPHAI(X0)RLDLAGRDLTDY(X)MKILTERGYSF(X0)TTAEREIVR(G/A)₀₋₂₀ |
| SEQ ID NO: 582(N10-X19): | (G/A)₀₋₂₀LPHAI(X0)RLDLAGRDLTDYL(X)KILTERGYSF(X0)TTAEREIVR(G/A)₀₋₂₀ |
| SEQ ID NO: 583(N10-X20): | (G/A)₀₋₂₀LPHAI(X0)RLDLAGRDLTDYLM(X)ILTERGYSF(X0)TTAEREIVR(G/A)₀₋₂₀ |
| SEQ ID NO: 584(N10-X21): | (G/A)₀₋₂₀LPHAI(X0)RLDLAGRDLTDYLMK(X)LTERGYSF(X0)TTAEREIVR(G/A)₀₋₂₀ |
| SEQ ID NO: 585(N10-X22): | (G/A)₀₋₂₀LPHAI(X0)RLDLAGRDLTDYLMKI(X)TERGYSF(X0)TTAEREIVR(G/A)₀₋₂₀ |
| SEQ ID NO: 586(N10-X23): | (G/A)₀₋₂₀LPHAI(X0)RLDLAGRDLTDYLMKIL(X)ERGYSF(X0)TTAEREIVR(G/A)₀₋₂₀ |
| SEQ ID NO: 587(N10-X24): | (G/A)₀₋₂₀LPHAI(X0)RLDLAGRDLTDYLMKILT(X)RGYSF(X0)TTAEREIVR(G/A)₀₋₂₀ |
| SEQ ID NO: 588(N10-X25): | (G/A)₀₋₂₀LPHAI(X0)RLDLAGRDLTDYLMKILTE(X)GYSF(X0)TTAEREIVR(G/A)₀₋₂₀ |
| SEQ ID NO: 589(N10-X26): | (G/A)₀₋₂₀LPHAI(X0)RLDLAGRDLTDYLMKILTER(X)YSF(X0)TTAEREIVR(G/A)₀₋₂₀ |
| SEQ ID NO: 590(N10-X27): | (G/A)₀₋₂₀LPHAI(X0)RLDLAGRDLTDYLMKILTERG(X)SF(X0)TTAEREIVR(G/A)₀₋₂₀ |
| SEQ ID NO: 591(N10-X28): | (G/A)₀₋₂₀LPHAI(X0)RLDLAGRDLTDYLMKILTERGY(X)F(X0)TTAEREIVR(G/A)₀₋₂₀ |
| SEQ ID NO: 592(N10-X29): | (G/A)₀₋₂₀LPHAI(X0)RLDLAGRDLTDYLMKILTERGYS(X)(X0)TTAEREIVR(G/A)₀₋₂₀ |
| SEQ ID NO: 593(N10-X30): | (G/A)₀₋₂₀LPHAI(X0)RLDLAGRDLTDYLMKILTERGYSF(X0)(X)TAEREIVR(G/A)₀₋₂₀ |
| SEQ ID NO: 594(N10-X31): | (G/A)₀₋₂₀LPHAI(X0)RLDLAGRDLTDYLMKILTERGYSF(X0)T(X)AEREIVR(G/A)₀₋₂₀ |
| SEQ ID NO: 595(N10-X32): | (G/A)₀₋₂₀LPHAI(X0)RLDLAGRDLTDYLMKILTERGYSF(X0)TT(X)EREIVR(G/A)₀₋₂₀ |
| SEQ ID NO: 596(N10-X33): | (G/A)₀₋₂₀LPHAI(X0)RLDLAGRDLTDYLMKILTERGYSF(X0)TTA(X)REIVR(G/A)₀₋₂₀ |
| SEQ ID NO: 597(N10-X34): | (G/A)₀₋₂₀LPHAI(X0)RLDLAGRDLTDYLMKILTERGYSF(X0)TTAE(X)EIVR(G/A)₀₋₂₀ |
| SEQ ID NO: 598(N10-X35): | (G/A)₀₋₂₀LPHAI(X0)RLDLAGRDLTDYLMKILTERGYSF(X0)TTAER(X)IVR(G/A)₀₋₂₀ |
| SEQ ID NO: 599(N10-X36): | (G/A)₀₋₂₀LPHAI(X0)RLDLAGRDLTDYLMKILTERGYSF(X0)TTAERE(X)VR(G/A)₀₋₂₀ |
| SEQ ID NO: 600(N10-X37): | (G/A)₀₋₂₀LPHAI(X0)RLDLAGRDLTDYLMKILTERGYSF(X0)TTAEREI(X)R(G/A)₀₋₂₀ |
| SEQ ID NO: 601(N10-X38): | (G/A)₀₋₂₀LPHAI(X0)RLDLAGRDLTDYLMKILTERGYSF(X0)TTAEREIV(X)(G/A)₀₋₂₀ |
| SEQ ID NO: 602(B11-core): | (X)₀₋₂₀LCYVALDFEQ(X)₀₋₂₀ |
| SEQ ID NO: 603(B11-NV01): | (G/A)₀₋₂₀(X)CYVALDFE(X0)(G/A)₀₋₂₀ |
| SEQ ID NO: 604(B11-NV02): | (G/A)₀₋₂₀L(X)YVALDFE(X0)(G/A)₀₋₂₀ |
| SEQ ID NO: 605(B11-NV03): | (G/A)₀₋₂₀LC(X)VALDFE(X0)(G/A)₀₋₂₀ |
| SEQ ID NO: 606(B11-NV04): | (G/A)₀₋₂₀LCY(X)ALDFE(X0)(G/A)₀₋₂₀ |
| SEQ ID NO: 607(B11-NV05): | (G/A)₀₋₂₀LCYV(X)LDFE(X0)(G/A)₀₋₂₀ |
| SEQ ID NO: 608(B11-NV06): | (G/A)₀₋₂₀LCYVA(X)DFE(X0)(G/A)₀₋₂₀ |
| SEQ ID NO: 609(B11-NV07): | (G/A)₀₋₂₀LCYVAL(X)FE(X0)(G/A)₀₋₂₀ |
| SEQ ID NO: 610(B11-NV08): | (G/A)₀₋₂₀LCYVALD(X)E(X0)(G/A)₀₋₂₀ |
| SEQ ID NO: 611(B11-NV09): | (G/A)₀₋₂₀LCYVALDF(X)(X0)(G/A)₀₋₂₀ |
| SEQ ID NO: 612(B11-T01): | (G/A)₀₋₂₀F(X0)TTAEREIV(G/A)₀₋₂₀ |
| SEQ ID NO: 613(B11-T02): | (G/A)₀₋₂₀(X0)TTAEREIVR(G/A)₀₋₂₀ |
| SEQ ID NO: 614(B11-T03): | (G/A)₀₋₂₀(X0)TAEREIVRD(G/A)₀₋₂₀ |
| SEQ ID NO: 615(B11-T04): | (G/A)₀₋₂₀(X0)AEREIVRDI(G/A)₀₋₂₀ |
| SEQ ID NO: 616(B11-T05): | (G/A)₀₋₂₀(X0)EREIVRDIK(G/A)₀₋₂₀ |
| SEQ ID NO: 617(B11-T06): | (G/A)₀₋₂₀(X0)REIVRDIKE(G/A)₀₋₂₀ |
| SEQ ID NO: 618(B11-T07): | (G/A)₀₋₂₀(X0)EIVRDIKEK(G/A)₀₋₂₀ |
| SEQ ID NO: 619(B11-T08): | (G/A)₀₋₂₀(X0)IVRDIKEKL(G/A)₀₋₂₀ |
| SEQ ID NO: 620(B11-T09): | (G/A)₀₋₂₀(X0)VRDIKEKLC(G/A)₀₋₂₀ |
| SEQ ID NO: 621(B11-T10): | (G/A)₀₋₂₀(X0)RDIKEKLCY(G/A)₀₋₂₀ |
| SEQ ID NO: 622(B11-T11): | (G/A)₀₋₂₀(X0)DIKEKLCYV(G/A)₀₋₂₀ |
| SEQ ID NO: 623(B11-T12): | (G/A)₀₋₂₀(X0)IKEKLCYVA(G/A)₀₋₂₀ |
| SEQ ID NO: 624(B11-T13): | (G/A)₀₋₂₀(X0)KEKLCYVAL(G/A)₀₋₂₀ |
| SEQ ID NO: 625(B11-T14): | (G/A)₀₋₂₀(X0)EKLCYVALD(G/A)₀₋₂₀ |
| SEQ ID NO: 626(B11-T15): | (G/A)₀₋₂₀(X0)KLCYVALDF(G/A)₀₋₂₀ |
| SEQ ID NO: 627(B11-T16): | (G/A)₀₋₂₀(X0)LCYVALDFE(G/A)₀₋₂₀ |
| SEQ ID NO: 628(B11-T17): | (G/A)₀₋₂₀LCYVALDFE(X0)(G/A)₀₋₂₀ |
| SEQ ID NO: 629(B11-T18): | (G/A)₀₋₂₀CYVALDFE(X0)E(G/A)₀₋₂₀ |
| SEQ ID NO: 630(B11-T19): | (G/A)₀₋₂₀YVALDFE(X0)EM(G/A)₀₋₂₀ |
| SEQ ID NO: 631(B11-T20): | (G/A)₀₋₂₀VALDFE(X0)EMA(G/A)₀₋₂₀ |
| SEQ ID NO: 632(B11-T21): | (G/A)₀₋₂₀ALDFE(X0)EMAT(G/A)₀₋₂₀ |
| SEQ ID NO: 633(B11-T22): | (G/A)₀₋₂₀LDFE(X0)EMATA(G/A)₀₋₂₀ |
| SEQ ID NO: 634(B11-T23): | (G/A)₀₋₂₀DFE(X0)EMATAA(G/A)₀₋₂₀ |
| SEQ ID NO: 635(B11-T24): | (G/A)₀₋₂₀FE(X0)EMATAAS(G/A)₀₋₂₀ |
| SEQ ID NO: 636(B11-T25): | (G/A)₀₋₂₀E(X0)EMATAASS(G/A)₀₋₂₀ |
| SEQ ID NO: 637(B11-T26): | (G/A)₀₋₂₀(X0)EMATAASSS(G/A)₀₋₂₀ |
| SEQ ID NO: 638(B11-T27): | (G/A)₀₋₂₀(X0)MATAASSSS(G/A)₀₋₂₀ |
| SEQ ID NO: 639(B11-T28): | (G/A)₀₋₂₀(X0)ATAASSSSL(G/A)₀₋₂₀ |
| SEQ ID NO: 640(B11-T29): | (G/A)₀₋₂₀(X0)TAASSSSLE(G/A)₀₋₂₀ |
| SEQ ID NO: 641(B11-T30): | (G/A)₀₋₂₀(X0)AASSSSLEK(G/A)₀₋₂₀ |
| SEQ ID NO: 642(B11-T31): | (G/A)₀₋₂₀(X0)ASSSSLEKS(G/A)₀₋₂₀ |
| SEQ ID NO: 643(B11-T32): | (G/A)₀₋₂₀(X0)SSSSLEKSY(G/A)₀₋₂₀ |
| SEQ ID NO: 644(B11-M01): | (G/A)₀₋₂₀F(X0)TTAEREIVRDIKEKLCYV(G/A)₀₋₂₀ |
| SEQ ID NO: 645(B11-M02): | (G/A)₀₋₂₀(X0)TTAEREIVRDIKEKLCYVA(G/A)₀₋₂₀ |
| SEQ ID NO: 646(B11-M03): | (G/A)₀₋₂₀(X0)TAEREIVRDIKEKLCYVAL(G/A)₀₋₂₀ |
| SEQ ID NO: 647(B11-M04): | (G/A)₀₋₂₀(X0)AEREIVRDIKEKLCYVALD(G/A)₀₋₂₀ |
| SEQ ID NO: 648(B11-M05): | (G/A)₀₋₂₀(X0)EREIVRDIKEKLCYVALDF(G/A)₀₋₂₀ |
| SEQ ID NO: 649(B11-M06): | (G/A)₀₋₂₀(X0)REIVRDIKEKLCYVALDFE(G/A)₀₋₂₀ |
| SEQ ID NO: 650(B11-M07): | (G/A)₀₋₂₀EIVRDIKEKLCYVALDFE(X0)(G/A)₀₋₂₀ |
| SEQ ID NO: 651(B11-M08): | (G/A)₀₋₂₀IVRDIKEKLCYVALDFE(X0)EM(G/A)₀₋₂₀ |
| SEQ ID NO: 652(B11-M09): | (G/A)₀₋₂₀VRDIKEKLCYVALDFE(X0)EMA(G/A)₀₋₂₀ |
| SEQ ID NO: 653(B11-M10): | (G/A)₀₋₂₀RDIKEKLCYVALDFE(X0)EMAT(G/A)₀₋₂₀ |
| SEQ ID NO: 654(B11 ): | (G/A)₀₋₂₀DIKEKLCYVALDFE(X0)EMATA(G/A)₀₋₂₀ |
| SEQ ID NO: 655(B11-P01): | (G/A)₀₋₂₀IKEKLCYVALDFE(X0)EMATAA(G/A)₀₋₂₀ |
| SEQ ID NO: 656(B11-P02): | (G/A)₀₋₂₀KEKLCYVALDFE(X0)EMATAAS(G/A)₀₋₂₀ |
| SEQ ID NO: 657(B11-P03): | (G/A)₀₋₂₀EKLCYVALDFE(X0)EMATAASS(G/A)₀₋₂₀ |
| SEQ ID NO: 658(B11-P04): | (G/A)₀₋₂₀KLCYVALDFE(X0)EMATAASSS(G/A)₀₋₂₀ |
| SEQ ID NO: 659(B11-P05): | (G/A)₀₋₂₀LCYVALDFE(X0)EMATAASSSS(G/A)₀₋₂₀ |
| SEQ ID NO: 660(B11-P06): | (G/A)₀₋₂₀CYVALDFE(X0)EMATAASSSSL(G/A)₀₋₂₀ |
| SEQ ID NO: 661(B11-P07): | (G/A)₀₋₂₀YVALDFE(X0)EMATAASSSSLE(G/A)₀₋₂₀ |
| SEQ ID NO: 662(B11-P08): | (G/A)₀₋₂₀VALDFE(X0)EMATAASSSSLEK(G/A)₀₋₂₀ |
| SEQ ID NO: 663(B11-P09): | (G/A)₀₋₂₀ALDFE(X0)EMATAASSSSLEKS(G/A)₀₋₂₀ |
| SEQ ID NO: 664(B11-P10): | (G/A)₀₋₂₀LDFE(X0)EMATAASSSSLEKSY(G/A)₀₋₂₀ |
| SEQ ID NO: 665(B11-P11): | (G/A)₀₋₂₀DFE(X0)EMATAASSSSLEKSYE(G/A)₀₋₂₀ |
| SEQ ID NO: 666(B11-V01): | (G/A)₀₋₂₀(X)IKEKLCYVALDFE(X0)EMATA(G/A)₀₋₂₀ |
| SEQ ID NO: 667(B11-V02): | (G/A)₀₋₂₀D(X)KEKLCYVALDFE(X0)EMATA(G/A)₀₋₂₀ |
| SEQ ID NO: 668(B11-V03): | (G/A)₀₋₂₀DI(X)EKLCYVALDFE(X0)EMATA(G/A)₀₋₂₀ |
| SEQ ID NO: 669(B11-V04): | (G/A)₀₋₂₀DIK(X)KLCYVALDFE(X0)EMATA(G/A)₀₋₂₀ |
| SEQ ID NO: 670(B11-V05): | (G/A)₀₋₂₀DIKE(X)LCYVALDFE(X0)EMATA(G/A)₀₋₂₀ |
| SEQ ID NO: 671(B11-V06): | (G/A)₀₋₂₀DIKEK(X)CYVALDFE(X0)EMATA(G/A)₀₋₂₀ |
| SEQ ID NO: 672(B11-V07): | (G/A)₀₋₂₀DIKEKL(X)YVALDFE(X0)EMATA(G/A)₀₋₂₀ |
| SEQ ID NO: 673(B11-V08): | (G/A)₀₋₂₀DIKEKLC(X)VALDFE(X0)EMATA(G/A)₀₋₂₀ |
| SEQ ID NO: 674(B11-V09): | (G/A)₀₋₂₀DIKEKLCY(X)ALDFE(X0)EMATA(G/A)₀₋₂₀ |
| SEQ ID NO: 675(B11-V10): | (G/A)₀₋₂₀DIKEKLCYV(X)LDFE(X0)EMATA(G/A)₀₋₂₀ |
| SEQ ID NO: 676(B11-V11): | (G/A)₀₋₂₀DIKEKLCYVA(X)DFE(X0)EMATA(G/A)₀₋₂₀ |
| SEQ ID NO: 677(B11-V12): | (G/A)₀₋₂₀DIKEKLCYVAL(X)FE(X0)EMATA(G/A)₀₋₂₀ |
| SEQ ID NO: 678(B11-V13): | (G/A)₀₋₂₀DIKEKLCYVALD(X)E(X0)EMATA(G/A)₀₋₂₀ |
| SEQ ID NO: 679(B11-V14): | (G/A)₀₋₂₀DIKEKLCYVALDF(X)(X0)EMATA(G/A)₀₋₂₀ |
| SEQ ID NO: 680(B11-V15): | (G/A)₀₋₂₀DIKEKLCYVALDFE(X0)(X)MATA(G/A)₀₋₂₀ |
| SEQ ID NO: 681(B11-V16): | (G/A)₀₋₂₀DIKEKLCYVALDFE(X0)E(X)ATA(G/A)₀₋₂₀ |
| SEQ ID NO: 682(B11-V17): | (G/A)₀₋₂₀DIKEKLCYVALDFE(X0)EM(X)TA(G/A)₀₋₂₀ |
| SEQ ID NO: 683(B11-V18): | (G/A)₀₋₂₀DIKEKLCYVALDFE(X0)EMA(X)A(G/A)₀₋₂₀ |
| SEQ ID NO: 684(B11-V19): | (G/A)₀₋₂₀DIKEKLCYVALDFE(X0)EMAT(X)(G/A)₀₋₂₀ |
| SEQ ID NO: 685(B11-X01): | (G/A)₀₋₂₀(X)(X0)TTAEREIVRDIKEKLCYVALDFE(X0)EMATAASSSSLEKSY(G/A)₀₋₂₀ |
| SEQ ID NO: 686(B11-X02): | (G/A)₀₋₂₀F(X0)(X)TAEREIVRDIKEKLCYVALDFE(X0)EMATAASSSSLEKSY(G/A)₀₋₂₀ |
| SEQ ID NO: 687(B11-X03): | (G/A)₀₋₂₀F(X0)T(X)AEREIVRDIKEKLCYVALDFE(X0)EMATAASSSSLEKSY(G/A)₀₋₂₀ |
| SEQ ID NO: 688(B11-X04): | (G/A)₀₋₂₀F(X0)TT(X)EREIVRDIKEKLCYVALDFE(X0)EMATAASSSSLEKSY(G/A)₀₋₂₀ |
| SEQ ID NO: 689(B11-X05): | (G/A)₀₋₂₀F(X0)TTA(X)REIVRDIKEKLCYVALDFE(X0)EMATAASSSSLEKSY(G/A)₀₋₂₀ |
| SEQ ID NO: 690(B11-X06): | (G/A)₀₋₂₀F(X0)TTAE(X)EIVRDIKEKLCYVALDFE(X0)EMATAASSSSLEKSY(G/A)₀₋₂₀ |
| SEQ ID NO: 691(B11-X07): | (G/A)₀₋₂₀F(X0)TTAER(X)IVRDIKEKLCYVALDFE(X0)EMATAASSSSLEKSY(G/A)₀₋₂₀ |
| SEQ ID NO: 692(B11-X08): | (G/A)₀₋₂₀F(X0)TTAERE(X)VRDIKEKLCYVALDFE(X0)EMATAASSSSLEKSY(G/A)₀₋₂₀ |
| SEQ ID NO: 693(B11-X09): | (G/A)₀₋₂₀F(X0)TTAEREI(X)RDIKEKLCYVALDFE(X0)EMATAASSSSLEKSY(G/A)₀₋₂₀ |
| SEQ ID NO: 694(B11-X10): | (G/A)₀₋₂₀F(X0)TTAEREIV(X)DIKEKLCYVALDFE(X0)EMATAASSSSLEKSY(G/A)₀₋₂₀ |
| SEQ ID NO: 695(B11-X11): | (G/A)₀₋₂₀F(X0)TTAEREIVR(X)IKEKLCYVALDFE(X0)EMATAASSSSLEKSY(G/A)₀₋₂₀ |
| SEQ ID NO: 696(B11-X12): | (G/A)₀₋₂₀F(X0)TTAEREIVRD(X)KEKLCYVALDFE(X0)EMATAASSSSLEKSY(G/A)₀₋₂₀ |
| SEQ ID NO: 697(B11-X13): | (G/A)₀₋₂₀F(X0)TTAEREIVRDI(X)EKLCYVALDFE(X0)EMATAASSSSLEKSY(G/A)₀₋₂₀ |
| SEQ ID NO: 698(B11-X14): | (G/A)₀₋₂₀F(X0)TTAEREIVRDIK(X)KLCYVALDFE(X0)EMATAASSSSLEKSY(G/A)₀₋₂₀ |
| SEQ ID NO: 699(B11-X15): | (G/A)₀₋₂₀F(X0)TTAEREIVRDIKE(X)LCYVALDFE(X0)EMATAASSSSLEKSY(G/A)₀₋₂₀ |
| SEQ ID NO: 700(B11-X16): | (G/A)₀₋₂₀F(X0)TTAEREIVRDIKEK(X)CYVALDFE(X0)EMATAASSSSLEKSY(G/A)₀₋₂₀ |
| SEQ ID NO: 701(B11-X17): | (G/A)₀₋₂₀F(X0)TTAEREIVRDIKEKL(X)YVALDFE(X0)EMATAASSSSLEKSY(G/A)₀₋₂₀ |
| SEQ ID NO: 702(B11-X18): | (G/A)₀₋₂₀F(X0)TTAEREIVRDIKEKLC(X)VALDFE(X0)EMATAASSSSLEKSY(G/A)₀₋₂₀ |
| SEQ ID NO: 703(B11-X19): | (G/A)₀₋₂₀F(X0)TTAEREIVRDIKEKLCY(X)ALDFE(X0)EMATAASSSSLEKSY(G/A)₀₋₂₀ |
| SEQ ID NO: 704(B11-X20): | (G/A)₀₋₂₀F(X0)TTAEREIVRDIKEKLCYV(X)LDFE(X0)EMATAASSSSLEKSY(G/A)₀₋₂₀ |
| SEQ ID NO: 705(B11-X21): | (G/A)₀₋₂₀F(X0)TTAEREIVRDIKEKLCYVA(X)DFE(X0)EMATAASSSSLEKSY(G/A)₀₋₂₀ |
| SEQ ID NO: 706(B11-X22): | (G/A)₀₋₂₀F(X0)TTAEREIVRDIKEKLCYVAL(X)FE(X0)EMATAASSSSLEKSY(G/A)₀₋₂₀ |
| SEQ ID NO: 707(B11-X23): | (G/A)₀₋₂₀F(X0)TTAEREIVRDIKEKLCYVALD(X)E(X0)EMATAASSSSLEKSY(G/A)₀₋₂₀ |
| SEQ ID NO: 708(B11-X24): | (G/A)₀₋₂₀F(X0)TTAEREIVRDIKEKLCYVALDF(X)(X0)EMATAASSSSLEKSY(G/A)₀₋₂₀ |
| SEQ ID NO: 709(B11-X25): | (G/A)₀₋₂₀F(X0)TTAEREIVRDIKEKLCYVALDFE(X0)(X)MATAASSSSLEKSY(G/A)₀₋₂₀ |
| SEQ ID NO: 710(B11-X26): | (G/A)₀₋₂₀F(X0)TTAEREIVRDIKEKLCYVALDFE(X0)E(X)ATAASSSSLEKSY(G/A)₀₋₂₀ |
| SEQ ID NO: 711(B11-X27): | (G/A)₀₋₂₀F(X0)TTAEREIVRDIKEKLCYVALDFE(X0)EM(X)TAASSSSLEKSY(G/A)₀₋₂₀ |
| SEQ ID NO: 712(B11-X28): | (G/A)₀₋₂₀F(X0)TTAEREIVRDIKEKLCYVALDFE(X0)EMA(X)AASSSSLEKSY(G/A)₀₋₂₀ |
| SEQ ID NO: 713(B11-X29): | (G/A)₀₋₂₀F(X0)TTAEREIVRDIKEKLCYVALDFE(X0)EMAT(X)ASSSSLEKSY(G/A)₀₋₂₀ |
| SEQ ID NO: 714(B11-X30): | (G/A)₀₋₂₀F(X0)TTAEREIVRDIKEKLCYVALDFE(X0)EMATA(X)SSSSLEKSY(G/A)₀₋₂₀ |
| SEQ ID NO: 715(B11-X31): | (G/A)₀₋₂₀F(X0)TTAEREIVRDIKEKLCYVALDFE(X0)EMATAS(X)SSSLEKSY(G/A)₀₋₂₀ |
| SEQ ID NO: 716(B11-X32): | (G/A)₀₋₂₀F(X0)TTAEREIVRDIKEKLCYVALDFE(X0)EMATASS(X)SSLEKSY(G/A)₀₋₂₀ |
| SEQ ID NO: 717(B11-X33): | (G/A)₀₋₂₀F(X0)TTAEREIVRDIKEKLCYVALDFE(X0)EMATASSS(X)SLEKSY(G/A)₀₋₂₀ |
| SEQ ID NO: 718(B11-X34): | (G/A)₀₋₂₀F(X0)TTAEREIVRDIKEKLCYVALDFE(X0)EMATASSSS(X)LEKSY(G/A)₀₋₂₀ |
| SEQ ID NO: 719(B11-X35): | (G/A)₀₋₂₀F(X0)TTAEREIVRDIKEKLCYVALDFE(X0)EMATAASSSS(X)EKSY(G/A)₀₋₂₀ |
| SEQ ID NO: 720(B11-X36): | (G/A)₀₋₂₀F(X0)TTAEREIVRDIKEKLCYVALDFE(X0)EMATAASSSSL(X)KSY(G/A)₀₋₂₀ |
| SEQ ID NO: 721(B11-X37): | (G/A)₀₋₂₀F(X0)TTAEREIVRDIKEKLCYVALDFE(X0)EMATAASSSSLE(X)SY(G/A)₀₋₂₀ |
| SEQ ID NO: 722(B11-X38): | (G/A)₀₋₂₀F(X0)TTAEREIVRDIKEKLCYVALDFE(X0)EMATAASSSSLEK(X)Y(G/A)₀₋₂₀ |
| SEQ ID NO: 723(B11-X39): | (G/A)₀₋₂₀F(X0)TTAEREIVRDIKEKLCYVALDFE(X0)EMATAASSSSLEKS(X)(G/A)₀₋₂₀ |
| SEQ ID NO: 724(B17-core): | (X)₀₋₂₀KYSVWIGGSI(X)₀₋₂₀ |
| SEQ ID NO: 725(B17-TV01): | (G/A)₀₋₂₀(X)YSVWIGGSI(G/A)₀₋₂₀ |
| SEQ ID NO: 726(B17-TV02): | (G/A)₀₋₂₀K(X)SVWIGGSI(G/A)₀₋₂₀ |
| EQ ID NO: 727(B17-TV03): | (G/A)₀₋₂₀KY(X)VWIGGSI(G/A)₀₋₂₀ |
| SEQ ID NO: 728(B17-TV04): | (G/A)₀₋₂₀KYS(X)WIGGSI(G/A)₀₋₂₀ |
| SEQ ID NO: 729(B17-TV05): | (G/A)₀₋₂₀KYSV(X)IGGSI(G/A)₀₋₂₀ |
| SEQ ID NO: 730(B17-TV06): | (G/A)₀₋₂₀KYSVW(X)GGSI(G/A)₀₋₂₀ |
| SEQ ID NO: 731(B17-TV07): | (G/A)₀₋₂₀KYSVWI(X)GSI(G/A)₀₋₂₀ |
| SEQ ID NO: 732(B17-TV08): | (G/A)₀₋₂₀KYSVWIG(X)SI(G/A)₀₋₂₀ |
| SEQ ID NO: 733(B17-TV09): | (G/A)₀₋₂₀KYSVWIGG(X)I(G/A)₀₋₂₀ |
| SEQ ID NO: 734(B17-TV10): | (G/A)₀₋₂₀KYSVWIGGS(X)(G/A)₀₋₂₀ |
| SEQ ID NO: 735(B17-T01): | (G/A)₀₋₂₀(X0)PSTMKIKII(G/A)₀₋₂₀ |
| SEQ ID NO: 736(B17-T02): | (G/A)₀₋₂₀(X0)STMKIKIIA(G/A)₀₋₂₀ |
| SEQ ID NO: 737(B17-T03): | (G/A)₀₋₂₀(X0)TMKIKIIAP(G/A)₀₋₂₀ |
| SEQ ID NO: 738(B17-T04): | (G/A)₀₋₂₀(X0)MKIKIIAPP(G/A)₀₋₂₀ |
| SEQ ID NO: 739(B17-T05): | (G/A)₀₋₂₀(X0)KIKIIAPPE(G/A)₀₋₂₀ |
| SEQ ID NO: 740(B17-T06): | (G/A)₀₋₂₀(X0)IKIIAPPER(G/A)₀₋₂₀ |
| SEQ ID NO: 741(B17-T07): | (G/A)₀₋₂₀(X0)KIIAPPERK(G/A)₀₋₂₀ |
| SEQ ID NO: 742(B17-T08): | (G/A)₀₋₂₀(X0)IIAPPERKY(G/A)₀₋₂₀ |
| SEQ ID NO: 743(B17-T09): | (G/A)₀₋₂₀(X0)IAPPERKYS(G/A)₀₋₂₀ |
| SEQ ID NO: 744(B17-T10): | (G/A)₀₋₂₀(X0)APPERKYSV(G/A)₀₋₂₀ |
| SEQ ID NO: 745(B17-T11): | (G/A)₀₋₂₀(X0)PPERKYSVW(G/A)₀₋₂₀ |
| SEQ ID NO: 746(B17-T12): | (G/A)₀₋₂₀(X0)PERKYSVWI(G/A)₀₋₂₀ |
| SEQ ID NO: 747(B17-T13): | (G/A)₀₋₂₀(X0)ERKYSVWIG(G/A)₀₋₂₀ |
| SEQ ID NO: 748(B17-T14): | (G/A)₀₋₂₀(X0)RKYSVWIGG(G/A)₀₋₂₀ |
| SEQ ID NO: 749(B17-T15): | (G/A)₀₋₂₀(X0)KYSVWIGGS(G/A)₀₋₂₀ |
| SEQ ID NO: 750(B17-T16): | (G/A)₀₋₂₀(X0)YSVWIGGSI(G/A)₀₋₂₀ |
| SEQ ID NO: 751(B17-T17): | (G/A)₀₋₂₀(X0)SVWIGGSIL(G/A)₀₋₂₀ |
| SEQ ID NO: 752(B17-T18): | (G/A)₀₋₂₀(X0)VWIGGSILA(G/A)₀₋₂₀ |
| SEQ ID NO: 753(B17-T19): | (G/A)₀₋₂₀(X0)WIGGSILAS(G/A)₀₋₂₀ |
| SEQ ID NO: 754(B17-T20): | (G/A)₀₋₂₀(X0)IGGSILASL(G/A)₀₋₂₀ |
| SEQ ID NO: 755(B17-T21): | (G/A)₀₋₂₀(X0)GGSILASLS(G/A)₀₋₂₀ |
| SEQ ID NO: 756(B17-T22): | (G/A)₀₋₂₀(X0)GSILASLST(G/A)₀₋₂₀ |
| SEQ ID NO: 757(B17-T23): | (G/A)₀₋₂₀(X0)SILASLSTF(G/A)₀₋₂₀ |
| SEQ ID NO: 758(B17-T24): | (G/A)₀₋₂₀(X0)ILASLSTFQ(G/A)₀₋₂₀ |
| SEQ ID NO: 759(B17-T25): | (G/A)₀₋₂₀(X0)LASLSTFQQ(G/A)₀₋₂₀ |
| SEQ ID NO: 760(B17-T26): | (G/A)₀₋₂₀(X0)ASLSTFQQM(G/A)₀₋₂₀ |
| SEQ ID NO: 761(B17-T27): | (G/A)₀₋₂₀(X0)SLSTFQQMW(G/A)₀₋₂₀ |
| SEQ ID NO: 762(B17-T28): | (G/A)₀₋₂₀(X0)LSTFQQMWI(G/A)₀₋₂₀ |
| SEQ ID NO: 763(B17-T29): | (G/A)₀₋₂₀LSTFQQMWI(X0)(G/A)₀₋₂₀ |
| SEQ ID NO: 764(B17-T30): | (G/A)₀₋₂₀STFQQMWI(X0)K(G/A)₀₋₂₀ |
| SEQ ID NO: 765(B17-T31): | (G/A)₀₋₂₀TFQQMWI(X0)KQ(G/A)₀₋₂₀ |
| SEQ ID NO: 766(B17-M01): | (G/A)₀₋₂₀APSTMKIKIIAPPERKYSVW(G/A)₀₋₂₀ |
| SEQ ID NO: 767(B17-M02): | (G/A)₀₋₂₀PSTMKIKIIAPPERKYSVWI(G/A)₀₋₂₀ |
| SEQ ID NO: 768(B17-M03): | (G/A)₀₋₂₀STMKIKIIAPPERKYSVWIG(G/A)₀₋₂₀ |
| SEQ ID NO: 769(B17-M04): | (G/A)₀₋₂₀TMKIKIIAPPERKYSVWIGG(G/A)₀₋₂₀ |
| SEQ ID NO: 770(B17-M05): | (G/A)₀₋₂₀MKIKIIAPPERKYSVWIGGS(G/A)₀₋₂₀ |
| SEQ ID NO: 771(B17-M06): | (G/A)₀₋₂₀KIKIIAPPERKYSVWIGGSI(G/A)₀₋₂₀ |
| SEQ ID NO: 772(B17-M07): | (G/A)₀₋₂₀IKIIAPPERKYSVWIGGSIL(G/A)₀₋₂₀ |
| SEQ ID NO: 773(B17-M08): | (G/A)₀₋₂₀KIIAPPERKYSVWIGGSILA(G/A)₀₋₂₀ |
| SEQ ID NO: 774(B17-M09): | (G/A)₀₋₂₀IIAPPERKYSVWIGGSILAS(G/A)₀₋₂₀ |
| SEQ ID NO: 775(B17-M10): | (G/A)₀₋₂₀IAPPERKYSVWIGGSILASL(G/A)₀₋₂₀ |
| SEQ ID NO: 776(B17 ): | (G/A)₀₋₂₀APPERKYSVWIGGSILASLS(G/A)₀₋₂₀ |
| SEQ ID NO: 777(B17-P01): | (G/A)₀₋₂₀PPERKYSVWIGGSILASLST(G/A)₀₋₂₀ |
| SEQ ID NO: 778(B17-P02): | (G/A)₀₋₂₀PERKYSVWIGGSILASLSTF(G/A)₀₋₂₀ |
| SEQ ID NO: 779(B17-P03): | (G/A)₀₋₂₀ERKYSVWIGGSILASLSTFQ(G/A)₀₋₂₀ |
| SEQ ID NO: 780(B17-P04): | (G/A)₀₋₂₀RKYSVWIGGSILASLSTFQQ(G/A)₀₋₂₀ |
| SEQ ID NO: 781(B17-P05): | (G/A)₀₋₂₀KYSVWIGGSILASLSTFQQM(G/A)₀₋₂₀ |
| SEQ ID NO: 782(B17-P06): | (G/A)₀₋₂₀YSVWIGGSILASLSTFQQMW(G/A)₀₋₂₀ |
| SEQ ID NO: 783(B17-P07): | (G/A)₀₋₂₀SVWIGGSILASLSTFQQMWI(G/A)₀₋₂₀ |
| SEQ ID NO: 784(B17-P08): | (G/A)₀₋₂₀VWIGGSILASLSTFQQMWI(X0)(G/A)₀₋₂₀ |
| SEQ ID NO: 785(B17-P09): | (G/A)₀₋₂₀WIGGSILASLSTFQQMWI(X0)K(G/A)₀₋₂₀ |
| SEQ ID NO: 786(B17-P10): | (G/A)₀₋₂₀IGGSILASLSTFQQMWI(X0)KQ(G/A)₀₋₂₀ |
| SEQ ID NO: 787(B17-V01): | (G/A)₀₋₂₀(X)PPERKYSVWIGGSILASLS(G/A)₀₋₂₀ |
| SEQ ID NO: 788(B17-V02): | (G/A)₀₋₂₀A(X)PERKYSVWIGGSILASLS(G/A)₀₋₂₀ |
| SEQ ID NO: 789(B17-V03): | (G/A)₀₋₂₀AP(X)ERKYSVWIGGSILASLS(G/A)₀₋₂₀ |
| SEQ ID NO: 790(B17-V04): | (G/A)₀₋₂₀APP(X)RKYSVWIGGSILASLS(G/A)₀₋₂₀ |
| SEQ ID NO: 791(B17-V05): | (G/A)₀₋₂₀APPE(X)KYSVWIGGSILASLS(G/A)₀₋₂₀ |
| SEQ ID NO: 792(B17-V06): | (G/A)₀₋₂₀APPER(X)YSVWIGGSILASLS(G/A)₀₋₂₀ |
| SEQ ID NO: 793(B17-V07): | (G/A)₀₋₂₀APPERK(X)SVWIGGSILASLS(G/A)₀₋₂₀ |
| SEQ ID NO: 794(B17-V08): | (G/A)₀₋₂₀APPERKY(X)VWIGGSILASLS(G/A)₀₋₂₀ |
| SEQ ID NO: 795(B17-V09): | (G/A)₀₋₂₀APPERKYS(X)WIGGSILASLS(G/A)₀₋₂₀ |
| SEQ ID NO: 796(B17-V10): | (G/A)₀₋₂₀APPERKYSV(X)IGGSILASLS(G/A)₀₋₂₀ |
| SEQ ID NO: 797(B17-V11): | (G/A)₀₋₂₀APPERKYSVW(X)GGSILASLS(G/A)₀₋₂₀ |
| SEQ ID NO: 798(B17-V12): | (G/A)₀₋₂₀APPERKYSVWI(X)GSILASLS(G/A)₀₋₂₀ |
| SEQ ID NO: 799(B17-V13): | (G/A)₀₋₂₀APPERKYSVWIG(X)SILASLS(G/A)₀₋₂₀ |
| SEQ ID NO: 800(B17-V14): | (G/A)₀₋₂₀APPERKYSVWIGG(X)ILASLS(G/A)₀₋₂₀ |
| SEQ ID NO: 801(B17-V15): | (G/A)₀₋₂₀APPERKYSVWIGGS(X)LASLS(G/A)₀₋₂₀ |
| SEQ ID NO: 802(B17-V16): | (G/A)₀₋₂₀APPERKYSVWIGGSI(X)ASLS(G/A)₀₋₂₀ |
| SEQ ID NO: 803(B17-V17): | (G/A)₀₋₂₀APPERKYSVWIGGSIL(X)SLS(G/A)₀₋₂₀ |
| SEQ ID NO: 804(B17-V18): | (G/A)₀₋₂₀APPERKYSVWIGGSILA(X)LS(G/A)₀₋₂₀ |
| SEQ ID NO: 805(B17-V19): | (G/A)₀₋₂₀APPERKYSVWIGGSILAS(X)S(G/A)₀₋₂₀ |
| SEQ ID NO: 806(B17-V20): | (G/A)₀₋₂₀APPERKYSVWIGGSILASL(X)(G/A)₀₋₂₀ |
| SEQ ID NO: 807(B17-X01): | (G/A)₀₋₂₀(X)PSTMKIKIIAPPERKYSVWIGGSILASLSTFQQMWI(X0)KQ(G/A)₀₋₂₀ |
| SEQ ID NO: 808(B17-X02): | (G/A)₀₋₂₀A(X)STMKIKIIAPPERKYSVWIGGSILASLSTFQQMWI(X0)KQ(G/A)₀₋₂₀ |
| SEQ ID NO: 809(B17-X03): | (G/A)₀₋₂₀AP(X)TMKIKIIAPPERKYSVWIGGSILASLSTFQQMWI(X0)KQ(G/A)₀₋₂₀ |
| SEQ ID NO: 810(B17-X04): | (G/A)₀₋₂₀APS(X)MKIKIIAPPERKYSVWIGGSILASLSTFQQMWI(X0)KQ(G/A)₀₋₂₀ |
| SEQ ID NO: 811(B17-X05): | (G/A)₀₋₂₀APST(X)KIKIIAPPERKYSVWIGGSILASLSTFQQMWI(X0)KQ(G/A)₀₋₂₀ |
| SEQ ID NO: 812(B17-X06): | (G/A)₀₋₂₀APSTM(X)IKIIAPPERKYSVWIGGSILASLSTFQQMWI(X0)KQ(G/A)₀₋₂₀ |
| SEQ ID NO: 813(B17-X07): | (G/A)₀₋₂₀APSTMK(X)KIIAPPERKYSVWIGGSILASLSTFQQMWI(X0)KQ(G/A)₀₋₂₀ |
| SEQ ID NO: 814(B17-X08): | (G/A)₀₋₂₀APSTMKI(X)IIAPPERKYSVWIGGSILASLSTFQQMWI(X0)KQ(G/A)₀₋₂₀ |
| SEQ ID NO: 815(B17-X09): | (G/A)₀₋₂₀APSTMKIK(X)IAPPERKYSVWIGGSILASLSTFQQMWI(X0)KQ(G/A)₀₋₂₀ |
| SEQ ID NO: 816(B17-X10): | (G/A)₀₋₂₀APSTMKIKI(X)APPERKYSVWIGGSILASLSTFQQMWI(X0)KQ(G/A)₀₋₂₀ |
| SEQ ID NO: 817(B17-X11): | (G/A)₀₋₂₀APSTMKIKII(X)PPERKYSVWIGGSILASLSTFQQMWI(X0)KQ(G/A)₀₋₂₀ |
| SEQ ID NO: 818(B17-X12): | (G/A)₀₋₂₀APSTMKIKIIA(X)PERKYSVWIGGSILASLSTFQQMWI(X0)KQ(G/A)₀₋₂₀ |
| SEQ ID NO: 819(B17-X13): | (G/A)₀₋₂₀APSTMKIKIIAP(X)ERKYSVWIGGSILASLSTFQQMWI(X0)KQ(G/A)₀₋₂₀ |
| SEQ ID NO: 820(B17-X15): | (G/A)₀₋₂₀APSTMKIKIIAPP(X)RKYSVWIGGSILASLSTFQQMWI(X0)KQ(G/A)₀₋₂₀ |
| SEQ ID NO: 821(B17-X16): | (G/A)₀₋₂₀APSTMKIKIIAPPE(X)KYSVWIGGSILASLSTFQQMWI(X0)KQ(G/A)₀₋₂₀ |
| SEQ ID NO: 822(B17-X17): | (G/A)₀₋₂₀APSTMKIKIIAPPER(X)YSVWIGGSILASLSTFQQMWI(X0)KQ(G/A)₀₋₂₀ |
| SEQ ID NO: 823(B17-X18): | (G/A)₀₋₂₀APSTMKIKIIAPPERK(X)SVWIGGSILASLSTFQQMWI(X0)KQ(G/A)₀₋₂₀ |
| SEQ ID NO: 824(B17-X19): | (G/A)₀₋₂₀APSTMKIKIIAPPERKY(X)VWIGGSILASLSTFQQMWI(X0)KQ(G/A)₀₋₂₀ |
| SEQ ID NO: 825(B17-X20): | (G/A)₀₋₂₀APSTMKIKIIAPPERKYS(X)WIGGSILASLSTFQQMWI(X0)KQ(G/A)₀₋₂₀ |
| SEQ ID NO: 826(B17-X21): | (G/A)₀₋₂₀APSTMKIKIIAPPERKYSV(X)IGGSILASLSTFQQMWI(X0)KQ(G/A)₀₋₂₀ |
| SEQ ID NO: 827(B17-X22): | (G/A)₀₋₂₀APSTMKIKIIAPPERKYSVW(X)GGSILASLSTFQQMWI(X0)KQ(G/A)₀₋₂₀ |
| SEQ ID NO: 828(B17-X23): | (G/A)₀₋₂₀APSTMKIKIIAPPERKYSVWI(X)GSILASLSTFQQMWI(X0)KQ(G/A)₀₋₂₀ |
| SEQ ID NO: 829(B17-X24): | (G/A)₀₋₂₀APSTMKIKIIAPPERKYSVWIG(X)SILASLSTFQQMWI(X0)KQ(G/A)₀₋₂₀ |
| SEQ ID NO: 830(B17-X25): | (G/A)₀₋₂₀APSTMKIKIIAPPERKYSVWIGG(X)ILASLSTFQQMWI(X0)KQ(G/A)₀₋₂₀ |
| SEQ ID NO: 831(B17-X26): | (G/A)₀₋₂₀APSTMKIKIIAPPERKYSVWIGGS(X)LASLSTFQQMWI(X0)KQ(G/A)₀₋₂₀ |
| SEQ ID NO: 832(B17-X27): | (G/A)₀₋₂₀APSTMKIKIIAPPERKYSVWIGGSI(X)ASLSTFQQMWI(X0)KQ(G/A)₀₋₂₀ |
| SEQ ID NO: 833(B17-X28): | (G/A)₀₋₂₀APSTMKIKIIAPPERKYSVWIGGSIL(X)SLSTFQQMWI(X0)KQ(G/A)₀₋₂₀ |
| SEQ ID NO: 834(B17-X29): | (G/A)₀₋₂₀APSTMKIKIIAPPERKYSVWIGGSILA(X)LSTFQQMWI(X0)KQ(G/A)₀₋₂₀ |
| SEQ ID NO: 835(B17-X30): | (G/A)₀₋₂₀APSTMKIKIIAPPERKYSVWIGGSILAS(X)STFQQMWI(X0)KQ(G/A)₀₋₂₀ |
| SEQ ID NO: 836(B17-X31): | (G/A)₀₋₂₀APSTMKIKIIAPPERKYSVWIGGSILASL(X)TFQQMWI(X0)KQ(G/A)₀₋₂₀ |
| SEQ ID NO: 837(B17-X32): | (G/A)₀₋₂₀APSTMKIKIIAPPERKYSVWIGGSILASLS(X)FQQMWI(X0)KQ(G/A)₀₋₂₀ |
| SEQ ID NO: 838(B17-X33): | (G/A)₀₋₂₀APSTMKIKIIAPPERKYSVWIGGSILASLST(X)QQMWI(X0)KQ(G/A)₀₋₂₀ |
| SEQ ID NO: 839(B17-X34): | (G/A)₀₋₂₀APSTMKIKIIAPPERKYSVWIGGSILASLSTF(X)QMWI(X0)KQ(G/A)₀₋₂₀ |
| SEQ ID NO: 840(B17-X35): | (G/A)₀₋₂₀APSTMKIKIIAPPERKYSVWIGGSILASLSTFQ(X)MWI(X0)KQ(G/A)₀₋₂₀ |
| SEQ ID NO: 841(B17-X36): | (G/A)₀₋₂₀APSTMKIKIIAPPERKYSVWIGGSILASLSTFQQ(X)WI(X0)KQ(G/A)₀₋₂₀ |
| SEQ ID NO: 842(B17-X37): | (G/A)₀₋₂₀APSTMKIKIIAPPERKYSVWIGGSILASLSTFQQM(X)I(X0)KQ(G/A)₀₋₂₀ |
| SEQ ID NO: 843(B17-X38): | (G/A)₀₋₂₀APSTMKIKIIAPPERKYSVWIGGSILASLSTFQQMW(X)(X0)KQ(G/A)₀₋₂₀ |
| SEQ ID NO: 844(B17-X39): | (G/A)₀₋₂₀APSTMKIKIIAPPERKYSVWIGGSILASLSTFQQMWI(X0)(X)Q(G/A)₀₋₂₀ |
| SEQ ID NO: 845(B17-X40): | (G/A)₀₋₂₀APSTMKIKIIAPPERKYSVWIGGSILASLSTFQQMWI(X0)K(X)(G/A)₀₋₂₀ |

In another embodiment, a peptide according to formula SEQ ID No:1 to SEQ ID No:845is a cyclic peptide. A cyclic peptide can be made by covalently crosslinking amino acid residues together via a disulfide bond (e.g., thiol group of cysteine) or a side chain of amino acid residues (e.g., Lye, Arg, Ser, Tyr). Furthermore, a functionalized linker (e.g., polyethylene glycol or PEG) can be introduced to generate a cyclic peptide.

Although the sequences enumerated here comprise 40 AA, 20 AA, and 10 AA, it is to be understood that peptides of the invention may include any length from 5 to 40 AA.

Numerous permutations and variations of the present invention are readily apparent to a person of ordinary skill in the art in view of this disclosure. Therefore it is to be understood that the invention may be practiced otherwise than as specifically described herein.

TABLE 1
Actin alignment
As one can see below from the actin protein sequences of human,
mouse, rabbit, and fly, the actin sequence is highly conserved.

| | |
|---|---|
| SEQ ID NO: 846: | Human ACTA1: Human ACTA1 (alpha skeletal muscle actin) |
| SEQ ID NO: 847: | Human ACTAC1: Human ACTC1 (alpha cardiac muscle actin) |
| SEQ ID NO: 848: | Human ACTAB: Human ACTB (cytoplasmic 1 actin) |
| SEQ ID NO: 849: | Human ACTABG1: Human ACTG1 (cytoplasmic 2 actin) |
| SEQ ID NO: 850: | Mouse ACTB: Mus musculus Actin, beta |
| SEQ ID NO: 851: | Rabbit ACTB: Orycytolagus cuniculus ACTB (cytoplasmic 1 actin |
| SEQ ID NO: 852: | Fly ACTB: Drosophila actin Actin5C, (Isoform B) |
| | 10 20 30 40 50 |
| SEQ ID NO: 846: | MCDEDETTALVC DNGSGLVKAG FAGDDAPRAV FPSIVGRPRH QGVMVGMGQK |
| SEQ ID NO: 847: | MCDDEETTALVC DNGSGLVKAG FAGDDAPRAV FPSIVGRPRH QGVMVGMGQK |
| SEQ ID NO: 848: | MDDDIAALVV DNGSGMCKAG FAGDDAPRAV FPSIVGRPRH QGVMVGMGQK |
| SEQ ID NO: 849: | MEEEIAALVI DNGSGMCKAG FAGDDAPRAV FPSIVGRPRH QGVMVGMGQK |
| SEQ ID NO: 850: | MDDDIAALVV DNGSGMCKAG FAGDDAPRAV FPSIVGRPRH QGVMVGMGQK |
| SEQ ID NO: 851: | MDDDIAALVV DNGSGMCKAG FAGDDAPRAV FPSIVGRPRH QGVMVGMGQK |
| SEQ ID NO: 852: | MCDEEVAALVV DNGSGMCKAG FAGDDAPRAV FPSIVGRPRH QGVMVGMGQK |
| | 60 70 80 90 100 |
| SEQ ID NO: 846: | DSYVGDEAQS KRGILTLKYP IEHGIITNWD DMEKIWHHTF YNELRVAPEE |
| SEQ ID NO: 847: | DSYVGDEAQS KRGILTLKYP IEHGIITNWD DMEKIWHHTF YNELRVAPEE |
| SEQ ID NO: 848: | DSYVGDEAQS KRGILTLKYP IEHGIVTNWD DMEKIWHHTF YNELRVAPEE |
| SEQ ID NO: 849: | DSYVGDEAQS KRGILTLKYP IEHGIVTNWD DMEKIWHHTF YNELRVAPEE |
| SEQ ID NO: 850: | DSYVGDEAQS KRGILTLKYP IEHGIVTNWD DMEKIWHHTF YNELRVAPEE |
| SEQ ID NO: 851: | DSYVGDEAQS KRGILTLKYP IEHGIVTNWD DMEKIWHHTF YNELRVAPEE |
| SEQ IE NO: 852: | DSYVGDEAQS KRGILTLKYP IEHGIVTNWD DMEKIWHHTF YNELRVAPEE |
| | 110 120 130 140 150 |
| SEQ ID NO: 846: | HPTLLTEAPL NPKANREKMT QIMFETFNVP AMYVAIQAVL SLYASGRTTG |
| SEQ ID NO: 847: | HPTLLTEAPL NPKANREKMT QIMFETFNVP AMYVAIQAVL SLYASGRTTG |
| SEQ ID NO: 848: | HPVLLTEAPL NPKANREKMT QIMFETFNIP AMYVAIQAVL SLYASGRTTG |
| SEQ ID NO: 849: | HPVLLTEAPL NPKANREKMT QIMFETFNIP AMYVAIQAVL SLYASGRTTG |
| SEQ ID NO: 850: | HPVLLTEAPL NPKANREKMT QIMFETFNIP AMYVAIQAVL SLYASGRTTG |
| SEQ ID NO: 851: | RPVLLTEAPL NPKANREKMT QIMFETFNIP AMYVAIQAVL SLYASGRTTG |
| SEQ ID NO: 852: | HPVLLTEAPL NPKANREKMT QIMFETFNIP AMYVAIQAVL SLYASGRTTG |
| | 160 170 180 190 200 |
| SEQ ID NO: 846: | IVLDSGDGVT HNVPIYEGYA LPHAIMRLDL AGRDLTDYLM KILTERGYSF |
| SEQ ID NO: 847: | IVLDSGDGVT HNVPIYEGYA LPHAIMRLDL AGRDLTDYLM KILTERGYSF |
| SEQ ID NO: 848: | IVMDSGDGVT HTVPIYEGYA LPHAILRLDL AGRDLTDYLM KILTERGYSF |
| SEQ ID NO: 849: | IVMDSGDGVT HTVPIYEGYA LPHAILRLDL AGRDLTDYLM KILTERGYSF |
| SEQ ID NO: 850: | IVMDSGDGVT HTVPIYEGYA LPHAILRLDL AGRDLTDYLM KILTERGYSF |
| SEQ ID NO: 851: | IVMDSGDGVT HTVPIYEGYA LPHAILRLDL AGRDLTDYLM KILTERGYSF |
| SEQ ID NO: 852: | IVLDSGDGVS HTVPIYEGYA LPHAILRLDL AGRDLTDYLM KILTERGYSF |
| | 210 220 230 240 250 |
| SEQ ID NO: 846: | VTTAEREIVR DIKEKLCYVA LDFENEMATA ASSSSLEKSY ELPDGQVITI |
| SEQ ID NO: 847: | VTTAEREIVR DIKEKLCYVA LDFENEMATA ASSSSLEKSY ELPDGQVITI |
| SEQ ID NO: 848: | ITTAEREIVR DIKEKLCYVA LDFEQEMATA ASSSSLEKSY ELPDGQVITI |
| SEQ ID NO: 849: | ITTAEREIVR DIKEKLCYVA LDFEQEMATA ASSSSLEKSY ELPDGQVITI |
| SEQ ID NO: 850: | ITTAEREIVR DIKEKLCYVA LDFEQEMATA ASSSSLEKSY ELPDGQVITI |
| SEQ ID NO: 851: | ITTAEREIVR DIKEKLCYVA LDFEQEMATA ASSSSLEKSY ELPDGQVITI |
| SEQ ID NO: 852: | ITTAEREIVR DIKEKLCYVA LDFEQEMATA ASSSSLEKSY ELPDGQVITI |
| | 260 270 280 290 300 |
| SEQ ID NO: 846: | GNERFRCPET LFQPSFIGME SAGIHETTYN SIMKCDIDIR KDLYANNVMS |
| SEQ ID NO: 847: | GNERFRCPET LFQPSFIGME SAGIHETTYN SIMKCDIDIR KDLYANNVLS |
| SEQ ID NO: 848: | GNERFRCPEA LFQPSFLGME SCGIHETTFN SIMKCDVDIR KDLYANTVLS |
| SEQ ID NO: 849: | GNERFRCPEA LFQPSFLGME SCGIHETTFN SIMKCDVDIR KDLYANTVLS |
| SEQ ID NO: 850: | GNERFRCPEA LFQPSFLGME SCGIHETTFN SIMKCDVDIR KDLYANTVLS |
| SEQ ID NO: 851: | GNERFRCPEA LFQPSFLGME SCGIHETTFN SIMKCDVDIR KDLYANTVLS |
| SEQ ID NO: 852: | GNERFRCPEA LFQPSFLGME ACGIHETTYN SIMKCDVDIR KDLYANTVLS |
| | 310 320 330 340 350 |
| SEQ ID NO: 846: | GGITMYPGIA DRMQKEITAL APSTMKIKII APPERKYSVW IGGSILASLS |
| SEQ ID NO: 847: | GGITMYPGIA DRMQKEITAL APSTMKIKII APPERKYSVW IGGSILASLS |
| SEQ ID NO: 848: | GGITMYPGIA DRMQKEITAL APSTMKIKII APPERKYSVW IGGSILASLS |
| SEQ ID NO: 849: | GGITMYPGIA DRMQKEITAL APSTMKIKII APPERKYSVW IGGSILASLS |
| SEQ ID NO: 850: | GGITMYPGIA DRMQKEITAL APSTMKIKII APPERKYSVW IGGSILASLS |
| SEQ ID NO: 851: | GGITMYPGIA DRMQKEITAL APSTMKIKII APPERKYSVW IGGSILASLS |
| SEQ ID NO: 852: | GGITMYPGIA DRMQKEITAL APSTMKIKII APPERKYSVW IGGSILASLS |
| | 360 370 |
| SEQ ID NO: 846: | TFQQMWITKQ EYDEAGPSIV HRKCF |
| SEQ ID NO: 847: | TFQQMWISKQ EYDEAGPSIV HRKCF |
| SEQ ID NO: 848: | TFQQMWISKQ EYDESGPSIV HRKCF |

1-21. (canceled)

22. A method for controlling viral or bacterial infection, comprising administering an actin-based peptide to a cell, virus, or bacteria, in an amount sufficient to inhibit or enhance infection of said virus or bacteria, wherein the actin-based peptide comprises:
an actin core domain and a cell penetration domain, operably linked together by a a spacer domain,
wherein an N-terminus or C-terminus may have either the actin core domain or the cell penetration domain, and
wherein the actin core domain is a N7 core domain having the sequence IMFETFN, which are the amino acids 2-8 of SEQ ID NO:854.

23. The method of claim 22, wherein the actin core domain functions to control viral or bacterial infection of a cell by inhibiting or enhancing viral or bacterial infection or bacterial vector delivery into the cell.

24. The method of claim 22, wherein the actin core domain is labeled with a traceable dye or marker.

25. The method of claim 22, comprising a N7 core selected from the sequences set forth in SEQ ID NO: 2, 8-9, 139, 147-148, 172-175, 194-207, 212-213, 220-242, 250-282, and 290.

26. A method of claim 22 for controlling viral infection, comprising administering the actin-based peptide to a cell, virus, or bacteria, in an amount sufficient to inhibit or enhance infection of said virus.

27. The method of claim 26, wherein HIV infection is increased by administering said actin-based peptide to a cell.

28. The method of claim 27, wherein said actin-based peptide comprises an actin core domain selected from N7 (SEQ ID NO: 2, 8, 9, or 139-290).

29. A method for modulating actin dynamics, comprising delivering into a cell one or more actin-based peptides in an amount effective to modulate actin dynamics, wherein the actin-based peptide comprises:
an actin core domain and a cell penetration domain, operably linked together by a a spacer domain,
wherein an N-terminus or C-terminus may have either the actin core domain or the cell penetration domain, and
wherein the actin core domain is a N7 core domain having the sequence IMFETFN, which are the amino acids 2-8 of SEQ ID NO:854.

30. A method for modulating cellular bioactivity, comprising delivering into a cell one or more actin-based peptides in an amount effective to modulate cellular bioactivity, wherein the actin-based peptide comprises:
an actin core domain and a cell penetration domain, operably linked together by a a spacer domain,
wherein an N-terminus or C-terminus may have either the actin core domain or the cell penetration domain, and
wherein the actin core domain is a N7 core domain having the sequence IMFETFN, which are the amino acids 2-8 of SEQ ID NO:854.

31. The method of claim 30, wherein said cellular bioactivity is: cytoplasmic and nuclear actin dynamics, cell morphology, cell motility and mobility, cell surface receptor density, cell adhesion processes, gene expression, cell division, growth, differentiation, and apoptosis, the processes of endocytosis and exocytosis, smooth and heart muscle cell contraction and relaxation, blood vessel elasticity and capacitance, a process of inflammation, cellular susceptibility to intracellular pathogens, or cellular uptake of chemical compounds, including small and macro molecules, medical drugs, nucleic acids, and proteins.

32. The method of claim 22, wherein said cell penetration domain is a cell penetration peptide.

33. The method of claim 22, wherein said actin core domain modulates a cellular bioactivity selected from the group consisting of cytoplasmic and nuclear actin dynamics; cell morphology; cell motility and mobility; cell surface receptor density; cell adhesion processes;
gene expression; cell division, growth, differentiation, and apoptosis; the processes of endocytosis and exocytosis; smooth and heart muscle cell contraction and relaxation, blood vessel elasticity and capacitance; a process of inflammation; cellular susceptibility to intracellular pathogens, or cellular uptake of chemical compounds, including small and macro molecules, medical drugs, nucleic acids, and proteins

34. The method of claim 33, wherein said actin-based peptide is labeled with a traceable dye or marker.

35. A method of claim 22, comprising an effective amount of a therapeutic compound, a diagnostic compound, or a compound for modulating cellular activity.

36. The method of claim 22, wherein the actin-based peptide comprises the amino acid sequence of SEQ ID NOs: 2, 8-9, 139, or 147-148.

37. The method of claim 22, wherein the actin-based peptide is selected from the group consisting of SEQ ID NOs: 172-175, 194-207, and 212-213.

38. The method of claim 22, wherein the actin-based peptide comprises the amino acid of SEQ ID NOs: 220-242, 250-282, or 290.

39. An method of claim 22, wherein said actin core domain modulates cell adhesion processes.

40. The method of claim 22, wherein the actin core domain is a N7 core domain having the sequence QIMPETFN, which are the amino acids 1-8 of SEQ ID NO: 854.

41. The method of claim 22, wherein the actin core domain is a N7 core domain having the sequence QIMFETFNT, which are the amino acids 1-9 of SEQ ID NO: 854.
